(19) United States
(12) Patent Application Publication
(10) Pub. No.: US 2026/0117225 A1
(43) Pub. Date: Apr. 30, 2026

(54) COMPOSITIONS FOR TREATING MUSCULAR DYSTROPHY

(71) Applicant: Sarepta Therapeutics, Inc., Cambridge, MA (US)

(72) Inventor: Edward M. KAYE, Cambridge, MA (US)

(73) Assignee: Sarepta Therapeutics, Inc., Cambridge, MA (US)

(21) Appl. No.: 18/814,323

(22) Filed: Aug. 23, 2024

Related U.S. Application Data
(63) Continuation of application No. 18/541,286, filed on Dec. 15, 2023, now abandoned.
Application No. 18/541,286 is a continuation of application No. 18/193,311, filed on Mar. 30, 2023, now abandoned.
Application No. 18/193,311 is a continuation of application No. 17/819,554, filed on Aug. 12, 2022, now abandoned.
Application No. 17/819,554 is a continuation of application No. 17/567,252, filed on Jan. 3, 2022, now abandoned.
Application No. 17/567,252 is a continuation of application No. 16/434,799, filed on Jun. 7, 2019, now abandoned.
Application No. 16/434,799 is a continuation of application No. 15/604,335, filed on May 24, 2017, now Pat. No. 10,364,431.
Application No. 15/604,335 is a continuation of application No. 15/292,553, filed on Oct. 13, 2016, now abandoned.
Application No. 15/292,553 is a continuation of application No. 14/214,567, filed on Mar. 14, 2014, now Pat. No. 9,506,058.
(60) Provisional application No. 61/793,463, filed on Mar. 15, 2013.

(51) Int. Cl. C12N 15/113; A61K 31/7125

(52) U.S. Cl. CPC C12N 15/113; A61K 31/7125; C12N 2310/33; C12N 2320/30

(57) ABSTRACT

Improved compositions and methods for treating muscular dystrophy by administering antisense molecules capable of binding to a selected target site in the human dystrophin gene to induce exon skipping are described.

### CROSS-REFERENCE RELATED APPLICATIONS

This application is a continuation of U.S. patent application Ser. No. 18/541,286, filed Dec. 15, 2023, which is a continuation of U.S. patent application Ser. No. 18/193,311, filed Mar. 30, 2023, abandoned, which application is a continuation of U.S. patent application Ser. No. 17/819,554, filed Aug. 12, 2022, abandoned, which application is a continuation of U.S. patent application Ser. No. 17/567,252, filed Jan. 3, 2022, abandoned, which application is a continuation of U.S. patent application Ser. No. 16/434,799, filed Jun. 7, 2019, abandoned, which application is a continuation of U.S. patent application Ser. No. 15/604,335, filed May 24, 2017, now issued as U.S. Pat. No. 10,364,431, which application is a continuation of U.S. patent application Ser. No. 15/292,553, filed Oct. 13, 2016, abandoned, which application is a continuation of U.S. patent application Ser. No. 14/214,567, filed Mar. 14, 2014, now issued as U.S. Pat. No. 9,506,058, which claims the benefit of U.S. Provisional Patent Application Ser. No. 61/793,463, filed Mar. 15, 2013, which applications are each incorporated herein by reference in their entireties.

### STATEMENT REGARDING SEQUENCE LISTING

The Sequence Listing associated with this application has been submitted electronically in ASCII format, and is hereby incorporated by reference into the specification in its entirety. The name of the text file containing the sequence Listing is 4140_003000G_Sequencelisting_ST26. The text file is 1,176,036 bytes, was created on Aug. 23, 2024 and is being submitted electronically via Patent Center.

### FIELD OF THE INVENTION

The present invention relates to improved methods for treating muscular dystrophy in a patient. It also provides compositions suitable for facilitating exon skipping in the human dystrophin gene.

### BACKGROUND OF THE INVENTION

Antisense technologies are being developed using a range of chemistries to affect gene expression at a variety of different levels (transcription, splicing, stability, translation). Much of that research has focused on the use of antisense compounds to correct or compensate for abnormal or disease-associated genes in a wide range of indications. Antisense molecules are able to inhibit gene expression with specificity, and because of this, many research efforts concerning oligonucleotides as modulators of gene expression have focused on inhibiting the expression of targeted genes or the function of cis-acting elements. The antisense oligonucleotides are typically directed against RNA, either the sense strand (e.g., mRNA), or minus-strand in the case of some viral RNA targets. To achieve a desired effect of specific gene down-regulation, the oligonucleotides generally either promote the decay of the targeted mRNA, block translation of the mRNA or block the function of cis-acting RNA elements, thereby effectively preventing either de novo synthesis of the target protein or replication of the viral RNA.

However, such techniques are not useful where the object is to up-regulate production of the native protein or compensate for mutations that induce premature termination of translation, such as nonsense or frame-shifting mutations. In these cases, the defective gene transcript should not be subjected to targeted degradation or steric inhibition, so the antisense oligonucleotide chemistry should not promote target mRNA decay or block translation.

In a variety of genetic diseases, the effects of mutations on the eventual expression of a gene can be modulated through a process of targeted exon skipping during the splicing process. The splicing process is directed by complex multi-component machinery that brings adjacent exon-intron junctions in pre-mRNA into close proximity and performs cleavage of phosphodiester bonds at the ends of the introns with their subsequent reformation between exons that are to be spliced together. This complex and highly precise process is mediated by sequence motifs in the pre-mRNA that are relatively short, semi-conserved RNA segments to which various nuclear splicing factors that are then involved in the splicing reactions bind. By changing the way the splicing machinery reads or recognizes the motifs involved in pre-mRNA processing, it is possible to create differentially spliced mRNA molecules. It has now been recognized that the majority of human genes are alternatively spliced during normal gene expression, although the mechanisms involved have not been identified. Bennett et al. (U.S. Pat. No. 6,210,892) describe antisense modulation of wild-type cellular mRNA processing using antisense oligonucleotide analogs that do not induce RNAse H-mediated cleavage of the target RNA. This finds utility in being able to generate alternatively spliced mRNAs that lack specific exons (e.g., as described by (Sazani, Kole, et al. 2007) for the generation of soluble TNF superfamily receptors that lack exons encoding membrane spanning domains.

In cases where a normally functional protein is prematurely terminated because of mutations therein, a means for restoring some functional protein production through antisense technology has been shown to be possible through intervention during the splicing processes, and that if exons associated with disease-causing mutations can be specifically deleted from some genes, a shortened protein product can sometimes be produced that has similar biological properties of the native protein or has sufficient biological activity to ameliorate the disease caused by mutations associated with the exon (see e.g., Sierakowska, Sambade et al. 1996; Wilton, Lloyd et al. 1999; van Deutekom, Bremmer-Bout et al. 2001; Lu, Mann et al. 2003; Aartsma-Rus, Janson et al. 2004). Kole et al. (U.S. Pat. Nos. 5,627,274; 5,916,808; 5,976,879; and 5,665,593) disclose methods of combating aberrant splicing using modified antisense oligonucleotide analogs that do not promote decay of the targeted pre-mRNA. Bennett et al. (U.S. Pat. No. 6,210,892) describe antisense modulation of wild-type cellular mRNA processing also using antisense oligonucleotide analogs that do not induce RNAse H-mediated cleavage of the target RNA.

The process of targeted exon skipping is likely to be particularly useful in long genes where there are many exons and introns, where there is redundancy in the genetic constitution of the exons or where a protein is able to function without one or more particular exons. Efforts to redirect gene processing for the treatment of genetic diseases associated with truncations caused by mutations in various genes have focused on the use of antisense oligonucleotides that either: (1) fully or partially overlap with the elements involved in the splicing process; or (2) bind to the pre-mRNA at a position sufficiently close to the element to disrupt the binding and function of the splicing factors that would normally mediate a particular splicing reaction which occurs at that element.

Duchenne muscular dystrophy (DMD) is caused by a defect in the expression of the protein dystrophin. The gene encoding the protein contains 79 exons spread out over more than 2 million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD.

Disease onset can be documented at birth with elevated creatine kinase levels, and significant motor deficits may be present in the first year of life. By the age of seven or eight, most patients with DMD have an increasingly labored gait and are losing the ability to rise from the floor and climb stairs; by ages 10 to 14, most are wheelchair-dependent. DMD is uniformly fatal; affected individuals typically die of respiratory and/or cardiac failure in their late teens or early 20s. The continuous progression of DMD allows for therapeutic intervention at all stages of the disease; however, treatment is currently limited to glucocorticoids, which are associated with numerous side effects including weight gain, behavioral changes, pubertal changes, osteoporosis, Cushingoid facies, growth inhibition, and cataracts. Consequently, developing better therapies to treat the underlying cause of this disease is imperative.

A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity, resulting in a Becker phenotype.

For many years it has been known that deletions of an exon or exons which do not alter the reading frame of a dystrophin protein would give rise to a BMD phenotype, whereas an exon deletion that causes a frame-shift will give rise to DMD (Monaco, Bertelson et al. 1988). In general, dystrophin mutations including point mutations and exon deletions that change the reading frame and thus interrupt proper protein translation result in DMD. It should also be noted that some BMD and DMD patients have exon deletions covering multiple exons.

Modulation of mutant dystrophin pre-mRNA splicing with antisense oligoribonucleotides has been reported both in vitro and in vivo (see e.g., Matsuo, Masumura et al. 1991; Takeshima, Nishio et al. 1995; Pramono, Takeshima et al. 1996; Dunckley, Eperon et al. 1997; Dunckley, Manoharan et al. 1998; Errington, Mann et al. 2003).

The first example of specific and reproducible exon skipping in the mdx mouse model was reported by Wilton et al. (Wilton, Lloyd et al. 1999). By directing an antisense molecule to the donor splice site, consistent and efficient exon 23 skipping was induced in the dystrophin mRNA within 6 hours of treatment of the cultured cells. Wilton et al. also describe targeting the acceptor region of the mouse dystrophin pre-mRNA with longer antisense oligonucleotides. While the first antisense oligonucleotide directed at the intron 23 donor splice site induced consistent exon skipping in primary cultured myoblasts, this compound was found to be much less efficient in immortalized cell cultures expressing higher levels of dystrophin. However, with refined targeting and antisense oligonucleotide design, the efficiency of specific exon removal was increased by almost an order of magnitude (Mann, Honeyman et al. 2002).

Recent studies have begun to address the challenge of achieving sustained dystrophin expression accompanied by minimal adverse effects in tissues affected by the absence of dystrophin. Intramuscular injection of an antisense oligonucleotide targeted to exon 51 (PRO051) into the tibialis anterior muscle in four patients with DMD resulted in specific skipping of exon 51 without any clinically apparent adverse effects (Mann, Honeyman et al. 2002; van Deutekom, Janson et al. 2007). Studies looking at systemic delivery of an antisense phosphorodiamidate morpholino oligomer conjugated to a cell-penetrating peptide (PPMO) targeted to exon 23 in mdx mice produced high and sustained dystrophin protein production in skeletal and cardiac muscles without detectable toxicity (Jearawiriyapaisarn, Moulton et al. 2008; Wu, Moulton et al. 2008; Yin, Moulton et al. 2008).

Recent clinical trials testing the safety and efficacy of splice switching oligonucleotides (SSOs) for the treatment of DMD are based on SSO technology to induce alternative splicing of pre-mRNAs by steric blockade of the spliceosome (Cirak et al., 2011; Goemans et al., 2011; Kinali et al., 2009; van Deutekom et al., 2007). However, despite these successes, the pharmacological options available for treating DMD are limited. Notably, an antisense oligonucleotide (drisapersen), which utilizes a negatively charged phosphorothioate backbone, has been associated in clinical trials with proteinuria, increased urinary al-microglobulin, thrombocytopenia and injection site reactions, such as erythema and inflammation.

Eteplirsen, which is being developed by the assignee of this application, has been the subject of clinical studies to test its safety and efficacy and clinical development is ongoing. Eteplirsen is an oligonucleotide structurally distinct from drisapersen. Specifically, the chemical backbone of eteplirsen is phosphorodiamidate mopholino (PMO), whereas the chemical backbone of drisapersen is 2′O-methyl phosphorothioate (2′-OMe). These structural differences and their potential impact on clinical outcomes were recently described. See Molecular Therapy Nucleic Acids (2014) 3, e152; doi: 10.1038/mtna.2014.6 (Published online 11 Mar. 2014).

The sequence of eteplirsen has previously been described. See, for example, U.S. Pat. No. 7,807,816, which is exclusively licensed to Applicants. U.S. Pat. No. 7,807,816, however, does not explicitly discuss optimum dosing schedules and routes of administration for eteplirsen.

Thus, there remains a need for improved compositions and methods for treating muscular dystrophy, such as DMD and BMD in patients.

### SUMMARY OF THE INVENTION

The present invention is based, at least in part, on compelling evidence of a therapeutic effect of an exon skipping antisense oligonucleotide, eteplirsen, which represents a major advance in the treatment of DMD by addressing the underlying cause of the disease. The novel finding that treatment with an exon 51 skipping antisense oligonucleotide, eteplirsen, produced reliable increases in novel dystrophin and stabilized walking ability (e.g., stabilization of ambulation), as measured by the 6 Minute Walk Test (6MWT), underscores the potential to alter the course of the disease. Significantly, no drug-related adverse events were seen in 576 infusions administered over one year. When applied to other exons, the use of exon skipping antisense oligonucleotides could treat an estimated 70% to 80% of patients who have DMD due to a deletion in the dystrophin gene.

Accordingly, in one aspect the present invention relates to a method for treating Duchenne muscular dystrophy a patient in need thereof comprising administering to the patient a dose of eteplirsen of about 30 mg/kg once a week. In some embodiments, eteplirsen is administered in a single dose. In some embodiments eteplirsen is administered intravenously. In some embodiments, the patient has an out-of-frame deletion(s) that may be corrected by skipping exon 51 of the dystrophin gene. In some embodiments, the patient is a pediatric patient.

In another aspect, the present invention relates to a method for treating Duchenne muscular dystrophy a patient in need thereof comprising administering to the patient a dose of eteplirsen of about 30 mg/kg once a week, wherein the patient is administered an oral corticosteroid for at least 24 weeks prior to the first dose of eteplirsen.

In yet another aspect, the present invention provides a method for treating Duchenne muscular dystrophy a patient in need thereof comprising administering intravenously to the patient a single dose of eteplirsen of about 30 mg/kg once a week, wherein the patient has an out-of-frame deletion(s) that may be corrected by skipping exon 51 of the dystrophin gene.

Other embodiments of the invention relate to a method for treating Duchenne muscular dystrophy a patient in need thereof comprising administering to the patient a dose of eteplirsen of about 50 mg/kg once a week. In some embodiments, eteplirsen is administered in a single dose. In some embodiments, eteplirsen is administered intravenously. In some embodiments the patient has an out-of-frame deletion(s) that may be corrected by skipping exon 51 of the dystrophin gene. In some embodiments, the patient is a pediatric patient. In yet other embodiments, the patient is administered an oral corticosteroid for at least 24 weeks prior to the first dose of eteplirsen.

In another aspect, the invention relates to a method for treating Duchenne muscular dystrophy a patient in need thereof comprising administering intravenously to the patient a single dose of eteplirsen of about 50 mg/kg once a week, wherein the patient has an out-of-frame deletion(s) that may be corrected by skipping exon 51 of the dystrophin gene.

In yet another aspect, the invention provides a method for increasing dystrophin production in a patient having Duchenne muscular dystrophy comprising administering to the patient a dose of eteplirsen of about 30 mg/kg once a week. In some embodiments, a dose of eteplirsen of about 50 mg/kg once a week is administered. In some embodiments, eteplirsen is administered in a single dose. In some embodiments, eteplirsen is administered intravenously. In other embodiments, the patient has an out-of-frame deletion(s) that may be corrected by skipping exon 51 of the dystrophin gene. In some embodiments the patient is a pediatric patient. In other embodiments, the patient is administered an oral corticosteroid for at least 24 weeks prior to the first dose of eteplirsen.

Accordingly, the present invention relates to methods of treating Duchenne muscular dystrophy (DMD) or Becker muscular dystrophy (BMD) in patients by administering an effective amount of a composition comprising an antisense oligonucleotide of 20 to 50 nucleotides in length comprising at least 10 consecutive nucleotides complementary to a target region in an exon of the human dystrophin gene to specifically hybridize to the target region, induce exon skipping, and thereby treat the disease. In one embodiment, an effective amount is at least 20 mg/kg for a period of time sufficient to increase the number of dystrophin-positive fibers in a subject to at least 20% of normal, and stabilize, maintain, or improve walking distance from a 20% deficit, for example in a 6 MWT, in the patient, relative to a healthy peer. In another embodiment, an effective amount is at least 20 mg/kg to about 30 mg/kg, about 25 mg/kg to about 30 mg/kg, or about 30 mg/kg to about 50 mg/kg. In yet another embodiment, an effective amount is about 30 mg/kg or about 50 mg/kg.

In another aspect, an effective amount is at least 20 mg/kg, about 25 mg/kg, about 30 mg/kg, or about 30 mg/kg to about 50 mg/kg, for at least 24 weeks, at least 36 weeks, or at least 48 weeks, to thereby increase the number of dystrophin-positive fibers in a subject to at least 20%, about 30%, about 40%, about 50%, about 60%, about 70%, about 80%, about 90%, about 95% of normal, and stabilize or improve walking distance from a 20% deficit, for example in a 6 MWT, in the patient relative to a healthy peer. In one embodiment, treatment increases the number of dystrophin-positive fibers to 20-60%, or 30-50% of normal in the patient. In some embodiments, treatment is by systemic administration, such as once weekly by infusion. In other embodiments, treatment includes administering another therapeutic agent, such as a steroid to the subject.

In another aspect, the present invention provides a method of treating DMD or BMD in a patient by administering about 30 mg/kg to about 50 mg/kg of a composition comprising an antisense oligonucleotide of 20 to 50 nucleotides in length comprising at least 10 consecutive nucleotides complementary to a target region in an exon of the human dystrophin gene, wherein the antisense oligonucleotide specifically hybridizes to the target region inducing exon skipping, thereby treating the subject. In one embodiment, the antisense oligonucleotide is substantially uncharged. In another embodiment, the antisense oligonucleotide comprises morpholino subunits linked by phosphorus-containing intersubunit linkages joining a morpholino nitrogen of one subunit to a 5′ exocyclic carbon of an adjacent subunit. In yet another embodiment, the antisense oligonucleotide comprises morpholino subunits linked by substantially uncharged phosphorus-containing intersubunit linkages joining a morpholino nitrogen of one subunit to a 5′ exocyclic carbon of an adjacent subunit. In other aspects, the antisense oligonucleotide comprises morpholino subunits and phosphorodiamidate intersubunit linkages.

In some embodiments, the antisense oligonucleotide is 20 to 50, 30 to 50, or 20 to 30 nucleotides in length comprising at least 10, 12, 15, 17, or 20 consecutive nucleotides complementary to a target region in an exon of the human dystrophin gene selected from the group consisting of exon 51, exon 50, exon 53, exon 45, exon 46, exon 44, exon 52, exon 55 and exon 8. In one embodiment, the antisense is 20 to 50, 30 to 50, or 20 to 30 nucleotides in length and includes at least 20 consecutive nucleotides eteplirsen (SEQ ID NO: 1). In another embodiment, the antisense oligonucleotide is 20 to 50, 30 to 50, or 20 to 30 nucleotides in length and includes at least 10, 12, 15, 17, or 20 consecutive nucleotides of the antisense oligonucleotide set forth as SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. In yet another embodiment, the antisense oligonucleotide is 20 to 50, 30 to 50, or 20 to 30 nucleotides in length and includes at least 10, 12, 15, 17, or 20 consecutive nucleotides of a nucleotide sequences set forth in Tables 3 and 4, wherein uracil bases in the antisense oligonucleotide are optionally thymine bases.

In one embodiment, the composition includes eteplirsen (SEQ ID NO: 1), and, optionally, a pharmaceutically acceptable carrier. In another embodiment, the composition includes an antisense oligonucleotide selected from the group consisting of SEQ ID NOS: 1-9, such as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9. In yet another embodiment, the antisense oligonucleotide is any one or a combination of the nucleotide sequences set forth in Tables 3 and 4, wherein uracil bases in the antisense oligonucleotide are optionally thymine bases. In some aspects, the antisense oligonucleotide is chemically linked to one or more moieties or conjugates that enhance the activity, cellular distribution, or cellular uptake of the antisense oligonucleotide, such as an arginine-rich peptide.

In another aspect, the present invention provides a method of treating DMD or BMD in a patient by administering at least 20 mg/kg of a composition comprising eteplirsen (SEQ ID NO: 1) for a period of time sufficient to increase the number of dystrophin-positive fibers in a subject to at least about 20% of normal, and stabilize or improve walking distance from a 20% deficit, for example in a 6 MWT, in the patient, relative to a healthy peer. In another embodiment, an effective amount is at least 20 mg/kg to about 30 mg/kg, about 25 mg/kg to about 30 mg/kg, or about 30 mg/kg to about 50 mg/kg of a composition comprising eteplirsen (SEQ ID NO: 1), and, optionally, a pharmaceutically acceptable carrier, such as phosphate-buffered saline.

In another aspect, an effective amount of a composition comprising eteplirsen (SEQ ID NO: 1) is at least 20 mg/kg, about 25 mg/kg, about 30 mg/kg, or about 30 mg/kg to about 50 mg/kg, for at least 24 weeks, at least 36 weeks, or at least 48 weeks, to thereby increase the number of dystrophin-positive fibers in a subject to at least about 20%, about 30%, about 40%, about 50%, about 60%, about 70%, about 80%, about 90%, about 95% of normal, and stabilize or improve walking distance from a 20% deficit, for example in a 6 MWT, in the patient relative to a healthy peer. In some embodiments, treatment with antisense oligonucleotides of the present invention slows or reduces the loss of ambulation that would be expected without treatment. In some embodiments, treatment with the antisense oligonucleotides of the present invention stabilizes, maintains, or increases a stable walking distance in a patient. For example, treatment may increase the stable walking distance in the patient from baseline to greater than 3, 5, 6, 7, 8, 9, 10, 15, 20, 25, 30 or 50 meters (including all integers in between).

Other aspects of the invention relate to treatment with an antisense oligonucleotide of the invention, such as eteplirsen, which slows or reduces the progressive respiratory muscle dysfunction and/or failure in patients with DMD that would be expected without treatment. In one embodiment, treatment with an antisense oligonucleotide of the invention may reduce or eliminate the need for ventilation assistance that would be expected without treatment. In one embodiment, measurements of respiratory function for tracking the course of the disease, as well as the evaluation of potential therapeutic interventions include Maximum inspiratory pressure (MIP), maximum expiratory pressure (MEP) and forced vital capacity (FVC).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows an exemplary morpholino oligomer structure with a phosphorodiamidate linkage.

FIG. 1B shows a conjugate of an arginine-rich peptide and an antisense oligomer, in accordance with an embodiment of the invention.

FIG. 1C shows a conjugate as in FIG. 1B, wherein the backbone linkages contain one or more positively charged groups.

FIGS. 1D-G show the repeating subunit segment of exemplary morpholino oligonucleotides, designated D through G.

FIG. 2 is a schematic representation of the study design for treating DMD patients. Twelve DMD patients were randomized to one of three cohorts in the double-blind, placebo-controlled study, 201: Cohort 1, eteplirsen 30 mg/kg/wk; Cohort 2, eteplirsen 50 mg/kg/wk; and Cohort 3, placebo/delayed eteplirsen. At week 25, placebo-treated patients in Cohort 3 switched to open-label treatment with 30 or 50 mg/kg/week eteplirsen. Patients were maintained on their same dose of eteplirsen under the open-label extension study, 202. Muscle Biopsies. Patients underwent biceps biopsies at baseline and deltoid biopsies at week 48 for analysis of dystrophin. Additional biceps biopsies were obtained at week 12 (from patients in Cohort 2 and two patients in Cohort 3) or week 24 (from patients in Cohort 1 and two patients in Cohort 3). Efficacy Evaluations. The 6MWT was used as a functional outcome measure and was performed pre-treatment and every 12 weeks post treatment through week 48.

FIG. 3 depicts dystrophin-positive muscle fibers after 12, 24, and 48 weeks of eteplirsen. Panels A and B show the mean absolute change from baseline in the percentage of dystrophin-positive fibers at weeks 12, 24, and week 48 by treatment group. In Panel A: *P-value is for comparison between eteplirsen and placebo using the pooled results from weeks 12 and 24, and is based on an analysis of covariance model for ranked data with treatment as a fixed effect and baseline value and time since DMD diagnosis as covariates. Mean changes shown are based on descriptive statistics. †P-value is from a paired t-test comparing the week 48 value to baseline. ‡Results from the placebo-treated patients biopsied at weeks 12 and 24 are pooled. § Placebo/delayed eteplirsen patients began receiving eteplirsen at week 25 and had received a total of 24 doses at week 48. Abbreviations: BL=baseline; NA=not applicable; ND=not done; NS=not significant; SE=standard error.

FIG. 4 shows the effects of eteplirsen on the dystrophin-associated glycoprotein complex. (A) Representative examples of time-dependent increases in dystrophin-positive fibers in relation to treatment for all participating study patients. (B) nNOS μ staining in muscle from DMD (a) and normal (c) control patients (not in study), and from patient 6 at baseline (b) and week 48 (d), demonstrates restoration of nNOSμ binding with eteplirsen. β-sarcoglycan (e) and γ-sarcoglycan (f) staining in patient 6 at week 48 demonstrate restoration of the sarcoglycan complex with eteplirsen. (C) RT-PCR shows skipped product (289 bp) post-treatment in the muscle of patient 12.

FIG. 5 graphically depicts the functional efficacy of eteplirsen. The dark purple line shows the change from baseline in distance walked on the 6MWT over time for the 6 evaluable patients who received eteplirsen from the start of 201 (two boys were unable to at or beyond week 24 were excluded from this analysis). The gray line shows change from baseline in distance walked on the 6MWT for the 4 patients who received placebo for the first 24 weeks and eteplirsen for the last 24 weeks.

### DETAILED DESCRIPTION

Embodiments of the present invention relate to improved methods for treating muscular dystrophy, such as DMD and BMD, by administering antisense compounds that are specifically designed to induce exon skipping in the human dystrophin gene. Dystrophin plays a vital role in muscle function, and various muscle-related diseases are characterized by mutated forms of this gene. Hence, in certain embodiments, the improved methods described herein may be used for inducing exon skipping in mutated forms of the human dystrophin gene, such as the mutated dystrophin genes found in DMD and BMD.

Due to aberrant mRNA splicing events caused by mutations, these mutated human dystrophin genes either express defective dystrophin protein or express no measurable dystrophin at all, a condition that leads to various forms of muscular dystrophy. To remedy this condition, the antisense compounds of the present invention hybridize to selected regions of a pre-processed RNA of a mutated human dystrophin gene, induce exon skipping and differential splicing in that otherwise aberrantly spliced dystrophin mRNA, and thereby allow muscle cells to produce an mRNA transcript that encodes a functional dystrophin protein. In certain embodiments, the resulting dystrophin protein is not necessarily the “wild-type” form of dystrophin, but is rather a truncated, yet functional or semi-functional, form of dystrophin.

By increasing the levels of functional dystrophin protein in muscle cells, these and related embodiments are useful in the prophylaxis and treatment of muscular dystrophy, especially those forms of muscular dystrophy, such as DMD and BMD, that are characterized by the expression of defective dystrophin proteins due to aberrant mRNA splicing. The methods described herein further provide improved treatment options for patients with muscular dystrophy and offer significant and practical advantages over alternate methods of treating relevant forms of muscular dystrophy. For example, in some embodiments, the improved methods relate to the administration of an antisense compound for inducing exon skipping in the human dystrophin gene at a higher dose and/or for a longer duration than prior approaches.

Thus, the invention relates to improved methods for treating muscular dystrophy such as DMD and BMD, by inducing exon skipping in a patient. In some embodiments, exon skipping is induced by administering an effective amount of a composition which includes a charge-neutral, phosphorodiamidate morpholino oligomer (PMO), such as eteplirsen, which selectively binds to a target sequence in an exon of dystrophin pre-mRNA. In some embodiments, the invention relates to methods of treating DMD or BMD in which an effective amount of a composition e.g., at least 20 mg/kg, about 25 mg/kg, about 30 mg/kg or about 30 mg/kg to about 50 mg/kg, which includes an antisense as described herein, such as eteplirsen, over a period of time sufficient to treat the disease.

Some embodiments of the present invention relate to the use of eteplirsen as a disease-modifying therapy for treating DMD. Without being bound by theory, the clinical efficacy seen to date with eteplirsen may be derived from its safety profile due to its unique chemical composition, which is characterized by nucleotides bound to morpholine rings linked through charge-neutral phosphorodiamidate moieties.

In DMD patients treated with eteplirsen for one year, the mean percentage of dystrophin-positive fibers was increased to 47% of normal, relative to baseline. The magnitude of the increase was dependent upon treatment duration. Significant increases in dystrophin levels were observed in the 24-week biopsies taken from patients in Cohort 1 (30 mg/kg) and in the 48-week biopsies from patients in Cohort 3 (who started eteplirsen at week 25).

Eteplirsen's clinical benefit mirrored its ability to induce exon skipping and restore functional dystrophin production. Clinical effect was assessed with the 6MWT, a measure of endurance and muscular capacity that goes beyond the assessment of strength in individual muscle groups. Patients who received 30 or 50 mg/kg eteplirsen from the beginning maintained a stable walking distance over 48 weeks, consistent with eteplirsen-induced increases in novel dystrophin expression between weeks 12 and 24. In contrast, patients in the placebo/delayed eteplirsen cohort lost 70 meters by week 36, but appeared to stabilize by week 48 (24 weeks after initiating eteplirsen). This is the same timeframe in which a clinical impact was seen in patients who received 30 or 50 mg/kg eteplirsen once a week from the start of the study. Both cohorts have maintained stable 6MWT results over 120 weeks as described below.

At 120 weeks, patients in the 30 mg/kg and 50 mg/kg eteplirsen cohorts who were able to perform the 6MWT (modified Intent-to-Treat or mITT population; n=6) experienced a general stability with a slight decline of 13.9 meters, or less than 5 percent, from baseline in walking ability. A statistically significant treatment benefit of 64.9 meters (p≤0.006) was observed for the mITT population compared with the placebo/delayed-treatment cohort (n=4), which initiated treatment at Week 25 following 24 weeks of placebo. After experiencing a substantial decline earlier in the study (prior to treatment with etiplersen), the placebo/delayed-treatment cohort also demonstrated stabilization in walking ability for more than 1.5 years, from Week 36 through 120, the period from which meaningful levels of dystrophin were likely produced, with a decline of 9.5 meters over this timeframe. These analyses were based on the maximum 6MWT score when the test was performed on two consecutive days.

Respiratory muscle function from baseline through Week 120 in both dosing cohorts, as measured by maximum inspiratory and expiratory pressure (MIP and MEP), showed a 14.6 percent mean increase in MIP and a 15.0 percent mean increase in MEP. Analyses of MIP percent predicted (MIP adjusted for weight) and MEP percent predicted (MEP adjusted for age) demonstrated a mean increase from 90.2 percent at baseline to 95.2 percent at Week 120 in MIP percent predicted, and a slight mean increase from 79.3 percent at baseline to 79.6 percent at Week 120 in MEP percent predicted. In addition, there was a mean increase in forced vital capacity (FVC), a measure of lung volume, of 8.7 percent from baseline to Week 120, and FVC percent predicted (FVC adjusted for age and height) was maintained above a mean of 90 percent through Week 120, with 101 percent at baseline and 93 percent at Week 120.

The present invention is based, at least in part, on the evidence of a therapeutic effect of eteplirsen, which represents a major advance in the treatment of DMD by addressing the underlying cause of the disease. Accordingly, the invention relates to methods of treating DMD or BMD in patients by administering an effective amount of a composition which includes an antisense oligonucleotide, such as eteplirsen, which is complementary to a target region in an exon of the human dystrophin gene to specifically hybridize to the target region, induce exon skipping, and treat the disease. In one embodiment, treatment is by administering one or more antisense oligonucleotides of the present invention (e.g., a nucleotide sequence shown in Tables 3 and 4), optionally as part of a pharmaceutical formulation or dosage form, to a subject in need thereof. Treatment includes inducing exon-skipping in a subject by administering an effective amount of one or more antisense oligonucleotides, in which the exon is any one or more of exons 1-79 from the dystrophin gene. Preferably, the exon is exon 44, 45, 46, 47, 48, 49, 50, 51, 52, 53, 54, 55, 56 or 8 from the human dystrophin gene.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

### I. DEFINITIONS

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The terms “complementary” and “complementarity” refer to polynucleotides (i.e., a sequence of nucleotides) related by base-pairing rules. For example, the sequence “T-G-A (5′-3′),” is complementary to the sequence “T-C-A (5′-3′).” Complementarity may be “partial,” in which only some of the nucleic acids' bases are matched according to base pairing rules. Or, there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

The terms “cell penetrating peptide” and “CPP” are used interchangeably and refer to cationic cell penetrating peptides, also called transport peptides, carrier peptides, or peptide transduction domains. The peptides, as shown herein, have the capability of inducing cell penetration within 100% of cells of a given cell culture population and allow macromolecular translocation within multiple tissues in vivo upon systemic administration. A preferred CPP embodiment is an arginine-rich peptide as described further below.

The terms “antisense oligomer” and “antisense compound” and “antisense oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. The cyclic subunits are based on ribose or another pentose sugar or, in a preferred embodiment, a morpholino group (see description of morpholino oligomers below). The oligomer may have exact or near sequence complementarity to the target sequence; variations in sequence near the termini of an oligomer are generally preferable to variations in the interior.

Such an antisense oligomer can be designed to block or inhibit translation of mRNA or to inhibit natural pre-mRNA splice processing, and may be said to be “directed to” or “targeted against” a target sequence with which it hybridizes. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a preprocessed mRNA. A preferred target sequence is any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

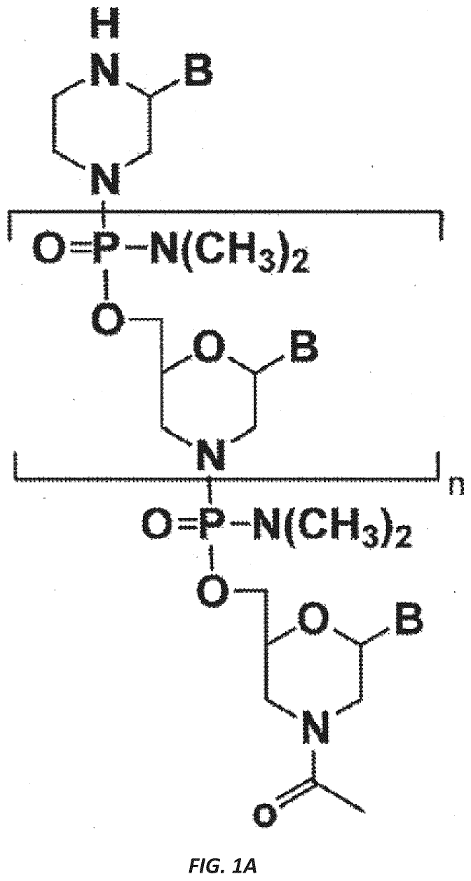

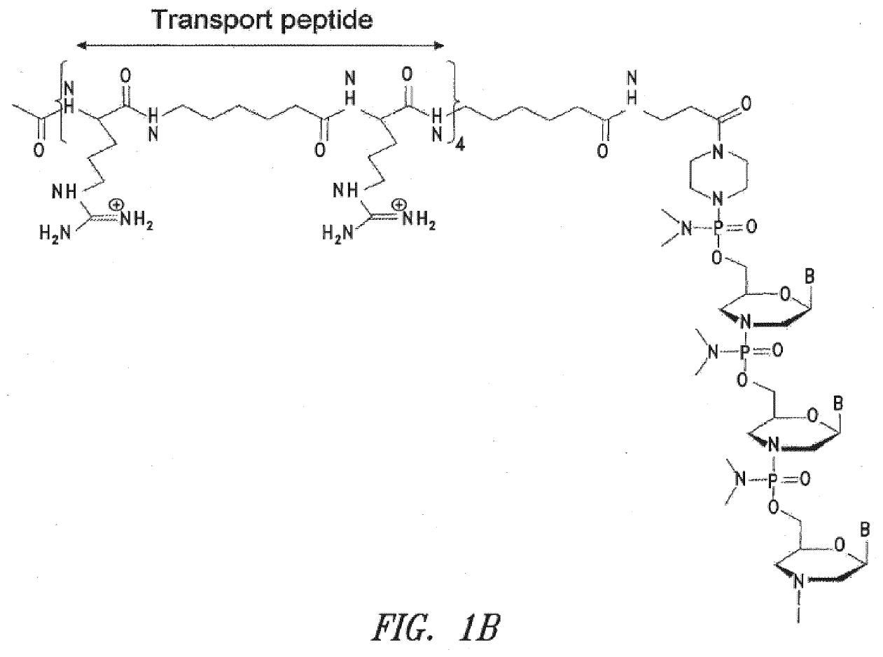

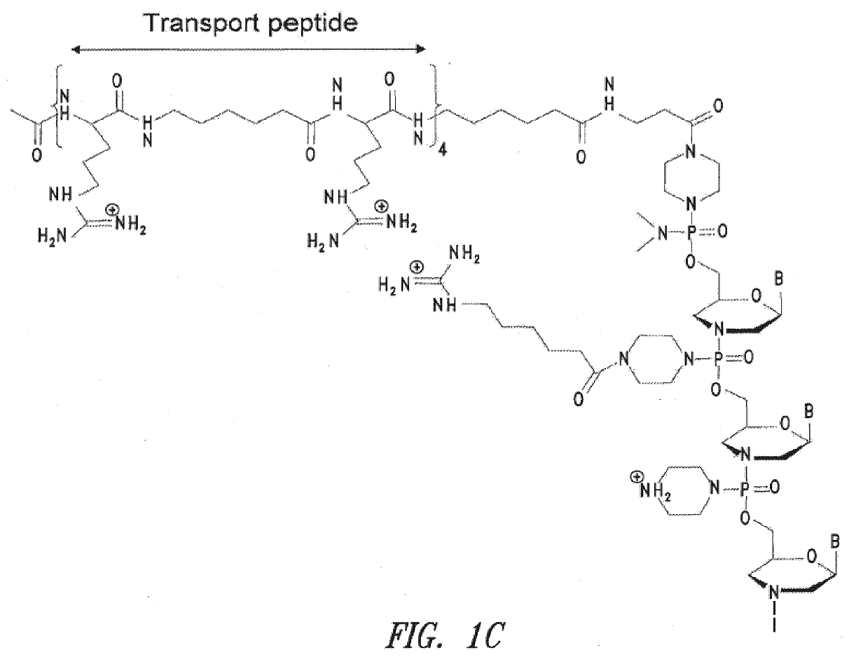

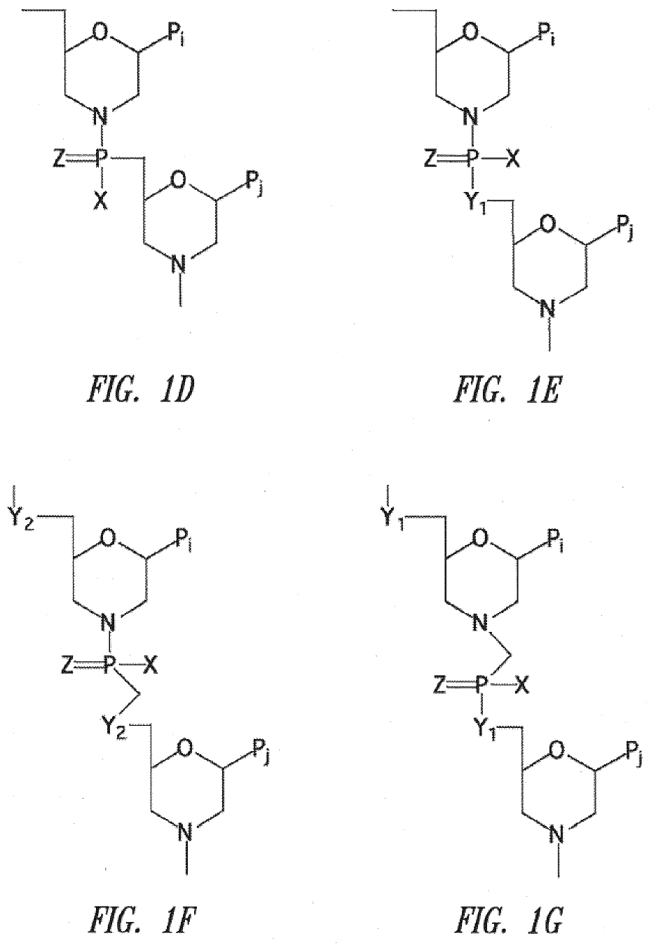

The terms “morpholino oligomer” or “PMO” (phosphoramidate- or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. See, for example, the structure in FIG. 1A, which shows a preferred phosphorodiamidate linkage type. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. The purine or pyrimidine base pairing moiety is typically adenine, cytosine, guanine, uracil, thymine or inosine. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, 8,299,206 and 7,943,762 (cationic linkages), all of which are incorporated herein by reference. Modified intersubunit linkages and terminal groups are detailed in PCT application US2011/038459 and publication WO/2011/150408 which are incorporated herein by reference in their entirety.

“Eteplirsen”, also known as “AVN-4658” is a PMO having the base sequence 5′-CTCCAACATCAAGGAAGATGGCATTTCTAG-3′ (SEQ ID NO:1). Eteplirsen is registered under CAS Registry Number 1173755-55-9. Chemical names include:
   |RNA, [P-deoxy-P-(dimethylamino)](2′,3′-dideoxy-2′,3′-imino-2′,3′-seco) (2′a→5′)(C-m5U-C-C-A-A-C-A-m5U-C-A-A-G-G-A-A-G-A-m5U-G-G-C-A-m5U-m5U-mSU-C-m5U-A-G), 5-IP-[4-[[2-[2-(2-hydroxyethoxy) ethoxy]ethoxy]carbonyl]-1-piperazinyl]-N,N-dimethylphosphonamidate]
   and
P,2′,3′-trideoxy-P-(dimethylamino)-5′-O-{P-[4-(10-hydroxy-2,5,8-trioxadecanoyl) piperazin-1-yl]-N,N-dimethylphosphonamidoyl}-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a-+5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoadenylyl-(2′a→5′)-2′,3′-dideoxy-2′,3′-imino-2′,3′-secoguanosine

Eteplirsen has the following structure:

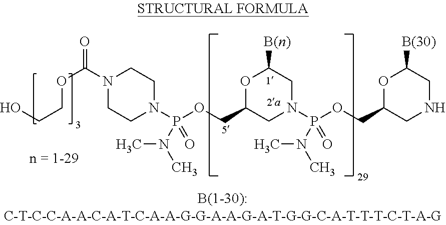

[symbol]

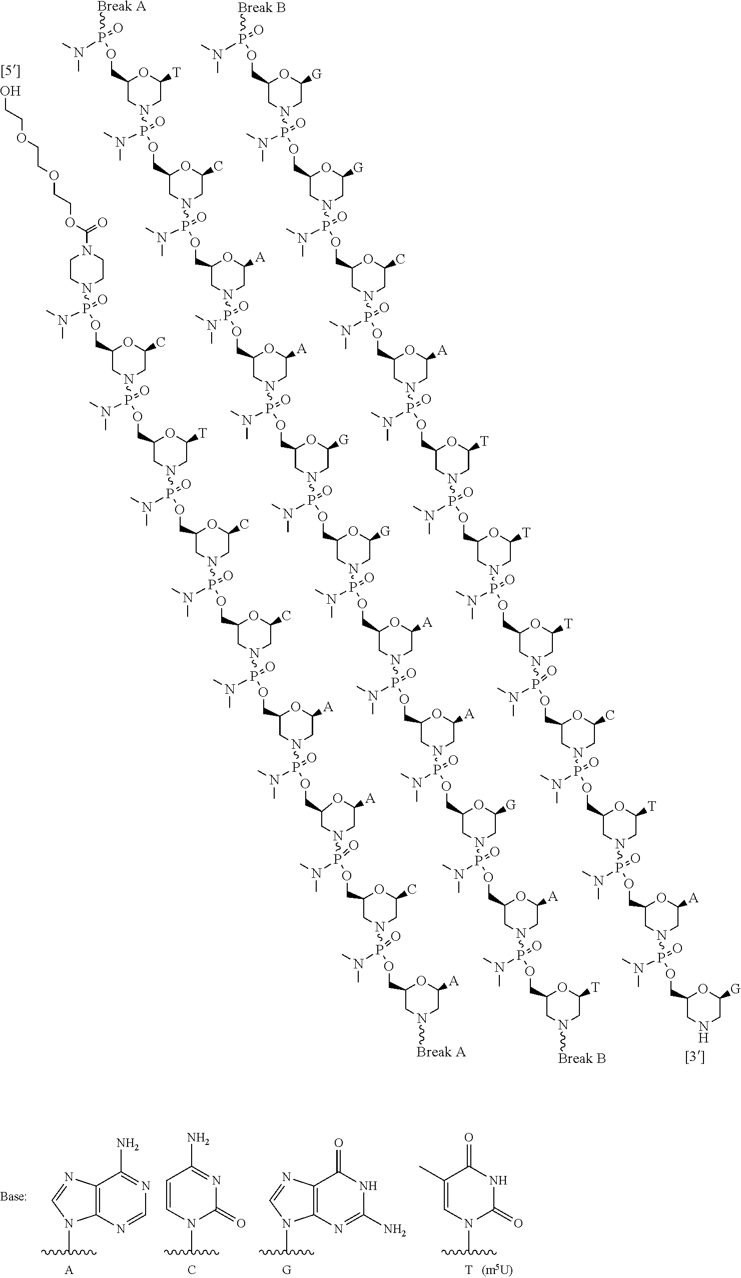

An “amino acid subunit” or “amino acid residue” can refer to an a-amino acid residue (—CO—CHR—NH—) or a β- or other amino acid residue (e.g. —CO—(CH₂)ₙCHR—NH—), where R is a side chain (which may include hydrogen) and n is 1 to 6, preferably 1 to 4.

The term “naturally occurring amino acid” refers to an amino acid present in proteins found in nature. The term “non-natural amino acids” refers to those amino acids not present in proteins found in nature, examples include beta-alanine (B-Ala), 6-aminohexanoic acid (Ahx) and 6-aminopentanoic acid.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 79 exons.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

An “effective amount” or “therapeutically effective amount” refers to an amount of therapeutic compound, such as an antisense oligonucleotide, administered to a human subject, either as a single dose or as part of a series of doses, which is effective to produce a desired therapeutic effect. For an antisense oligonucleotide, this effect is typically brought about by inhibiting translation or natural splice-processing of a selected target sequence. In some embodiments, an effective amount is at least 20 mg/kg of a composition including an antisense oligonucleotide for a period of time to treat the subject. In one embodiment, an effective amount is at least 20 mg/kg of a composition including an antisense oligonucleotide to increase the number of dystrophin-positive fibers in a subject to at least 20% of normal. In another embodiment, an effective amount is at least 20 mg/kg of a composition including an antisense oligonucleotide to stabilize, maintain, or improve walking distance from a 20% deficit, for example in a 6 MWT, in a patient, relative to a healthy peer. In another embodiment, an effective amount is at least 20 mg/kg to about 30 mg/kg, about 25 mg/kg to about 30 mg/kg, or about 30 mg/kg to about 50 mg/kg. In yet another embodiment, an effective amount is about 30 mg/kg or about 50 mg/kg. In another aspect, an effective amount is at least 20 mg/kg, about 25 mg/kg, about 30 mg/kg, or about 30 mg/kg to about 50 mg/kg, for at least 24 weeks, at least 36 weeks, or at least 48 weeks, to thereby increase the number of dystrophin-positive fibers in a subject to at least 20%, about 30%, about 40%, about 50%, about 60%, about 70%, about 80%, about 90%, about 95% of normal, and stabilize or improve walking distance from a 20% deficit, for example in a 6 MWT, in the patient relative to a healthy peer. In one embodiment, treatment increases the number of dystrophin-positive fibers to 20-60%, or 30-50% of normal in the patient.

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In certain embodiments, the exon being skipped is an aberrant exon from the human dystrophin gene, which may contain a mutation or other alteration in its sequence that otherwise causes aberrant splicing. In certain embodiments, the exon being skipped is any one or more of exons 1-79 of the human dystrophin gene, such as 3-8, 10-16, 19-40, 42-47, and 50-55, though exons 44, 45, 46, 47, 48, 49, 50, 51, 52, 53, 54, 55, 56 and 8 of the human dystrophin gene are preferred.

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins. Included are truncated forms of dystrophin, such as those forms that are produced by certain of the exon-skipping antisense compounds of the present invention.

The term “restoration” of dystrophin synthesis or production refers generally to the production of a dystrophin protein including truncated forms of dystrophin in a patient with muscular dystrophy following treatment with an antisense oligonucleotide as described herein. In some embodiments, treatment results in an increase in novel dystrophin production in a patient by 1%, 5%, 10%, 20%, 30%, 40%, 50%, 60%, 70%, 80%, 90% or 100% (including all integers in between). In some embodiments, treatment increases the number of dystrophin-positive fibers to at least 20%, about 30%, about 40%, about 50%, about 60%, about 70%, about 80%, about 90% or about 95% to 100% of normal in the subject. In other embodiments, treatment increases the number of dystrophin-positive fibers to about 20% to about 60%, or about 30% to about 50% of normal in the subject. The percent of dystrophin-positive fibers in a patient following treatment can be determined by a muscle biopsy using known techniques. For example, a muscle biopsy may be taken from a suitable muscle, such as the biceps brachii muscle in a patient.

Analysis of the percentage of positive dystrophin fibers may be performed pre-treatment and/or post-treatment or at time points throughout the course of treatment. In some embodiments, a post-treatment biopsy is taken from the contralateral muscle from the pre-treatment biopsy. Pre- and post-treatment dystrophin expression studies may be performed using any suitable assay for dystrophin. In one embodiment, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody may be used.

In some embodiments, the percent dystrophin-positive fibers are calculated by dividing the number of positive fibers by the total fibers counted. Normal muscle samples have 100% dystrophin-positive fibers. Therefore, the percent dystrophin-positive fibers can be expressed as a percentage of normal. To control for the presence of trace levels of dystrophin in the pretreatment muscle as well as revertant fibers a baseline can be set using sections of pre-treatment muscles from each patient when counting dystrophin-positive fibers in post-treatment muscles. This may be used as a threshold for counting dystrophin-positive fibers in sections of post-treatment muscle in that patient. In other embodiments, antibody-stained tissue sections can also be used for dystrophin quantification using Bioquant image analysis software (Bioquant Image Analysis Corporation, Nashville, TN). The total dystrophin fluorescence signal intensity can be reported as a percentage of normal. In addition, Western blot analysis with monoclonal or polyclonal anti-dystrophin antibodies can be used to determine the percentage of dystrophin positive fibers. For example, the anti-dystrophin antibody NCL-Dys1 from Novacastra may be used. The percentage of dystrophin-positive fibers can also be analyzed by determining the expression of the components of the sarcoglycan complex (β,γ) and/or neuronal NOS.

In some embodiments, treatment with an antisense oligonucleotide of the invention, such as eteplirsen, slows or reduces the progressive respiratory muscle dysfunction and/or failure in patients with DMD that would be expected without treatment. In one embodiment, treatment with an antisense oligonucleotide of the invention may reduce or eliminate the need for ventilation assistance that would be expected without treatment. In one embodiment, measurements of respiratory function for tracking the course of the disease, as well as the evaluation of potential therapeutic interventions include Maximum inspiratory pressure (MIP), maximum expiratory pressure (MEP) and forced vital capacity (FVC). MIP and MEP measure the level of pressure a person can generate during inhalation and exhalation, respectively, and are sensitive measures of respiratory muscle strength. MIP is a measure of diaphragm muscle weakness.

In one embodiment, MEP may decline before changes in other pulmonary function tests, including MIP and FVC. In another embodiment, MEP may be an early indicator of respiratory dysfunction. In another embodiment, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In another embodiment, MIP percent predicted (MIP adjusted for weight), MEP percent predicted (MEP adjusted for age) and FVC percent predicted (FVC adjusted for age and height) are supportive analyses.

By “isolated” is meant material that is substantially or essentially free from components that normally accompany it in its native state. For example, an “isolated polynucleotide,” as used herein, may refer to a polynucleotide that has been purified or removed from the sequences that flank it in a naturally-occurring state, e.g., a DNA fragment that has been removed from the sequences that are normally adjacent to the fragment.

As used herein, “sufficient length” refers to an antisense oligonucleotide that is complementary to at least 8, more typically 8-30, contiguous nucleobases in a target dystrophin pre-mRNA. In some embodiments, an antisense of sufficient length includes at least 8, 9, 10, 11, 12, 13, 14, or 15 contiguous nucleobases in the target dystrophin pre-mRNA. In other embodiments an antisense of sufficient length includes at least 16, 17, 18, 19, 20, 21, 22, 23, 24, or 25 contiguous nucleobases in the target dystrophin pre-mRNA. An antisense oligonucleotide of sufficient length has at least a minimal number of nucleotides to be capable of specifically hybridizing to any one or more of exons 1-79 of the dystrophin gene. Preferably, the antisense oligonucleotide of the invention has a minimal number of nucleotides to be capable of specifically hybridizing to any one or more of exons 44, 45, 46, 47, 48, 49, 50, 51, 52, 53, 54, 55, 56 or 8 of the human dystrophin gene. Preferably an oligonucleotide of sufficient length is from about 10 to about 50 nucleotides in length, including oligonucleotides of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 or more nucleotides. In one embodiment, an oligonucleotide of sufficient length is from 10 to about 30 nucleotides in length. In another embodiment, an oligonucleotide of sufficient length is from 15 to about 25 nucleotides in length. In yet another embodiment, an oligonucleotide of sufficient length is from 20 to 30, or 20 to 50, nucleotides in length. In yet another embodiment, an oligonucleotide of sufficient length is from 25 to 28 nucleotides in length.

By “enhance” or “enhancing,” or “increase” or “increasing,” or “stimulate” or “stimulating,” refers generally to the ability of one or antisense compounds or compositions to produce or cause a greater physiological response (i.e., downstream effects) in a cell or a subject, as compared to the response caused by either no antisense compound or a control compound. A measurable physiological response may include increased expression of a functional form of a dystrophin protein, or increased dystrophin-related biological activity in muscle tissue, among other responses apparent from the understanding in the art and the description herein. Increased muscle function can also be measured, including increases or improvements in muscle function by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. The percentage of muscle fibers that express a functional dystrophin can also be measured, including increased dystrophin expression in about 1%, 2%, %, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibers. For instance, it has been shown that around 40% of muscle function improvement can occur if 25-30% of fibers express dystrophin (see, e.g., DelloRusso et al, Proc Natl Acad Sci USA 99: 12979-12984, 2002). An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1), e.g., 1.5, 1.6, 1.7, 1.8, etc.) the amount produced by no antisense compound (the absence of an agent) or a control compound.

The term “reduce” or “inhibit” may relate generally to the ability of one or more antisense compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of muscular dystrophy, or reductions in the expression of defective forms of dystrophin, such as the altered forms of dystrophin that are expressed in individuals with DMD or BMD. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

Also included are vector delivery systems that are capable of expressing the oligomeric, dystrophin-targeting sequences of the present invention, such as vectors that express a polynucleotide sequence comprising any one or more of the sequences shown in Tables 3 and 4, and variants thereof, as described herein. By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

“Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In one embodiment, treatment with an antisense oligonucleotide of the invention increases novel dystrophin production and slows or reduces the loss of ambulation that would be expected without treatment. For example, treatment may stabilize, maintain, improve or increase walking ability (e.g., stabilization of ambulation) in the subject. In some embodiments, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) may be expressed as an absolute value, a percentage change or a change in the %-predicted value. In some embodiments, treatment maintains or improves a stable walking distance in a 6MWT from a 20% deficit in the subject relative to a healthy peer. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age²)+(132.28×height in meters). For females, the %-predicted 6MWD may be calculated using the following equation: 188.61+(51.50×age)−(1.86×age²)+(86.10×height in meters) (Henricson et al. PLOS Curr., 2012, version 2, herein incorporated by reference). In some embodiments, treatment with an antisense oligonucleotide increases the stable walking distance in the patient from baseline to greater than 3, 5, 6, 7, 8, 9, 10, 15, 20, 25, 30 or 50 meters (including all integers in between).

Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects. In some embodiments, normal growth and development can be accounted for using an age and height based equation fitted to normative data. Such an equation can be used to convert 6MWD to a percent-predicted (%-predicted) value in subjects with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLOS Curr., 2012, version 2, herein incorporated by reference).

A “subject,” as used herein, includes any animal that exhibits a symptom, or is at risk for exhibiting a symptom, which can be treated with an antisense compound of the invention, such as a subject that has or is at risk for having DMD or BMD, or any of the symptoms associated with these conditions (e.g., muscle fibre loss). Suitable subjects (patients) include laboratory animals (such as mouse, rat, rabbit, or guinea pig), farm animals, and domestic animals or pets (such as a cat or dog). Non-human primates and, preferably, human patients, are included.

A “pediatric patient” as used herein is a patient from age 1 to 21, inclusive.

“Alkyl” or “alkylene” both refer to a saturated straight or branched chain hydrocarbon radical containing from 1 to 18 carbons. Examples include without limitation methyl, ethyl, propyl, iso-propyl, butyl, iso-butyl, tert-butyl, n-pentyl and n-hexyl. The term “lower alkyl” refers to an alkyl group, as defined herein, containing between 1 and 8 carbons.

“Alkenyl” refers to an unsaturated straight or branched chain hydrocarbon radical containing from 2 to 18 carbons and comprising at least one carbon to carbon double bond. Examples include without limitation ethenyl, propenyl, iso-propenyl, butenyl, iso-butenyl, tert-butenyl, n-pentenyl and n-hexenyl. The term “lower alkenyl” refers to an alkenyl group, as defined herein, containing between 2 and 8 carbons.

“Alkynyl” refers to an unsaturated straight or branched chain hydrocarbon radical containing from 2 to 18 carbons comprising at least one carbon to carbon triple bond. Examples include without limitation ethynyl, propynyl, iso-propynyl, butynyl, iso-butynyl, tert-butynyl, pentynyl and hexynyl. The term “lower alkynyl” refers to an alkynyl group, as defined herein, containing between 2 and 8 carbons.

“Cycloalkyl” refers to a mono- or poly-cyclic alkyl radical. Examples include without limitation cyclobutyl, cyclopentyl, cyclohexyl, cycloheptyl and cyclooctyl.

“Aryl” refers to a cyclic aromatic hydrocarbon moiety containing from to 18 carbons having one or more closed ring(s). Examples include without limitation phenyl, benzyl, naphthyl, anthracenyl, phenanthracenyl and biphenyl.

“Aralkyl” refers to a radical of the formula RaRb where Ra is an alkylene chain as defined above and Rb is one or more aryl radicals as defined above, for example, benzyl, diphenylmethyl and the like.

“Thioalkoxy” refers to a radical of the formula —SRc where Rc is an alkyl radical as defined herein. The term “lower thioalkoxy” refers to an alkoxy group, as defined herein, containing between 1 and 8 carbons.

“Alkoxy” refers to a radical of the formula —ORda where Rd is an alkyl radical as defined herein. The term “lower alkoxy” refers to an alkoxy group, as defined herein, containing between 1 and 8 carbons. Examples of alkoxy groups include, without limitation, methoxy and ethoxy.

“Alkoxyalkyl” refers to an alkyl group substituted with an alkoxy group.

“Carbonyl” refers to the C(═O)-radical.

“Guanidynyl” refers to the H₂N(C═NH₂)—NH— radical.

“Amidinyl” refers to the H₂N(C═NH₂)CH— radical.

“Amino” refers to the NH₂ radical.

“Alkylamino” refers to a radical of the formula —NHRd or —NRdRd where each Rd is, independently, an alkyl radical as defined herein. The term “lower alkylamino” refers to an alkylamino group, as defined herein, containing between 1 and 8 carbons.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Thus, in addition to the heteroaryls listed below, heterocycles also include morpholinyl, pyrrolidinonyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydrothiophenyl, tetrahydrothiopyranyl, tetrahydropyrimidinyl, tetrahydrothiopyranyl, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

The terms “optionally substituted alkyl”, “optionally substituted alkenyl”, “optionally substituted alkoxy”, “optionally substituted thioalkoxy”, “optionally substituted alkyl amino”, “optionally substituted lower alkyl”, “optionally substituted lower alkenyl”, “optionally substituted lower alkoxy”, “optionally substituted lower thioalkoxy”, “optionally substituted lower alkyl amino” and “optionally substituted heterocyclyl” mean that, when substituted, at least one hydrogen atom is replaced with a substituent. In the case of an oxo substituent (═O) two hydrogen atoms are replaced. In this regard, substituents include: deuterium, optionally substituted alkyl, optionally substituted alkenyl, optionally substituted alkynyl, optionally substituted aryl, optionally substituted heterocycle, optionally substituted cycloalkyl, oxo, halogen, —CN, —ORx, NRxRy, NRxC(═O)Ry, NRxSO2Ry, —NRxC(═O)NRxRy, C(═O)Rx, C(═O)ORx, C(═O)NRxRy, —SOmRx and —SOmNRxRy, wherein m is 0, 1 or 2, Rx and Ry are the same or different and independently hydrogen, optionally substituted alkyl, optionally substituted alkenyl, optionally substituted alkynyl, optionally substituted aryl, optionally substituted heterocycle or optionally substituted cycloalkyl and each of said optionally substituted alkyl, optionally substituted alkenyl, optionally substituted alkynyl, optionally substituted aryl, optionally substituted heterocycle and optionally substituted cycloalkyl substituents may be further substituted with one or more of oxo, halogen, —CN, —ORx, NRxRy, NRxC(═O)Ry, NRxSO2Ry, —NRxC(═O)NRxRy, C(═O)Rx, C(═O)ORx, C(═O)NRxRy, —SOmRx and —SOmNRxRy.

An antisense molecule nomenclature system was proposed and published to distinguish between the different antisense molecules (see Mann et al., (2002) J Gen Med 4, 644-654). This nomenclature became especially relevant when testing several slightly different antisense molecules, all directed at the same target region, as shown below:
H#A/D(x:y).

The first letter designates the species (e.g. H: human, M: murine, C: canine). “#” designates target dystrophin exon number. “A/D” indicates acceptor or donor splice site at the beginning and end of the exon, respectively. (x y) represents the annealing coordinates where “−” or “+” indicate intronic or exonic sequences respectively. For example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

### II. CONSTRUCTING THE ANTISENSE OLIGONUCLEOTIDE

Exemplary embodiments of the invention relate to morpholino oligonucleotides having phosphorus-containing backbone linkages are illustrated in FIGS. 1A-1C. Preferred is a phosphorodiamidate-linked morpholino oligonucleotide such as shown in FIG. 1C, which is modified, in accordance with one aspect of the present invention, to contain positively charged groups at preferably 10%-50% of its backbone linkages. Morpholino oligonucleotides with uncharged backbone linkages, including antisense oligonucleotides, are detailed, for example, in (Summerton and Weller 1997) and in co-owned U.S. Pat. Nos. 5,698,685, 5,217,866, 5,142,047, 5,034,506, 5,166,315, 5,185, 444, 5,521,063, 5,506,337, 8,076,476, 8,299,206 and 7,943,762 all of which are expressly incorporated by reference herein.

Important properties of the morpholino-based subunits include: 1) the ability to be linked in a oligomeric form by stable, uncharged or positively charged backbone linkages; 2) the ability to support a nucleotide base (e.g. adenine, cytosine, guanine, thymidine, uracil and inosine) such that the polymer formed can hybridize with a complementary-base target nucleic acid, including target RNA, Tm values above about 45° C. in relatively short oligonucleotides (e.g., 10-15 bases); 3) the ability of the oligonucleotide to be actively or passively transported into mammalian cells; and 4) the ability of the antisense oligonucleotide:RNA heteroduplex to resist RNAse and RNase H degradation, respectively.

Exemplary backbone structures for antisense oligonucleotides of the claimed subject matter include the morpholino subunit types shown in FIGS. 1D-G, each linked by an uncharged or positively charged, phosphorus-containing subunit linkage. FIG. 1D shows a phosphorus-containing linkage which forms the five atom repeating-unit backbone, where the morpholino rings are linked by a 1-atom phosphoamide linkage. FIG. 1E shows a linkage which produces a 6-atom repeating-unit backbone. In this structure, the atom Y linking the 5′ morpholino carbon to the phosphorus group may be sulfur, nitrogen, carbon or, preferably, oxygen. The X moiety pendant from the phosphorus may be fluorine, an alkyl or substituted alkyl, an alkoxy or substituted alkoxy, a thioalkoxy or substituted thioalkoxy, or unsubstituted, monosubstituted, or disubstituted nitrogen, including cyclic structures, such as morpholines or piperidines. Alkyl, alkoxy and thioalkoxy preferably include 1-6 carbon atoms. The Z moieties are sulfur or oxygen, and are preferably oxygen.

The linkages shown in FIGS. 1F and 1G are designed for 7-atom unit-length backbones. In structure 1F, the X moiety is as in Structure 1E, and the Y moiety may be methylene, sulfur, or, preferably, oxygen. In Structure 1G, the X and Y moieties are as in Structure 1E. Particularly preferred morpholino oligonucleotides include those composed of morpholino subunit structures of the form shown in FIG. 1E, where X═NH₂, N(CH₃)₂, or 1-piperazine or other charged group, Y═O, and Z═O.

A substantially uncharged oligonucleotide may be modified, in accordance with an aspect of the invention, to include charged linkages, e.g., up to about 1 per every 2-5 uncharged linkages, such as about 4-5 per every 10 uncharged linkages. In certain embodiments, optimal improvement in antisense activity may be seen when about 25% of the backbone linkages are cationic. In certain embodiments, enhancement may be seen with a small number e.g., 10-20% cationic linkages, or where the number of cationic linkages are in the range 50-80%, such as about 60%.

Oligomers having any number of cationic linkages are provided, including fully cationic-linked oligomers. Preferably, however, the oligomers are partially charged, having, for example, 10%-80%. In preferred embodiments, about 10% to 60%, and preferably 20% to 50% of the linkages are cationic.

In one embodiment, the cationic linkages are interspersed along the backbone. The partially charged oligomers preferably contain at least two consecutive uncharged linkages; that is, the oligomer preferably does not have a strictly alternating pattern along its entire length.

Also considered are oligomers having blocks of cationic linkages and blocks of uncharged linkages; for example, a central block of uncharged linkages may be flanked by blocks of cationic linkages, or vice versa. In one embodiment, the oligomer has approximately equal-length 5′, 3′ and center regions, and the percentage of cationic linkages in the center region is greater than about 50%, preferably greater than about 70%.

In certain embodiments, the antisense compounds can be prepared by stepwise solid-phase synthesis, employing methods detailed in the references cited above, and below with respect to the synthesis of oligonucleotides having a mixture or uncharged and cationic backbone linkages. In some cases, it may be desirable to add additional chemical moieties to the antisense compound, e.g., to enhance pharmacokinetics or to facilitate capture or detection of the compound. Such a moiety may be covalently attached, according to standard synthetic methods. For example, addition of a polyethylene glycol moiety or other hydrophilic polymer, e.g., one having 1-100 monomeric subunits, may be useful in enhancing solubility.

A reporter moiety, such as fluorescein or a radiolabeled group, may be attached for purposes of detection. Alternatively, the reporter label attached to the oligomer may be a ligand, such as an antigen or biotin, capable of binding a labeled antibody or streptavidin. In selecting a moiety for attachment or modification of an antisense compound, it is generally of course desirable to select chemical compounds of groups that are biocompatible and likely to be tolerated by a subject without undesirable side effects.

Oligomers for use in antisense applications generally range in length from about 10 to about 50 subunits, more preferably about 10 to 30 subunits, and typically 15-25 bases. For example, an oligomer of the invention having 19-20 subunits, a useful length for an antisense compound, may ideally have two to ten, e.g., four to eight, cationic linkages, and the remainder uncharged linkages. An oligomer having 14-15 subunits may ideally have two to seven, e.g., 3, 4, or 5, cationic linkages and the remainder uncharged linkages. In a preferred embodiment, the oligomers have 25 to 28 subunits.

Each morpholino ring structure supports a base pairing moiety, to form a sequence of base pairing moieties which is typically designed to hybridize to a selected antisense target in a cell or in a subject being treated. The base pairing moiety may be a purine or pyrimidine found in native DNA or RNA (e.g., A, G, C, T or U) or an analog, such as hypoxanthine (the base component of the nucleoside inosine) or 5-methyl cytosine.

As noted above, certain embodiments are directed to oligomers comprising novel intersubunit linkages, including PMO-X oligomers and those having modified terminal groups. In some embodiments, these oligomers have higher affinity for DNA and RNA than do the corresponding unmodified oligomers and demonstrate improved cell delivery, potency, and/or tissue distribution properties compared to oligomers having other intersubunit linkages. The structural features and properties of the various linkage types and oligomers are described in more detail in the following discussion. The synthesis of these and related oligomers is described in co-owned U.S. application Ser. No. 13/118,298, which is incorporated by reference in its entirety.

In certain embodiments, the invention provides for an oligonucleotide having a sequence complementary to the target sequence which is associated with a human disease, and comprises a sequence of nucleotides having a formula:

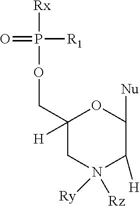

wherein Nu is a nucleobase;
R₁ has the formula

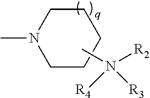

q is 0, 1, or 2;
R₂ is selected from the group consisting of hydrogen, C₁-C₅alkyl, C₁-C₅ aralkyl, and a formamidinyl group, and
R₃ is selected from the group consisting of hydrogen, C₁-C₁₀ acyl, C₁-C₁₀ aminoacyl, acyl moiety of a natural or unnatural alpha or beta amino acid, C₁-C₁₀ aralkyl, and C₁-C₁₀ alkyl, or
R₂ and R₃ are joined to form a 5-7 membered ring where the ring may be optionally substituted with a substituent selected from the group consisting of C₁-C₁₀ alkyl, phenyl, halogen, and C₁-C₁₀ aralkyl;
R₄ is selected from the group consisting of an electron pair, hydrogen, a C₁-C₆ alkyl and C₁-C₆ aralkyl;
Rx is selected from the group consisting of sarcosinamide, hydroxyl, a nucleotide, a cell penetrating peptide moiety, and piperazinyl;
Ry is selected from the group consisting of hydrogen, a C₁-C₆ alkyl, a nucleotide a cell penetrating peptide moiety, an amino acid, a formamidinyl group, and C₁-C₆ acyl; and,
Rz is selected from the group consisting of an electron pair, hydrogen, a C₁-C₆ alkyl, and C₁-C₆ acyl pharmaceutically acceptable salts thereof.
Nu may be selected from the group consisting of adenine, guanine, thymine, uracil, cytosine, and hypoxanthine. More preferably Nu is thymine or uracil.

In preferred embodiments, the invention provides an oligonucleotide having a sequence of nucleotides having a formula:

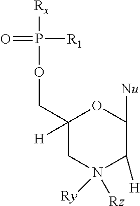

wherein Nu is a nucleobase;
R₁ is selected from the group consisting of R₁′ and R₁″ wherein R₁′ is dimethyl-amino and R₁″ has the formula

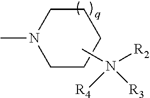

wherein at least one R₁ is R₁″;
q is 0, 1, or 2; with the proviso that at least one of R₁ is a piperidinyl moiety;
R₂ is selected from the group consisting of hydrogen, C₁-C₅ alkyl, C₁-C₅ aralkyl, and a formamidinyl group, and
R₃ is selected from the group consisting of hydrogen, C₁-C₁₀ acyl, C₁-C₁₀ aminoacyl, acyl moiety of a natural or unnatural alpha or beta amino acid, C₁-C₁₀ aralkyl, and C₁-C₁₀ alkyl, or
R₂ and R₃ are joined to form a 5-7 membered ring where the ring may be optionally substituted with a substituent selected from the group consisting of C₁-C₁₀ alkyl, phenyl, halogen, and C₁-C₁₀ aralkyl;
R₄ is selected from the group consisting of an electron pair, hydrogen, a C₁-C₆ alkyl and aralkyl;
Rx is selected from the group consisting of sarcosinamide, hydroxyl, a nucleotide, a cell penetrating peptide moiety, and piperazinyl;
Ry is selected from the group consisting of hydrogen, a C₁-C₆ alkyl, a nucleotide a cell penetrating peptide moiety, an amino acid, a formamidinyl group, and C₁-C₆ acyl; and,
Rz is selected from the group consisting of an electron pair, hydrogen, a C₁-C₆ alkyl, and C₁-C₆ acyl pharmaceutically acceptable salts thereof.
Nu may be selected from the group consisting of adenine, guanine, thymine, uracil, cytosine, and hypoxanthine. More preferably Nu is thymine or uracil.

About 90-50% of the R₁ groups are dimethylamino (i.e. R₁′). More, preferably, 90-50% of the R₁ groups are dimethylamino. Most, preferably about 66% of the R₁ groups are dimethylamino.

R₁″ may be selected from the group consisting of

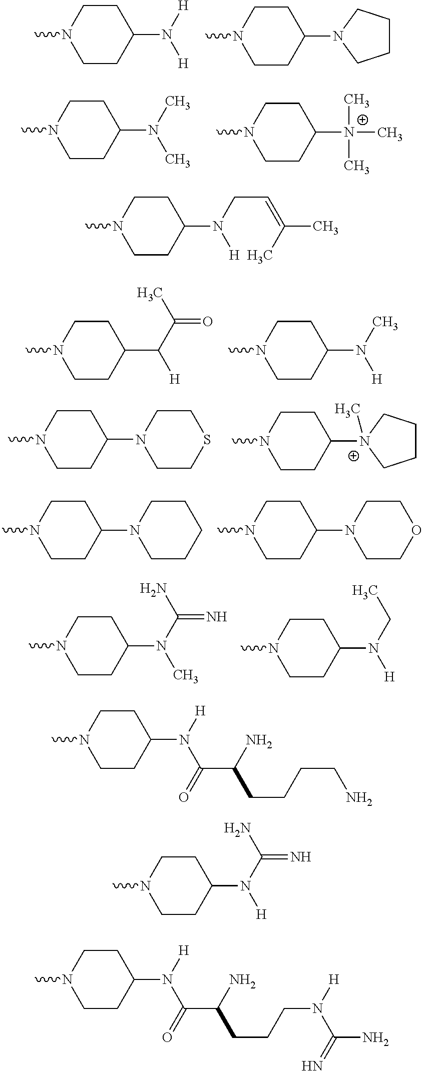

Preferably, at least one nucleotide of the oligonucleotide has the formula:

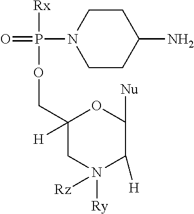

wherein Rx, Ry, Rz, and Nu are as stated above. Most preferably, Nu is thymine or uracil.

Although thymine (T) is the preferred base pairing moiety (Nu or Pi) containing the chemical modifications described above, any base subunit known to a person of skill in the art can be used as the base pairing moiety.

The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense molecule need not be 100% complementary to that of its target sequence to be specifically hybridizable. An antisense molecule is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

While the above method may be used to select antisense molecules capable of deleting any exon from within a protein that is capable of being shortened without affecting its biological function, the exon deletion should not lead to a reading frame shift in the shortened transcribed mRNA. Thus, if in a linear sequence of three exons the end of the first exon encodes two of three nucleotides in a codon and the next exon is deleted then the third exon in the linear sequence must start with a single nucleotide that is capable of completing the nucleotide triplet for a codon. If the third exon does not commence with a single nucleotide there will be a reading frame shift that would lead to the generation of truncated or a non-functional protein.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligonucleotides may need to be selected by the method of the invention wherein each is directed to a different region responsible for inducing splicing in the exons that are to be deleted.

The length of an antisense molecule may vary so long as it is capable of binding selectively to the intended location within the pre-mRNA molecule. The length of such sequences can be determined in accordance with selection procedures described herein. Generally, the antisense molecule will be from about 10 nucleotides in length up to about 50 nucleotides in length. It will be appreciated however that any length of nucleotides within this range may be used in the method. Preferably, the length of the antisense molecule is between 10-30 nucleotides in length.

The most common method for producing antisense molecules is the methylation of the 2′ hydroxyribose position and the incorporation of a phosphorothioate backbone produces molecules that superficially resemble RNA but that are much more resistant to nuclease degradation.

To avoid degradation of pre-mRNA during duplex formation with the antisense molecules, the antisense molecules used in the method may be adapted to minimize or prevent cleavage by endogenous RNase H. This property is highly preferred as the treatment of the RNA with the unmethylated oligonucleotides either intracellularly or in crude extracts that contain RNase H leads to degradation of the pre-mRNA: antisense oligonucleotide duplexes. Any form of modified antisense molecules that is capable of by-passing or not inducing such degradation may be used in the present method. An example of antisense molecules which when duplexed with RNA are not cleaved by cellular RNase H is 2′-O-methyl derivatives. 2′-O-methyl-oligoribonucleotides are very stable in a cellular environment and in animal tissues, and their duplexes with RNA have higher Tm values than their ribo- or deoxyribo-counterparts.

Antisense molecules that do not activate RNase H can be made in accordance with known techniques (see, e.g., U.S. Pat. No. 5,149,797). Such antisense molecules, which may be deoxyribonucleotide or ribonucleotide sequences, simply contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous antisense molecules that do not activate RNase H are available. For example, such antisense molecules may be oligonucleotides wherein at least one, or all, of the inter-nucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

While antisense oligonucleotides are a preferred form of the antisense molecules, the present invention comprehends other oligomeric antisense molecules, including but not limited to oligonucleotide mimetics such as are described below.

Specific examples of preferred antisense compounds useful in this invention include oligonucleotides containing modified backbones or non-natural inter-nucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their inter-nucleoside backbone can also be considered to be oligonucleosides.

In other preferred oligonucleotide mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleo-bases are retained and are bound directly or indirectly to aza nitrogen atoms of the amide portion of the backbone.

Modified oligonucleotides may also contain one or more substituted sugar moieties. Oligonucleotides may also include nucleobase (often referred to in the art simply as “base”) modifications or substitutions. Certain nucleo-bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of this invention, are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

### III. PEPTIDE TRANSPORTERS

The antisense compounds of the invention may include an oligonucleotide moiety conjugated to a CPP, preferably an arginine-rich peptide transport moiety effective to enhance transport of the compound into cells. The transport moiety is preferably attached to a terminus of the oligomer, as shown, for example, in FIGS. 1B and 1C. The peptides have the capability of inducing cell penetration within 30%, 40%, 50%, 60%, 70%, 80%, 90% or 100% of cells of a given cell culture population, including all integers in between, and allow macromolecular translocation within multiple tissues in vivo upon systemic administration. In one embodiment, the cell-penetrating peptide may be an arginine-rich peptide transporter. In another embodiment, the cell-penetrating peptide may be Penetratin or the Tat peptide. These peptides are well known in the art and are disclosed, for example, in US Publication No. 2010-0016215 A1, incorporated by reference in its entirety. A particularly preferred approach to conjugation of peptides to antisense oligonucleotides can be found in PCT publication WO2012/150960, which is incorporated by reference in its entirety. A preferred embodiment of a peptide conjugated oligonucleotide of the present invention utilizes glycine as the linker between the CPP and the antisense oligonucleotide. For example, a preferred peptide conjugated PMO consists of R₆-G-PMO.

The transport moieties as described above have been shown to greatly enhance cell entry of attached oligomers, relative to uptake of the oligomer in the absence of the attached transport moiety. Uptake is preferably enhanced at least ten fold, and more preferably twenty fold, relative to the unconjugated compound.

The use of arginine-rich peptide transporters (i.e., cell-penetrating peptides) are particularly useful in practicing the present invention. Certain peptide transporters have been shown to be highly effective at delivery of antisense compounds into primary cells including muscle cells (Marshall, Oda et al. 2007; Jearawiriyapaisarn, Moulton et al. 2008; Wu, Moulton et al. 2008). Furthermore, compared to other known peptide transporters such as Penetratin and the Tat peptide, the peptide transporters described herein, when conjugated to an antisense PMO, demonstrate an enhanced ability to alter splicing of several gene transcripts (Marshall, Oda et al. 2007).

Exemplary peptide transporters, excluding linkers are given below in Table 1.

TABLE 1
Exemplary peptide transporters

| NAME | | SEQ ID |
|---|---|---|
| (DESIGNATION) | SEQUENCE | NO^{A} |
| rTAT | RRRQRRKKR | 876 |
| Tat | RKKRRQRRR | 877 |
| R₉F₂ | RRRRRRRRRFF | 878 |
| R₅F₂R₄ | RRRRRFFRRRR | 879 |
| R₄ | RRRR | 880 |
| R₅ | RRRRR | 881 |
| R₆ | RRRRRR | 882 |
| R₇ | RRRRRRR | 883 |
| R₈ | RRRRRRRR | 884 |
| R₉ | RRRRRRRRR | 885 |
| (RX)₈ | RXRXRXRXRXRXRXRX | 886 |
| (RAhxR)₄; (P007) | RAhxRRAhxRRAhxRRAhxR | 887 |
| (RAhxR)₅; (CP04057) | RAhxRRAhxRRAhxRRAhxRRAhxR | 888 |
| (RAhxRRBR)₂; | RAhxRRBRRAhxRRBR | 889 |
| (CP06062) | | |
| (RAR)₄F₂ | RARRARRARRARFF | 890 |
| (RGR)₄F₂ | RGRRGRRGRRGRFF | 891 |

^{A}Sequences assigned to SEQ ID NOs do not include the linkage portion (e.g., C, G, P, Ahx, B, AhxB where Ahx and B refer to 6-aminohexanoic acid and beta-alanine, respectively).

### IV. FORMULATIONS AND TREATMENT

In certain embodiments, the present invention provides formulations or compositions suitable for the therapeutic delivery of antisense oligomers, as described herein. Hence, in certain embodiments, the present invention provides pharmaceutically acceptable compositions that comprise a therapeutically-effective amount of one or more of the oligomers described herein, formulated together with one or more pharmaceutically acceptable carriers (additives) and/or diluents. While it is possible for an oligomer of the present invention to be administered alone, it is preferable to administer the compound as a pharmaceutical formulation (composition).

The compositions of the present invention may be administered alone or in combination with another therapeutic. The additional therapeutic may be administered prior, concurrently or subsequently to the administration of the composition of the present invention. For example, the compositions may be administered in combination with a steroid and/or an antibiotic. The steroid may be a glucocorticoid or prednisone. Glucocorticoids such as cortisol control carbohydrate, fat and protein metabolism, and are anti-inflammatory by preventing phospholipid release, decreasing eosinophil action and a number of other mechanisms. Mineralocorticoids such as aldosterone control electrolyte and water levels, mainly by promoting sodium retention in the kidney. Corticosteroids are a class of chemicals that includes steroid hormones naturally produced in the adrenal cortex of vertebrates and analogues of these hormones that are synthesized in laboratories. Corticosteroids are involved in a wide range of physiological processes, including stress response, immune response, and regulation of inflammation, carbohydrate metabolism, protein catabolism, blood electrolyte levels, and behavior. Corticosteroids include Betamethasone, Budesonide, Cortisone, Dexamethasone, Hydrocortisone, Methylprednisolone. Prednisolone, and Prednisone.

Other agents which can be administered include an antagonist of the ryanodine receptor, such as dantrolene, which has been shown to enhance antisense-mediated exon skipping in patient cells and a mouse model of DMD (G. Kendall et al. Sci Tranl Med 4 164ra160 (2012), incorporated herein by reference).

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including the isolated oligomers of the present invention.

As detailed below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligomers of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly(DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The invention also features the use of the composition comprising surface-modified liposomes containing poly(ethylene glycol) lipids (PEG-modified, branched and unbranched or combinations thereof, or long-circulating liposomes or stealth liposomes). Oligomers of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present invention includes oligomer compositions prepared for delivery as described in U.S. Pat. Nos. 6,692,911, 7,163,695 and 7,070,807. In this regard, in one embodiment, the present invention provides an oligomer of the present invention in a composition comprising copolymers of lysine and histidine (HK) (as described in U.S. Pat. Nos. 7,163,695, 7,070,807, and 6,692,911) either alone or in combination with PEG (e.g., branched or unbranched PEG or a mixture of both), in combination with PEG and a targeting moiety or any of the foregoing in combination with a crosslinking agent. In certain embodiments, the present invention provides antisense oligomers in compositions comprising gluconic-acid-modified polyhistidine or gluconylated-polyhistidine/transferrin-polylysine. One skilled in the art will also recognize that amino acids with properties similar to His and Lys may be substituted within the composition.

Certain embodiments of the oligomers described herein may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject oligomers include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the oligomers of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from cyclodextrins, celluloses, liposomes, micelle forming agents, e.g., bile acids, and polymeric carriers, e.g., polyesters and polyanhydrides; and an oligomer of the present invention. In certain embodiments, an aforementioned formulation renders orally bioavailable an oligomer of the present invention.

Methods of preparing these formulations or compositions include the step of bringing into association an oligomer of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. An oligomer of the present invention may also be administered as a bolus, electuary or paste.

In solid dosage forms of the invention for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The active oligomers may be mixed under sterile conditions with a pharmaceutically-acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an oligomer of the present invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligomer of the present invention to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more oligomers of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject oligomers in biodegradable polymers such as polylactide-polyglycolide. Depending on the ratio of oligomer to polymer, and the nature of the particular polymer employed, the rate of oligomer release can be controlled. Examples of other biodegradable polymers include poly(orthoesters) and poly(anhydrides). Depot injectable formulations may also prepared by entrapping the drug in liposomes or microemulsions that are compatible with body tissues.

When the oligomers of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

As noted above, the formulations or preparations of the present invention may be given orally, parenterally, systemically, topically, rectally or intramuscular administration. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the oligomers of the present invention, which may be used in a suitable hydrated form, and/or the pharmaceutical compositions of the present invention, may be formulated into pharmaceutically-acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligomer of the present invention employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular, intramuscular and subcutaneous doses of the compounds of this invention for a patient, when used for the indicated effects, will range from about 0.0001 to about 100 mg per kilogram of body weight per day.

Preferred doses of the oligomers of the present invention (e.g., phosphorodiamidate morpholino oligomers; eteplirsen) are administered generally from about 20-100 mg/kg. In some cases, doses of greater than 100 mg/kg may be necessary. For i.v. administration, preferred doses are from about 0.5 mg to 100 mg/kg. In some embodiments, the oligomers are administered at doses of about 20 mg/kg, 21 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 65 mg/kg, 70 mg/kg, 75 mg/kg, 80 mg/kg, 85 mg/kg, 90 mg/kg, 95 mg/kg, 100 mg/kg, including all integers in between. In a preferred embodiment, the oligomer is administered at 30 mg/kg. In another preferred embodiment, the oligomer is administered at 50 mg/kg.

If desired, the effective daily dose of the active compound may be administered as two, three, four, five, six or more sub-doses administered separately at appropriate intervals throughout the day, optionally, in unit dosage forms. In certain situations, dosing is one administration per day. In certain embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired expression of a functional dystrophin protein.

In some embodiments, the oligomers of the present invention (e.g., phosphorodiamidate morpholino oligomers; eteplirsen) are administered, generally at regular intervals (e.g., daily, weekly, biweekly, monthly, bimonthly). The oligomers may be administered at regular intervals, e.g., daily; once every two days; once every three days; once every 3 to 7 days; once every 3 to 10 days; once every 7 to 10 days; once every week; once every two weeks; once monthly. For example, the oligomers may be administered once weekly by intravenous infusion. The oligomers may be administered intermittently over a longer period of time, e.g., for several weeks, months or years. For example, the oligomers may be administered once every one, two, three, four, five, six, seven, eight, nine, ten, eleven or twelve months. In addition, the oligomers may be administered once every one, two, three, four or five years. Administration may be followed by, or concurrent with, administration of an antibiotic, steroid or other therapeutic agent. The treatment regimen may be adjusted (dose, frequency, route, etc.) as indicated, based on the results of immunoassays, other biochemical tests and physiological examination of the subject under treatment.

Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17 (12), 1685-1713, 1991 and REV 5901 (Sheen, P. C., et al., J Pharm Sci 80 (7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from an oligomer as provided herein and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG (750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by a-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17a-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present invention relates to formulations comprising liposomes containing an oligomer of the present invention, where the liposome membrane is formulated to provide a liposome with increased carrying capacity. Alternatively or in addition, the compound of the present invention may be contained within, or adsorbed onto, the liposome bilayer of the liposome. An oligomer of the present invention may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the present invention.

Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D D, Liposomes from physics to applications, Elsevier Science Publishers BV, Amsterdam, 1993. For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An oligomer may be formulated to be contained within, or, adapted to release by a surgical or medical device or implant. In certain aspects, an implant may be coated or otherwise treated with an oligomer. For example, hydrogels, or other polymers, such as biocompatible and/or biodegradable polymers, may be used to coat an implant with the compositions of the present invention (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the oligomers for use according to the invention may be formulated for administration in any convenient way for use in human or veterinary medicine, by analogy with other pharmaceuticals. The antisense oligomers and their corresponding formulations may be administered alone or in combination with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (e.g., adeno-associated viral vectors) (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

### V. KITS

The invention also provides kits for treatment of a patient with a genetic disease which kit comprises at least an antisense molecule (e.g., one or more antisense oligonucleotides capable of specifically hybridizing to any one or more of exons 1-79 of the dystrophin gene; for example, Exon 51 as set forth in Tables 3 and 4 herein), packaged in a suitable container, together with instructions for its use. The kits may also contain peripheral reagents such as buffers, stabilizers, etc. Those of ordinary skill in the field should appreciate that applications of the above method has wide application for identifying antisense molecules suitable for use in the treatment of many other diseases.

### VII. EXAMPLES

### Materials and Methods

### Patients

Eligible patients were between 7 and 13 years of age (inclusive), with out-of-frame deletions of the DMD gene that could be corrected by skipping exon 51. Patients were confirmed to have stable cardiac and pulmonary function and a stable dose of glucocorticoids for at least 24 weeks prior to enrollment. Only patients who could walk between 200 and 400 meters (+10%) on the 6-Minute Walk Test (6MWT) at baseline were enrolled.

### Study Design

This one-year trial was conducted in two phases: (1) treatment was double-blind through week 24 and (2) open-label thereafter. Primary endpoints were change in percent dystrophin fibers and ambulation as measured by the 6-Minute Walk Test (6MWT).

Study 201 was a single-site, randomized, double-blind, placebo-controlled, multiple-dose efficacy, safety and tolerability trial of eteplirsen. Twelve patients with DMD were randomized to one of three groups: eteplirsen 30 mg/kg/week (Cohort 1); eteplirsen 50 mg/kg/week (Cohort 2); or placebo/delayed eteplirsen (Cohort 3). All patients received weekly intravenous eteplirsen or placebo/delayed eteplirsen dosing. Placebo-treated patients crossed over to weekly eteplirsen 30 (n=2) or 50 mg/kg (n=2) at week 25. Efficacy and safety were assessed at scheduled visits, and an independent Data Safety Monitoring Board ensured the welfare of all patients. All patients had bicep biopsies at baseline. Follow-up biopsies were performed in the opposite arm (biceps) at week 12 for the 50 mg/kg group and two placebo-treated patients and at week 24 for the 30 mg/kg group and two placebo-treated patients.

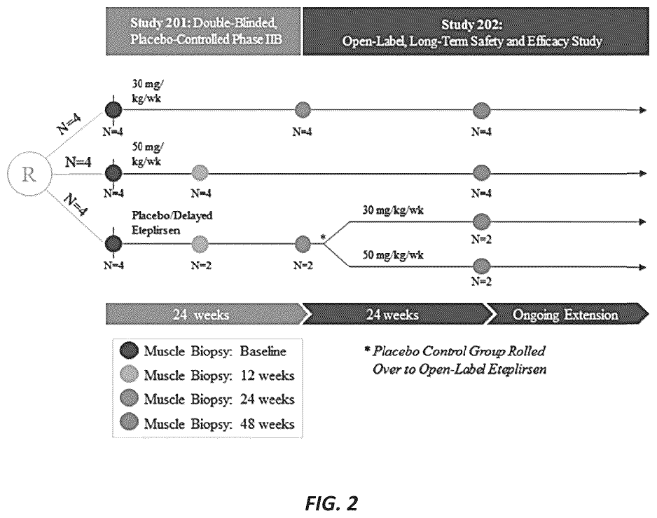

Patients continued weekly dosing with 30 or 50 mg/kg eteplirsen under Study 202, a long-term, open-label extension study. All efficacy assessments continued to be performed during Study 202, including a third biopsy (in the left deltoid muscle) in all patients at week 48. Monitoring of adverse events continued throughout the study. A schematic of the study design is shown in FIG. 2.

### Study Drug

Eteplirsen [sequence 5′-CTCCAACATCAAGGAAGATGGCATTTCTAG-3′] (SEQ ID NO: 1) was supplied by Sarepta Therapeutics, Inc. in single-use vials of phosphate-buffered saline (100 mg/ml). Eteplirsen was reconstituted with 150 ml normal saline and infused over 60 minutes. Placebo, administered during the first 24 weeks of Study 201, was supplied as identical vials of phosphate-buffered saline and was administered in the same manner as eteplirsen.

### Safety and Tolerability Monitoring

Safety was assessed by evaluation of adverse events, vital signs, physical examinations, electrocardiograms, echocardiograms, and clinical laboratory testing. In addition, kidney function was monitored via regular assessments of serum cystatin C and urine cystatin C and KIM-1.

### Pharmacokinetic and Immune Assessments

Pharmacokinetic parameters of eteplirsen were established from plasma and urine taken after the twelfth dose using a validated and sensitive anion exchange high-performance liquid chromatography with fluorescence detection bioanalytical method. Single samples for analysis of plasma concentrations were taken at weeks 24, 25, and 36. Immune response to novel dystrophin protein was measured every six weeks through week 24 with ELISPOT following methods previously published.

### Biochemical Efficacy Assessments

Pre- and post-treatment dystrophin expression studies were based on MANDYS106 [a gift from Glen Morris, MDA Monoclonal Antibody Library], a highly sensitive marker for dystrophin used in prior studies of eteplirsen and other exon skipping candidates. Three 10 μm frozen sections, separated by at least 200 μm, were stained with MANDYS106, followed by a secondary antibody (Alexa Fluor 594 goat antimouse antibody). Percent dystrophin-positive fibers were calculated by dividing the number of positive fibers by the total fibers counted. As normal muscle samples have 100% dystrophin-positive fibers, percent dystrophin-positive fibers is expressed as a percentage of normal. The same antibody-stained sections were used for dystrophin quantification using Bioquant image analysis software. The total dystrophin fluorescence signal intensity was reported as a percentage of normal.

Supportive measurements included expression of the components of the sarcoglycan complex (β,γ), neuronal NOS, and Western blot (with the anti-dystrophin antibody NCL-Dys1 from Novacastra). RT-PCR analysis, for confirmation of exon skipping, was performed on 400 ng of total RNA using dystrophin-specific reverse primers as previously described.

### Clinical Efficacy Assessments

The 6MWT was administered using the protocol established for patients with DMD by McDonald, et al. (Muscle Nerve, 2010; 42:966-74, herein incorporated by reference). Exploratory functional outcomes included the North Star Ambulatory Assessment, quantitative muscle testing, the 9-Hole Peg Test, pulmonary function testing (PFT), timed function tests, and assessment of quality of life.

### Statistical Analysis

SAS version 9.3 (Cary, NC) was used for all statistical analyses. Mixed model with treatment as fixed effect, subject nested within treatment as random effect, with the baseline value and time since DMD diagnosis as covariates for the analysis of muscle biopsy data was used. Mixed model repeated measures (MMRM) with treatment, time, and treatment-by-time interaction terms as fixed effect, subject nested within treatment as random effect, and with the baseline value and time since DMD diagnosis as covariates for analysis of the 6MWT data was used. Safety and muscle biopsy analyses were performed on the intent-to-treat population; analysis of ambulation-related outcomes, including the 6MWT, used a modified intent-to-treat (mITT) population that excluded two patients in Cohort 1 who showed signs of disease progression and significant decline on the 6MWT within weeks of enrollment and could not perform measures of ambulation at week 24 or beyond.

### Example 1: Subject Characteristics

Baseline characteristics of the 12 patients in this study are summarized in Table 2. Five different genotypes amenable to exon 51 skipping were represented in the study population. Mean distances on the 6-Minute Walk Test (6MWT) at baseline were similar to those in other studies of children with DMD, and as expected, were well below the 600 plus meters typically observed in age-matched healthy children. Due to the stochastic nature of the sampling, the 30 mg/kg cohort was slightly older, heavier, and taller, relative to the other cohorts, and had a lower mean 6MWT distance at baseline. All patients received all infusions of study medication as planned and completed all assessments.

TABLE 2
Baseline Demography and Disease Characteristics

| | | | |
|---|---|---|---|
| | Placebo/ | Eteplirsen | Eteplirsen |
| | Delayed Eteplirsen | 30 mg/kg | 50 mg/kg |
| Treatment Arm | N = 4 | N = 4 | N = 4 |

Mutation

| | | | |
|---|---|---|---|
| 45-50 n(%) | 0 | 2 (50) | 1 (25) |
| 48-50 n(%) | 0 | 1 (25) | 0 |
| 49-50 n(%) | 3 (75) | 0 | 2 (50) |
| 50 n(%) | 1 (25) | 0 | 0 |
| 52 n(%) | 0 | 1 (25) | 1 (25) |

Gender n(%)

| | | | |
|---|---|---|---|
| Male | 4 (100) | 4 (100) | 4 (100) |

Age, years

| | | | |
|---|---|---|---|
| Mean | 8.5 | 9.3 | 8.5 |
| SD | 1.73 | 0.50 | 1.29 |
| Min, Max | 7, 10 | 9, 10 | 7, 10 |

Height, cm

| | | | |
|---|---|---|---|
| Mean | 119.3 | 130.5 | 121.3 |
| SD | 3.40 | 9.47 | 7.85 |
| Min, Max | 116, 124 | 117, 138 | 117, 133 |

Weight, kg

| | | | |
|---|---|---|---|
| Mean | 30.6 | 34.8 | 29.0 |
| SD | 6.04 | 7.05 | 6.38 |
| Min, Max | 22.1, 36.2 | 24.8, 39.8 | 23.7, 38.3 |

Race, n(%)

| | | | |
|---|---|---|---|
| Asian | 0 | 1 (25) | 0 |
| White | 4 (100) | 3 (75) | 4 (100) |

6MWT*, meters

| | | | |
|---|---|---|---|
| Mean | 394.5 | 355.3 | 396.0 |
| SD | 42.25 | 74.78 | 26.61 |
| Min, Max | 364, 456 | 261, 442 | 365, 429 |

*6MWT results are the maximum observed value of two tests administered on two consecutive days during screening.
Abbreviations: 6MWT = 6-Minute Walk Test; min = minimum; max = maximum; SD = standard deviation.

### Example 2: Safety and Lack of Adverse Events

Eteplirsen was well tolerated with no treatment-related adverse events, serious adverse events, discontinuations or missed doses through 48 weeks of treatment. Moreover, no clinically significant changes were observed on physical examination or in vital signs. Electrocardiograms, echocardiograms, and PFTs remained stable, and chemistries showed no clinically significant changes in hematologic, renal, coagulation or liver functions. Mild and transient proteinuria was observed in a single placebo-treated subject.

### Example 3: Pharmacokinetic Profile

Analysis of PK parameters at week 12 revealed rapid absorption. Plasma clearance averaged 339±75.8 mL/hr/kg for 30 mg/kg and 319±125 mL/hr/kg for 50 mg/kg. Half-life averaged 3.30±0.341 hr for 30 mg/kg and 3.17±0.249 hr for 50 mg/kg, with renal clearance accounting for approximately 65-70% of total systemic clearance.

### Example 4: Efficacy

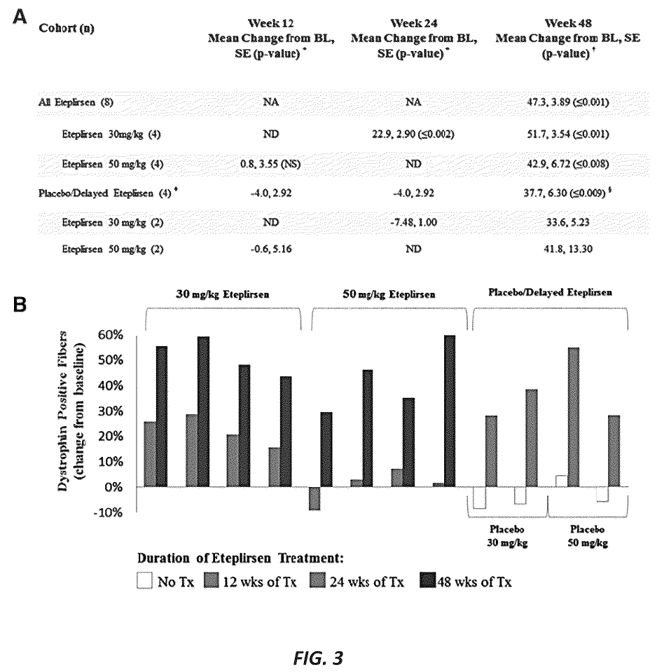

At week 48, eteplirsen produced robust increases in the number and intensity of dystrophin-positive fibers. As shown in FIG. 3, patients who received 30 or 50 mg/kg eteplirsen without interruption for 48 weeks showed a mean increase in the percentage of dystrophin-positive fibers to 47% of normal (p≤0.001), relative to baseline. Increases were similar when the 30 (52%; p≤0.001) and 50 (43%; p≤0.008) mg/kg cohorts were analyzed separately, suggesting that eteplirsen's effect on the production of novel dystrophin is independent of dose within this range of doses.

Biopsies were taken at staggered time points (see FIG. 2) to evaluate the impact of treatment duration on novel dystrophin production. At week 12, the 50 mg/kg cohort had undetectable levels of novel dystrophin. At week 24, the 30 mg/kg cohort demonstrated an increase in the percentage of dystrophin-positive fibers to 23% of normal (p≤0.002), and at week 48, after 24 weeks of treatment with 30 or 50 mg/kg eteplirsen, the 4 patients in the placebo/delayed eteplirsen cohort showed an increase to 38% of normal, relative to baseline (p≤0.009). Together these data suggest that treatment duration plays an important role in eteplirsen's ability to uniformly restore novel dystrophin production. Consistent with these findings, eteplirsen also significantly increased mean fluorescence signal intensity at week 48 in all three treatment groups (all p-values≤0.023).

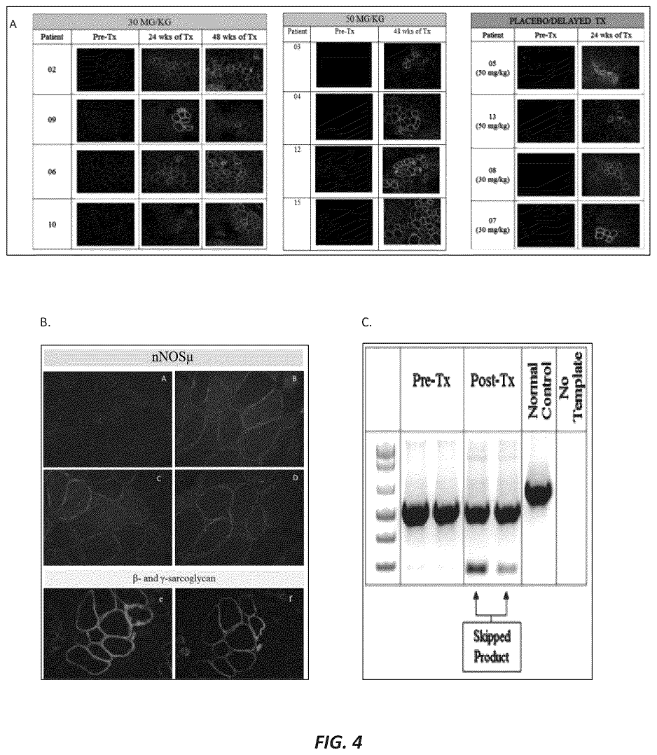

FIG. 4 illustrates eteplirsen's time-dependent effect on the percentage of dystrophin-positive fibers (Panel A), which was accompanied by restoration of β- and γ-sarcoglycan and nNOSμ at the sarcolemma (Panel B). Dystrophin expression and exon skipping were confirmed by Western blot and RT-PCR in all patients. RT-PCR results from a representative patient are shown in Panel C. These data confirmed the increase in functional dystrophin in the patients.

### Example 5: Functional Outcomes

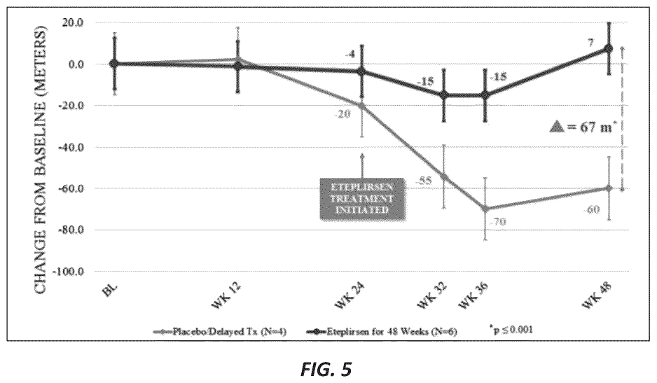

The progressive loss of walking ability is a universal hallmark of DMD, with most patients showing functional compromise by 7 or 8 years of age and becoming wheelchair dependent by 10 to 14 years of age. Consistent with this, boys assigned to the placebo/delayed eteplirsen cohort in this study showed a decline in walking ability after week 12 at a rate predicted by prior studies, culminating in a loss of approximately 60 meters by week 48 (FIG. 5). In marked contrast, eteplirsen-treated patients maintained a stable walking distance over the duration of the study, with a mean increase from baseline of about 7 meters by week 48. The difference between the eteplirsen-treated patients and those in the placebo/delayed eteplirsen cohort first became statistically significant at week 32 (39-meter difference; p≤0.05). Interestingly, patients in the placebo/delayed eteplirsen cohort appeared to stabilize after week 36, i.e., between 12 and 24 weeks after initiating treatment with eteplirsen at week 25. As previously noted, two boys who showed signs of rapid disease progression and significant decline on the 6MWT within weeks of enrollment and were unable to perform measures of ambulation at 24 weeks or beyond, were excluded from this analysis. However, both remained on eteplirsen through week 48 with no treatment-related adverse events and maintained stable pulmonary and upper limb function as measured by PFT and the 9-Hole Peg Test, respectively.

Notably, patients receiving eteplirsen for 48 weeks, evaluable on the 6MWT (n=6), significantly (p≤0.001) improved on the 6MWT (67.3m) compared to the placebo/delayed cohort.

Both cohorts have maintained stable 6MWT results over 120 weeks as described below. At 120 weeks, patients in the 30 mg/kg and 50 mg/kg eteplirsen cohorts who were able to perform the 6MWT (modified Intent-to-Treat or mITT population; n=6) experienced a general stability with a slight decline of 13.9 meters, or less than 5 percent, from baseline in walking ability. A statistically significant treatment benefit of 64.9 meters (p≤0.006) was observed for the mITT population compared with the placebo/delayed-treatment cohort (n=4), which initiated treatment at Week 25 following 24 weeks of placebo. After experiencing a substantial decline earlier in the study (prior to treatment with etiplersen), the placebo/delayed-treatment cohort also demonstrated stabilization in walking ability for more than 1.5 years, from Week 36 through 120, the period from which meaningful levels of dystrophin were likely produced, with a decline of 9.5 meters over this timeframe. These analyses were based on the maximum 6MWT score when the test was performed on two consecutive days.

### Example 6: Immune Response

There were no differences between the eteplirsen- and placebo-treated patients in the number of interferon-y-induced spot forming colonies to dystrophin peptide pools (extended over the entire protein) at any time point assessed, including week 24, indicating that the newly expressed dystrophin in the eteplirsen-treated patients did not elicit a T-cell response.

### Example 7: Pulmonary Function

Respiratory muscle function from baseline through Week 120 in both dosing cohorts, as measured by maximum inspiratory and expiratory pressure (MIP and MEP), showed a 14.6 percent mean increase in MIP and a 15.0 percent mean increase in MEP. Analyses of MIP percent predicted (MIP adjusted for weight) and MEP percent predicted (MEP adjusted for age) demonstrated a mean increase from 90.2 percent at baseline to 95.2 percent at Week 120 in MIP percent predicted, and a slight mean increase from 79.3 percent at baseline to 79.6 percent at Week 120 in MEP percent predicted. In addition, there was a mean increase in forced vital capacity (FVC), a measure of lung volume, of 8.7 percent from baseline to Week 120, and FVC percent predicted (FVC adjusted for age and height) was maintained above a mean of 90 percent through Week 120, with 101 percent at baseline and 93 percent at Week 120.

All publications and patent applications cited in this specification are herein incorporated by reference as if each individual publication or patent application were specifically and individually indicated to be incorporated by reference.

### REFERENCES

1. Emery A E H. Population frequencies of inherited neuromuscular diseases-a world survey. Neuromuscul Disord 1991; 1:19-29.
2. Mendell J R, Shilling C, Leslie N D, Flanigan K M, al-Dahhak R, Gastier-Foster, J, et al. Evidence-based path to newborn screening for Duchenne muscular dystrophy. Ann Neurol 2012; 71:304-13.
3. McDonald C M, Abresch R T, Carter G T, Fowler W M Jr, Johnson E R, Kilmer D D, et al. Profiles of neuromuscular diseases. Duchenne muscular dystrophy. Am J Phys Med Rehabil 1995; 74: S70-S92.
4. Bushby K, Finkel R, Birnkrant D J, Case L E, Clemens P R, Cripe L, et al. Diagnosis and management of Duchenne muscular dystrophy, part 1: diagnosis, and pharmacological and psychosocial management. Lancet Neurol 2010; 9:77-93.
5. Kohler M, Clarenbach C F, Böni L, Brack T, Russi E W, Bloch K E. Quality of life, physical disability and respiratory impairment in Duchenne muscular dystrophy. Am J Respir Crit Care Med 2005; 172:1032-6.
6. Mendell J R, Moxley R T, Griggs R C, Brooke M H, Fenichel G M, Miller J P, et al. Randomized, double-blind six-month trial of prednisone in Duchenne's muscular dystrophy. N Engl J Med 1989; 320:1592-97.
7. Manzur A Y, Kuntzer T, Pike M, Swan A. Glucocorticoid corticosteroids for Duchenne muscular dystrophy. Cochrane Database Syst Rev. 2004; (2): CD003725.
8. van Deutekom J C, Janson A A, Ginjaar I B, Frankhuizen W S, Aartsma-Rus A, Bremmer-Bout M, et al. Local dystrophin restoration with antisense oligonucleotide PRO051. N Engl J Med 2007; 357:2677-86.
9. Kinali M, Arechavala-Gomeza V, Feng L, Cirak S, Hunt D, Adkin C, et al. Local restoration of dystrophin expression with the morpholino oligomer AVI-4658 in Duchenne muscular dystrophy: a single-blind, placebo-controlled, dose-escalation, proof-of-concept study. Lancet Neurol 2009; 8:918-28.
10. Goemans N M, Tulinius M, van den Akker J T, Burm B E, Ekhart P F, Heuvelmans N, et al. Systemic administration of PRO051 in Duchenne's muscular dystrophy. N Engl J Med 2011; 364:1513-22.
11. Cirak S, Arechavala-Gomeza V, Guglieri M, Feng L, Torelli S, Anthony K, et al. Exon skipping and dystrophin restoration in patients with Duchenne muscular dystrophy after systemic phosphorodiamidate morpholino oligomer treatment: an open-label, phase 2, dose-escalation study. Lancet 2011; 378:595-605.
12. Aartsma-Rus A, Fokkema I, Verschuuren J, Ginjaar I, van Deutekom J, van Ommen G J, et al. Theoretic applicability of antisense-mediated exon skipping for Duchenne muscular dystrophy mutations. Hum Mutat 2009; 30:293-99.
13. Muntoni F, Torelli S, Ferlini A. Dystrophin and mutations: one gene, several proteins, multiple phenotypes. Lancet Neurol. 2003; 2:731-40.
14. Bushby K M, Gardner-Medwin D. The clinical, genetic and dystrophin characteristics of Becker muscular dystrophy. I. Natural history. J Neurol 1993; 240:98-104.
15. Arechavala-Gomeza V, Graham I R, Popplewell L J, Adams A M, Aartsma-Rus A, Kinali M, et al. Comparative analysis of antisense oligonucleotide sequences for targeted skipping of exon 51 during dystrophin pre-mRNA splicing in human muscle. Hum Gene Ther 2007; 18:798-810.
16. Mendell J R, Campbell K, Rodino-Klapac L, Sahenk Z, Shilling C, Lewis S, et al. Dystrophin immunity revealed by gene therapy in Duchenne muscular dystrophy. N Engl J Med 2010; 363:1429-37.
17. Nguyen T M, Morris G E. Use of epitope libraries to identify exon-specific monoclonal antibodies for characterization of altered dystrophins in muscular dystrophy. Am J Hum Genet 1993; 52:1057-66.
18. Arechavala-Gomeza V, Kinali M, Feng L, Brown S C, Sewry C, Morgan J E, et al. Immunohistological intensity measurements as a tool to assess sarcolemma-associated protein expression. Neuropathol Appl Neurobiol 2010; 36:265-74.
19. McDonald C M, Henricson E K, Han J J, Abresch R T, Nicorici A, Elfring G L, et al. The 6-minute walk test as a new outcome measure in Duchenne muscular dystrophy. Muscle Nerve 2010; 41:500-10.
20. Mazzone E, Vasco G, Sormani M P, Torrente Y, Berardinelli A, Messina S, et al. Functional changes in Duchenne muscular dystrophy: a 12-month longitudinal cohort study. Neurology 2011; 77 (3): 250-6.
21. McDonald C M, Henricson E K, Han J J, Abresch R T, Nicorici A, Atkinson L, et al. The 6-minute walk test in Duchenne/Becker muscular dystrophy: longitudinal observations. Muscle Nerve 2010; 42:966-74.
22. Strober J B. Therapeutics in Duchenne muscular dystrophy. NeuroRX 2006; 3:225-34.
23. Hoffman E P, Fischbeck K H, Brown R H, Johnson M, Medori R, Loike J D, et al. Characterization of dystrophin in muscle-biopsy specimens from patients with Duchenne's or Becker's muscular dystrophy. N Engl J Med 1988; 318:1363-68.
24. Azofeifa J, Voit T, Hubner C, Cremer M. X-chromosome methylation in manifesting and healthy carriers of dystrophinopathies: concordance of activation ratios among first degree female relatives and skewed inactivation as cause of the affected phenotypes. Hum Genet 1995; 96:167-76.
25. van Putten M, Hulsker M, Nadarajah V D, van Heiningen S H, van Huizen E, van Iterson M, et al. The Effects of Low Levels of Dystrophin on Mouse Muscle Function and Pathology. PLOS ONE 2012; 7: e31937.
26. Brooke M H, Fenichel G M, Griggs R C, Mendell J R, Moxley R, Miller J P, et al. Clinical investigation in Duchenne dystrophy: 2. Determination of the “power” of therapeutic trials based on the natural history. Muscle Nerve. 1983; 6:91-103.
27. Ahmad A, Brinson M, Hodges B L, Chamberlain J S, Amalfitano A. Mdx mice inducibly expressing dystrophin provide insights into the potential of gene therapy for Duchenne muscular dystrophy. Hum Mol Genet 2000; 9:2507-15.
28. Hoffman E P, Bronson A, Levin A A, Takeda S, Yokota T, Baudy A R, Connor E M. Restoring dystrophin expression in Duchenne muscular dystrophy muscle: Progress in exon skipping and stop codon read through. Am J Path 2011; 179:12-22.
29. Merlini L, Gennari M, Malaspina E, Cecconi I, Armaroli A, Gnudi S, et al. Early corticosteroid treatment in 4 Duchenne muscular dystrophy patients: 14-year follow-up. Muscle Nerve 2012; 45:796-802.
30. Fletcher S, Honeyman K, Fall A M, Harding P L, Johnsen R D, Steinhaus J P, et al. Morpholino oligomer-mediated exon skipping averts the onset of dystrophic pathology in the mdx mouse. Mol Ther 2007; 15:1587-92.
31. Yokota T, Lu Q L, Partridge T, Kobayashi M, Nakamura A, Takeda S, et al. Efficacy of systemic morpholino exon-skipping in Duchenne dystrophy dogs. Ann Neurol 2009; 65:667-76.
32. Aartsma-Rus A, Janson A A, Kaman W E, Bremmer-Bout M, van Ommen G J, den Dunnen J T, et al. Antisense-induced multiexon skipping for Duchenne muscular dystrophy makes more sense. Am J Hum Genet 2004; 74:83-92.

### SEQUENCE LISTING

With respect to the nucleic acid sequences provided in the application, persons skilled in the art will appreciate that depending on the use of the oligomers, Ts and Us are interchangeable.

TABLE 3

| | SEQ | | NUCLEOTIDE SEQUENCE |
|---|---|---|---|
| EXON | ID | SEQUENCE | (5′-3′) |
| 51 | 1000 | eteplirsen | CTC CAA CAT CAA |
| | | H51A(+66+95) | GGA AGA TGG CAT |
| | | | TTC TAG |
| 51 | 1001 | H51A(+66+90) | ACA UCA AGG AAG |
| | | | AUG GCA UUU CUA |
| | | | G |
| 51 | 1002 | H51A(+61+90) | ACA UCA AGG AAG |
| | | | AUG GCA UUU CUA |
| | | | GUU UGG |
| 51 | 1003 | Hu.DMD.exon51. | GAG CAG GTA CCT |
| | | 25.001.2 | CCA ACA TCA AGG |
| | | | AA |
| 50 | 1004 | H50D(+07−18) | GGG AUC CAG UAU |
| | | | ACU UAC AGG CUC |
| | | | C |
| 50 | 1005 | AVI-4038/5038 | CTT ACA GGC TCC |
| | | | AAT AGT GGT CAG |
| | | | T |
| 53 | 1006 | H53A27(+30+56) | CCT CCG GTT CTG |
| | | | AAG GTG TTC TTG |
| | | | TAC |
| 53 | 1007 | H53A(+36+60) | GTT GCC TCC GGT |
| | | | TCT GAA GGT GTT |
| | | | C |
| 45 | 1008 | H45A (−03+19) | CAA TGC CAT CCT |
| | | | GGA GTT CCT G |

TABLE 4

| SEQ ID | SEQUENCE | NUCLEOTIDE SEQUENCE (5′-3′) |
|---|---|---|
| 1 | H8A(-06+18) | GAU AGG UGG UAU CAA CAU CUG UAA |
| 2 | H8A (−03+18) | GAU AGG UGG UAU CAA CAU CUG |
| 3 | H8A(−07+18) | GAU AGG UGG UAU CAA CAU CUG UAA G |
| 4 | H8A(−06+14) | GGU GGU AUC AAC AUC UGU AA |
| 5 | H8A(−10+10) | GUA UCA ACA UCU GUA AGC AC |
| 6 | H7A(+45+67) | UGC AUG UUC CAG UCG UUG UGU GG |
| 7 | H7A(+02+26) | CAC UAU UCC AGU CAA AUA GGU CUG G |
| 8 | H7D(+15−10) | AUU UAC CAA CCU UCA GGA UCG AGU A |
| 9 | H7A(−18+03) | GGC CUA AAA CAC AUA CAC AUA |
| 10 | C6A(−10+10) | CAU UUU UGA CCU ACA UGU GG |
| 11 | C6A(−14+06) | UUU GAC CUA CAU GUG GAA AG |
| 12 | C6A(−14+12) | UAC AUU UUU GAC CUA CAU GUG GAA AG |
| 13 | C6A(−13+09) | AUU UUU GAC CUA CAU GGG AAA G |
| 14 | CH6A(+69+91) | UAC GAG UUG AUU GUC GGA CCC AG |
| 15 | C6D(+12−13) | GUG GUC UCC UUA CCU AUG ACU GUG G |
| 16 | C6D(+06−11) | GGU CUC CUU ACC UAU GA |
| 17 | H6D(+04−21) | UGU CUC AGU AAU CUU CUU ACC UAU |
| 18 | H6D(+18−04) | UCU UAC CUA UGA CUA UGG AUG AGA |
| 19 | H4A(+13+32) | GCA UGA ACU CUU GUG GAU CC |
| 20 | H4D(+04−16) | CCA GGG UAC UAC UUA CAU UA |
| 21 | H4D(−24−44) | AUC GUG UGU CAC AGC AUC CAG |
| 22 | H4A(+11+40) | UGU UCA GGG CAU GAA CUC UUG UGG AUC |
| | | CUU |
| 23 | H3A(+30+60) | UAG GAG GCG CCU CCC AUC CUG UAG GUC |
| | | ACU G |
| 24 | H3A(+35+65) | AGG UCU AGG AGG CGC CUC CCA UCC UGU |
| | | AGG U |
| 25 | H3A(+30+54) | GCG CCU CCC AUC CUG UAG GUC ACU G |
| 26 | H3D(+46−21) | CUU CGA GGA GGU CUA GGA GGC GCC UC |
| 27 | H3A(+30+50) | CUC CCA UCC UGU AGG UCA CUG |
| 28 | H3D(+19−03) | UAC CAG UUU UUG CCC UGU CAG G |
| 29 | H3A(−06+20) | UCA AUA UGC UGC UUC CCA AAC UGA AA |
| 30 | H3A(+37+61) | CUA GGA GGC GCC UCC CAU CCU GUA G |
| 31 | H5A(+20+50) | UUA UGA UUU CCA UCU ACG AUG UCA GUA |
| | | CUU C |
| 32 | H5D(+25−05) | CUU ACC UGC CAG UGG AGG AUU AUA UUC |
| | | CAA A |
| 33 | H5D(+10−15) | CAU CAG GAU UCU UAC CUG CCA GUG G |
| 34 | H5A(+10+34) | CGA UGU CAG UAC UUC CAA UAU UCA C |
| 35 | H5D(−04−21) | ACC AUU CAU CAG GAU UCU |
| 36 | H5D(+16−02) | ACC UGC CAG UGG AGG AUU |
| 37 | H5A(−07+20) | CCA AUA UUC ACU AAA UCA ACC UGU UAA |
| 38 | H5D(+18−12) | CAG GAU UGU UAC CUG CCA GUG GAG GAU |
| | | UAU |
| 39 | H5A(+05+35) | ACG AUG UCA GUA CUU CCA AUA UUC ACU |
| | | AAA U |
| 40 | H5A(+15+45) | AUU UCC AUC UAC GAU GUC AGU ACU UCC |
| | | AAU A |
| 41 | H10A(−05+16) | CAG GAG CUU CCA AAU GCU GCA |
| 42 | H10A(−05+24) | CUU GUC UUC AGG AGC UUC CAA AUG CUG |
| | | CA |
| 43 | H10A(+98+119) | UCC UCA GCA GAA AGA AGC CAC G |
| 44 | H10A(+130+149) | UUA GAA AUC UCU CCU UGU GC |
| 45 | H10A(−33−14) | UAA AUU GGG UGU UAC ACA AU |
| 46 | H11D(+26+49) | CCC UGA GGC AUU CCC AUC UUG AAU |
| 47 | H11D(+11−09) | AGG ACU UAC UUG CUU UGU UU |
| 48 | H11A(+118+140) | CUU GAA UUU AGG AGA UUC AUC UG |
| 49 | H11A(+75+97) | CAU CUU CUG AUA AUU UUC CUG UU |
| 50 | H12A(+52+75) | UCU UCU GUU UUU GUU AGC CAG UCA |
| 51 | H12A(−10+10) | UCU AUG UAA ACU GAA AAU UU |
| 52 | H12A(+11+30) | UUC UGG AGA UCC AUU AAA AC |
| 53 | H13A(+77+100) | CAG CAG UUG CGU GAU CUC CAC UAG |
| 54 | H13A(+55+75) | UUC AUC AAC UAC CAC CAC CAU |
| 55 | H13D(+06−19) | CUA AGC AAA AUA AUC UGA CCU UAA G |
| 56 | H14A(+37+64) | CUU GUA AAA GAA CCC AGC GGU C?U CUG |
| | | U |
| 57 | H14A(+14+35) | CAU CUA CAG AUG UUU GCC CAU C |
| 58 | H14A(+51+73) | GAA GGA UGU CUU GUA AAA GAA CC |
| 59 | H14D(−02+18) | ACC UGU UCU UCA GUA AGA CG |
| 60 | H14D(+14−10) | CAU GAC ACA CCU GUU CUU CAG UAA |
| 61 | H14A(+61+80) | CAU UUG AGA AGG AUG UCU UG |
| 62 | H14A(−12+12) | AUC UCC CAA UAC CUG GAG AAG AGA |
| 63 | H15A(−12+19) | GCC AUG CAC UAA AAA GGC ACU GCA AGA |
| | | CAU U |
| 64 | H15A(+48+71) | UCU UUA AAG CCA GUU GUG UGA AUC |
| 65 | H15A(+08+28) | UUU CUG AAA GCC AUG CAC UAA |
| 66 | H15D(+17−08) | GUA CAU ACG GCC AGU UUU UGA AGA C |
| 67 | H16A(−12+19) | CUA GAU CCG CUU UUA AAA CCU GUU AAA |
| | | ACA A |
| 68 | H16A(−06+25) | UCU UUU CUA GAU CCG CUU UUA AAA CCU |
| | | GUU A |
| 69 | H16A(−06+19) | CUA GAU CCG CUU UUA AAA CCU GUU A |
| 70 | H16A(+87+109) | CCG UCU UCU GGG UCA CUG ACU UA |
| 71 | H16A(−07+19) | CUA GAU CCG CUU UUA AAA CCU GUU AA |
| 72 | H16A(−07+13) | CCG CUU UUA AAA CCU GUU AA |
| 73 | H16A(+12+37) | UGG AUU GCU UUU UCU UUU CUA GAU CC |
| 74 | H16A(+92+116) | CAU GCU UCC GUC UUC UGG GUC ACU G |
| 75 | H16A(+45+67) | G AUC UUG UUU GAG UGA AUA CAGU |
| 76 | H16A(+105+126) | GUU AUC CAG CCA UGC UUC CGU C |
| 77 | H16D(+05−20) | UGA UAA UUG GUA UCA CUA ACC UGU G |
| 78 | H16D(+12−11) | GUA UCA CUA ACC UGU GCU GUA C |
| 79 | H19A(+35+53) | CUG CUG GCA UCU UGC AGU U |
| 80 | H19A(+35+65) | GCC UGA GCU GAU CUG CUG GCA UCU UGC |
| | | AGU U |
| 81 | H20A(+44+71) | CUG GCA GAA UUC GAU CCA CCG GCU GUU |
| | | C |
| 82 | H20A(+147+168) | CAG CAG UAG UUG UCA UCU GCU C |
| 83 | H20A(+185+203) | UGA UGG GGU GGU GGG UUG G |
| 84 | H20A(−08+17) | AUC UGC AUU AAC ACC CUC UAG AAA G |
| 85 | H20A(+30+53) | CCG GCU GUU CAG UUG UUC UGA GGC |
| 86 | H20A(−11+17) | AUC UGC AUU AAC ACC CUC UAG AAA GAA |
| | | A |
| 87 | H20D(+08−20) | GAA GGA GAA GAG AUU CUU ACC UUA CAA |
| | | A |
| 88 | H20A(+44+63) | AUU CGA UCC ACC GGC UGU UC |
| 89 | H20A(+149+168) | CAG CAG UAG UUG UCA UCU GC |
| 90 | H21A(−06+16) | GCC GGU UGA CUU CAU CCU GUG C |
| 91 | H21A(+85+106) | CUG CAU CCA GGA ACA UGG GUC C |
| 92 | H21A(+85+108) | GUC UGC AUC CAG GAA CAU GGG UC |
| 93 | H21A(+08+31) | GUU GAA GAU CUG AUA GCC GGU UGA |
| 94 | H21D(+18−07) | UAC UUA CUG UCU GUA GCU CUU UCU |
| 95 | H22A(+22+45) | CAC UCA UGG UCU CCU GAU AGC GCA |
| 96 | H22A(+125+146) | CUG CAA UUC CCC GAG UCU CUG C |
| 97 | H22A(+47+69) | ACU GCU GGA CCC AUG UCC UGA UG |
| 98 | H22A(+80+101) | CUA AGU UGA GGU AUG GAG AGU |
| 99 | H22D(+13−11) | UAU UCA CAG ACC UGC AAU UCC CC |
| 100 | H23A(+34+59) | ACA GUG GUG CUG AGA UAG UAU AGG CC |
| 101 | H23A(+18+39) | UAG GCC ACU UUG UUG CUC UUG C |
| 102 | H23A(+72+90) | UUC AGA GGG CGC UUU CUU C |
| 103 | H24A(+48+70) | GGG CAG GCC AUU CCU CCU UCA GA |
| 104 | H24A(−02+22) | UCU UCA GGG UUU GUA UGU GAU UCU |
| 105 | H25A(+9+36) | CUG GGC UGA AUU GUC UGA AUA UCA CUG |
| 106 | H25A(+131+156) | CUG UUG GCA CAU GUG AUC CCA CUG AG |
| 107 | H25D(+16−08) | GUC UAU ACC UGU UGG CAC AUG UGA |
| 108 | H26A(+132+156) | UGC UUU CUG UAA UUC AUC UGG AGU U |
| 109 | H26A(−07+19) | CCU CCU UUC UGG CAU AGA CCU UCC AC |
| 110 | H26A(+68+92) | UGU GUC AUC CAU UCG UGC AUC UCU G |
| 111 | H27A(+82+106) | UUA AGG CCU CUU GUG CUA CAG GUG G |
| 112 | H27A(−4+19) | GGG GCU CUU CUU UAG CUC UCU GA |
| 113 | H27D(+19−03) | GAC UUC CAA AGU CUU GCA UUU C |
| 114 | H28A(−05+19) | GCC AAC AUG CCC AAA CUU CCU AAG |
| 115 | H28A(+99+124) | CAG AGA UUU CCU CAG CUC CGC CAG GA |
| 116 | H28D(+16−05) | CUU ACA UCU AGC ACC UCA GAG |
| 117 | H29A(+57+81) | UCC GCC AUC UGU UAG GGU CUG UGC C |
| 118 | H29A(+18+42) | AUU UGG GUU AUC CUC UGA AUG UCG C |
| 119 | H29D(+17−05) | CAU ACC UCU UCA UGU AGU UCC C |
| 120 | H30A(+122+147) | CAU UUG AGC UGC GUC CAC CUU GUC UG |
| 121 | H30A(+25+50) | UCC UGG GCA GAC UGG AUG CUC UGU UC |
| 122 | H30D(+19−04) | UUG CCU GGG CUU CCU GAG GCA UU |
| 123 | H31D(+06−18) | UUC UGA AAU AAC AUA UAC CUG UGC |
| 124 | H31D(+03−22) | UAG UUU CUG AAA UAA CAU AUA CCU G |
| 125 | H31A(+05+25) | GAC UUG UCA AAU CAG AUU GGA |
| 126 | H31D(+04−20) | GUU UCU GAA AUA ACA UAU ACC UGU |
| 127 | H32D(+04−16) | CAC CAG AAA UAC AUA CCA CA |
| 128 | H32A(+151+170) | CAA UGA UUU AGC UGU GAC UG |
| 129 | H32A(+10+32) | CGA AAC UUC AUG GAG ACA UCU UG |
| 130 | H32A(+49+73) | CUU GUA GAC GCU GCU CAA AAU UGG C |
| 131 | H33D(+09−11) | CAU GCA CAC ACC UUU GCU CC |
| 132 | H33A(+53+76) | UCU GUA CAA UCU GAC GUC CAG UCU |
| 133 | H33A(+30+56) | GUC UUU AUC ACC AUU UCC ACU UCA GAC |
| 134 | H33A(+64+88) | CCG UCU GCU UUU UCU GUA CAA UCU G |
| 135 | H34A(+83+104) | UCC AUA UCU GUA GCU GCC AGC C |
| 136 | H34A(+143+165) | CCA GGC AAC UUC AGA AUC CAA AU |
| 137 | H34A(−20+10) | UUU CUG UUA CCU GAA AAG AAU UAU AAU |
| | | GAA |
| 138 | H34A(+46+70) | CAU UCA UUU CCU UUC GCA UCU UAC G |
| 139 | H34A(+95+120) | UGA UCU CUU UGU CAA UUC CAU AUC UG |
| 140 | H34D(+10−20) | UUC AGU GAU AUA GGU UUU ACC UUU CCC |
| | | CAG |
| 141 | H34A(+72+96) | CUG UAG CUG CCA GCC AUU CUG UCA AG |
| 142 | H35A(+141+161) | UCU UCU GCU CGG GAG GUG ACA |
| 143 | H35A(+116+135) | CCA GUU ACU AUU CAG AAG AC |
| 144 | H35A(+24+43) | UCU UCA GGU GCA CCU UCU GU |
| 145 | H36A(+26+50) | UGU GAU GUG GUC CAC AUU CUG GUC A |
| 146 | H36A(−02+18) | CCA UGU GUU UCU GGU AUU CC |
| 147 | H37A(+26+50) | CGU GUA GAG UCC ACC UUU GGG CGU A |
| 148 | H37A(+82+105) | UAC UAA UUU CCU GCA GUG GUC ACC |
| 149 | H37A(+134+157) | UUC UGU GUG AAA UGG CUG CAA AUC |
| 150 | H38A(−01+19) | CCU UCA AAG GAA UGG AGG CC |
| 151 | H38A(+59+83) | UGC UGA AUU UCA GCC UCC AGU GGU U |
| 152 | H38A(+88+112) | UGA AGU CUU CCU CUU UCA GAU UCA C |
| 153 | H39A(+62+85) | CUG GCU UUC UCU CAU CUG UGA UUC |
| 154 | H39A(+39+58) | GUU GUA AGU UGU CUC CUC UU |
| 155 | H39A(+102+121) | UUG UCU GUA ACA GCU GCU GU |
| 156 | H39D(+10−10) | GCU CUA AUA CCU UGA GAG CA |
| 157 | H40A(−05+17) | CUU UGA GAC CUC AAA UCC UGU U |
| 158 | H40A(+129+153) | CUU UAU UUU CCU UUC AUC UCU GGG C |
| 159 | H42A(−04+23) | AUC GUU UCU UCA CGG ACA GUG UGC UGG |
| 160 | H42A(+86+109) | GGG CUU GUG AGA CAU GAG UGA UUU |
| 161 | H42D(+19−02) | A CCU UCA GAG GAC UCC UCU UGC |
| 162 | H43D(+10−15) | UAU GUG UUA CCU ACC CUU GUC GGU C |
| 163 | H43A(+101+120) | GGA GAG AGC UUC CUG UAG CU |
| 164 | H43A(+78+100) | UCA CCC UUU CCA CAG GCG UUG CA |
| 165 | H44A(+85+104) | UUU GUG UCU UUC UGA GAA AC |
| 166 | H44D(+10−10) | AAA GAC UUA CCU UAA GAU AC |
| 167 | H44A(−06+14) | AUC UGU CAA AUC GCC UGC AG |
| 168 | H46D(+16−04) | UUA CCU UGA CUU GCU CAA GC |
| 169 | H46A(+90+109) | UCC AGG UUC AAG UGG GAU AC |
| 170 | H47A(+76+100) | GCU CUU CUG GGC UUA UGG GAG CAC U |
| 171 | H47D(+25−02) | ACC UUU AUC CAC UGG AGA UUU GUC UGC |
| 172 | H47A(−9+12) | UUC CAC CAG UAA CUG AAA CAG |
| 173 | H50A(+02+30) | CCA CUC AGA GCU CAG AUC UUC UAA CUU |
| | | CC |
| 174 | H50A(+07+33) | CUU CCA CUC AGA GCU CAG AUC UUC UAA |
| 175 | H50D(+07−18) | GGG AUC CAG UAU ACU UAC AGG CUC C |
| 176 | H51A(−01+25) | ACC AGA GUA ACA GUC UGA GUA GGA GC |
| 177 | H51D(+16−07) | CUC AUA CCU UCU GCU UGA UGA UC |
| 178 | H51A(+111 +134) | UUC UGU CCA AGC CCG GUU GAA AUC |
| 179 | H51A(+61+90) | ACA UCA AGG AAG AUG GCA UUU CUA GUU |
| | | UGG |
| 180 | H51A(+66+90) | ACA UCA AGG AAG AUG GCA UUU CUA G |
| 181 | H51A(+66+95) | CUC CAA CAU CAA GGA AGA UGG CAU UUC |
| | | UAG |
| 182 | H51D(+08−17) | AUC AUU UUU UCU CAU ACC UUC UGC U |
| 183 | H51A/D(+08−17) | AUC AUU UUU UCU CAU ACC UUC UGC UAG |
| | & (−15+) | GAG CUA AAA |
| 184 | H51A(+175+195) | CAC CCA CCA UCA CCC UCU GUG |
| 185 | H51A(+199+220) | AUC AUC UCG UUG AUA UCC UCA A |
| 186 | H52A(−07+14) | UCC UGC AUU GUU GCC UGU AAG |
| 187 | H52A(+12+41) | UCC AAC UGG GGA CGC CUC UGU UCC AAA |
| | | UCC |
| 188 | H52A(+17+37) | ACU GGG GAC GCC UCU GUU CCA |
| 189 | H52A(+93+112) | CCG UAA UGA UUG UUC UAG CC |
| 190 | H52D(+05−15) | UGU UAA AAA ACU UAC UUC GA |
| 191 | H53A(+45+69) | CAU UCA ACU GUU GCC UCC GGU UCU G |
| 192 | H53A(+39+62) | CUG UUG CCU CCG GUU CUG AAG GUG |
| 193 | H53A(+39+69) | CAU UCA ACU GUU GCC UCC GGU UCU GAA |
| | | GGU G |
| 194 | H53D(+14−07) | UAC UAA CCU UGG UUU CUG UGA |
| 195 | H53A(+23+47) | CUG AAG GUG UUC UUG UAC UUC AUC C |
| 196 | H53A(+150+176) | UGU AUA GGG ACC CUC CUU CCA UGA CUC |
| 197 | H53D(+20−05) | CUA ACC UUG GUU UCU GUG AUU UUC U |
| 198 | H53D(+09−18) | GGU AUC UUU GAU ACU AAC CUU GGU UUC |
| 199 | H53A(−12+10) | AUU CUU UCA ACU AGA AUA AAA G |
| 200 | H53A(−07+18) | GAU UCU GAA UUC UUU CAA CUA GAA U |
| 201 | H53A(+07+26) | AUC CCA CUG AUU CUG AAU UC |
| 202 | H53A(+124+145) | UUG GCU CUG GCC UGU CCU AAG A |
| 203 | H46A(+86+115) | CUC UUU UCC AGG UUC AAG UGG GAU ACU |
| | | AGC |
| 204 | H46A(+107+137) | CAA GCU UUU CUU UUA GUU GCU GCU CUU |
| | | UUC C |
| 205 | H46A(−10+20) | UAU UCU UUU GUU CUU CUA GCC UGG AGA |
| | | AAG |
| 206 | H46A(+50+77) | CUG CUU CCU CCA ACC AUA AAA CAA AUU |
| | | C |
| 207 | H45A(−06+20) | CCA AUG CCA UCC UGG AGU UCC UGU AA |
| 208 | H45A(+91 +110) | UCC UGU AGA AUA CUG GCA UC |
| 209 | H45A(+125+151) | UGC AGA CCU CCU GCC ACC GCA GAU UCA |
| 210 | H45D(+16−04) | CUA CCU CUU UUU UCU GUC UG |
| 211 | H45A(+71+90) | UGU UUU UGA GGA UUG CUG AA |

| | | SEQ |
|---|---|---|
| | | ID |
| Description | Sequence | NO |
| H53A(+33+60) | GTTGCCTCCGGTTCTGAAGGTGTTCTTG | 1 |
| H53A(+23+47) | CTGAAGGTGTTCTTGTACTTCATCC | 6 |
| H53A(+33+62) | CTGTTGCCTCCGGTTCTGAAGGTGTTCTTG | 7 |
| H53A(+33+65) | CAACTGTTGCCTCCGGTTCTGAAGGTGTTCTTG | 8 |
| H53A(+31+55) | CTCCGGTTCTGAAGGTGTTCTTGTA | 9 |
| H53A(+46+73) | ATTTCATTCAACTGTTGCCTCCGGTTCT | 10 |
| H53A(+22+46) | TGAAGGTGTTCTTGTACTTCATCCC | 11 |
| H53A(+46+69) | CATTCAACTGTTGCCTCCGGTTCT | 12 |
| H53A(+40+61) | TGTTGCCTCCGGTTCTGAAGGT | 13 |
| H53A(+30+60) | GTTGCCTCCGGTTCTGAAGGTGTTC | 14 |
| H53A(+30+57) | GCCTCCGGTTCTGAAGGTGTTCTTGTAC | 15 |
| H53A(+30+56) | CCTCCGGTTCTGAAGGTGTTCTTGTAC | 16 |
| H53A(+30+55) | CTCCGGTTCTGAAGGTGTTCTTGTAC | 17 |
| H53A(+33+57) | GCCTCCGGTTCTGAAGGTGTTCTTG | 18 |
| H44A(−07+17) | CAGATCTGTCAAATCGCCTGCAGG | 1 |
| H44A(−07+20) | CAACAGATCTGTCAAATCGCCTGCAGG | 2 |
| H44A(−07+22) | CTCAACAGATCTGTCAAATCGCCTGCAGG | 3 |
| H44A(+77+101) | GTGTCTTTCTGAGAAACTGTTCAGC | 4 |
| H44A(+64+91) | GAGAAACTGTTCAGCTTCTGTTAGCCAC | 5 |
| H44A(+62+89) | GAAACTGTTCAGCTTCTGTTAGCCACTG | 6 |
| H44A(+62+85) | CTGTTCAGCTTCTGTTAGCCACTG | 7 |
| H44A(−06+14) | ATCTGTCAAATCGCCTGCAG | 8 |
| H44A(+85+104) | TTTGTGTCTTTCTGAGAAAC | 9 |
| H44A(+61+84) | TGTTCAGCTTCTGTTAGCCACTGA | 10 |
| H44A(−10+15) | GATCTGTCAAATCGCCTGCAGGTAA | 11 |
| H44A(+64+88) | AAACTGTTCAGCTTCTGTTAGCCAC | 12 |
| H44A(+79+103) | TTGTGTCTTTCTGAGAAACTGTTCA | 13 |
| H44A(−06+20) | CAACAGATCTGTCAAATCGCCTGCAG | 14 |
| H44A(−09+17) | CAGATCTGTCAAATCGCCTGCAGGTA | 15 |
| H44A(+59+85) | CTGTTCAGCTTCTGTTAGCCACTGATT | 16 |
| H44A(+59+89) | GAAACTGTTCAGCTTCTGTTAGCCACTGATT | 17 |
| H44A(+65+90) | AGAAACTGTTCAGCTTCTGTTAGCCA | 18 |
| | | SEQ |
| | | ID |
| Name | Sequences | NO |

Oligomer Targeting Sequences (5′ to 3′);

| | | |
|---|---|---|
| Hu.DMD.Exon44.25.001 | CTGCAGGTAAAAGCATATGGATCAA | 1 |
| Hu.DMD.Exon44.25.002 | ATCGCCTGCAGGTAAAAGCATATGG | 2 |
| Hu.DMD.Exon44.25.003 | GTCAAATCGCCTGCAGGTAAAAGCA | 3 |
| Hu.DMD.Exon44.25.004 | GATCTGTCAAATCGCCTGCAGGTAA | 4 |
| Hu.DMD.Exon44.25.005 | CAACAGATCTGTCAAATCGCCTGCA | 5 |
| Hu.DMD.Exon44.25.006 | TTTCTCAACAGATCTGTCAAATCGC | 6 |
| Hu.DMD.Exon44.25.007 | CCATTTCTCAACAGATOTGTCAAAT | 7 |
| Hu.DMD.Exon44.25.008 | ATAATGAAAACGCCGCCATTTCTCA | 8 |
| Hu.DMD.Exon44.25.009 | AAATATCTTTATATCATAATGAAAA | 9 |
| Hu.DMD.Exon44.25.010 | TGTTAGCCACTGATTAAATATCTTT | 10 |
| Hu.DMD.Exon44.25.011 | AAACTGTTCAGCTTCTGTTAGCCAC | 11 |
| Hu.DMD.Exon44.25.012 | TTGTGTCTTTCTGAGAAACTGTTCA | 12 |
| Hu.DMD.Exon44.25.013 | CCAATTCTCAGGAATTTGTGTCTTT | 13 |
| Hu.DMD.Exon44.25.014 | GTATTTAGCATGTTCCCAATTCTCA | 14 |
| Hu.DMD.Exon44.25.015 | CTTAAGATACCATTTGTATTTAGCA | 15 |
| Hu.DMD.Exon44.25.016 | CTTACCTTAAGATACCATTTGTATT | 16 |
| Hu.DMD.Exon44.25.017 | AAAGACTTACCTTAAGATACCATTT | 17 |
| Hu.DMD.Exon44.25.018 | AAATCAAAGACTTACCTTAAGATAC | 18 |
| Hu.DMD.Exon44.25.019 | AAAACAAATCAAAGACTTACCTTAA | 19 |
| Hu.DMD.Exon44.25.020 | TCGAAAAAACAAATCAAAGACTTAC | 20 |
| Hu.DMD.Exon45.25.001 | CTGTAAGATACCAAAAAGGCAAAAC | 21 |
| Hu.DMD.Exon45.25.002 | CCTGTAAGATACCAAAAAGGCAAAA | 22 |
| Hu.DMD.Exon45.25.002. | AGTTCCTGTAAGATACCAAAAAGGC | 23 |
| 2 | | |
| Hu.DMD.Exon45.25.003 | GAGTTCCTGTAAGATACCAAAAAGG | 24 |
| Hu.DMD.Exon45.25.003. | CCTGGAGTTCCTGTAAGATACCAAA | 25 |
| 2 | | |
| Hu.DMD.Exon45.25.004 | TCCTGGAGTTCCTGTAAGATACCAA | 26 |
| Hu.DMD.Exon45.25.004. | GCCATCCTGGAGTTCCTGTAAGATA | 27 |
| 2 | | |
| Hu.DMD.Exon45.25.005 | TGCCATCCTGGAGTTCCTGTAAGAT | 28 |
| Hu.DMD.Exon45.25.005. | CCAATGCCATCCTGGAGTTCCTGTA | 29 |
| 2 | | |
| Hu.DMD.Exon45.25.006 | CCCAATGCCATCCTGGAGTTCCTGT | 30 |
| Hu.DMD.Exon45.25.006. | GCTGCCCAATGCCATCCTGGAGTTC | 31 |
| 2 | | |
| Hu.DMD.Exon45.25.007 | CGCTGCCCAATGCCATCCTGGAGTT | 32 |
| Hu.DMD.Exon45.25.008 | AACAGTTTGCCGOTGCCCAATGCCA | 33 |
| Hu.DMD.Exon45.25.008. | CTGACAACAGTTTGCCGCTGCCCAA | 34 |
| 2 | | |
| Hu.DMD.Exon45.25.009 | GTTGCATTCAATGTTCTGACAACAG | 35 |
| Hu.DMD.Exon45.25.010 | GCTGAATTATTTCTTCCCCAGTTGC | 36 |
| Hu.DMD.Exon45.25.010. | ATTATTTCTTCCCCAGTTGCATTCA | 37 |
| 2 | | |
| Hu.DMD.Exon45.25.011 | GGCATCTGTTTTTGAGGATTGCTGA | 38 |
| Hu.DMD.Exon45.25.011. | TTTGAGGATTGCTGAATTATTTCTT | 39 |
| 2 | | |
| Hu.DMD.Exon45.25.012 | AATTTTTCCTGTAGAATACTGGCAT | 40 |
| Hu.DMD.Exon45.25.012. | ATACTGGCATCTGTTTTTGAGGATT | 41 |
| 2 | | |
| Hu.DMD.Exon45.25.013 | ACCGCAGATTCAGGCTTCCCAATTT | 42 |
| Hu.DMD.Exon45.25.013. | AATTTTTCCTGTAGAATACTGGCAT | 43 |
| 2 | | |
| Hu.DMD.Exon45.25.014 | CTGTTTGCAGACCTCCTGCCACCGC | 44 |
| Hu.DMD.Exon45.25.014. | AGATTCAGGCTTCCCAATTTTTCCT | 45 |
| 2 | | |
| Hu.DMD.Exon45.25.015 | CTCTTTTTTCTGTCTGACAGCTGTT | 46 |
| Hu.DMD.Exon45.25.015. | ACCTCCTGCCACCGCAGATTCAGGC | 47 |
| 2 | | |
| Hu.DMD.Exon45.25.016 | CCTACCTCTTTTTTCTGTCTGACAG | 48 |
| Hu.DMD.Exon45.25.016. | GACAGCTGTTTGCAGACCTCCTGCC | 49 |
| 2 | | |
| Hu.DMD.Exon45.25.017 | GTCGCCCTACCTCTTTTTTCTGTCT | 50 |
| Hu.DMD.Exon45.25.018 | GATCTGTCGCCCTACCTCTTTTTTC | 51 |
| Hu.DMD.Exon45.25.019 | TATTAGATCTGTCGCCCTACCTCTT | 52 |
| Hu.DMD.Exon45.25.020 | ATTCCTATTAGATCTGTCGCCCTAC | 53 |
| Hu.DMD.Exon45.20.001 | AGATACCAAAAAGGCAAAAC | 54 |
| Hu.DMD.Exon45.20.002 | AAGATACCAAAAAGGCAAAA | 55 |
| Hu.DMD.Exon45.20.003 | CCTGTAAGATACCAAAAAGG | 56 |
| Hu.DMD.Exon45.20.004 | GAGTTCCTGTAAGATACCAA | 57 |
| Hu.DMD.Exon45.20.005 | TCCTGGAGTTCCTGTAAGAT | 58 |
| Hu.DMD.Exon45.20.006 | TGCCATCCTGGAGTTCCTGT | 59 |
| Hu.DMD.Exon45.20.007 | CCCAATGCCATCCTGGAGTT | 60 |
| Hu.DMD.Exon45.20.008 | CGCTGCCCAATGCCATCCTG | 61 |
| Hu.DMD.Exon45.20.009 | CTGACAACAGTTTGCCGCTG | 62 |
| Hu.DMD.Exon45.20.010 | GTTGGATTCAATGTTCTGAC | 63 |
| Hu.DMD.Exon45.20.011 | ATTATTTCTTCCCCAGTTGC | 64 |
| Hu.DMD.Exon45.20.012 | TTTGAGGATTGCTGAATTAT | 65 |
| Hu.DMD.Exon45.20.013 | ATACTGGCATCTGTTTTTGA | 66 |
| Hu.DMD.Exon45.20.014 | AATTTTTCCTGTAGAATACT | 67 |
| Hu.DMD.Exon45.20.015 | AGATTCAGGCTTOCCAATTT | 68 |
| Hu.DMD.Exon45.20.016 | ACCTCCTGCCACCGCAGATT | 69 |
| Hu.DMD.Exon45.20.017 | GACAGCTGTTTGCAGACCTC | 70 |
| Hu.DMD.Exon45.20.018 | CTCTTTTTTCTGTCTGACAG | 71 |
| Hu.DMD.Exon45.20.019 | CCTACCTCTTTTTTCTGTCT | 72 |
| Hu.DMD.Exon45.20.020 | GTCGCCCTACCTCTTTTTTC | 73 |
| Hu.DMD.Exon45.20.021 | GATCTGTCGCCCTACCTCTT | 74 |
| Hu.DMD.Exon45.20.022 | TATTAGATCTGTCGCCCTAC | 75 |
| Hu.DMD.Exon45.20.023 | ATTCCTATTAGATCTGTCGC | 76 |
| Hu.DMD.Exon46.25.001 | GGGGGATTTGAGAAAATAAAATTAC | 77 |
| Hu.DMD.Exon46.25.002 | ATTTGAGAAAATAAAATTACCTTGA | 78 |
| Hu.DMD.Exon46.25.002. | CTAGCCTGGAGAAAGAAGAATAAAA | 79 |
| 2 | | |
| Hu.DMD.Exon46.25.003 | AGAAAATAAAATTACCTTGACTTGC | 80 |
| Hu.DMD.Exon46.25.003. | TTCTTCTAGCCTGGAGAAAGAAGAA | 81 |
| 2 | | |
| Hu.DMD.Exon46.25.004 | ATAAAATTACCTTGACTTGCTCAAG | 82 |
| Hu.DMD.Exon46.25.004. | TTTTGTTCTTCTAGCCTGGAGAAAG | 83 |
| 2 | | |
| Hu.DMD.Exon46.25.005 | ATTACCTTGACTTGCTCAAGCTTTT | 84 |
| Hu.DMD.Exon46.25.005. | TATTCTTTTGTTCTTCTAGCCTGGA | 85 |
| 2 | | |
| Hu.DMD.Exon46.25.006 | CTTGACTTGCTCAAGCTTTTCTTTT | 86 |
| Hu.DMD.Exon46.25.006. | CAAGATATTCTTTTGTTCTTCTAGC | 87 |
| 2 | | |
| Hu.DMD.Exon46.25.007 | CTTTTAGTTGCTGCTCTTTTCCAGG | 88 |
| Hu.DMD.Exon46.25.008 | CCAGGTTCAAGTGGGATACTAGCAA | 89 |
| Hu.DMD.Exon46.25.008. | ATCTCTTTGAAATTCTGACAAGATA | 90 |
| 2 | | |
| Hu.DMD.Exon46.25.009 | AGCAATGTTATCTGCTTCCTCCAAC | 91 |
| Hu.DMD.Exon46.25.009. | AACAAATTCATTTAAATCTOTTTGA | 92 |
| 2 | | |
| Hu.DMD.Exon46.25.010 | CCAACCATAAAACAAATTCATTTAA | 93 |
| Hu.DMD.Exon46.25.010. | TTCCTCCAACCATAAAACAAATTCA | 94 |
| 2 | | |
| Hu.DMD.Exon46.25.011 | TTTAAATCTCTTTGAAATTCTGACA | 95 |
| Hu.DMD.Exon46.25.012 | TGACAAGATATTCTTTTGTTCTTCT | 96 |
| Hu.DMD.Exon46.25.012. | TTCAAGTGGGATACTAGCAATGTTA | 97 |
| 2 | | |
| Hu.DMD.Exon46.25.013 | AGATATTCTTTTGTTCTTCTAGCCT | 98 |
| Hu.DMD.Exon46.25.013. | CTGCTCTTTTCCAGGTTCAAGTGGG | 99 |
| 2 | | |
| Hu.DMD.Exon46.25.014 | TTCTTTTGTTCTTCTAGCCTGGAGA | 100 |
| Hu.DMD.Exon46.25.014. | CTTTTCTTTTAGTTGCTGCTCTTTT | 101 |
| 2 | | |
| Hu.DMD.Exon46.25.015 | TTGTTCTTCTAGCCTGGAGAAAGAA | 102 |
| Hu.DMD.Exon46.25.016 | CTTCTAGCCTGGAGAAAGAAGAATA | 103 |
| Hu.DMD.Exon46.25.017 | AGCCTGGAGAAAGAAGAATAAAATT | 104 |
| Hu.DMD.Exon46.25.018 | CTGGAGAAAGAAGAATAAAATTGTT | 105 |
| Hu.DMD.Exon46.20.001 | GAAAGAAGAATAAAATTGTT | 106 |
| Hu.DMD.Exon46.20.002 | GGAGAAAGAAGAATAAAATT | 107 |
| Hu.DMD.Exon46.20.003 | AGCCTGGAGAAAGAAGAATA | 108 |
| Hu.DMD.Exon46.20.004 | CTTCTAGCCTGGAGAAAGAA | 109 |
| Hu.DMD.Exon46.20.005 | TTGTTCTTCTAGCCTGGAGA | 110 |
| Hu.DMD.Exon46.20.006 | TTCTTTTGTTCTTCTAGCCT | 111 |
| Hu.DMD.Exon46.20.007 | TGACAAGATATTCTTTTGTT | 112 |
| Hu.DMD.Exon46.20.008 | ATCTCTTTGAAATTCTGACA | 113 |
| Hu.DMD.Exon46.20.009 | AACAAATTCATTTAAATCTO | 114 |
| Hu.DMD.Exon46.20.010 | TTCCTCCAACCATAAAACAA | 115 |
| Hu.DMD.Exon46.20.011 | AGCAATGTTATCTGCTTCCT | 116 |
| Hu.DMD.Exon46.20.012 | TTCAAGTGGGATACTAGCAA | 117 |
| Hu.DMD.Exon46.20.013 | CTGCTCTTTTCCAGGTTCAA | 118 |
| Hu.DMD.Exon46.20.014 | CTTTTCTTTTAGTTGCTGCT | 119 |
| Hu.DMD.Exon46.20.015 | CTTGACTTGCTCAAGCTTTT | 120 |
| Hu.DMD.Exon46.20.016 | ATTACCTTGACTTGCTCAAG | 121 |
| Hu.DMD.Exon46.20.017 | ATAAAATTACCTTGACTTGC | 122 |
| Hu.DMD.Exon46.20.018 | AGAAAATAAAATTACCTTGA | 123 |
| Hu.DMD.Exon46.20.019 | ATTTGAGAAAATAAAATTAC | 124 |
| Hu.DMD.Exon46.20.020 | GGGGGATTTGAGAAAATAAA | 125 |
| Hu.DMD.Exon47.25.001 | CTGAAACAGACAAATGCAACAACGT | 126 |
| Hu.DMD.Exon47.25.002 | AGTAACTGAAACAGACAAATGCAAC | 127 |
| Hu.DMD.Exon47.25.003 | CCACCAGTAACTGAAACAGACAAAT | 128 |
| Hu.DMD.Exon47.25.004 | CTCTTCCACCAGTAACTGAAACAGA | 129 |
| Hu.DMD.Exon47.25.005 | GGCAACTCTTCCACCAGTAACTGAA | 130 |
| Hu.DMD.Exon47.25.006 | GCAGGGGCAACTCTTCCACCAGTAA | 131 |
| Hu.DMD.Exon47.25.007 | CTGGCGCAGGGGCAACTCTTCCACC | 132 |
| Hu.DMD.Exon47.25.008 | TTTAATTGTTTGAGAATTCCCTGGC | 133 |
| Hu.DMD.Exon47.25.008. | TTGTTTGAGAATTCCCTGGCGCAGG | 134 |
| 2 | | |
| Hu.DMD.Exon47.25.009 | GCACGGGTCCTCCAGTTTCATTTAA | 135 |
| Hu.DMD.Exon47.25.009. | TCCAGTTTCATTTAATTGTTTGAGA | 136 |
| 2 | | |
| Hu.DMD.Exon47.25.010 | GCTTATGGGAGCACTTACAAGCACG | 137 |
| Hu.DMD.Exon47.25.010. | TACAAGCACGGGTCCTCCAGTTTCA | 138 |
| 2 | | |
| Hu.DMD.Exon47.25.011 | AGTTTATCTTGCTCTTCTGGGCTTA | 139 |
| Hu.DMD.Exon47.25.012 | TCTGCTTGAGCTTATTTTCAAGTTT | 140 |
| Hu.DMD.Exon47.25.012. | ATCTTGCTCTTCTGGGCTTATGGGA | 141 |
| 2 | | |
| Hu.DMD.Exon47.25.013 | CTTTATCCACTGGAGATTTGTCTGC | 142 |
| Hu.DMD.Exon47.25.013. | CTTATTTTCAAGTTTATCTTGCTCT | 143 |
| 2 | | |
| Hu.DMD.Exon47.25.014 | CTAACCTTTATCCACTGGAGATTTG | 144 |
| Hu.DMD.Exon47.25.014. | ATTTGTCTGCTTGAGCTTATTTTCA | 145 |
| 2 | | |
| Hu.DMD.Exon47.25.015 | AATGTCTAACCTTTATCCACTGGAG | 146 |
| Hu.DMD.Exon47.25.016 | TGGTTAATGTCTAACCTTTATCCAC | 147 |
| Hu.DMD.Exon47.25.017 | AGAGATGGTTAATGTCTAACCTTTA | 148 |
| Hu.DMD.Exon47.25.018 | ACGGAAGAGATGGTTAATGTCTAAC | 149 |
| Hu.DMD.Exon47.20.001 | ACAGACAAATGCAACAACGT | 150 |
| Hu.DMD.Exon47.20.002 | CTGAAACAGACAAATGCAAC | 151 |
| Hu.DMD.Exon47.20.003 | AGTAACTGAAACAGACAAAT | 152 |
| Hu.DMD.Exon47.20.004 | CCACCAGTAACTGAAACAGA | 153 |
| Hu.DMD.Exon47.20.005 | CTCTTCCACCAGTAACTGAA | 154 |
| Hu.DMD.Exon47.20.006 | GGCAACTOTTCCACCAGTAA | 155 |
| Hu.DMD.Exon47.20.007 | CTGGCGCAGGGGCAACTCTT | 156 |
| Hu.DMD.Exon47.20.008 | TTGTTTGAGAATTCCCTGGC | 157 |
| Hu.DMD.Exon47.20.009 | TCCAGTTTCATTTAATTGTT | 158 |
| Hu.DMD.Exon47.20.010 | TACAAGCACGGGTCCTCCAG | 159 |
| Hu.DMD.Exon47.20.011 | GCTTATGGGAGCACTTACAA | 160 |
| Hu.DMD.Exon47.20.012 | ATCTTGCTCTTCTGGGOTTA | 161 |
| Hu.DMD.Exon47.20.013 | CTTATTTTCAAGTTTATCTT | 162 |
| Hu.DMD.Exon47.20.014 | ATTTGTCTGCTTGAGCTTAT | 163 |
| Hu.DMD.Exon47.20.015 | CTTTATCCACTGGAGATTTG | 164 |
| Hu.DMD.Exon47.20.016 | CTAACCTTTATCCACTGGAG | 165 |
| Hu.DMD.Exon47.20.017 | AATGTCTAACCTTTATCCAC | 166 |
| Hu.DMD.Exon47.20.018 | TGGTTAATGTCTAACCTTTA | 167 |
| Hu.DMD.Exon47.20.019 | AGAGATGGTTAATGTCTAAC | 168 |
| Hu.DMD.Exon47.20.020 | ACGGAAGAGATGGTTAATGT | 169 |
| Hu.DMD.Exon48.25.001 | CTGAAAGGAAAATACATTTTAAAAA | 170 |
| Hu.DMD.Exon48.25.002 | CCTGAAAGGAAAATACATTTTAAAA | 171 |
| Hu.DMD.Exon48.25.002. | GAAACCTGAAAGGAAAATACATTTT | 172 |
| 2 | | |
| Hu.DMD.Exon48.25.003 | GGAAACCTGAAAGGAAAATACATTT | 173 |
| Hu.DMD.Exon48.25.003. | CTCTGGAAACCTGAAAGGAAAATAC | 174 |
| 2 | | |
| Hu.DMD.Exon48.25.004 | GCTCTGGAAACCTGAAAGGAAAATA | 175 |
| Hu.DMD.Exon48.25.004. | TAAAGCTCTGGAAACCTGAAAGGAA | 634 |
| 2 | | |
| Hu.DMD.Exon48.25.005 | GTAAAGCTCTGGAAACCTGAAAGGA | 176 |
| Hu.DMD.Exon48.25.005. | TCAGGTAAAGCTCTGGAAACCTGAA | 177 |
| 2 | | |
| Hu.DMD.Exon48.25.006 | CTCAGGTAAAGCTCTGGAAACCTGA | 178 |
| Hu.DMD.Exon48.25.006. | GTTTCTCAGGTAAAGCTCTGGAAAC | 179 |
| 2 | | |
| Hu.DMD.Exon48.25.007 | TGTTTCTCAGGTAAAGCTCTGGAAA | 180 |
| Hu.DMD.Exon48.25.007. | AATTTCTCCTTGTTTCTCAGGTAAA | 181 |
| 2 | | |
| Hu.DMD.Exon48.25.008 | TTTGAGCTTCAATTTCTCCTTGTTT | 182 |
| Hu.DMD.Exon48.25.008 | TTTTATTTGAGCTTCAATTTCTOCT | 183 |
| Hu.DMD.Exon48.25.009 | AAGCTGCCCAAGGTCTTTTATTTGA | 184 |
| Hu.DMD.Exon48.25.010 | AGGTCTTCAAGCTTTTTTTCAAGCT | 185 |
| Hu.DMD.Exon48.25.010. | TTCAAGCTTTTTTTCAAGCTGCCCA | 186 |
| 2 | | |
| Hu.DMD.Exon48.25.011 | GATGATTTAACTGCTCTTCAAGGTC | 187 |
| Hu.DMD.Exon48.25.011. | CTGCTCTTCAAGGTCTTCAAGCTTT | 188 |
| 2 | | |
| Hu.DMD.Exon48.25.012 | AGGAGATAACCACAGCAGCAGATGA | 189 |
| Hu.DMD.Exon48.25.012. | CAGCAGATGATTTAACTGCTCTTCA | 190 |
| 2 | | |
| Hu.DMD.Exon48.25.013 | ATTTCCAACTGATTCCTAATAGGAG | 191 |
| Hu.DMD.Exon48.25.014 | CTTGGTTTGGTTGGTTATAAATTTC | 192 |
| Hu.DMD.Exon48.25.014. | CAACTGATTCCTAATAGGAGATAAC | 193 |
| 2 | | |
| Hu.DMD.Exon48.25.015 | CTTAACGTCAAATGGTCCTTCTTGG | 194 |
| Hu.DMD.Exon48.25.015. | TTGGTTATAAATTTCCAACTGATTC | 195 |
| 2 | | |
| Hu.DMD.Exon48.25.016 | CCTACCTTAACGTCAAATGGTCCTT | 196 |
| Hu.DMD.Exon48.25.016. | TCCTTCTTGGTTTGGTTGGTTATAA | 197 |
| 2 | | |
| Hu.DMD.Exon48.25.017 | AGTTCCCTACCTTAACGTCAAATGG | 198 |
| Hu.DMD.Exon48.25.018 | CAAAAAGTTCCCTACCTTAACGTCA | 199 |
| Hu.DMD.Exon48.25.019 | TAAAGCAAAAAGTTCCCTACCTTAA | 200 |
| Hu.DMD.Exon48.25.020 | ATATTTAAAGCAAAAAGTTCCCTAC | 201 |
| Hu.DMD.Exon48.20.001 | AGGAAAATACATTTTAAAAA | 202 |
| Hu.DMD.Exon48.20.002 | AAGGAAAATACATTTTAAAA | 203 |
| Hu.DMD.Exon48.20.003 | CCTGAAAGGAAAATACATTT | 204 |
| Hu.DMD.Exon48.20.004 | GGAAACCTGAAAGGAAAATA | 205 |
| Hu.DMD.Exon48.20.005 | GCTCTGGAAACCTGAAAGGA | 206 |
| Hu.DMD.Exon48.20.006 | GTAAAGCTCTGGAAACCTGA | 207 |
| Hu.DMD.Exon48.20.007 | CTCAGGTAAAGCTCTGGAAA | 208 |
| Hu.DMD.Exon48.20.008 | AATTTCTCCTTGTTTCTCAG | 209 |
| Hu.DMD.Exon48.20.009 | TTTTATTTGAGCTTCAATTT | 210 |
| Hu.DMD.Exon48.20.010 | AAGCTGCCCAAGGTOTTTTA | 211 |
| Hu.DMD.Exon48.20.011 | TTCAAGCTTTTTTTCAAGCT | 212 |
| Hu.DMD.Exon48.20.012 | CTGCTCTTCAAGGTCTTCAA | 213 |
| Hu.DMD.Exon48.20.013 | CAGCAGATGATTTAACTGCT | 214 |
| Hu.DMD.Exon48.20.014 | AGGAGATAACCACAGCAGCA | 215 |
| Hu.DMD.Exon48.20.015 | CAACTGATTCOTAATAGGAG | 216 |
| Hu.DMD.Exon48.20.016 | TTGGTTATAAATTTCCAACT | 217 |
| Hu.DMD.Exon48.20.017 | TCCTTCTTGGTTTGGTTGGT | 218 |
| Hu.DMD.Exon48.20.018 | CTTAACGTCAAATGGTCCTT | 219 |
| Hu.DMD.Exon48.20.019 | CCTACCTTAACGTCAAATGG | 220 |
| Hu.DMD.Exon48.20.020 | AGTTCCCTACCTTAACGTCA | 221 |
| Hu.DMD.Exon48.20.021 | CAAAAAGTTCCCTACCTTAA | 222 |
| Hu.DMD.Exon48.20.022 | TAAAGCAAAAAGTTCCCTAC | 223 |
| Hu.DMD.Exon48.20.023 | ATATTTAAAGCAAAAAGTTC | 224 |
| Hu.DMD.Exon49.25.001 | CTGGGGAAAAGAACCCATATAGTGC | 225 |
| Hu.DMD.Exon49.25.002 | TCCTGGGGAAAAGAACCCATATAGT | 226 |
| Hu.DMD.Exon49.25.002. | GTTTCCTGGGGAAAAGAACCCATAT | 227 |
| 2 | | |
| Hu.DMD.Exon49.25.003 | CAGTTTCCTGGGGAAAAGAACCCAT | 228 |
| Hu.DMD.Exon49.25.003. | TTTCAGTTTCCTGGGGAAAAGAACC | 229 |
| 2 | | |
| Hu.DMD.Exon49.25.004 | TATTTCAGTTTCCTGGGGAAAAGAA | 230 |
| Hu.DMD.Exon49.25.004. | TGCTATTTCAGTTTCCTGGGGAAAA | 231 |
| 2 | | |
| Hu.DMD.Exon49.25.005 | ACTGCTATTTCAGTTTCCTGGGGAA | 232 |
| Hu.DMD.Exon49.25.005. | TGAACTGCTATTTCAGTTTCCTGGG | 233 |
| 2 | | |
| Hu.DMD.Exon49.25.006 | CTTGAACTGCTATTTCAGTTTCCTG | 234 |
| Hu.DMD.Exon49.25.006. | TAGCTTGAACTGCTATTTCAGTTTC | 235 |
| 2 | | |
| Hu.DMD.Exon49.25.007 | TTTAGCTTGAACTGCTATTTCAGTT | 236 |
| Hu.DMD.Exon49.25.008 | TTCCACATCCGGTTGTTTAGCTTGA | 237 |
| Hu.DMD.Exon49.25.009 | TGCCCTTTAGACAAAATCTCTTCCA | 238 |
| Hu.DMD.Exon49.25.009. | TTTAGACAAAATCTCTTCCACATCC | 239 |
| 2 | | |
| Hu.DMD.Exon49.25.010 | GTTTTTCCTTGTACAAATGCTGCCC | 240 |
| Hu.DMD.Exon49.25.010. | GTACAAATGCTGCCCTTTAGACAAA | 241 |
| 2 | | |
| Hu.DMD.Exon49.25.011 | CTTCACTGGCTGAGTGGCTGGTTTT | 242 |
| Hu.DMD.Exon49.25.011. | GGCTGGTTTTTCCTTGTACAAATGC | 243 |
| 2 | | |
| Hu.DMD.Exon49.25.012 | ATTACCTTCACTGGCTGAGTGGCTG | 244 |
| Hu.DMD.Exon49.25.013 | GCTTCATTACCTTCACTGGCTGAGT | 245 |
| Hu.DMD.Exon49.25.014 | AGGTTGCTTCATTACCTTCACTGGC | 246 |
| Hu.DMD.Exon49.25.015 | GCTAGAGGTTGCTTCATTACCTTCA | 247 |
| Hu.DMD.Exon49.25.016 | ATATTGCTAGAGGTTGCTTCATTAC | 248 |
| Hu.DMD.Exon49.20.001 | GAAAAGAACCCATATAGTGC | 249 |
| Hu.DMD.Exon49.20.002 | GGGAAAAGAACCCATATAGT | 250 |
| Hu.DMD.Exon49.20.003 | TCCTGGGGAAAAGAACCCAT | 251 |
| Hu.DMD.Exon49.20.004 | CAGTTTCCTGGGGAAAAGAA | 252 |
| Hu.DMD.Exon49.20.005 | TATTTCAGTTTCCTGGGGAA | 253 |
| Hu.DMD.Exon49.20.006 | ACTGCTATTTCAGTTTCCTG | 254 |
| Hu.DMD.Exon49.20.007 | CTTGAACTGCTATTTCAGTT | 255 |
| Hu.DMD.Exon49.20.008 | TTTAGCTTGAACTGCTATTT | 256 |
| Hu.DMD.Exon49.20.009 | TTCCACATCCGGTTGTTTAG | 257 |
| Hu.DMD.Exon49.20.010 | TTTAGACAAAATCTCTTCCA | 258 |
| Hu.DMD.Exon49.20.011 | GTACAAATGCTGCCCTTTAG | 259 |
| Hu.DMD.Exon49.20.012 | GGCTGGTTTTTCCTTGTACA | 260 |
| Hu.DMD.Exon49.20.013 | CTTCACTGGCTGAGTGGCTG | 261 |
| Hu.DMD.Exon49.20.014 | ATTACCTTCACTGGCTGAGT | 262 |
| Hu.DMD.Exon49.20.015 | GCTTCATTACCTTCACTGGC | 263 |
| Hu.DMD.Exon49.20.016 | AGGTTGCTTCATTACCTTCA | 264 |
| Hu.DMD.Exon49.20.017 | GCTAGAGGTTGCTTCATTAC | 265 |
| Hu.DMD.Exon49.20.018 | ATATTGCTAGAGGTTGCTTC | 266 |
| Hu.DMD.Exon50.25.001 | CTTTAACAGAAAAGCATACACATTA | 267 |
| Hu.DMD.Exon50.25.002 | TCCTCTTTAACAGAAAAGCATACAC | 268 |
| Hu.DMD.Exon50.25.002. | TTCCTCTTTAACAGAAAAGCATACA | 269 |
| 2 | | |
| Hu.DMD.Exon50.25.003 | TAACTTCCTOTTTAACAGAAAAGCA | 270 |
| Hu.DMD.Exon50.25.003. | CTAACTTCCTOTTTAACAGAAAAGC | 271 |
| 2 | | |
| Hu.DMD.Exon50.25.004 | TCTTCTAACTTCCTCTTTAACAGAA | 272 |
| Hu.DMD.Exon50.25.004. | ATCTTCTAACTTCCTCTTTAACAGA | 273 |
| 2 | | |
| Hu.DMD.Exon50.25.005 | TCAGATCTTCTAACTTCCTCTTTAA | 274 |
| Hu.DMD.Exon50.25.005. | CTCAGATCTTCTAACTTCCTCTTTA | 275 |
| 2 | | |
| Hu.DMD.Exon50.25.006 | AGAGCTCAGATCTTCTAACTTCCTC | 276 |
| Hu.DMD.Exon50.25.006. | CAGAGCTCAGATCTTCTAACTTCCT | 277 |
| 2 | | |
| NG-08−0731 | | |
| Hu.DMD.Exon50.25.007 | CACTCAGAGCTCAGATCTTCTACT | 278 |
| Hu.DMD.Exon50.25.007. | CCTTCCACTCAGAGCTCAGATCTTC | 279 |
| 2 | | |
| Hu.DMD.Exon50.25.008 | GTAAACGGTTTACCGCCTTCCACTC | 280 |
| Hu.DMD.Exon50.25.009 | CTTTGCCCTCAGCTCTTGAAGTAAA | 281 |
| Hu.DMD.Exon50.25.009. | CCCTCAGCTCTTGAAGTAAACGGTT | 282 |
| 2 | | |
| Hu.DMD.Exon50.25.010 | CCAGGAGCTAGGTCAGGCTGCTTTG | 283 |
| Hu.DMD.Exon50.25.010. | GGTCAGGCTGCTTTGCCCTCAGCTC | 284 |
| 2 | | |
| Hu.DMD.Exon50.25.011 | AGGCTCCAATAGTGGTCAGTCCAGG | 285 |
| Hu.DMD.Exon50.25.011. | TCAGTCCAGGAGCTAGGTCAGGCTG | 286 |
| 2 | | |
| Hu.DMD.Exon50.25.012 | CTTACAGGCTCCAATAGTGGTCAGT | 287 |
| AVI-5038 | | |
| Hu.DMD.Exon50.25.013 | GTATACTTACAGGCTCCAATAGTGG | 288 |
| Hu.DMD.Exon50.25.014 | ATCCAGTATACTTACAGGCTCCAAT | 289 |
| Hu.DMD.Exon50.25.015 | ATGGGATCCAGTATACTTACAGGCT | 290 |
| NG-08−0741 | | |
| Hu.DMD.Exon50.25.016 | AGAGAATGGGATCCAGTATACTTAC | 291 |
| NG-08−0742 | | |
| Hu.DMD.Exon50.20.001 | ACAGAAAAGCATACACATTA | 292 |
| Hu.DMD.Exon50.20.002 | TTTAACAGAAAAGCATACAC | 293 |
| Hu.DMD.Exon50.20.003 | TCCTCTTTAACAGAAAAGCA | 294 |
| Hu.DMD.Exon50.20.004 | TAACTTCCTCTTTAACAGAA | 295 |
| Hu.DMD.Exon50.20.005 | TOTTCTAACTTCCTOTTTAA | 296 |
| Hu.DMD.Exon50.20.006 | TCAGATCTTCTAACTTCCTC | 297 |
| Hu.DMD.Exon50.20.007 | CCTTCCACTCAGAGCTCAGA | 298 |
| Hu.DMD.Exon50.20.008 | GTAAACGGTTTACCGCCTTC | 299 |
| Hu.DMD.Exon50.20.009 | CCCTCAGCTCTTGAAGTAAA | 300 |
| Hu.DMD.Exon50.20.010 | GGTCAGGCTGCTTTGCCCTC | 301 |
| Hu.DMD.Exon50.20.011 | TCAGTCCAGGAGCTAGGTCA | 302 |
| Hu.DMD.Exon50.20.012 | AGGCTCCAATAGTGGTCAGT | 303 |
| Hu.DMD.Exon50.20.013 | CTTACAGGCTCCAATAGTGG | 304 |
| Hu.DMD.Exon50.20.014 | GTATACTTACAGGCTCCAAT | 305 |
| Hu.DMD.Exon50.20.015 | ATCCAGTATACTTACAGGCT | 306 |
| Hu.DMD.Exon50.20.016 | ATGGGATCCAGTATACTTAC | 307 |
| Hu.DMD.Exon50.20.017 | AGAGAATGGGATCCAGTATA | 308 |
| Hu.DMD.Exon51.25.001− | CTAAAATATTTTGGGTTTTTGCAAAA | 309 |
| 44 | | |
| Hu.DMD.Exon51.25.002− | GCTAAAATATTTTGGGTTTTTGCAAA | 310 |
| 45 | | |
| Hu.DMD.Exon51.25.002. | TAGGAGCTAAAATATTTTGGGTTTTT | 311 |
| 2−46 | | |
| Hu.DMD.Exon51.25.003 | AGTAGGAGCTAAAATATTTTGGGTT | 312 |
| Hu.DMD.Exon51.25.003. | TGAGTAGGAGCTAAAATATTTTGGG | 313 |
| 2 | | |
| Hu.DMD.Exon51.25.004 | CTGAGTAGGAGCTAAAATATTTTGGG | 314 |
| Hu.DMD.Exon51.25.004. | CAGTCTGAGTAGGAGCTAAAATATT | 315 |
| 2 | | |
| Hu.DMD.Exon51.25.005 | ACAGTCTGAGTAGGAGCTAAAATATT | 316 |
| Hu.DMD.Exon51.25.005. | GAGTAACAGTCTGAGTAGGAGCTAAA | 317 |
| 2 | | |
| Hu.DMD.Exon51.25.006 | CAGAGTAACAGTCTGAGTAGGAGCT | 318 |
| Hu.DMD.Exon51.25.006. | CACCAGAGTAACAGTCTGAGTAGGAG | 319 |
| 2 | | |
| Hu.DMD.Exon51.25.007 | GTCACCAGAGTAACAGTCTGAGTAG | 320 |
| Hu.DMD.Exon51.25.007. | AACCACAGGTTGTGTCACCAGAGTAA | 321 |
| 2 | | |
| Hu.DMD.Exon51.25.008 | GTTGTGTCACCAGAGTAACAGTCTG | 322 |
| Hu.DMD.Exon51.25.009 | TGGCAGTTTCCTTAGTAACCACAGGT | 323 |
| Hu.DMD.Exon51.25.010 | ATTTCTAGTTTGGAGATGGCAGTTTC | 324 |
| Hu.DMD.Exon51.25.010. | GGAAGATGGCATTTCTAGTTTGGAG | 325 |
| 2 | | |
| Hu.DMD.Exon51.25.011 | CATCAAGGAAGATGGCATTTCTAGTT | 326 |
| Hu.DMD.Exon51.25.011. | GAGCAGGTACCTCCAACATCAAGGAA | 327 |
| 2 | | |
| Hu.DMD.Exon51.25.012 | ATCTGCCAGAGCAGGTACCTCCAAC | 328 |
| Hu.DMD.Exon51.25.013 | AAGTTCTGTCCAAGCCCGGTTGAAAT | 329 |
| Hu.DMD.Exon51.25.013. | CGGTTGAAATCTGCCAGAGCAGGTAC | 330 |
| 2 | | |
| Hu.DMD.Exon51.25.014 | GAGAAAGCCAGTCGGTAAGTTCTGTC | 331 |
| Hu.DMD.Exon51.25.014. | GTCGGTAAGTTCTGTCCAAGCCCGG | 332 |
| 2 | | |
| Hu.DMD.Exon51.25.015 | ATAACTTGATCAAGCAGAGAAAGCCA | 333 |
| Hu.DMD.Exon51.25.015. | AAGCAGAGAAAGCCAGTCGGTAAGT | 334 |
| 2 | | |
| Hu.DMD.Exon51.25.016 | CACCCTCTGTGATTTTATAACTTGAT | 335 |
| Hu.DMD.Exon51.25.017 | CAAGGTCACCCACCATCACCCTCTGT | 336 |
| Hu.DMD.Exon51.25.017. | CATCACCCTCTGTGATTTTATAACT | 337 |
| 2 | | |
| Hu.DMD.Exon51.25.018 | CTTCTGCTTGATGATCATCTCGTTGA | 338 |
| Hu.DMD.Exon51.25.019 | CCTTCTGCTTGATGATCATCTCGTTG | 339 |
| Hu.DMD.Exon51.25.019. | ATCTCGTTGATATCCTCAAGGTCACC | 340 |
| 2 | | |
| Hu.DMD.Exon51.25.020 | TCATACCTTCTGCTTGATGATCATCT | 341 |
| Hu.DMD.Exon51.25.020. | TCATTTTTTCTCATACCTTCTGCTTG | 342 |
| 2 | | |
| Hu.DMD.Exon51.25.021 | TTTTCTCATACCTTCTGCTTGATGAT | 343 |
| Hu.DMD.Exon51.25.022 | TTTTATCATTTTTTCTCATACCTTCT | 344 |
| Hu.DMD.Exon51.25.023 | CCAACTTTTATCATTTTTTCTCATAC | 345 |
| Hu.DMD.Exon51.20.001 | ATATTTTGGGTTTTTGCAAA | 346 |
| Hu.DMD.Exon51.20.002 | AAAATATTTTGGGTTTTTGC | 347 |
| Hu.DMD.Exon51.20.003 | GAGCTAAAATATTTTGGGTT | 348 |
| Hu.DMD.Exon51.20.004 | AGTAGGAGCTAAAATATTTT | 349 |
| Hu.DMD.Exon51.20.005 | GTCTGAGTAGGAGCTAAAAT | 350 |
| Hu.DMD.Exon51.20.006 | TAACAGTCTGAGTAGGAGCT | 351 |
| Hu.DMD.Exon51.20.007 | CAGAGTAACAGTCTGAGTAG | 352 |
| Hu.DMD.Exon51.20.008 | CACAGGTTGTGTCACCAGAG | 353 |
| Hu.DMD.Exon51.20.009 | AGTTTCCTTAGTAACCACAG | 354 |
| Hu.DMD.Exon51.20.010 | TAGTTTGGAGATGGCAGTTT | 355 |
| Hu.DMD.Exon51.20.011 | GGAAGATGGCATTTCTAGTT | 356 |
| Hu.DMD.Exon51.20.012 | TACCTCCAACATCAAGGAAG | 357 |
| Hu.DMD.Exon51.20.013 | ATCTGCCAGAGCAGGTACCT | 358 |
| Hu.DMD.Exon51.20.014 | CCAAGCCCGGTTGAAATCTG | 359 |
| Hu.DMD.Exon51.20.015 | GTCGGTAAGTTCTGTCCAAG | 360 |
| Hu.DMD.Exon51.20.016 | AAGCAGAGAAAGCCAGTCGG | 361 |
| Hu.DMD.Exon51.20.017 | TTTTATAACTTGATCAAGCA | 362 |
| Hu.DMD.Exon51.20.018 | CATCACCCTCTGTGATTTTA | 363 |
| Hu.DMD.Exon51.20.019 | CTCAAGGTCACCCACCATCA | 364 |
| Hu.DMD.Exon51.20.020 | CATCTCGTTGATATCCTCAA | 365 |
| Hu.DMD.Exon51.20.021 | CTTCTGCTTGATGATCATOT | 366 |
| Hu.DMD.Exon51.20.022 | CATACCTTCTGCTTGATGAT | 367 |
| Hu.DMD.Exon51.20.023 | TTTCTCATACCTTCTGCTTG | 368 |
| Hu.DMD.Exon51.20.024 | CATTTTTTCTCATACCTTCT | 369 |
| Hu.DMD.Exon51.20.025 | TTTATCATTTTTTCTCATAC | 370 |
| Hu.DMD.Exon51.20.026 | CAACTTTTATCATTTTTTCT | 371 |
| Hu.DMD.Exon52.25.001 | CTGTAAGAACAAATATCCCTTAGTA | 372 |
| Hu.DMD.Exon52.25.002 | TGCCTGTAAGAACAAATATCCCTTA | 373 |
| Hu.DMD.Exon52.25.002. | GTTGCCTGTAAGAACAAATATCCCT | 374 |
| 2 | | |
| Hu.DMD.Exon52.25.003 | ATTGTTGCCTGTAAGAACAAATATC | 375 |
| Hu.DMD.Exon52.25.003. | GCATTGTTGCCTGTAAGAACAAATA | 376 |
| 2 | | |
| Hu.DMD.Exon52.25.004 | CCTGCATTGTTGCCTGTAAGAACAA | 377 |
| Hu.DMD.Exon52.25.004. | ATCCTGCATTGTTGCCTGTAAGAAC | 378 |
| 2 | | |
| Hu.DMD.Exon52.25.005 | CAAATCCTGCATTGTTGCCTGTAAG | 379 |
| Hu.DMD.Exon52.25.005. | TCCAAATCCTGCATTGTTGCCTGTA | 380 |
| 2 | | |
| Hu.DMD.Exon52.25.006 | TGTTCCAAATCCTGCATTGTTGCCT | 381 |
| Hu.DMD.Exon52.25.006. | TCTGTTCCAAATCCTGCATTGTTGC | 382 |
| 2 | | |
| Hu.DMD.Exon52.25.007 | AACTGGGGACGCCTCTGTTCCAAAT | 383 |
| Hu.DMD.Exon52.25.007. | GCCTCTGTTCCAAATCCTGCATTGT | 384 |
| 2 | | |
| Hu.DMD.Exon52.25.008 | CAGCGGTAATGAGTTCTTCCAACTG | 385 |
| Hu.DMD.Exon52.25.008. | CTTCCAACTGGGGACGCCTCTGTTC | 386 |
| 2 | | |
| Hu.DMD.Exon52.25.009 | CTTGTTTTTCAAATTTTGGGCAGCG | 387 |
| Hu.DMD.Exon52.25.010 | CTAGCCTCTTGATTGCTGGTCTTGT | 388 |
| Hu.DMD.Exon52.25.010. | TTTTCAAATTTTGGGCAGCGGTAAT | 389 |
| 2 | | |
| Hu.DMD.Exon52.25.011 | TTCGATCCGTAATGATTGTTCTAGC | 390 |
| Hu.DMD.Exon52.25.011. | GATTGCTGGTCTTGTTTTTCAAATT | 391 |
| 2 | | |
| Hu.DMD.Exon52.25.012 | CTTACTTCGATCCGTAATGATTGTT | 392 |
| Hu.DMD.Exon52.25.012. | TTGTTCTAGCCTCTTGATTGCTGGT | 393 |
| 2 | | |
| Hu.DMD.Exon52.25.013 | AAAAACTTACTTCGATCCGTAATGA | 394 |
| Hu.DMD.Exon52.25.014 | TGTTAAAAAACTTACTTCGATCCGT | 395 |
| Hu.DMD.Exon52.25.015 | ATGCTTGTTAAAAAACTTACTTCGA | 396 |
| Hu.DMD.Exon52.25.016 | GTCCCATGCTTGTTAAAAAACTTAC | 397 |
| Hu.DMD.Exon52.20.001 | AGAACAAATATCCCTTAGTA | 398 |
| Hu.DMD.Exon52.20.002 | GTAAGAACAAATATCCCTTA | 399 |
| Hu.DMD.Exon52.20.003 | TGCCTGTAAGAACAAATATC | 400 |
| Hu.DMD.Exon52.20.004 | ATTGTTGCCTGTAAGAACAA | 401 |
| Hu.DMD.Exon52.20.005 | CCTGCATTGTTGCCTGTAAG | 402 |
| Hu.DMD.Exon52.20.006 | CAAATCCTGCATTGTTGCCT | 403 |
| Hu.DMD.Exon52.20.007 | GCCTCTGTTCCAAATCCTGC | 404 |
| Hu.DMD.Exon52.20.008 | CTTCCAACTGGGGACGCCTC | 405 |
| Hu.DMD.Exon52.20.009 | CAGCGGTAATGAGTTCTTCC | 406 |
| Hu.DMD.Exon52.20.010 | TTTTCAAATTTTGGGCAGCG | 407 |
| Hu.DMD.Exon52.20.011 | GATTGCTGGTCTTGTTTTTC | 408 |
| Hu.DMD.Exon52.20.012 | TTGTTCTAGCCTCTTGATTG | 409 |
| Hu.DMD.Exon52.20.013 | TTCGATCCGTAATGATTGTT | 410 |
| Hu.DMD.Exon52.20.014 | CTTACTTCGATCCGTAATGA | 411 |
| Hu.DMD.Exon52.20.015 | AAAAACTTACTTCGATCCGT | 412 |
| Hu.DMD.Exon52.20.016 | TGTTAAAAAACTTACTTCGA | 413 |
| Hu.DMD.Exon52.20.017 | ATGCTTGTTAAAAAACTTAC | 414 |
| Hu.DMD.Exon52.20.018 | GTCCCATGCTTGTTAAAAAA | 415 |
| Hu.DMD.Exon53.25.001 | CTAGAATAAAAGGAAAAATAAATAT | 416 |
| Hu.DMD.Exon53.25.002 | AACTAGAATAAAAGGAAAAATAAAT | 417 |
| Hu.DMD.Exon53.25.002. | TTCAACTAGAATAAAAGGAAAAATA | 418 |
| 2 | | |
| Hu.DMD.Exon53.25.003 | CTTTCAACTAGAATAAAAGGAAAAA | 419 |
| Hu.DMD.Exon53.25.003. | ATTCTTTCAACTAGAATAAAAGGAA | 420 |
| 2 | | |
| Hu.DMD.Exon53.25.004 | GAATTCTTTCAACTAGAATAAAAGG | 421 |
| Hu.DMD.Exon53.25.004. | TCTGAATTCTTTCAACTAGAATAAA | 422 |
| 2 | | |
| Hu.DMD.Exon53.25.005 | ATTCTGAATTCTTTCAACTAGAATA | 423 |
| Hu.DMD.Exon53.25.005. | CTGATTCTGAATTCTTTCAACTAGA | 424 |
| 2 | | |
| Hu.DMD.Exon53.25.006 | CACTGATTCTGAATTCTTTCAACTA | 425 |
| Hu.DMD.Exon53.25.006. | TCCCACTGATTCTGAATTCTTTCAA | 426 |
| 2 | | |
| Hu.DMD.Exon53.25.007 | CATCCCACTGATTCTGAATTCTTTC | 427 |
| Hu.DMD.Exon53.25.008 | TACTTCATCCCACTGATTCTGAATT | 428 |
| Hu.DMD.Exon53.25.008. | CTGAAGGTGTTCTTGTACTTCATCC | 429 |
| 2 | | |
| Hu.DMD.Exon53.25.009 | CGGTTCTGAAGGTGTTCTTGTACT | 430 |
| Hu.DMD.Exon53.25.009. | CTGTTGCCTCCGGTTCTGAAGGTGT | 431 |
| 2 | | |
| Hu.DMD.Exon53.25.010 | TTTCATTCAACTGTTGCCTCCGGTT | 432 |
| Hu.DMD.Exon53.25.010. | TAACATTTCATTCAACTGTTGCCTC | 433 |
| 2 | | |
| Hu.DMD.Exon53.25.011 | TTGTGTTGAATCCTTTAACATTTCA | 434 |
| Hu.DMD.Exon53.25.012 | TCTTCCTTAGCTTCCAGCCATTGTG | 435 |
| Hu.DMD.Exon53.25.012. | CTTAGCTTCCAGCCATTGTGTTGAA | 436 |
| 2 | | |
| Hu.DMD.Exon53.25.013 | GTCCTAAGACCTGCTCAGCTTCTTC | 437 |
| Hu.DMD.Exon53.25.013. | CTGCTCAGCTTCTTCCTTAGCTTCC | 438 |
| 2 | | |
| Hu.DMD.Exon53.25.014 | CTCAAGCTTGGCTCTGGCCTGTCCT | 439 |
| Hu.DMD.Exon53.25.014. | GGCCTGTCCTAAGACCTGCTCAGCT | 440 |
| 2 | | |
| Hu.DMD.Exon53.25.015 | TAGGGACCCTCCTTCCATGACTCAA | 441 |
| Hu.DMD.Exon53.25.016 | TTTGGATTGCATCTACTGTATAGGG | 442 |
| Hu.DMD.Exon53.25.016. | ACCCTCCTTCCATGACTCAAGCTTG | 443 |
| 2 | | |
| Hu.DMD.Exon53.25.017 | CTTGGTTTCTGTGATTTTCTTTTGG | 444 |
| Hu.DMD.Exon53.25.017. | ATCTACTGTATAGGGACCCTCCTTC | 445 |
| 2 | | |
| Hu.DMD.Exon53.25.018 | CTAACCTTGGTTTCTGTGATTTTCT | 446 |
| Hu.DMD.Exon53.25.018. | TTTCTTTTGGATTGCATCTACTGTA | 447 |
| 2 | | |
| Hu.DMD.Exon53.25.019 | TGATACTAACCTTGGTTTCTGTGAT | 448 |
| Hu.DMD.Exon53.25.020 | ATCTTTGATACTAACCTTGGTTTCT | 449 |
| Hu.DMD.Exon53.25.021 | AAGGTATCTTTGATACTAACCTTGG | 450 |
| Hu.DMD.Exon53.25.022 | TTAAAAAGGTATCTTTGATACTAAC | 451 |
| Hu.DMD.Exon53.20.001 | ATAAAAGGAAAAATAAATAT | 452 |
| Hu.DMD.Exon53.20.002 | GAATAAAAGGAAAAATAAAT | 453 |
| Hu.DMD.Exon53.20.003 | AACTAGAATAAAAGGAAAAA | 454 |
| Hu.DMD.Exon53.20.004 | CTTTCAACTAGAATAAAAGG | 455 |
| Hu.DMD.Exon53.20.005 | GAATTCTTTCAACTAGAATA | 456 |
| Hu.DMD.Exon53.20.006 | ATTCTGAATTCTTTCAACTA | 457 |
| Hu.DMD.Exon53.20.007 | TACTTCATCCCACTGATTCT | 458 |
| Hu.DMD.Exon53.20.008 | CTGAAGGTGTTCTTGTACT | 459 |
| Hu.DMD.Exon53.20.009 | CTGTTGCCTCCGGTTCTGAA | 460 |
| Hu.DMD.Exon53.20.010 | TAACATTTCATTCAACTGTT | 461 |
| Hu.DMD.Exon53.20.011 | TTGTGTTGAATCCTTTAACA | 462 |
| Hu.DMD.Exon53.20.012 | CTTAGCTTCCAGCCATTGTG | 463 |
| Hu.DMD.Exon53.20.013 | CTGCTCAGCTTCTTCCTTAG | 464 |
| Hu.DMD.Exon53.20.014 | GGCCTGTCCTAAGACCTGCT | 465 |
| Hu.DMD.Exon53.20.015 | CTCAAGCTTGGCTCTGGCCT | 466 |
| Hu.DMD.Exon53.20.016 | ACCCTCCTTCCATGACTCAA | 467 |
| Hu.DMD.Exon53.20.017 | ATCTACTGTATAGGGACCCT | 468 |
| Hu.DMD.Exon53.20.018 | TTTCTTTTGGATTGCATCTA | 469 |
| Hu.DMD.Exon53.20.019 | CTTGGTTTCTGTGATTTTCT | 470 |
| Hu.DMD.Exon53.20.020 | CTAACCTTGGTTTCTGTGAT | 471 |
| Hu.DMD.Exon53.20.021 | TGATACTAACCTTGGTTTCT | 472 |
| Hu.DMD.Exon53.20.022 | ATCTTTGATACTAACCTTGG | 473 |
| Hu.DMD.Exon53.20.023 | AAGGTATCTTTGATACTAAC | 474 |
| Hu.DMD.Exon53.20.024 | TTAAAAAGGTATCTTTGATA | 475 |
| Hu.DMD.Exon54.25.001 | CTATAGATTTTTATGAGAAAGAGA | 476 |
| Hu.DMD.Exon54.25.002 | AACTGCTATAGATTTTTATGAGAAA | 477 |
| Hu.DMD.Exon54.25.003 | TGGCCAACTGCTATAGATTTTTATG | 478 |
| Hu.DMD.Exon54.25.004 | GTCTTTGGCCAACTGCTATAGATTT | 479 |
| Hu.DMD.Exon54.25.005 | CGGAGGTCTTTGGCCAACTGCTATA | 480 |
| Hu.DMD.Exon54.25.006 | ACTGGCGGAGGTCTTTGGCCAACTG | 481 |
| Hu.DMD.Exon54.25.007 | TTTGTCTGCCACTGGCGGAGGTCTT | 482 |
| Hu.DMD.Exon54.25.008 | AGTCATTTGCCACATCTACATTTGT | 483 |
| Hu.DMD.Exon54.25.008. | TTTGCCACATCTACATTTGTCTGCC | 484 |
| 2 | | |
| Hu.DMD.Exon54.25.009 | CCGGAGAAGTTTCAGGGCCAAGTCA | 485 |
| Hu.DMD.Exon54.25.010 | GTATCATCTGCAGAATAATCCCGGA | 486 |
| Hu.DMD.Exon54.25.010. | TAATCCCGGAGAAGTTTCAGGGCCA | 487 |
| 2 | | |
| Hu.DMD.Exon54.25.011 | TTATCATGTGGACTTTTCTGGTATC | 488 |
| Hu.DMD.Exon54.25.012 | AGAGGCATTGATATTCTCTGTTATC | 489 |
| Hu.DMD.Exon54.25.012. | ATGTGGACTTTTCTGGTATCATCTG | 490 |
| 2 | | |
| Hu.DMD.Exon54.25.013 | CTTTTATGAATGCTTCTCCAAGAGG | 491 |
| Hu.DMD.Exon54.25.013. | ATATTCTCTGTTATCATGTGGACTT | 492 |
| 2 | | |
| Hu.DMD.Exon54.25.014 | CATACCTTTTATGAATGCTTCTCCA | 493 |
| Hu.DMD.Exon54.25.014. | CTCCAAGAGGCATTGATATTCTCTG | 494 |
| 2 | | |
| Hu.DMD.Exon54.25.015 | TAATTCATACCTTTTATGAATGCTT | 495 |
| Hu.DMD.Exon54.25.015. | CTTTTATGAATGCTTCTCCAAGAGG | 496 |
| 2 | | |
| Hu.DMD.Exon54.25.016 | TAATGTAATTCATACCTTTTATGAA | 497 |
| Hu.DMD.Exon54.25.017 | AGAAATAATGTAATTCATACCTTTT | 498 |
| Hu.DMD.Exon54.25.018 | GTTTTAGAAATAATGTAATTCATAC | 499 |
| Hu.DMD.Exon54.20.001 | GATTTTTATGAGAAAGAGA | 500 |
| Hu.DMD.Exon54.20.002 | CTATAGATTTTTATGAGAAA | 501 |
| Hu.DMD.Exon54.20.003 | AACTGCTATAGATTTTTATG | 502 |
| Hu.DMD.Exon54.20.004 | TGGCCAACTGCTATAGATTT | 503 |
| Hu.DMD.Exon54.20.005 | GTCTTTGGCCAACTGCTATA | 504 |
| Hu.DMD.Exon54.20.006 | CGGAGGTCTTTGGCCAACTG | 505 |
| Hu.DMD.Exon54.20.007 | TTTGTCTGCCACTGGCGGAG | 506 |
| Hu.DMD.Exon54.20.008 | TTTGCCACATCTACATTTGT | 507 |
| Hu.DMD.Exon54.20.009 | TTCAGGGCCAAGTCATTTGC | 508 |
| Hu.DMD.Exon54.20.010 | TAATCCCGGAGAAGTTTCAG | 509 |
| Hu.DMD.Exon54.20.011 | GTATCATCTGCAGAATAATC | 510 |
| Hu.DMD.Exon54.20.012 | ATGTGGACTTTTCTGGTATC | 511 |
| Hu.DMD.Exon54.20.013 | ATATTCTCTGTTATCATGTG | 512 |
| Hu.DMD.Exon54.20.014 | CTCCAAGAGGCATTGATATT | 513 |
| Hu.DMD.Exon54.20.015 | CTTTTATGAATGCTTCTCCA | 514 |
| Hu.DMD.Exon54.20.016 | CATACCTTTTATGAATGCTT | 515 |
| Hu.DMD.Exon54.20.017 | TAATTCATACCTTTTATGAA | 516 |
| Hu.DMD.Exon54.20.018 | TAATGTAATTCATACCTTTT | 517 |
| Hu.DMD.Exon54.20.019 | AGAAATAATGTAATTCATAC | 518 |
| Hu.DMD.Exon54.20.020 | GTTTTAGAAATAATGTAATT | 519 |
| Hu.DMD.Exon55.25.001 | CTGCAAAGGACCAAATGTTCAGATG | 520 |
| Hu.DMD.Exon55.25.002 | TCACCCTGCAAAGGACCAAATGTTC | 521 |
| Hu.DMD.Exon55.25.003 | CTCACTCACCCTGCAAAGGACCAAA | 522 |
| Hu.DMD.Exon55.25.004 | TCTCGCTCACTCACCCTGCAAAGGA | 523 |
| Hu.DMD.Exon55.25.005 | CAGCCTCTCGCTCACTCACCCTGCA | 524 |
| Hu.DMD.Exon55.25.006 | CAAAGCAGCCTCTCGCTCACTCACC | 525 |
| Hu.DMD.Exon55.25.007 | TCTTCCAAAGCAGCCTCTCGCTCAC | 526 |
| Hu.DMD.Exon55.25.007. | TCTATGAGTTTCTTCCAAAGCAGCC | 527 |
| 2 | | |
| Hu.DMD.Exon55.25.008 | GTTGCAGTAATCTATGAGTTTCTTC | 528 |
| Hu.DMD.Exon55.25.008. | GAACTGTTGCAGTAATCTATGAGTT | 529 |
| 2 | | |
| Hu.DMD.Exon55.25.009 | TTCCAGGTCCAGGGGGAACTGTTGC | 530 |
| Hu.DMD.Exon55.25.010 | GTAAGCCAGGCAAGAAACTTTTCCA | 531 |
| Hu.DMD.Exon55.25.010. | CCAGGCAAGAAACTTTTCCAGGTCC | 532 |
| 2 | | |
| Hu.DMD.Exon55.25.011 | TGGCAGTTGTTTCAGCTTCTGTAAG | 533 |
| Hu.DMD.Exon55.25.011. | TTCAGCTTCTGTAAGCCAGGCAAGA | 635 |
| 2 | | |
| Hu.DMD.Exon55.25.012 | GGTAGCATCCTGTAGGACATTGGCA | 534 |
| Hu.DMD.Exon55.25.012. | GACATTGGCAGTTGTTTCAGCTTCT | 535 |
| 2 | | |
| Hu.DMD.Exon55.25.013 | TCTAGGAGCCTTTCCTTACGGGTAG | 536 |
| Hu.DMD.Exon55.25.014 | CTTTTACTCCCTTGGAGTCTTCTAG | 537 |
| Hu.DMD.Exon55.25.014. | GAGCCTTTCCTTACGGGTAGCATCC | 538 |
| 2 | | |
| Hu.DMD.Exon55.25.015 | TTGCCATTGTTTCATCAGCTCTTTT | 539 |
| Hu.DMD.Exon55.25.015. | CTTGGAGTCTTCTAGGAGCCTTTCC | 540 |
| 2 | | |
| Hu.DMD.Exon55.25.016 | CTTACTTGCCATTGTTTCATCAGCT | 541 |
| Hu.DMD.Exon55.25.016. | CAGCTCTTTTACTCCCTTGGAGTCT | 542 |
| 2 | | |
| Hu.DMD.Exon55.25.017 | CCTGACTTACTTGCCATTGTTTCAT | 543 |
| Hu.DMD.Exon55.25.018 | AAATGCCTGACTTACTTGCCATTGT | 544 |
| Hu.DMD.Exon55.25.019 | AGCGGAAATGCCTGACTTACTTGCC | 545 |
| Hu.DMD.Exon55.25.020 | GCTAAAGCGGAAATGCCTGACTTAC | 546 |
| Hu.DMD.Exon55.20.001 | AAGGACCAAATGTTCAGATG | 547 |
| Hu.DMD.Exon55.20.002 | CTGCAAAGGACCAAATGTTC | 548 |
| Hu.DMD.Exon55.20.003 | TCACCCTGCAAAGGACCAAA | 549 |
| Hu.DMD.Exon55.20.004 | CTCACTCACCCTGCAAAGGA | 550 |
| Hu.DMD.Exon55.20.005 | TCTCGCTCACTCACCCTGCA | 551 |
| Hu.DMD.Exon55.20.006 | CAGCCTCTCGCTCACTCACC | 552 |
| Hu.DMD.Exon55.20.007 | CAAAGCAGCCTCTCGCTCAC | 553 |
| Hu.DMD.Exon55.20.008 | TCTATGAGTTTCTTCCAAAG | 554 |
| Hu.DMD.Exon55.20.009 | GAACTGTTGCAGTAATCTAT | 555 |
| Hu.DMD.Exon55.20.010 | TTCCAGGTCCAGGGGGAACT | 556 |
| Hu.DMD.Exon55.20.011 | CCAGGCAAGAAACTTTTCCA | 557 |
| Hu.DMD.Exon55.20.012 | TTCAGCTTCTGTAAGCCAGG | 558 |
| Hu.DMD.Exon55.20.013 | GACATTGGCAGTTGTTTCAG | 559 |
| Hu.DMD.Exon55.20.014 | GGTAGCATCCTGTAGGACAT | 560 |
| Hu.DMD.Exon55.20.015 | GAGCCTTTCCTTACGGGTAG | 561 |
| Hu.DMD.Exon55.20.016 | CTTGGAGTCTTCTAGGAGCC | 562 |
| Hu.DMD.Exon55.20.017 | CAGCTCTTTTACTCCCTTGG | 563 |
| Hu.DMD.Exon55.20.018 | TTGCCATTGTTTCATCAGCT | 564 |
| Hu.DMD.Exon55.20.019 | CTTACTTGCCATTGTTTCAT | 565 |
| Hu.DMD.Exon55.20.020 | CCTGACTTACTTGCCATTGT | 566 |
| Hu.DMD.Exon55.20.021 | AAATGCCTGACTTACTTGCC | 567 |
| Hu.DMD.Exon55.20.022 | AGCGGAAATGCCTGACTTAC | 568 |
| Hu.DMD.Exon55.20.023 | GCTAAAGCGGAAATGCCTGA | 569 |
| H50A(+02+30)-AVI-5656 | CCACTCAGAGCTCAGATCTTCTAACTTC | 584 |
| | C | |
| H50D(+07−18)-AVI-5915 | GGGATCCAGTATACTTACAGGCTCC | 585 |
| H50A(+07+33) | CTTCCACTCAGAGCTCAGATCTTCTAA | 586 |
| H51A(+61+90)-AVI-4657 | ACATCAAGGAAGATGGCATTTCTAGTTT | 587 |
| | GG | |
| H51A(+66+95)-AVI-4658 | CTCCAACATCAAGGAAGATGGCATTTCT | 588 |
| | AG | |
| H51A(+111+134) | TTCTGTCCAAGCCCGGTTGAAATC | 589 |
| H51A(+175+195) | CACCCACCATCACCCTCYGTG | 590 |
| H51A(+199+220) | ATCATCTCGTTGATATCCTCAA | 591 |
| H51A(+66+90) | ACATCAAGGAAGATGGCATTTCTAG | 592 |
| H51A(−01+25) | ACCAGAGTAACAGTCTGAGTAGGAGC | 593 |
| h51AON1 | TCAAGGAAGATGGCATTTCT | 594 |
| h51AON2 | CCTCTGTGATTTTATAACTTGAT | 595 |
| H51D(+08−17) | ATCATTTTTTCTCATACCTTCTGCT | 596 |
| H51D(+16−07) | CTCATACCTTCTGCTTGATGATC | 597 |
| hAON#23 | TGGCATTTCTAGTTTGG | 598 |
| hAON#24 | CCAGAGCAGGTACCTCCAACATC | 599 |
| H44A(+61+84) | TGTTCAGCTTCTGTTAGCCACTGA | 600 |
| H44A(+85+104) | TTTGTGTCTTTCTGAGAAAC | 601 |
| h44AON1 | CGCCGCCATTTCTCAACAG | 602 |
| H44A(−06+14) | ATCTGTCAAATCGCCTGCAG | 603 |
| H45A(+71+90) | TGTTTTTGAGGATTGCTGAA | 604 |
| h45AON1 | GCTGAATTATTTCTTCCCC | 605 |
| h45AON5 | GCCCAATGCCATCCTGG | 606 |
| H45A(−06+20) | CCAATGCCATCCTGGAGTTCCTGTAA | 607 |
| H53A(+39+69) | CATTCAACTGTTGCCTCCGGTTCTGAAG | 608 |
| | GTG | |
| H53A(+23+47) | CTGAAGGTGTTCTTGTACTTCATCC | 609 |
| h53AON1 | CTGTTGCCTCCGGTTCTG | 610 |
| H53A(−12+10) | ATTCTTTCAACTAGAATAAAAG | 611 |
| huEx45.30.66 | GCCATCCTGGAGTTCCTGTAAGATACC | 612 |
| | AAA | |
| huEx45.30.71 | CCAATGCCATCCTGGAGTTCCTGTAAG | 613 |
| | ATA | |
| huEx45.30.79 | GCCGCTGCCCAATGCCATCCTGGAGTT | 614 |
| | CCT | |
| huEx45.30.83 | GTTTGCCGCTGCCCAATGCCATCCTGG | 615 |
| | AGT | |
| huEx45.30.88 | CAACAGTTTGCCGCTGCCCAATGCCAT | 616 |
| | CCT | |
| huEx45.30.92 | CTGACAACAGTTTGCCGCTGCCCAATG | 617 |
| | CCA | |
| huEx45.30.96 | TGTTCTGACAACAGTTTGCCGCTGCCC | 618 |
| | AAT | |
| huEx45.30.99 | CAATGTTCTGACAACAGTTTGCCGCTG | 619 |
| | CCC | |
| huEx45.30.103 | CATTCAATGTTCTGACAACAGTTTGCCG | 620 |
| | CT | |
| huEx45.30.120 | TATTTCTTCCCCAGTTGCATTCAATGTT | 621 |
| | CT | |
| huEx45.30.127 | GCTGAATTATTTCTTCCCCAGTTGCATT | 622 |
| | CA | |
| huEx45.30.132 | GGATTGCTGAATTATTTCTTCCCCAGTT | 623 |
| | GC | |
| huEx45.30.137 | TTTGAGGATTGCTGAATTATTTCTTCCC | 624 |
| | CA | |
| huEx53.30.84 | GTACTTCATCCCACTGATTCTGAATTCT | 625 |
| | TT | |
| huEx53.30.88 | TCTTGTACTTCATCCCACTGATTCTGAA | 626 |
| | TT | |
| huEx53.30.91 | TGTTCTTGTACTTCATCCCACTGATTCT | 627 |
| | GA | |
| huEx53.30.103 | CGGTTCTGAAGGTGTTCTTGTACTTCAT | 628 |
| | CC | |
| huEx53.30.106 | CTCCGGTTCTGAAGGTGTTCTTGTACTT | 629 |
| | CA | |
| huEx53.30.109 | TGCCTCCGGTTCTGAAGGTGTTCTTGTA | 630 |
| | CT | |
| huEx53.30.112 | TGTTGCCTCCGGTTCTGAAGGTGTTCTT | 631 |
| | GT | |
| huEx53.30.115 | AACTGTTGCCTCCGGTTCTGAAGGTGT | 632 |
| | TCT | |
| huEx53.30.118 | TTCAACTGTTGCCTCCGGTTCTGAAGGT | 633 |
| | GT | |
| h50AON1 | | |
| h50AON2 | | |

Peptide Transporters (NH₂ to COOH)*:

| | | |
|---|---|---|
| rTAT | RRRQRRKKRC | 570 |
| R₉F₂ | RRRRRRRRRFFC | 571 |
| (RRAhx)₄B | RRAhxRRAhxRRAhxRRAhxB | 572 |
| (RAhxR)₄AhxB; (P007) | RAhxRRAhxRRAhxRRAhxRAhxB | 573 |
| (AhxRR)₄AhxB | AhxRRAhxRRAhxRRAhxRRAhxB | 574 |
| (RAhx)₆B | RAhxRAhxRAhxRAhxRAhxRAhxB | 575 |
| (RAhx)₈B | RAhxRAhxRAhxRAhxRAhxRAhxRAhxRAhx | 576 |
| | B | |
| (RAhxR)₅AhxB | RAhxRRAhxRRAhxRRAhxRRAhxRAhxB | 577 |
| (RAhxRRBR)₂AhxB; | RAhxRRBRRAhxRRBRAhxB | 578 |
| (CPO6062) | | |
| MSP | ASSLNIA | 579 |

Cell Penetrating Peptide/Homing Peptide/PMO Conjugates
(NH₂ to COOH and 5′ to 3′)

| | | |
|---|---|---|
| MSP-PMO | ASSLNIA-XB- | 580 |
| | GGCCAAACCTCGGCTTACCTGAAAT | 636 |
| CP06062−MSP-PMO | RXRRBRRXRRBR-XB-ASSLNIA-X- | 581 |
| | GGCCAAACCTCGGCTTACCTGAAAT | 636 |
| MSP-CP06062−PMO | ASSLNIA-X-RXRRBRRXRRBR-B- | 582 |
| | GGCCAAACCTCGGCTTACCTGAAAT | 636 |
| CP06062−PMO | RXRRBRRXRRBR-XB- | 583 |
| | GGCCAAACCTCGGCTTACCTGAAAT | 636 |

*Ahx is 6−aminohexanoic acid and B is beta-alanine.

1. A method for treating Duchenne muscular dystrophy in a patient in need thereof comprising administering to the patient a dose of eteplirsen of about 30 mg/kg once a week.


SEQUENCE LISTING (891 sequences)

SEQ ID NO: 1 - DNA, 30 bp, synthetic construct
  source 1..30: mol_type = other DNA
  misc_feature 1..30: note = Synthetic (eteplirsen H51A(+66+95))
ctccaacatcaaggaagatggcatttctag

SEQ ID NO: 2 - RNA, 25 nt, synthetic construct
  source 1..25: mol_type = other RNA
  misc_feature 1..25: note = Synthetic (H51A(+66+90))
acatcaaggaagatggcatttctag

SEQ ID NO: 3 - RNA, 30 nt, synthetic construct
  source 1..30: mol_type = other RNA
  misc_feature 1..30: note = Synthetic (H51A(+61+90))
acatcaaggaagatggcatttctagtttgg

SEQ ID NO: 4 - DNA, 26 bp, synthetic construct
  source 1..26: mol_type = other DNA
  misc_feature 1..26: note = Synthetic (Hu.DMD.exon51.25.001.2)
gagcaggtacctccaacatcaaggaa

SEQ ID NO: 5 - RNA, 25 nt, synthetic construct
  source 1..25: mol_type = other RNA
  misc_feature 1..25: note = Synthetic (H50D(+07-18))
gggatccagtatacttacaggctcc

SEQ ID NO: 6 - DNA, 25 bp, synthetic construct
  source 1..25: mol_type = other DNA
  misc_feature 1..25: note = Synthetic (AVI-4038/5038 )
cttacaggctccaatagtggtcagt

SEQ ID NO: 7 - DNA, 27 bp, synthetic construct
  source 1..27: mol_type = other DNA
  misc_feature 1..27: note = Synthetic (H53A27(+30+56))
cctccggttctgaaggtgttcttgtac

SEQ ID NO: 8 - DNA, 25 bp, synthetic construct
  source 1..25: mol_type = other DNA
  misc_feature 1..25: note = Synthetic (H53A(+36+60) )
gttgcctccggttctgaaggtgttc

SEQ ID NO: 9 - DNA, 22 bp, synthetic construct
  source 1..22: mol_type = other DNA
  misc_feature 1..22: note = Synthetic (H45A (-03+19))
caatgccatcctggagttcctg

SEQ ID NO: 10 - RNA, 24 nt, synthetic construct
  source 1..24: mol_type = other RNA
  misc_feature 1..24: note = Synthetic (H8A(-06+18))
gataggtggtatcaacatctgtaa

SEQ ID NO: 11 - RNA, 21 nt, synthetic construct
  source 1..21: mol_type = other RNA
  misc_feature 1..21: note = Synthetic (H8A (-03+18))
gataggtggtatcaacatctg

SEQ ID NO: 12 - RNA, 25 nt, synthetic construct
  source 1..25: mol_type = other RNA
  misc_feature 1..25: note = Synthetic (H8A(-07+18))
gataggtggtatcaacatctgtaag

SEQ ID NO: 13 - RNA, 20 nt, synthetic construct
  source 1..20: mol_type = other RNA
  misc_feature 1..20: note = Synthetic (H8A(-06+14))
ggtggtatcaacatctgtaa

SEQ ID NO: 14 - RNA, 20 nt, synthetic construct
  source 1..20: mol_type = other RNA
  misc_feature 1..20: note = Synthetic (H8A(-10+10))
gtatcaacatctgtaagcac

SEQ ID NO: 15 - RNA, 23 nt, synthetic construct
  source 1..23: mol_type = other RNA
  misc_feature 1..23: note = Synthetic (H7A(+45+67))
tgcatgttccagtcgttgtgtgg

SEQ ID NO: 16 - RNA, 25 nt, synthetic construct
  source 1..25: mol_type = other RNA
  misc_feature 1..25: note = Synthetic (H7A(+02+26))
cactattccagtcaaataggtctgg

SEQ ID NO: 17 - RNA, 25 nt, synthetic construct
  source 1..25: mol_type = other RNA
  misc_feature 1..25: note = Synthetic (H7D(+15-10))
atttaccaaccttcaggatcgagta

SEQ ID NO: 18 - RNA, 21 nt, synthetic construct
  source 1..21: mol_type = other RNA
  misc_feature 1..21: note = Synthetic (H7A(-18+03))
ggcctaaaacacatacacata

SEQ ID NO: 19 - RNA, 20 nt, synthetic construct
  source 1..20: mol_type = other RNA
  misc_feature 1..20: note = Synthetic (C6A(-10+10))
catttttgacctacatgtgg

SEQ ID NO: 20 - RNA, 20 nt, synthetic construct
  source 1..20: mol_type = other RNA
  misc_feature 1..20: note = Synthetic (C6A(-14+06))
tttgacctacatgtggaaag

SEQ ID NO: 21 - RNA, 26 nt, synthetic construct
  source 1..26: mol_type = other RNA
  misc_feature 1..26: note = Synthetic (C6A(-14+12))
tacatttttgacctacatgtggaaag

SEQ ID NO: 22 - RNA, 22 nt, synthetic construct
  source 1..22: mol_type = other RNA
  misc_feature 1..22: note = Synthetic (C6A(-13+09))
atttttgacctacatgggaaag

SEQ ID NO: 23 - RNA, 23 nt, synthetic construct
  source 1..23: mol_type = other RNA
  misc_feature 1..23: note = Synthetic (CH6A(+69+91))
tacgagttgattgtcggacccag

SEQ ID NO: 24 - RNA, 25 nt, synthetic construct
  source 1..25: mol_type = other RNA
  misc_feature 1..25: note = Synthetic (C6D(+12-13))
gtggtctccttacctatgactgtgg

SEQ ID NO: 25 - RNA, 17 nt, synthetic construct
  source 1..17: mol_type = other RNA
  misc_feature 1..17: note = Synthetic (C6D(+06-11))
ggtctccttacctatga

SEQ ID NO: 26 - RNA, 24 nt, synthetic construct
  source 1..24: mol_type = other RNA
  misc_feature 1..24: note = Synthetic (H6D(+04-21))
tgtctcagtaatcttcttacctat

SEQ ID NO: 27 - RNA, 24 nt, synthetic construct
  source 1..24: mol_type = other RNA
  misc_feature 1..24: note = Synthetic (H6D(+18-04))
tcttacctatgactatggatgaga

SEQ ID NO: 28 - RNA, 20 nt, synthetic construct
  source 1..20: mol_type = other RNA
  misc_feature 1..20: note = Synthetic (H4A(+13+32))
gcatgaactcttgtggatcc

SEQ ID NO: 29 - RNA, 20 nt, synthetic construct
  source 1..20: mol_type = other RNA
  misc_feature 1..20: note = Synthetic (H4D(+04-16))
ccagggtactacttacatta

SEQ ID NO: 30 - RNA, 21 nt, synthetic construct
  source 1..21: mol_type = other RNA
  misc_feature 1..21: note = Synthetic (H4D(-24-44))
atcgtgtgtcacagcatccag

SEQ ID NO: 31 - RNA, 30 nt, synthetic construct
  source 1..30: mol_type = other RNA
  misc_feature 1..30: note = Synthetic (H4A(+11+40))
tgttcagggcatgaactcttgtggatcctt

SEQ ID NO: 32 - RNA, 31 nt, synthetic construct
  source 1..31: mol_type = other RNA
  misc_feature 1..31: note = Synthetic (H3A(+30+60))
taggaggcgcctcccatcctgtaggtcactg

SEQ ID NO: 33 - RNA, 31 nt, synthetic construct
  source 1..31: mol_type = other RNA
  misc_feature 1..31: note = Synthetic (H3A(+35+65))
aggtctaggaggcgcctcccatcctgtaggt

SEQ ID NO: 34 - RNA, 25 nt, synthetic construct
  source 1..25: mol_type = other RNA
  misc_feature 1..25: note = Synthetic (H3A(+30+54))
gcgcctcccatcctgtaggtcactg

SEQ ID NO: 35 - RNA, 26 nt, synthetic construct
  source 1..26: mol_type = other RNA
  misc_feature 1..26: note = Synthetic (H3D(+46-21))
cttcgaggaggtctaggaggcgcctc

SEQ ID NO: 36 - RNA, 21 nt, synthetic construct
  source 1..21: mol_type = other RNA
  misc_feature 1..21: note = Synthetic (H3A(+30+50))
ctcccatcctgtaggtcactg

SEQ ID NO: 37 - RNA, 22 nt, synthetic construct
  source 1..22: mol_type = other RNA
  misc_feature 1..22: note = Synthetic (H3D(+19-03))
taccagtttttgccctgtcagg

SEQ ID NO: 38 - RNA, 26 nt, synthetic construct
  source 1..26: mol_type = other RNA
  misc_feature 1..26: note = Synthetic (H3A(-06+20))
tcaatatgctgcttcccaaactgaaa

SEQ ID NO: 39 - RNA, 25 nt, synthetic construct
  source 1..25: mol_type = other RNA
  misc_feature 1..25: note = Synthetic (H3A(+37+61))
ctaggaggcgcctcccatcctgtag

SEQ ID NO: 40 - RNA, 31 nt, synthetic construct
  source 1..31: mol_type = other RNA
  misc_feature 1..31: note = Synthetic (H5A(+20+50))
ttatgatttccatctacgatgtcagtacttc

SEQ ID NO: 41 - RNA, 31 nt, synthetic construct
  source 1..31: mol_type = other RNA
  misc_feature 1..31: note = Synthetic (H5D(+25-05))
cttacctgccagtggaggattatattccaaa

SEQ ID NO: 42 - RNA, 25 nt, synthetic construct
  source 1..25: mol_type = other RNA
  misc_feature 1..25: note = Synthetic (H5D(+10-15))
catcaggattcttacctgccagtgg

SEQ ID NO: 43 - RNA, 25 nt, synthetic construct
  source 1..25: mol_type = other RNA
  misc_feature 1..25: note = Synthetic (H5A(+10+34))
cgatgtcagtacttccaatattcac

SEQ ID NO: 44 - RNA, 18 nt, synthetic construct
  source 1..18: mol_type = other RNA
  misc_feature 1..18: note = Synthetic (H5D(-04-21))
accattcatcaggattct

SEQ ID NO: 45 - RNA, 18 nt, synthetic construct
  source 1..18: mol_type = other RNA
  misc_feature 1..18: note = Synthetic (H5D(+16-02))
acctgccagtggaggatt

SEQ ID NO: 46 - RNA, 27 nt, synthetic construct
  source 1..27: mol_type = other RNA
  misc_feature 1..27: note = Synthetic (H5A(-07+20))
ccaatattcactaaatcaacctgttaa

SEQ ID NO: 47 - RNA, 30 nt, synthetic construct
  source 1..30: mol_type = other RNA
  misc_feature 1..30: note = Synthetic (H5D(+18-12))
caggattgttacctgccagtggaggattat

SEQ ID NO: 48 - RNA, 31 nt, synthetic construct
  source 1..31: mol_type = other RNA
  misc_feature 1..31: note = Synthetic (H5A(+05+35))
acgatgtcagtacttccaatattcactaaat

SEQ ID NO: 49 - RNA, 31 nt, synthetic construct
  source 1..31: mol_type = other RNA
  misc_feature 1..31: note = Synthetic (H5A(+15+45))
atttccatctacgatgtcagtacttccaata

SEQ ID NO: 50 - RNA, 21 nt, synthetic construct
  source 1..21: mol_type = other RNA
  misc_feature 1..21: note = Synthetic (H10A(-05+16))
caggagcttccaaatgctgca

SEQ ID NO: 51 - RNA, 29 nt, synthetic construct
  source 1..29: mol_type = other RNA
  misc_feature 1..29: note = Synthetic (H10A(-05+24))
cttgtcttcaggagcttccaaatgctgca

SEQ ID NO: 52 - RNA, 22 nt, synthetic construct
  source 1..22: mol_type = other RNA
  misc_feature 1..22: note = Synthetic (H10A(+98+119))
tcctcagcagaaagaagccacg

SEQ ID NO: 53 - RNA, 20 nt, synthetic construct
  source 1..20: mol_type = other RNA
  misc_feature 1..20: note = Synthetic (H10A(+130+149))
ttagaaatctctccttgtgc

SEQ ID NO: 54 - RNA, 20 nt, synthetic construct
  source 1..20: mol_type = other RNA
  misc_feature 1..20: note = Synthetic (H10A(-33-14))
taaattgggtgttacacaat

SEQ ID NO: 55 - RNA, 24 nt, synthetic construct
  source 1..24: mol_type = other RNA
  misc_feature 1..24: note = Synthetic (H11D(+26+49))
ccctgaggcattcccatcttgaat

SEQ ID NO: 56 - RNA, 20 nt, synthetic construct
  source 1..20: mol_type = other RNA
  misc_feature 1..20: note = Synthetic (H11D(+11-09))
aggacttacttgctttgttt

SEQ ID NO: 57 - RNA, 23 nt, synthetic construct
  source 1..23: mol_type = other RNA
  misc_feature 1..23: note = Synthetic (H11A(+118+140))
cttgaatttaggagattcatctg

SEQ ID NO: 58 - RNA, 23 nt, synthetic construct
  source 1..23: mol_type = other RNA
  misc_feature 1..23: note = Synthetic (H11A(+75+97))
catcttctgataattttcctgtt

SEQ ID NO: 59 - RNA, 24 nt, synthetic construct
  source 1..24: mol_type = other RNA
  misc_feature 1..24: note = Synthetic (H12A(+52+75))
tcttctgtttttgttagccagtca

SEQ ID NO: 60 - RNA, 20 nt, synthetic construct
  source 1..20: mol_type = other RNA
  misc_feature 1..20: note = Synthetic (H12A(-10+10))
tctatgtaaactgaaaattt

SEQ ID NO: 61 - RNA, 20 nt, synthetic construct
  source 1..20: mol_type = other RNA
  misc_feature 1..20: note = Synthetic (H12A(+11+30))
ttctggagatccattaaaac

SEQ ID NO: 62 - RNA, 24 nt, synthetic construct
  source 1..24: mol_type = other RNA
  misc_feature 1..24: note = Synthetic (H13A(+77+100))
cagcagttgcgtgatctccactag

SEQ ID NO: 63 - RNA, 21 nt, synthetic construct
  source 1..21: mol_type = other RNA
  misc_feature 1..21: note = Synthetic (H13A(+55+75))
ttcatcaactaccaccaccat

SEQ ID NO: 64 - RNA, 25 nt, synthetic construct
  source 1..25: mol_type = other RNA
  misc_feature 1..25: note = Synthetic (H13D(+06-19))
ctaagcaaaataatctgaccttaag

SEQ ID NO: 65 - RNA, 28 nt, synthetic construct
  source 1..28: mol_type = other RNA
  misc_feature 1..28: note = Synthetic (H14A(+37+64))
cttgtaaaagaacccagcggtcttctgt

SEQ ID NO: 66 - RNA, 22 nt, synthetic construct
  source 1..22: mol_type = other RNA
  misc_feature 1..22: note = Synthetic (H14A(+14+35))
catctacagatgtttgcccatc

SEQ ID NO: 67 - RNA, 23 nt, synthetic construct
  source 1..23: mol_type = other RNA
  misc_feature 1..23: note = Synthetic (H14A(+51+73))
gaaggatgtcttgtaaaagaacc

SEQ ID NO: 68 - RNA, 20 nt, synthetic construct
  source 1..20: mol_type = other RNA
  misc_feature 1..20: note = Synthetic (H14D(-02+18))
acctgttcttcagtaagacg

SEQ ID NO: 69 - RNA, 24 nt, synthetic construct
  source 1..24: mol_type = other RNA
  misc_feature 1..24: note = Synthetic (H14D(+14-10))
catgacacacctgttcttcagtaa

SEQ ID NO: 70 - RNA, 20 nt, synthetic construct
  source 1..20: mol_type = other RNA
  misc_feature 1..20: note = Synthetic (H14A(+61+80))
catttgagaaggatgtcttg

SEQ ID NO: 71 - RNA, 24 nt, synthetic construct
  source 1..24: mol_type = other RNA
  misc_feature 1..24: note = Synthetic (H14A(-12+12))
atctcccaatacctggagaagaga

SEQ ID NO: 72 - RNA, 31 nt, synthetic construct
  source 1..31: mol_type = other RNA
  misc_feature 1..31: note = Synthetic (H15A(-12+19))
gccatgcactaaaaaggcactgcaagacatt

SEQ ID NO: 73 - RNA, 24 nt, synthetic construct
  source 1..24: mol_type = other RNA
  misc_feature 1..24: note = Synthetic (H15A(+48+71))
tctttaaagccagttgtgtgaatc

SEQ ID NO: 74 - RNA, 21 nt, synthetic construct
  source 1..21: mol_type = other RNA
  misc_feature 1..21: note = Synthetic (H15A(+08+28))
tttctgaaagccatgcactaa

SEQ ID NO: 75 - RNA, 25 nt, synthetic construct
  source 1..25: mol_type = other RNA
  misc_feature 1..25: note = Synthetic (H15D(+17-08))
gtacatacggccagtttttgaagac

SEQ ID NO: 76 - RNA, 31 nt, synthetic construct
  source 1..31: mol_type = other RNA
  misc_feature 1..31: note = Synthetic (H16A(-12+19))
ctagatccgcttttaaaacctgttaaaacaa

SEQ ID NO: 77 - RNA, 31 nt, synthetic construct
  source 1..31: mol_type = other RNA
  misc_feature 1..31: note = Synthetic (H16A(-06+25))
tcttttctagatccgcttttaaaacctgtta

SEQ ID NO: 78 - RNA, 25 nt, synthetic construct
  source 1..25: mol_type = other RNA
  misc_feature 1..25: note = Synthetic (H16A(-06+19))
ctagatccgcttttaaaacctgtta

SEQ ID NO: 79 - RNA, 23 nt, synthetic construct
  source 1..23: mol_type = other RNA
  misc_feature 1..23: note = Synthetic (H16A(+87+109))
ccgtcttctgggtcactgactta

SEQ ID NO: 80 - RNA, 26 nt, synthetic construct
  source 1..26: mol_type = other RNA
  misc_feature 1..26: note = Synthetic (H16A(-07+19))
ctagatccgcttttaaaacctgttaa

SEQ ID NO: 81 - RNA, 20 nt, synthetic construct
  source 1..20: mol_type = other RNA
  misc_feature 1..20: note = Synthetic (H16A(-07+13))
ccgcttttaaaacctgttaa

SEQ ID NO: 82 - RNA, 26 nt, synthetic construct
  source 1..26: mol_type = other RNA
  misc_feature 1..26: note = Synthetic (H16A(+12+37))
tggattgctttttcttttctagatcc

SEQ ID NO: 83 - RNA, 25 nt, synthetic construct
  source 1..25: mol_type = other RNA
  misc_feature 1..25: note = Synthetic (H16A(+92+116))
catgcttccgtcttctgggtcactg

SEQ ID NO: 84 - RNA, 23 nt, synthetic construct
  source 1..23: mol_type = other RNA
  misc_feature 1..23: note = Synthetic (H16A(+45+67))
gatcttgtttgagtgaatacagt

SEQ ID NO: 85 - RNA, 22 nt, synthetic construct
  source 1..22: mol_type = other RNA
  misc_feature 1..22: note = Synthetic (H16A(+105+126))
gttatccagccatgcttccgtc

SEQ ID NO: 86 - RNA, 25 nt, synthetic construct
  source 1..25: mol_type = other RNA
  misc_feature 1..25: note = Synthetic (H16D(+05-20))
tgataattggtatcactaacctgtg

SEQ ID NO: 87 - RNA, 22 nt, synthetic construct
  source 1..22: mol_type = other RNA
  misc_feature 1..22: note = Synthetic (H16D(+12-11))
gtatcactaacctgtgctgtac

SEQ ID NO: 88 - RNA, 19 nt, synthetic construct
  source 1..19: mol_type = other RNA
  misc_feature 1..19: note = Synthetic (H19A(+35+53))
ctgctggcatcttgcagtt

SEQ ID NO: 89 - RNA, 31 nt, synthetic construct
  source 1..31: mol_type = other RNA
  misc_feature 1..31: note = Synthetic (H19A(+35+65))
gcctgagctgatctgctggcatcttgcagtt

SEQ ID NO: 90 - RNA, 28 nt, synthetic construct
  source 1..28: mol_type = other RNA
  misc_feature 1..28: note = Synthetic (H20A(+44+71))
ctggcagaattcgatccaccggctgttc

SEQ ID NO: 91 - RNA, 22 nt, synthetic construct
  source 1..22: mol_type = other RNA
  misc_feature 1..22: note = Synthetic (H20A(+147+168))
cagcagtagttgtcatctgctc

SEQ ID NO: 92 - RNA, 19 nt, synthetic construct
  source 1..19: mol_type = other RNA
  misc_feature 1..19: note = Synthetic (H20A(+185+203))
tgatggggtggtgggttgg

SEQ ID NO: 93 - RNA, 25 nt, synthetic construct
  source 1..25: mol_type = other RNA
  misc_feature 1..25: note = Synthetic (H20A(-08+17))
atctgcattaacaccctctagaaag

SEQ ID NO: 94 - RNA, 24 nt, synthetic construct
  source 1..24: mol_type = other RNA
  misc_feature 1..24: note = Synthetic (H20A(+30+53))
ccggctgttcagttgttctgaggc

SEQ ID NO: 95 - RNA, 28 nt, synthetic construct
  source 1..28: mol_type = other RNA
  misc_feature 1..28: note = Synthetic (H20A(-11+17))
atctgcattaacaccctctagaaagaaa

SEQ ID NO: 96 - RNA, 28 nt, synthetic construct
  source 1..28: mol_type = other RNA
  misc_feature 1..28: note = Synthetic (H20D(+08-20))
gaaggagaagagattcttaccttacaaa

SEQ ID NO: 97 - RNA, 20 nt, synthetic construct
  source 1..20: mol_type = other RNA
  misc_feature 1..20: note = Synthetic (H20A(+44+63))
attcgatccaccggctgttc

SEQ ID NO: 98 - RNA, 20 nt, synthetic construct
  source 1..20: mol_type = other RNA
  misc_feature 1..20: note = Synthetic (H20A(+149+168))
cagcagtagttgtcatctgc

SEQ ID NO: 99 - RNA, 22 nt, synthetic construct
  source 1..22: mol_type = other RNA
  misc_feature 1..22: note = Synthetic (H21A(-06+16))
gccggttgacttcatcctgtgc

SEQ ID NO: 100 - RNA, 22 nt, synthetic construct
  source 1..22: mol_type = other RNA
  misc_feature 1..22: note = Synthetic (H21A(+85+106))
ctgcatccaggaacatgggtcc

SEQ ID NO: 101 - RNA, 23 nt, synthetic construct
  source 1..23: mol_type = other RNA
  misc_feature 1..23: note = Synthetic (H21A(+85+108))
gtctgcatccaggaacatgggtc

SEQ ID NO: 102 - RNA, 24 nt, synthetic construct
  source 1..24: mol_type = other RNA
  misc_feature 1..24: note = Synthetic (H21A(+08+31))
gttgaagatctgatagccggttga

SEQ ID NO: 103 - RNA, 24 nt, synthetic construct
  source 1..24: mol_type = other RNA
  misc_feature 1..24: note = Synthetic (H21D(+18-07))
tacttactgtctgtagctctttct

SEQ ID NO: 104 - RNA, 24 nt, synthetic construct
  source 1..24: mol_type = other RNA
  misc_feature 1..24: note = Synthetic (H22A(+22+45))
cactcatggtctcctgatagcgca

SEQ ID NO: 105 - RNA, 22 nt, synthetic construct
  source 1..22: mol_type = other RNA
  misc_feature 1..22: note = Synthetic (H22A(+125+146))
ctgcaattccccgagtctctgc

SEQ ID NO: 106 - RNA, 23 nt, synthetic construct
  source 1..23: mol_type = other RNA
  misc_feature 1..23: note = Synthetic (H22A(+47+69))
actgctggacccatgtcctgatg

SEQ ID NO: 107 - RNA, 21 nt, synthetic construct
  source 1..21: mol_type = other RNA
  misc_feature 1..21: note = Synthetic (H22A(+80+101))
ctaagttgaggtatggagagt

SEQ ID NO: 108 - RNA, 23 nt, synthetic construct
  source 1..23: mol_type = other RNA
  misc_feature 1..23: note = Synthetic (H22D(+13-11))
tattcacagacctgcaattcccc

SEQ ID NO: 109 - RNA, 26 nt, synthetic construct
  source 1..26: mol_type = other RNA
  misc_feature 1..26: note = Synthetic (H23A(+34+59))
acagtggtgctgagatagtataggcc

SEQ ID NO: 110 - RNA, 22 nt, synthetic construct
  source 1..22: mol_type = other RNA
  misc_feature 1..22: note = Synthetic (H23A(+18+39))
taggccactttgttgctcttgc

SEQ ID NO: 111 - RNA, 19 nt, synthetic construct
  source 1..19: mol_type = other RNA
  misc_feature 1..19: note = Synthetic (H23A(+72+90))
ttcagagggcgctttcttc

SEQ ID NO: 112 - RNA, 23 nt, synthetic construct
  source 1..23: mol_type = other RNA
  misc_feature 1..23: note = Synthetic (H24A(+48+70))
gggcaggccattcctccttcaga

SEQ ID NO: 113 - RNA, 24 nt, synthetic construct
  source 1..24: mol_type = other RNA
  misc_feature 1..24: note = Synthetic (H24A(-02+22))
tcttcagggtttgtatgtgattct

SEQ ID NO: 114 - RNA, 27 nt, synthetic construct
  source 1..27: mol_type = other RNA
  misc_feature 1..27: note = Synthetic (H25A(+9+36))
ctgggctgaattgtctgaatatcactg

SEQ ID NO: 115 - RNA, 26 nt, synthetic construct
  source 1..26: mol_type = other RNA
  misc_feature 1..26: note = Synthetic (H25A(+131+156))
ctgttggcacatgtgatcccactgag

SEQ ID NO: 116 - RNA, 24 nt, synthetic construct
  source 1..24: mol_type = other RNA
  misc_feature 1..24: note = Synthetic (H25D(+16-08))
gtctatacctgttggcacatgtga

SEQ ID NO: 117 - RNA, 25 nt, synthetic construct
  source 1..25: mol_type = other RNA
  misc_feature 1..25: note = Synthetic (H26A(+132+156))
tgctttctgtaattcatctggagtt

SEQ ID NO: 118 - RNA, 26 nt, synthetic construct
  source 1..26: mol_type = other RNA
  misc_feature 1..26: note = Synthetic (H26A(-07+19))
cctcctttctggcatagaccttccac

SEQ ID NO: 119 - RNA, 25 nt, synthetic construct
  source 1..25: mol_type = other RNA
  misc_feature 1..25: note = Synthetic (H26A(+68+92))
tgtgtcatccattcgtgcatctctg

SEQ ID NO: 120 - RNA, 25 nt, synthetic construct
  source 1..25: mol_type = other RNA
  misc_feature 1..25: note = Synthetic (H27A(+82+106))
ttaaggcctcttgtgctacaggtgg

SEQ ID NO: 121 - RNA, 23 nt, synthetic construct
  source 1..23: mol_type = other RNA
  misc_feature 1..23: note = Synthetic (H27A(-4+19))
ggggctcttctttagctctctga

SEQ ID NO: 122 - RNA, 22 nt, synthetic construct
  source 1..22: mol_type = other RNA
  misc_feature 1..22: note = Synthetic (H27D(+19-03))
gacttccaaagtcttgcatttc

SEQ ID NO: 123 - RNA, 24 nt, synthetic construct
  source 1..24: mol_type = other RNA
  misc_feature 1..24: note = Synthetic (H28A(-05+19))
gccaacatgcccaaacttcctaag

SEQ ID NO: 124 - RNA, 26 nt, synthetic construct
  source 1..26: mol_type = other RNA
  misc_feature 1..26: note = Synthetic (H28A(+99+124))
cagagatttcctcagctccgccagga

SEQ ID NO: 125 - RNA, 21 nt, synthetic construct
  source 1..21: mol_type = other RNA
  misc_feature 1..21: note = Synthetic (H28D(+16-05))
cttacatctagcacctcagag

SEQ ID NO: 126 - RNA, 25 nt, synthetic construct
  source 1..25: mol_type = other RNA
  misc_feature 1..25: note = Synthetic (H29A(+57+81))
tccgccatctgttagggtctgtgcc

SEQ ID NO: 127 - RNA, 25 nt, synthetic construct
  source 1..25: mol_type = other RNA
  misc_feature 1..25: note = Synthetic (H29A(+18+42))
atttgggttatcctctgaatgtcgc

SEQ ID NO: 128 - RNA, 22 nt, synthetic construct
  source 1..22: mol_type = other RNA
  misc_feature 1..22: note = Synthetic (H29D(+17-05))
catacctcttcatgtagttccc

SEQ ID NO: 129 - RNA, 26 nt, synthetic construct
  source 1..26: mol_type = other RNA
  misc_feature 1..26: note = Synthetic (H30A(+122+147))
catttgagctgcgtccaccttgtctg

SEQ ID NO: 130 - RNA, 26 nt, synthetic construct
  source 1..26: mol_type = other RNA
  misc_feature 1..26: note = Synthetic (H30A(+25+50))
tcctgggcagactggatgctctgttc

SEQ ID NO: 131 - RNA, 23 nt, synthetic construct
  source 1..23: mol_type = other RNA
  misc_feature 1..23: note = Synthetic (H30D(+19-04))
ttgcctgggcttcctgaggcatt

SEQ ID NO: 132 - RNA, 24 nt, synthetic construct
  source 1..24: mol_type = other RNA
  misc_feature 1..24: note = Synthetic (H31D(+06-18))
ttctgaaataacatatacctgtgc

SEQ ID NO: 133 - RNA, 25 nt, synthetic construct
  source 1..25: mol_type = other RNA
  misc_feature 1..25: note = Synthetic (H31D(+03-22))
tagtttctgaaataacatatacctg

SEQ ID NO: 134 - RNA, 21 nt, synthetic construct
  source 1..21: mol_type = other RNA
  misc_feature 1..21: note = Synthetic (H31A(+05+25))
gacttgtcaaatcagattgga

SEQ ID NO: 135 - RNA, 24 nt, synthetic construct
  source 1..24: mol_type = other RNA
  misc_feature 1..24: note = Synthetic (H31D(+04-20))
gtttctgaaataacatatacctgt

SEQ ID NO: 136 - RNA, 20 nt, synthetic construct
  source 1..20: mol_type = other RNA
  misc_feature 1..20: note = Synthetic (H32D(+04-16))
caccagaaatacataccaca

SEQ ID NO: 137 - RNA, 20 nt, synthetic construct
  source 1..20: mol_type = other RNA
  misc_feature 1..20: note = Synthetic (H32A(+151+170))
caatgatttagctgtgactg

SEQ ID NO: 138 - RNA, 23 nt, synthetic construct
  source 1..23: mol_type = other RNA
  misc_feature 1..23: note = Synthetic (H32A(+10+32))
cgaaacttcatggagacatcttg

SEQ ID NO: 139 - RNA, 25 nt, synthetic construct
  source 1..25: mol_type = other RNA
  misc_feature 1..25: note = Synthetic (H32A(+49+73))
cttgtagacgctgctcaaaattggc

SEQ ID NO: 140 - RNA, 20 nt, synthetic construct
  source 1..20: mol_type = other RNA
  misc_feature 1..20: note = Synthetic (H33D(+09-11))
catgcacacacctttgctcc

SEQ ID NO: 141 - RNA, 24 nt, synthetic construct
  source 1..24: mol_type = other RNA
  misc_feature 1..24: note = Synthetic (H33A(+53+76))
tctgtacaatctgacgtccagtct

SEQ ID NO: 142 - RNA, 27 nt, synthetic construct
  source 1..27: mol_type = other RNA
  misc_feature 1..27: note = Synthetic (H33A(+30+56))
gtctttatcaccatttccacttcagac

SEQ ID NO: 143 - RNA, 25 nt, synthetic construct
  source 1..25: mol_type = other RNA
  misc_feature 1..25: note = Synthetic (H33A(+64+88))
ccgtctgctttttctgtacaatctg

SEQ ID NO: 144 - RNA, 22 nt, synthetic construct
  source 1..22: mol_type = other RNA
  misc_feature 1..22: note = Synthetic (H34A(+83+104))
tccatatctgtagctgccagcc

SEQ ID NO: 145 - RNA, 23 nt, synthetic construct
  source 1..23: mol_type = other RNA
  misc_feature 1..23: note = Synthetic (H34A(+143+165))
ccaggcaacttcagaatccaaat

SEQ ID NO: 146 - RNA, 30 nt, synthetic construct
  source 1..30: mol_type = other RNA
  misc_feature 1..30: note = Synthetic (H34A(-20+10))
tttctgttacctgaaaagaattataatgaa

SEQ ID NO: 147 - RNA, 25 nt, synthetic construct
  source 1..25: mol_type = other RNA
  misc_feature 1..25: note = Synthetic (H34A(+46+70))
cattcatttcctttcgcatcttacg

SEQ ID NO: 148 - RNA, 26 nt, synthetic construct
  source 1..26: mol_type = other RNA
  misc_feature 1..26: note = Synthetic (H34A(+95+120))
tgatctctttgtcaattccatatctg

SEQ ID NO: 149 - RNA, 30 nt, synthetic construct
  source 1..30: mol_type = other RNA
  misc_feature 1..30: note = Synthetic (H34D(+10-20))
ttcagtgatataggttttacctttccccag

SEQ ID NO: 150 - RNA, 26 nt, synthetic construct
  source 1..26: mol_type = other RNA
  misc_feature 1..26: note = Synthetic (H34A(+72+96))
ctgtagctgccagccattctgtcaag

SEQ ID NO: 151 - RNA, 21 nt, synthetic construct
  source 1..21: mol_type = other RNA
  misc_feature 1..21: note = Synthetic (H35A(+141+161))
tcttctgctcgggaggtgaca

SEQ ID NO: 152 - RNA, 20 nt, synthetic construct
  source 1..20: mol_type = other RNA
  misc_feature 1..20: note = Synthetic (H35A(+116+135))
ccagttactattcagaagac

SEQ ID NO: 153 - RNA, 20 nt, synthetic construct
  source 1..20: mol_type = other RNA
  misc_feature 1..20: note = Synthetic (H35A(+24+43))
tcttcaggtgcaccttctgt

SEQ ID NO: 154 - RNA, 25 nt, synthetic construct
  source 1..25: mol_type = other RNA
  misc_feature 1..25: note = Synthetic (H36A(+26+50))
tgtgatgtggtccacattctggtca

SEQ ID NO: 155 - RNA, 20 nt, synthetic construct
  source 1..20: mol_type = other RNA
  misc_feature 1..20: note = Synthetic (H36A(-02+18))
ccatgtgtttctggtattcc

SEQ ID NO: 156 - RNA, 25 nt, synthetic construct
  source 1..25: mol_type = other RNA
  misc_feature 1..25: note = Synthetic (H37A(+26+50))
cgtgtagagtccacctttgggcgta

SEQ ID NO: 157 - RNA, 24 nt, synthetic construct
  source 1..24: mol_type = other RNA
  misc_feature 1..24: note = Synthetic (H37A(+82+105))
tactaatttcctgcagtggtcacc

SEQ ID NO: 158 - RNA, 24 nt, synthetic construct
  source 1..24: mol_type = other RNA
  misc_feature 1..24: note = Synthetic (H37A(+134+157))
ttctgtgtgaaatggctgcaaatc

SEQ ID NO: 159 - RNA, 20 nt, synthetic construct
  source 1..20: mol_type = other RNA
  misc_feature 1..20: note = Synthetic (H38A(-01+19))
ccttcaaaggaatggaggcc

SEQ ID NO: 160 - RNA, 25 nt, synthetic construct
  source 1..25: mol_type = other RNA
  misc_feature 1..25: note = Synthetic (H38A(+59+83))
tgctgaatttcagcctccagtggtt

SEQ ID NO: 161 - RNA, 25 nt, synthetic construct
  source 1..25: mol_type = other RNA
  misc_feature 1..25: note = Synthetic (H38A(+88+112))
tgaagtcttcctctttcagattcac

SEQ ID NO: 162 - RNA, 24 nt, synthetic construct
  source 1..24: mol_type = other RNA
  misc_feature 1..24: note = Synthetic (H39A(+62+85))
ctggctttctctcatctgtgattc

SEQ ID NO: 163 - RNA, 20 nt, synthetic construct
  source 1..20: mol_type = other RNA
  misc_feature 1..20: note = Synthetic (H39A(+39+58))
gttgtaagttgtctcctctt

SEQ ID NO: 164 - RNA, 20 nt, synthetic construct
  source 1..20: mol_type = other RNA
  misc_feature 1..20: note = Synthetic (H39A(+102+121))
ttgtctgtaacagctgctgt

SEQ ID NO: 165 - RNA, 20 nt, synthetic construct
  source 1..20: mol_type = other RNA
  misc_feature 1..20: note = Synthetic (H39D(+10-10))
gctctaataccttgagagca

SEQ ID NO: 166 - RNA, 22 nt, synthetic construct
  source 1..22: mol_type = other RNA
  misc_feature 1..22: note = Synthetic (H40A(-05+17))
ctttgagacctcaaatcctgtt

SEQ ID NO: 167 - RNA, 25 nt, synthetic construct
  source 1..25: mol_type = other RNA
  misc_feature 1..25: note = Synthetic (H40A(+129+153))
ctttattttcctttcatctctgggc

SEQ ID NO: 168 - RNA, 27 nt, synthetic construct
  source 1..27: mol_type = other RNA
  misc_feature 1..27: note = Synthetic (H42A(-04+23))
atcgtttcttcacggacagtgtgctgg

SEQ ID NO: 169 - RNA, 24 nt, synthetic construct
  source 1..24: mol_type = other RNA
  misc_feature 1..24: note = Synthetic (H42A(+86+109))
gggcttgtgagacatgagtgattt

SEQ ID NO: 170 - RNA, 22 nt, synthetic construct
  source 1..22: mol_type = other RNA
  misc_feature 1..22: note = Synthetic (H42D(+19-02))
accttcagaggactcctcttgc

SEQ ID NO: 171 - RNA, 25 nt, synthetic construct
  source 1..25: mol_type = other RNA
  misc_feature 1..25: note = Synthetic (H43D(+10-15))
tatgtgttacctacccttgtcggtc

SEQ ID NO: 172 - RNA, 20 nt, synthetic construct
  source 1..20: mol_type = other RNA
  misc_feature 1..20: note = Synthetic (H43A(+101+120))
ggagagagcttcctgtagct

SEQ ID NO: 173 - RNA, 23 nt, synthetic construct
  source 1..23: mol_type = other RNA
  misc_feature 1..23: note = Synthetic (H43A(+78+100))
tcaccctttccacaggcgttgca

SEQ ID NO: 174 - RNA, 20 nt, synthetic construct
  source 1..20: mol_type = other RNA
  misc_feature 1..20: note = Synthetic (H44A(+85+104))
tttgtgtctttctgagaaac

SEQ ID NO: 175 - RNA, 20 nt, synthetic construct
  source 1..20: mol_type = other RNA
  misc_feature 1..20: note = Synthetic (H44D(+10-10))
aaagacttaccttaagatac

SEQ ID NO: 176 - RNA, 20 nt, synthetic construct
  source 1..20: mol_type = other RNA
  misc_feature 1..20: note = Synthetic (H44A(-06+14))
atctgtcaaatcgcctgcag

SEQ ID NO: 177 - RNA, 20 nt, synthetic construct
  source 1..20: mol_type = other RNA
  misc_feature 1..20: note = Synthetic (H46D(+16-04))
ttaccttgacttgctcaagc

SEQ ID NO: 178 - RNA, 20 nt, synthetic construct
  source 1..20: mol_type = other RNA
  misc_feature 1..20: note = Synthetic (H46A(+90+109))
tccaggttcaagtgggatac

SEQ ID NO: 179 - RNA, 25 nt, synthetic construct
  source 1..25: mol_type = other RNA
  misc_feature 1..25: note = Synthetic (H47A(+76+100))
gctcttctgggcttatgggagcact

SEQ ID NO: 180 - RNA, 27 nt, synthetic construct
  source 1..27: mol_type = other RNA
  misc_feature 1..27: note = Synthetic (H47D(+25-02))
acctttatccactggagatttgtctgc

SEQ ID NO: 181 - RNA, 21 nt, synthetic construct
  source 1..21: mol_type = other RNA
  misc_feature 1..21: note = Synthetic (H47A(-9+12))
ttccaccagtaactgaaacag

SEQ ID NO: 182 - RNA, 29 nt, synthetic construct
  source 1..29: mol_type = other RNA
  misc_feature 1..29: note = Synthetic (H50A(+02+30))
ccactcagagctcagatcttctaacttcc

SEQ ID NO: 183 - RNA, 27 nt, synthetic construct
  source 1..27: mol_type = other RNA
  misc_feature 1..27: note = Synthetic (H50A(+07+33))
cttccactcagagctcagatcttctaa

SEQ ID NO: 184 - RNA, 26 nt, synthetic construct
  source 1..26: mol_type = other RNA
  misc_feature 1..26: note = Synthetic (H51A(-01+25))
accagagtaacagtctgagtaggagc

SEQ ID NO: 185 - RNA, 23 nt, synthetic construct
  source 1..23: mol_type = other RNA
  misc_feature 1..23: note = Synthetic (H51D(+16-07))
ctcataccttctgcttgatgatc

SEQ ID NO: 186 - RNA, 24 nt, synthetic construct
  source 1..24: mol_type = other RNA
  misc_feature 1..24: note = Synthetic (H51A(⇐+134))
ttctgtccaagcccggttgaaatc

SEQ ID NO: 187 - RNA, 30 nt, synthetic construct
  source 1..30: mol_type = other RNA
  misc_feature 1..30: note = Synthetic (H51A(+66+95))
ctccaacatcaaggaagatggcatttctag

SEQ ID NO: 188 - RNA, 25 nt, synthetic construct
  source 1..25: mol_type = other RNA
  misc_feature 1..25: note = Synthetic (H51D(+08-17))
atcattttttctcataccttctgct

SEQ ID NO: 189 - RNA, 36 nt, synthetic construct
  source 1..36: mol_type = other RNA
  misc_feature 1..36: note = Synthetic (H51A/D(+08-17)& (-15+))
atcattttttctcataccttctgctaggagctaaaa

SEQ ID NO: 190 - RNA, 21 nt, synthetic construct
  source 1..21: mol_type = other RNA
  misc_feature 1..21: note = Synthetic (H51A(+175+195))
cacccaccatcaccctctgtg

SEQ ID NO: 191 - RNA, 22 nt, synthetic construct
  source 1..22: mol_type = other RNA
  misc_feature 1..22: note = Synthetic (H51A(+199+220))
atcatctcgttgatatcctcaa

SEQ ID NO: 192 - RNA, 21 nt, synthetic construct
  source 1..21: mol_type = other RNA
  misc_feature 1..21: note = Synthetic (H52A(-07+14))
tcctgcattgttgcctgtaag

SEQ ID NO: 193 - RNA, 30 nt, synthetic construct
  source 1..30: mol_type = other RNA
  misc_feature 1..30: note = Synthetic (H52A(+12+41))
tccaactggggacgcctctgttccaaatcc

SEQ ID NO: 194 - RNA, 21 nt, synthetic construct
  source 1..21: mol_type = other RNA
  misc_feature 1..21: note = Synthetic (H52A(+17+37))
actggggacgcctctgttcca

SEQ ID NO: 195 - RNA, 20 nt, synthetic construct
  source 1..20: mol_type = other RNA
  misc_feature 1..20: note = Synthetic (H52A(+93+112))
ccgtaatgattgttctagcc

SEQ ID NO: 196 - RNA, 20 nt, synthetic construct
  source 1..20: mol_type = other RNA
  misc_feature 1..20: note = Synthetic (H52D(+05-15))
tgttaaaaaacttacttcga

SEQ ID NO: 197 - RNA, 25 nt, synthetic construct
  source 1..25: mol_type = other RNA
  misc_feature 1..25: note = Synthetic (H53A(+45+69))
cattcaactgttgcctccggttctg

SEQ ID NO: 198 - RNA, 24 nt, synthetic construct
  source 1..24: mol_type = other RNA
  misc_feature 1..24: note = Synthetic (H53A(+39+62))
ctgttgcctccggttctgaaggtg

SEQ ID NO: 199 - RNA, 31 nt, synthetic construct
  source 1..31: mol_type = other RNA
  misc_feature 1..31: note = Synthetic (H53A(+39+69))
cattcaactgttgcctccggttctgaaggtg

SEQ ID NO: 200 - RNA, 21 nt, synthetic construct
  source 1..21: mol_type = other RNA
  misc_feature 1..21: note = Synthetic (H53D(+14-07))
tactaaccttggtttctgtga

SEQ ID NO: 201 - RNA, 25 nt, synthetic construct
  source 1..25: mol_type = other RNA
  misc_feature 1..25: note = Synthetic (H53A(+23+47))
ctgaaggtgttcttgtacttcatcc

SEQ ID NO: 202 - RNA, 27 nt, synthetic construct
  source 1..27: mol_type = other RNA
  misc_feature 1..27: note = Synthetic (H53A(+150+176))
tgtatagggaccctccttccatgactc

SEQ ID NO: 203 - RNA, 25 nt, synthetic construct
  source 1..25: mol_type = other RNA
  misc_feature 1..25: note = Synthetic (H53D(+20-05))
ctaaccttggtttctgtgattttct

SEQ ID NO: 204 - RNA, 27 nt, synthetic construct
  source 1..27: mol_type = other RNA
  misc_feature 1..27: note = Synthetic (H53D(+09-18))
ggtatctttgatactaaccttggtttc

SEQ ID NO: 205 - RNA, 22 nt, synthetic construct
  source 1..22: mol_type = other RNA
  misc_feature 1..22: note = Synthetic (H53A(-12+10))
attctttcaactagaataaaag

SEQ ID NO: 206 - RNA, 25 nt, synthetic construct
  source 1..25: mol_type = other RNA
  misc_feature 1..25: note = Synthetic (H53A(-07+18))
gattctgaattctttcaactagaat

SEQ ID NO: 207 - RNA, 20 nt, synthetic construct
  source 1..20: mol_type = other RNA
  misc_feature 1..20: note = Synthetic (H53A(+07+26))
atcccactgattctgaattc

SEQ ID NO: 208 - RNA, 22 nt, synthetic construct
  source 1..22: mol_type = other RNA
  misc_feature 1..22: note = Synthetic (H53A(+124+145))
ttggctctggcctgtcctaaga

SEQ ID NO: 209 - RNA, 30 nt, synthetic construct
  source 1..30: mol_type = other RNA
  misc_feature 1..30: note = Synthetic (H46A(+86+115))
ctcttttccaggttcaagtgggatactagc

SEQ ID NO: 210 - RNA, 31 nt, synthetic construct
  source 1..31: mol_type = other RNA
  misc_feature 1..31: note = Synthetic (H46A(+107+137))
caagcttttcttttagttgctgctcttttcc

SEQ ID NO: 211 - RNA, 30 nt, synthetic construct
  source 1..30: mol_type = other RNA
  misc_feature 1..30: note = Synthetic (H46A(-10+20))
tattcttttgttcttctagcctggagaaag

SEQ ID NO: 212 - RNA, 28 nt, synthetic construct
  source 1..28: mol_type = other RNA
  misc_feature 1..28: note = Synthetic (H46A(+50+77))
ctgcttcctccaaccataaaacaaattc

SEQ ID NO: 213 - RNA, 26 nt, synthetic construct
  source 1..26: mol_type = other RNA
  misc_feature 1..26: note = Synthetic (H45A(-06+20))
ccaatgccatcctggagttcctgtaa

SEQ ID NO: 214 - RNA, 20 nt, synthetic construct
  source 1..20: mol_type = other RNA
  misc_feature 1..20: note = Synthetic (H45A(+91 +110))
tcctgtagaatactggcatc

SEQ ID NO: 215 - RNA, 27 nt, synthetic construct
  source 1..27: mol_type = other RNA
  misc_feature 1..27: note = Synthetic (H45A(+125+151))
tgcagacctcctgccaccgcagattca

SEQ ID NO: 216 - RNA, 20 nt, synthetic construct
  source 1..20: mol_type = other RNA
  misc_feature 1..20: note = Synthetic (H45D(≠-04))
ctacctcttttttctgtctg

SEQ ID NO: 217 - RNA, 20 nt, synthetic construct
  source 1..20: mol_type = other RNA
  misc_feature 1..20: note = Synthetic (H45A(+71+90))
tgtttttgaggattgctgaa

SEQ ID NO: 218 - DNA, 28 bp, synthetic construct
  source 1..28: mol_type = other DNA
  misc_feature 1..28: note = Synthetic (H53A(+33+60))
gttgcctccggttctgaaggtgttcttg

SEQ ID NO: 219 - DNA, 25 bp, synthetic construct
  source 1..25: mol_type = other DNA
  misc_feature 1..25: note = Synthetic (H53A(+23+47))
ctgaaggtgttcttgtacttcatcc

SEQ ID NO: 220 - DNA, 30 bp, synthetic construct
  source 1..30: mol_type = other DNA
  misc_feature 1..30: note = Synthetic (H53A(+33+62))
ctgttgcctccggttctgaaggtgttcttg

SEQ ID NO: 221 - DNA, 33 bp, synthetic construct
  source 1..33: mol_type = other DNA
  misc_feature 1..33: note = Synthetic (H53A(+33+65))
caactgttgcctccggttctgaaggtgttcttg

SEQ ID NO: 222 - DNA, 25 bp, synthetic construct
  source 1..25: mol_type = other DNA
  misc_feature 1..25: note = Synthetic (H53A(+31+55))
ctccggttctgaaggtgttcttgta

SEQ ID NO: 223 - DNA, 28 bp, synthetic construct
  source 1..28: mol_type = other DNA
  misc_feature 1..28: note = Synthetic (H53A(+46+73))
atttcattcaactgttgcctccggttct

SEQ ID NO: 224 - DNA, 25 bp, synthetic construct
  source 1..25: mol_type = other DNA
  misc_feature 1..25: note = Synthetic (H53A(+22+46))
tgaaggtgttcttgtacttcatccc

SEQ ID NO: 225 - DNA, 24 bp, synthetic construct
  source 1..24: mol_type = other DNA
  misc_feature 1..24: note = Synthetic (H53A(+46+69))
cattcaactgttgcctccggttct

SEQ ID NO: 226 - DNA, 22 bp, synthetic construct
  source 1..22: mol_type = other DNA
  misc_feature 1..22: note = Synthetic (H53A(+40+61))
tgttgcctccggttctgaaggt

SEQ ID NO: 227 - DNA, 25 bp, synthetic construct
  source 1..25: mol_type = other DNA
  misc_feature 1..25: note = Synthetic (H53A(+30+60))
gttgcctccggttctgaaggtgttc

SEQ ID NO: 228 - DNA, 28 bp, synthetic construct
  source 1..28: mol_type = other DNA
  misc_feature 1..28: note = Synthetic (H53A(+30+57))
gcctccggttctgaaggtgttcttgtac

SEQ ID NO: 229 - DNA, 27 bp, synthetic construct
  source 1..27: mol_type = other DNA
  misc_feature 1..27: note = Synthetic (H53A(+30+56))
cctccggttctgaaggtgttcttgtac

SEQ ID NO: 230 - DNA, 26 bp, synthetic construct
  source 1..26: mol_type = other DNA
  misc_feature 1..26: note = Synthetic (H53A(+30+55))
ctccggttctgaaggtgttcttgtac

SEQ ID NO: 231 - DNA, 25 bp, synthetic construct
  source 1..25: mol_type = other DNA
  misc_feature 1..25: note = Synthetic (H53A(+33+57))
gcctccggttctgaaggtgttcttg

SEQ ID NO: 232 - DNA, 24 bp, synthetic construct
  source 1..24: mol_type = other DNA
  misc_feature 1..24: note = Synthetic (H44A(-07+17))
cagatctgtcaaatcgcctgcagg

SEQ ID NO: 233 - DNA, 27 bp, synthetic construct
  source 1..27: mol_type = other DNA
  misc_feature 1..27: note = Synthetic (H44A(-07+20))
caacagatctgtcaaatcgcctgcagg

SEQ ID NO: 234 - DNA, 29 bp, synthetic construct
  source 1..29: mol_type = other DNA
  misc_feature 1..29: note = Synthetic (H44A(-07+22))
ctcaacagatctgtcaaatcgcctgcagg

SEQ ID NO: 235 - DNA, 25 bp, synthetic construct
  source 1..25: mol_type = other DNA
  misc_feature 1..25: note = Synthetic (H44A(+77+101))
gtgtctttctgagaaactgttcagc

SEQ ID NO: 236 - DNA, 28 bp, synthetic construct
  source 1..28: mol_type = other DNA
  misc_feature 1..28: note = Synthetic (H44A(+64+91))
gagaaactgttcagcttctgttagccac

SEQ ID NO: 237 - DNA, 28 bp, synthetic construct
  source 1..28: mol_type = other DNA
  misc_feature 1..28: note = Synthetic (H44A(+62+89))
gaaactgttcagcttctgttagccactg

SEQ ID NO: 238 - DNA, 24 bp, synthetic construct
  source 1..24: mol_type = other DNA
  misc_feature 1..24: note = Synthetic (H44A(+62+85))
ctgttcagcttctgttagccactg

SEQ ID NO: 239 - DNA, 20 bp, synthetic construct
  source 1..20: mol_type = other DNA
  misc_feature 1..20: note = Synthetic (H44A(-06+14))
atctgtcaaatcgcctgcag

SEQ ID NO: 240 - DNA, 20 bp, synthetic construct
  source 1..20: mol_type = other DNA
  misc_feature 1..20: note = Synthetic (H44A(+85+104))
tttgtgtctttctgagaaac

SEQ ID NO: 241 - DNA, 24 bp, synthetic construct
  source 1..24: mol_type = other DNA
  misc_feature 1..24: note = Synthetic (H44A(+61+84))
tgttcagcttctgttagccactga

SEQ ID NO: 242 - DNA, 25 bp, synthetic construct
  source 1..25: mol_type = other DNA
  misc_feature 1..25: note = Synthetic (H44A(-10+15))
gatctgtcaaatcgcctgcaggtaa

SEQ ID NO: 243 - DNA, 25 bp, synthetic construct
  source 1..25: mol_type = other DNA
  misc_feature 1..25: note = Synthetic (H44A(+64+88))
aaactgttcagcttctgttagccac

SEQ ID NO: 244 - DNA, 25 bp, synthetic construct
  source 1..25: mol_type = other DNA
  misc_feature 1..25: note = Synthetic (H44A(+79+103))
ttgtgtctttctgagaaactgttca

SEQ ID NO: 245 - DNA, 26 bp, synthetic construct
  source 1..26: mol_type = other DNA
  misc_feature 1..26: note = Synthetic (H44A(-06+20))
caacagatctgtcaaatcgcctgcag

SEQ ID NO: 246 - DNA, 26 bp, synthetic construct
  source 1..26: mol_type = other DNA
  misc_feature 1..26: note = Synthetic (H44A(-09+17))
cagatctgtcaaatcgcctgcaggta

SEQ ID NO: 247 - DNA, 27 bp, synthetic construct
  source 1..27: mol_type = other DNA
  misc_feature 1..27: note = Synthetic (H44A(+59+85))
ctgttcagcttctgttagccactgatt

SEQ ID NO: 248 - DNA, 31 bp, synthetic construct
  source 1..31: mol_type = other DNA
  misc_feature 1..31: note = Synthetic (H44A(+59+89))
gaaactgttcagcttctgttagccactgatt

SEQ ID NO: 249 - DNA, 26 bp, synthetic construct
  source 1..26: mol_type = other DNA
  misc_feature 1..26: note = Synthetic (H44A(+65+90))
agaaactgttcagcttctgttagcca

SEQ ID NO: 250 - DNA, 25 bp, synthetic construct
  source 1..25: mol_type = other DNA
  misc_feature 1..25: note = Synthetic (Hu.DMD.Exon44.25.001)
ctgcaggtaaaagcatatggatcaa

SEQ ID NO: 251 - DNA, 25 bp, synthetic construct
  source 1..25: mol_type = other DNA
  misc_feature 1..25: note = Synthetic (Hu.DMD.Exon44.25.002)
atcgcctgcaggtaaaagcatatgg

SEQ ID NO: 252 - DNA, 25 bp, synthetic construct
  source 1..25: mol_type = other DNA
  misc_feature 1..25: note = Synthetic (Hu.DMD.Exon44.25.003)
gtcaaatcgcctgcaggtaaaagca

SEQ ID NO: 253 - DNA, 25 bp, synthetic construct
  source 1..25: mol_type = other DNA
  misc_feature 1..25: note = Synthetic (Hu.DMD.Exon44.25.005)
caacagatctgtcaaatcgcctgca

SEQ ID NO: 254 - DNA, 25 bp, synthetic construct
  source 1..25: mol_type = other DNA
  misc_feature 1..25: note = Synthetic (Hu.DMD.Exon44.25.006)
tttctcaacagatctgtcaaatcgc

SEQ ID NO: 255 - DNA, 25 bp, synthetic construct
  source 1..25: mol_type = other DNA
  misc_feature 1..25: note = Synthetic (Hu.DMD.Exon44.25.007)
ccatttctcaacagatctgtcaaat

SEQ ID NO: 256 - DNA, 25 bp, synthetic construct
  source 1..25: mol_type = other DNA
  misc_feature 1..25: note = Synthetic (Hu.DMD.Exon44.25.008)
ataatgaaaacgccgccatttctca

SEQ ID NO: 257 - DNA, 25 bp, synthetic construct
  source 1..25: mol_type = other DNA
  misc_feature 1..25: note = Synthetic (Hu.DMD.Exon44.25.009)
aaatatctttatatcataatgaaaa

SEQ ID NO: 258 - DNA, 25 bp, synthetic construct
  source 1..25: mol_type = other DNA
  misc_feature 1..25: note = Synthetic (Hu.DMD.Exon44.25.010)
tgttagccactgattaaatatcttt

SEQ ID NO: 259 - DNA, 25 bp, synthetic construct
  source 1..25: mol_type = other DNA
  misc_feature 1..25: note = Synthetic (Hu.DMD.Exon44.25.013)
ccaattctcaggaatttgtgtcttt

SEQ ID NO: 260 - DNA, 25 bp, synthetic construct
  source 1..25: mol_type = other DNA
  misc_feature 1..25: note = Synthetic (Hu.DMD.Exon44.25.014)
gtatttagcatgttcccaattctca

SEQ ID NO: 261 - DNA, 25 bp, synthetic construct
  source 1..25: mol_type = other DNA
  misc_feature 1..25: note = Synthetic (Hu.DMD.Exon44.25.015)
cttaagataccatttgtatttagca

SEQ ID NO: 262 - DNA, 25 bp, synthetic construct
  source 1..25: mol_type = other DNA
  misc_feature 1..25: note = Synthetic (Hu.DMD.Exon44.25.016)
cttaccttaagataccatttgtatt

SEQ ID NO: 263 - DNA, 25 bp, synthetic construct
  source 1..25: mol_type = other DNA
  misc_feature 1..25: note = Synthetic (Hu.DMD.Exon44.25.017)
aaagacttaccttaagataccattt

SEQ ID NO: 264 - DNA, 25 bp, synthetic construct
  source 1..25: mol_type = other DNA
  misc_feature 1..25: note = Synthetic (Hu.DMD.Exon44.25.018)
aaatcaaagacttaccttaagatac

SEQ ID NO: 265 - DNA, 25 bp, synthetic construct
  source 1..25: mol_type = other DNA
  misc_feature 1..25: note = Synthetic (Hu.DMD.Exon44.25.019)
aaaacaaatcaaagacttaccttaa

SEQ ID NO: 266 - DNA, 25 bp, synthetic construct
  source 1..25: mol_type = other DNA
  misc_feature 1..25: note = Synthetic (Hu.DMD.Exon44.25.020)
tcgaaaaaacaaatcaaagacttac

SEQ ID NO: 267 - DNA, 25 bp, synthetic construct
  source 1..25: mol_type = other DNA
  misc_feature 1..25: note = Synthetic (Hu.DMD.Exon45.25.001)
ctgtaagataccaaaaaggcaaaac

SEQ ID NO: 268 - DNA, 25 bp, synthetic construct
  source 1..25: mol_type = other DNA
  misc_feature 1..25: note = Synthetic (Hu.DMD.Exon45.25.002)
cctgtaagataccaaaaaggcaaaa

SEQ ID NO: 269 - DNA, 25 bp, synthetic construct
  source 1..25: mol_type = other DNA
  misc_feature 1..25: note = Synthetic (Hu.DMD.Exon45.25.002.2)
agttcctgtaagataccaaaaaggc

SEQ ID NO: 270 - DNA, 25 bp, synthetic construct
  source 1..25: mol_type = other DNA
  misc_feature 1..25: note = Synthetic (Hu.DMD.Exon45.25.003)
gagttcctgtaagataccaaaaagg

SEQ ID NO: 271 - DNA, 25 bp, synthetic construct
  source 1..25: mol_type = other DNA
  misc_feature 1..25: note = Synthetic (Hu.DMD.Exon45.25.003.2)
cctggagttcctgtaagataccaaa

SEQ ID NO: 272 - DNA, 25 bp, synthetic construct
  source 1..25: mol_type = other DNA
  misc_feature 1..25: note = Synthetic (Hu.DMD.Exon45.25.004)
tcctggagttcctgtaagataccaa

SEQ ID NO: 273 - DNA, 25 bp, synthetic construct
  source 1..25: mol_type = other DNA
  misc_feature 1..25: note = Synthetic (Hu.DMD.Exon45.25.004.2)
gccatcctggagttcctgtaagata

SEQ ID NO: 274 - DNA, 25 bp, synthetic construct
  source 1..25: mol_type = other DNA
  misc_feature 1..25: note = Synthetic (Hu.DMD.Exon45.25.005)
tgccatcctggagttcctgtaagat

SEQ ID NO: 275 - DNA, 25 bp, synthetic construct
  source 1..25: mol_type = other DNA
  misc_feature 1..25: note = Synthetic (Hu.DMD.Exon45.25.005.2)
ccaatgccatcctggagttcctgta

SEQ ID NO: 276 - DNA, 25 bp, synthetic construct
  source 1..25: mol_type = other DNA
  misc_feature 1..25: note = Synthetic (Hu.DMD.Exon45.25.006)
cccaatgccatcctggagttcctgt

SEQ ID NO: 277 - DNA, 25 bp, synthetic construct
  source 1..25: mol_type = other DNA
  misc_feature 1..25: note = Synthetic (Hu.DMD.Exon45.25.006.2)
gctgcccaatgccatcctggagttc

SEQ ID NO: 278 - DNA, 25 bp, synthetic construct
  source 1..25: mol_type = other DNA
  misc_feature 1..25: note = Synthetic (Hu.DMD.Exon45.25.007)
cgctgcccaatgccatcctggagtt

SEQ ID NO: 279 - DNA, 25 bp, synthetic construct
  source 1..25: mol_type = other DNA
  misc_feature 1..25: note = Synthetic (Hu.DMD.Exon45.25.008)
aacagtttgccgctgcccaatgcca

SEQ ID NO: 280 - DNA, 25 bp, synthetic construct
  source 1..25: mol_type = other DNA
  misc_feature 1..25: note = Synthetic (Hu.DMD.Exon45.25.008.2)
ctgacaacagtttgccgctgcccaa

SEQ ID NO: 281 - DNA, 25 bp, synthetic construct
  source 1..25: mol_type = other DNA
  misc_feature 1..25: note = Synthetic (Hu.DMD.Exon45.25.009)
gttgcattcaatgttctgacaacag

SEQ ID NO: 282 - DNA, 25 bp, synthetic construct
  source 1..25: mol_type = other DNA
  misc_feature 1..25: note = Synthetic (Hu.DMD.Exon45.25.010)
gctgaattatttcttccccagttgc

SEQ ID NO: 283 - DNA, 25 bp, synthetic construct
  source 1..25: mol_type = other DNA
  misc_feature 1..25: note = Synthetic (Hu.DMD.Exon45.25.010.2)
attatttcttccccagttgcattca

SEQ ID NO: 284 - DNA, 25 bp, synthetic construct
  source 1..25: mol_type = other DNA
  misc_feature 1..25: note = Synthetic (Hu.DMD.Exon45.25.011)
ggcatctgtttttgaggattgctga

SEQ ID NO: 285 - DNA, 25 bp, synthetic construct
  source 1..25: mol_type = other DNA
  misc_feature 1..25: note = Synthetic (Hu.DMD.Exon45.25.011.2)
tttgaggattgctgaattatttctt

SEQ ID NO: 286 - DNA, 25 bp, synthetic construct
  source 1..25: mol_type = other DNA
  misc_feature 1..25: note = Synthetic (Hu.DMD.Exon45.25.012)
aatttttcctgtagaatactggcat

SEQ ID NO: 287 - DNA, 25 bp, synthetic construct
  source 1..25: mol_type = other DNA
  misc_feature 1..25: note = Synthetic (Hu.DMD.Exon45.25.012.2)
atactggcatctgtttttgaggatt

SEQ ID NO: 288 - DNA, 25 bp, synthetic construct
  source 1..25: mol_type = other DNA
  misc_feature 1..25: note = Synthetic (Hu.DMD.Exon45.25.013)
accgcagattcaggcttcccaattt

SEQ ID NO: 289 - DNA, 25 bp, synthetic construct
  source 1..25: mol_type = other DNA
  misc_feature 1..25: note = Synthetic (Hu.DMD.Exon45.25.014)
ctgtttgcagacctcctgccaccgc

SEQ ID NO: 290 - DNA, 25 bp, synthetic construct
  source 1..25: mol_type = other DNA
  misc_feature 1..25: note = Synthetic (Hu.DMD.Exon45.25.014.2)
agattcaggcttcccaatttttcct

SEQ ID NO: 291 - DNA, 25 bp, synthetic construct
  source 1..25: mol_type = other DNA
  misc_feature 1..25: note = Synthetic (Hu.DMD.Exon45.25.015)
ctcttttttctgtctgacagctgtt

SEQ ID NO: 292 - DNA, 25 bp, synthetic construct
  source 1..25: mol_type = other DNA
  misc_feature 1..25: note = Synthetic (Hu.DMD.Exon45.25.015.2)
acctcctgccaccgcagattcaggc

SEQ ID NO: 293 - DNA, 25 bp, synthetic construct
  source 1..25: mol_type = other DNA
  misc_feature 1..25: note = Synthetic (Hu.DMD.Exon45.25.016)
cctacctcttttttctgtctgacag

SEQ ID NO: 294 - DNA, 25 bp, synthetic construct
  source 1..25: mol_type = other DNA
  misc_feature 1..25: note = Synthetic (Hu.DMD.Exon45.25.016.2)
gacagctgtttgcagacctcctgcc

SEQ ID NO: 295 - DNA, 25 bp, synthetic construct
  source 1..25: mol_type = other DNA
  misc_feature 1..25: note = Synthetic (Hu.DMD.Exon45.25.017)
gtcgccctacctcttttttctgtct

SEQ ID NO: 296 - DNA, 25 bp, synthetic construct
  source 1..25: mol_type = other DNA
  misc_feature 1..25: note = Synthetic (Hu.DMD.Exon45.25.018)
gatctgtcgccctacctcttttttc

SEQ ID NO: 297 - DNA, 25 bp, synthetic construct
  source 1..25: mol_type = other DNA
  misc_feature 1..25: note = Synthetic (Hu.DMD.Exon45.25.019)
tattagatctgtcgccctacctctt

SEQ ID NO: 298 - DNA, 25 bp, synthetic construct
  source 1..25: mol_type = other DNA
  misc_feature 1..25: note = Synthetic (Hu.DMD.Exon45.25.020)
attcctattagatctgtcgccctac

SEQ ID NO: 299 - DNA, 20 bp, synthetic construct
  source 1..20: mol_type = other DNA
  misc_feature 1..20: note = Synthetic (Hu.DMD.Exon45.20.001)
agataccaaaaaggcaaaac

SEQ ID NO: 300 - DNA, 20 bp, synthetic construct
  source 1..20: mol_type = other DNA
  misc_feature 1..20: note = Synthetic (Hu.DMD.Exon45.20.002)
aagataccaaaaaggcaaaa

SEQ ID NO: 301 - DNA, 20 bp, synthetic construct
  source 1..20: mol_type = other DNA
  misc_feature 1..20: note = Synthetic (Hu.DMD.Exon45.20.003)
cctgtaagataccaaaaagg

SEQ ID NO: 302 - DNA, 20 bp, synthetic construct
  source 1..20: mol_type = other DNA
  misc_feature 1..20: note = Synthetic (Hu.DMD.Exon45.20.004)
gagttcctgtaagataccaa

SEQ ID NO: 303 - DNA, 20 bp, synthetic construct
  source 1..20: mol_type = other DNA
  misc_feature 1..20: note = Synthetic (Hu.DMD.Exon45.20.005)
tcctggagttcctgtaagat

SEQ ID NO: 304 - DNA, 20 bp, synthetic construct
  source 1..20: mol_type = other DNA
  misc_feature 1..20: note = Synthetic (Hu.DMD.Exon45.20.006)
tgccatcctggagttcctgt

SEQ ID NO: 305 - DNA, 20 bp, synthetic construct
  source 1..20: mol_type = other DNA
  misc_feature 1..20: note = Synthetic (Hu.DMD.Exon45.20.007)
cccaatgccatcctggagtt

SEQ ID NO: 306 - DNA, 20 bp, synthetic construct
  source 1..20: mol_type = other DNA
  misc_feature 1..20: note = Synthetic (Hu.DMD.Exon45.20.008)
cgctgcccaatgccatcctg

SEQ ID NO: 307 - DNA, 20 bp, synthetic construct
  source 1..20: mol_type = other DNA
  misc_feature 1..20: note = Synthetic (Hu.DMD.Exon45.20.009)
ctgacaacagtttgccgctg

SEQ ID NO: 308 - DNA, 20 bp, synthetic construct
  source 1..20: mol_type = other DNA
  misc_feature 1..20: note = Synthetic (Hu.DMD.Exon45.20.010)
gttgcattcaatgttctgac

SEQ ID NO: 309 - DNA, 20 bp, synthetic construct
  source 1..20: mol_type = other DNA
  misc_feature 1..20: note = Synthetic (Hu.DMD.Exon45.20.011)
attatttcttccccagttgc

SEQ ID NO: 310 - DNA, 20 bp, synthetic construct
  source 1..20: mol_type = other DNA
  misc_feature 1..20: note = Synthetic (Hu.DMD.Exon45.20.012)
tttgaggattgctgaattat

SEQ ID NO: 311 - DNA, 20 bp, synthetic construct
  source 1..20: mol_type = other DNA
  misc_feature 1..20: note = Synthetic (Hu.DMD.Exon45.20.013)
atactggcatctgtttttga

SEQ ID NO: 312 - DNA, 20 bp, synthetic construct
  source 1..20: mol_type = other DNA
  misc_feature 1..20: note = Synthetic (Hu.DMD.Exon45.20.014)
aatttttcctgtagaatact

SEQ ID NO: 313 - DNA, 20 bp, synthetic construct
  source 1..20: mol_type = other DNA
  misc_feature 1..20: note = Synthetic (Hu.DMD.Exon45.20.015)
agattcaggcttcccaattt

SEQ ID NO: 314 - DNA, 20 bp, synthetic construct
  source 1..20: mol_type = other DNA
  misc_feature 1..20: note = Synthetic (Hu.DMD.Exon45.20.016)
acctcctgccaccgcagatt

SEQ ID NO: 315 - DNA, 20 bp, synthetic construct
  source 1..20: mol_type = other DNA
  misc_feature 1..20: note = Synthetic (Hu.DMD.Exon45.20.017)
gacagctgtttgcagacctc

SEQ ID NO: 316 - DNA, 20 bp, synthetic construct
  source 1..20: mol_type = other DNA
  misc_feature 1..20: note = Synthetic (Hu.DMD.Exon45.20.018)
ctcttttttctgtctgacag

SEQ ID NO: 317 - DNA, 20 bp, synthetic construct
  source 1..20: mol_type = other DNA
  misc_feature 1..20: note = Synthetic (Hu.DMD.Exon45.20.019)
cctacctcttttttctgtct

SEQ ID NO: 318 - DNA, 20 bp, synthetic construct
  source 1..20: mol_type = other DNA
  misc_feature 1..20: note = Synthetic (Hu.DMD.Exon45.20.020)
gtcgccctacctcttttttc

SEQ ID NO: 319 - DNA, 20 bp, synthetic construct
  source 1..20: mol_type = other DNA
  misc_feature 1..20: note = Synthetic (Hu.DMD.Exon45.20.021)
gatctgtcgccctacctctt

SEQ ID NO: 320 - DNA, 20 bp, synthetic construct
  source 1..20: mol_type = other DNA
  misc_feature 1..20: note = Synthetic (Hu.DMD.Exon45.20.022)
tattagatctgtcgccctac

SEQ ID NO: 321 - DNA, 20 bp, synthetic construct
  source 1..20: mol_type = other DNA
  misc_feature 1..20: note = Synthetic (Hu.DMD.Exon45.20.023)
attcctattagatctgtcgc

SEQ ID NO: 322 - DNA, 25 bp, synthetic construct
  source 1..25: mol_type = other DNA
  misc_feature 1..25: note = Synthetic (Hu.DMD.Exon46.25.001)
gggggatttgagaaaataaaattac

SEQ ID NO: 323 - DNA, 25 bp, synthetic construct
  source 1..25: mol_type = other DNA
  misc_feature 1..25: note = Synthetic (Hu.DMD.Exon46.25.002)
atttgagaaaataaaattaccttga

SEQ ID NO: 324 - DNA, 25 bp, synthetic construct
  source 1..25: mol_type = other DNA
  misc_feature 1..25: note = Synthetic (Hu.DMD.Exon46.25.002.2)
ctagcctggagaaagaagaataaaa

SEQ ID NO: 325 - DNA, 25 bp, synthetic construct
  source 1..25: mol_type = other DNA
  misc_feature 1..25: note = Synthetic (Hu.DMD.Exon46.25.003)
agaaaataaaattaccttgacttgc

SEQ ID NO: 326 - DNA, 25 bp, synthetic construct
  source 1..25: mol_type = other DNA
  misc_feature 1..25: note = Synthetic (Hu.DMD.Exon46.25.003.2)
ttcttctagcctggagaaagaagaa

SEQ ID NO: 327 - DNA, 25 bp, synthetic construct
  source 1..25: mol_type = other DNA
  misc_feature 1..25: note = Synthetic (Hu.DMD.Exon46.25.004)
ataaaattaccttgacttgctcaag

SEQ ID NO: 328 - DNA, 25 bp, synthetic construct
  source 1..25: mol_type = other DNA
  misc_feature 1..25: note = Synthetic (Hu.DMD.Exon46.25.004.2)
ttttgttcttctagcctggagaaag

SEQ ID NO: 329 - DNA, 25 bp, synthetic construct
  source 1..25: mol_type = other DNA
  misc_feature 1..25: note = Synthetic (Hu.DMD.Exon46.25.005)
attaccttgacttgctcaagctttt

SEQ ID NO: 330 - DNA, 25 bp, synthetic construct
  source 1..25: mol_type = other DNA
  misc_feature 1..25: note = Synthetic (Hu.DMD.Exon46.25.005.2)
tattcttttgttcttctagcctgga

SEQ ID NO: 331 - DNA, 25 bp, synthetic construct
  source 1..25: mol_type = other DNA
  misc_feature 1..25: note = Synthetic (Hu.DMD.Exon46.25.006)
cttgacttgctcaagcttttctttt

SEQ ID NO: 332 - DNA, 25 bp, synthetic construct
  source 1..25: mol_type = other DNA
  misc_feature 1..25: note = Synthetic (Hu.DMD.Exon46.25.006.2)
caagatattcttttgttcttctagc

SEQ ID NO: 333 - DNA, 25 bp, synthetic construct
  source 1..25: mol_type = other DNA
  misc_feature 1..25: note = Synthetic (Hu.DMD.Exon46.25.007)
cttttagttgctgctcttttccagg

SEQ ID NO: 334 - DNA, 25 bp, synthetic construct
  source 1..25: mol_type = other DNA
  misc_feature 1..25: note = Synthetic (Hu.DMD.Exon46.25.008)
ccaggttcaagtgggatactagcaa

SEQ ID NO: 335 - DNA, 25 bp, synthetic construct
  source 1..25: mol_type = other DNA
  misc_feature 1..25: note = Synthetic (Hu.DMD.Exon46.25.008.2)
atctctttgaaattctgacaagata

SEQ ID NO: 336 - DNA, 25 bp, synthetic construct
  source 1..25: mol_type = other DNA
  misc_feature 1..25: note = Synthetic (Hu.DMD.Exon46.25.009)
agcaatgttatctgcttcctccaac

SEQ ID NO: 337 - DNA, 25 bp, synthetic construct
  source 1..25: mol_type = other DNA
  misc_feature 1..25: note = Synthetic (Hu.DMD.Exon46.25.009.2)
aacaaattcatttaaatctctttga

SEQ ID NO: 338 - DNA, 25 bp, synthetic construct
  source 1..25: mol_type = other DNA
  misc_feature 1..25: note = Synthetic (Hu.DMD.Exon46.25.010)
ccaaccataaaacaaattcatttaa

SEQ ID NO: 339 - DNA, 25 bp, synthetic construct
  source 1..25: mol_type = other DNA
  misc_feature 1..25: note = Synthetic (Hu.DMD.Exon46.25.010.2)
ttcctccaaccataaaacaaattca

SEQ ID NO: 340 - DNA, 25 bp, synthetic construct
  source 1..25: mol_type = other DNA
  misc_feature 1..25: note = Synthetic (Hu.DMD.Exon46.25.011)
tttaaatctctttgaaattctgaca

SEQ ID NO: 341 - DNA, 25 bp, synthetic construct
  source 1..25: mol_type = other DNA
  misc_feature 1..25: note = Synthetic (Hu.DMD.Exon46.25.012)
tgacaagatattcttttgttcttct

SEQ ID NO: 342 - DNA, 25 bp, synthetic construct
  source 1..25: mol_type = other DNA
  misc_feature 1..25: note = Synthetic (Hu.DMD.Exon46.25.012.2)
ttcaagtgggatactagcaatgtta

SEQ ID NO: 343 - DNA, 25 bp, synthetic construct
  source 1..25: mol_type = other DNA
  misc_feature 1..25: note = Synthetic (Hu.DMD.Exon46.25.013)
agatattcttttgttcttctagcct

SEQ ID NO: 344 - DNA, 25 bp, synthetic construct
  source 1..25: mol_type = other DNA
  misc_feature 1..25: note = Synthetic (Hu.DMD.Exon46.25.013.2)
ctgctcttttccaggttcaagtggg

SEQ ID NO: 345 - DNA, 25 bp, synthetic construct
  source 1..25: mol_type = other DNA
  misc_feature 1..25: note = Synthetic (Hu.DMD.Exon46.25.014)
ttcttttgttcttctagcctggaga

SEQ ID NO: 346 - DNA, 25 bp, synthetic construct
  source 1..25: mol_type = other DNA
  misc_feature 1..25: note = Synthetic (Hu.DMD.Exon46.25.014.2)
cttttcttttagttgctgctctttt

SEQ ID NO: 347 - DNA, 25 bp, synthetic construct
  source 1..25: mol_type = other DNA
  misc_feature 1..25: note = Synthetic (Hu.DMD.Exon46.25.015)
ttgttcttctagcctggagaaagaa

SEQ ID NO: 348 - DNA, 25 bp, synthetic construct
  source 1..25: mol_type = other DNA
  misc_feature 1..25: note = Synthetic (Hu.DMD.Exon46.25.016)
cttctagcctggagaaagaagaata

SEQ ID NO: 349 - DNA, 25 bp, synthetic construct
  source 1..25: mol_type = other DNA
  misc_feature 1..25: note = Synthetic (Hu.DMD.Exon46.25.017)
agcctggagaaagaagaataaaatt

SEQ ID NO: 350 - DNA, 25 bp, synthetic construct
  source 1..25: mol_type = other DNA
  misc_feature 1..25: note = Synthetic (Hu.DMD.Exon46.25.018)
ctggagaaagaagaataaaattgtt

SEQ ID NO: 351 - DNA, 20 bp, synthetic construct
  source 1..20: mol_type = other DNA
  misc_feature 1..20: note = Synthetic (Hu.DMD.Exon46.20.001)
gaaagaagaataaaattgtt

SEQ ID NO: 352 - DNA, 20 bp, synthetic construct
  source 1..20: mol_type = other DNA
  misc_feature 1..20: note = Synthetic (Hu.DMD.Exon46.20.002)
ggagaaagaagaataaaatt

SEQ ID NO: 353 - DNA, 20 bp, synthetic construct
  source 1..20: mol_type = other DNA
  misc_feature 1..20: note = Synthetic (Hu.DMD.Exon46.20.003)
agcctggagaaagaagaata

SEQ ID NO: 354 - DNA, 20 bp, synthetic construct
  source 1..20: mol_type = other DNA
  misc_feature 1..20: note = Synthetic (Hu.DMD.Exon46.20.004)
cttctagcctggagaaagaa

SEQ ID NO: 355 - DNA, 20 bp, synthetic construct
  source 1..20: mol_type = other DNA
  misc_feature 1..20: note = Synthetic (Hu.DMD.Exon46.20.005)
ttgttcttctagcctggaga

SEQ ID NO: 356 - DNA, 20 bp, synthetic construct
  source 1..20: mol_type = other DNA
  misc_feature 1..20: note = Synthetic (Hu.DMD.Exon46.20.006)
ttcttttgttcttctagcct

SEQ ID NO: 357 - DNA, 20 bp, synthetic construct
  source 1..20: mol_type = other DNA
  misc_feature 1..20: note = Synthetic (Hu.DMD.Exon46.20.007)
tgacaagatattcttttgtt

SEQ ID NO: 358 - DNA, 20 bp, synthetic construct
  source 1..20: mol_type = other DNA
  misc_feature 1..20: note = Synthetic (Hu.DMD.Exon46.20.008)
atctctttgaaattctgaca

SEQ ID NO: 359 - DNA, 20 bp, synthetic construct
  source 1..20: mol_type = other DNA
  misc_feature 1..20: note = Synthetic (Hu.DMD.Exon46.20.009)
aacaaattcatttaaatctc

SEQ ID NO: 360 - DNA, 20 bp, synthetic construct
  source 1..20: mol_type = other DNA
  misc_feature 1..20: note = Synthetic (Hu.DMD.Exon46.20.010)
ttcctccaaccataaaacaa

SEQ ID NO: 361 - DNA, 20 bp, synthetic construct
  source 1..20: mol_type = other DNA
  misc_feature 1..20: note = Synthetic (Hu.DMD.Exon46.20.011)
agcaatgttatctgcttcct

SEQ ID NO: 362 - DNA, 20 bp, synthetic construct
  source 1..20: mol_type = other DNA
  misc_feature 1..20: note = Synthetic (Hu.DMD.Exon46.20.012)
ttcaagtgggatactagcaa

SEQ ID NO: 363 - DNA, 20 bp, synthetic construct
  source 1..20: mol_type = other DNA
  misc_feature 1..20: note = Synthetic (Hu.DMD.Exon46.20.013)
ctgctcttttccaggttcaa

SEQ ID NO: 364 - DNA, 20 bp, synthetic construct
  source 1..20: mol_type = other DNA
  misc_feature 1..20: note = Synthetic (Hu.DMD.Exon46.20.014)
cttttcttttagttgctgct

SEQ ID NO: 365 - DNA, 20 bp, synthetic construct
  source 1..20: mol_type = other DNA
  misc_feature 1..20: note = Synthetic (Hu.DMD.Exon46.20.015)
cttgacttgctcaagctttt

SEQ ID NO: 366 - DNA, 20 bp, synthetic construct
  source 1..20: mol_type = other DNA
  misc_feature 1..20: note = Synthetic (Hu.DMD.Exon46.20.016)
attaccttgacttgctcaag

SEQ ID NO: 367 - DNA, 20 bp, synthetic construct
  source 1..20: mol_type = other DNA
  misc_feature 1..20: note = Synthetic (Hu.DMD.Exon46.20.017)
ataaaattaccttgacttgc

SEQ ID NO: 368 - DNA, 20 bp, synthetic construct
  source 1..20: mol_type = other DNA
  misc_feature 1..20: note = Synthetic (Hu.DMD.Exon46.20.018)
agaaaataaaattaccttga

SEQ ID NO: 369 - DNA, 20 bp, synthetic construct
  source 1..20: mol_type = other DNA
  misc_feature 1..20: note = Synthetic (Hu.DMD.Exon46.20.019)
atttgagaaaataaaattac

SEQ ID NO: 370 - DNA, 20 bp, synthetic construct
  source 1..20: mol_type = other DNA
  misc_feature 1..20: note = Synthetic (Hu.DMD.Exon46.20.020)
gggggatttgagaaaataaa

SEQ ID NO: 371 - DNA, 25 bp, synthetic construct
  source 1..25: mol_type = other DNA
  misc_feature 1..25: note = Synthetic (Hu.DMD.Exon47.25.001)
ctgaaacagacaaatgcaacaacgt

SEQ ID NO: 372 - DNA, 25 bp, synthetic construct
  source 1..25: mol_type = other DNA
  misc_feature 1..25: note = Synthetic (Hu.DMD.Exon47.25.002)
agtaactgaaacagacaaatgcaac

SEQ ID NO: 373 - DNA, 25 bp, synthetic construct
  source 1..25: mol_type = other DNA
  misc_feature 1..25: note = Synthetic (Hu.DMD.Exon47.25.003)
ccaccagtaactgaaacagacaaat

SEQ ID NO: 374 - DNA, 25 bp, synthetic construct
  source 1..25: mol_type = other DNA
  misc_feature 1..25: note = Synthetic (Hu.DMD.Exon47.25.004)
ctcttccaccagtaactgaaacaga

SEQ ID NO: 375 - DNA, 25 bp, synthetic construct
  source 1..25: mol_type = other DNA
  misc_feature 1..25: note = Synthetic (Hu.DMD.Exon47.25.005)
ggcaactcttccaccagtaactgaa

SEQ ID NO: 376 - DNA, 25 bp, synthetic construct
  source 1..25: mol_type = other DNA
  misc_feature 1..25: note = Synthetic (Hu.DMD.Exon47.25.006)
gcaggggcaactcttccaccagtaa

SEQ ID NO: 377 - DNA, 25 bp, synthetic construct
  source 1..25: mol_type = other DNA
  misc_feature 1..25: note = Synthetic (Hu.DMD.Exon47.25.007)
ctggcgcaggggcaactcttccacc

SEQ ID NO: 378 - DNA, 25 bp, synthetic construct
  source 1..25: mol_type = other DNA
  misc_feature 1..25: note = Synthetic (Hu.DMD.Exon47.25.008)
tttaattgtttgagaattccctggc

SEQ ID NO: 379 - DNA, 25 bp, synthetic construct
  source 1..25: mol_type = other DNA
  misc_feature 1..25: note = Synthetic (Hu.DMD.Exon47.25.008.2)
ttgtttgagaattccctggcgcagg

SEQ ID NO: 380 - DNA, 25 bp, synthetic construct
  source 1..25: mol_type = other DNA
  misc_feature 1..25: note = Synthetic (Hu.DMD.Exon47.25.009)
gcacgggtcctccagtttcatttaa

SEQ ID NO: 381 - DNA, 25 bp, synthetic construct
  source 1..25: mol_type = other DNA
  misc_feature 1..25: note = Synthetic (Hu.DMD.Exon47.25.009.2)
tccagtttcatttaattgtttgaga

SEQ ID NO: 382 - DNA, 25 bp, synthetic construct
  source 1..25: mol_type = other DNA
  misc_feature 1..25: note = Synthetic (Hu.DMD.Exon47.25.010)
gcttatgggagcacttacaagcacg

SEQ ID NO: 383 - DNA, 25 bp, synthetic construct
  source 1..25: mol_type = other DNA
  misc_feature 1..25: note = Synthetic (Hu.DMD.Exon47.25.010.2)
tacaagcacgggtcctccagtttca

SEQ ID NO: 384 - DNA, 25 bp, synthetic construct
  source 1..25: mol_type = other DNA
  misc_feature 1..25: note = Synthetic (Hu.DMD.Exon47.25.011)
agtttatcttgctcttctgggctta

SEQ ID NO: 385 - DNA, 25 bp, synthetic construct
  source 1..25: mol_type = other DNA
  misc_feature 1..25: note = Synthetic (Hu.DMD.Exon47.25.012)
tctgcttgagcttattttcaagttt

SEQ ID NO: 386 - DNA, 25 bp, synthetic construct
  source 1..25: mol_type = other DNA
  misc_feature 1..25: note = Synthetic (Hu.DMD.Exon47.25.012.2)
atcttgctcttctgggcttatggga

SEQ ID NO: 387 - DNA, 25 bp, synthetic construct
  source 1..25: mol_type = other DNA
  misc_feature 1..25: note = Synthetic (Hu.DMD.Exon47.25.013)
ctttatccactggagatttgtctgc

SEQ ID NO: 388 - DNA, 25 bp, synthetic construct
  source 1..25: mol_type = other DNA
  misc_feature 1..25: note = Synthetic (Hu.DMD.Exon47.25.013.2)
cttattttcaagtttatcttgctct

SEQ ID NO: 389 - DNA, 25 bp, synthetic construct
  source 1..25: mol_type = other DNA
  misc_feature 1..25: note = Synthetic (Hu.DMD.Exon47.25.014)
ctaacctttatccactggagatttg

SEQ ID NO: 390 - DNA, 25 bp, synthetic construct
  source 1..25: mol_type = other DNA
  misc_feature 1..25: note = Synthetic (Hu.DMD.Exon47.25.014.2)
atttgtctgcttgagcttattttca

SEQ ID NO: 391 - DNA, 25 bp, synthetic construct
  source 1..25: mol_type = other DNA
  misc_feature 1..25: note = Synthetic (Hu.DMD.Exon47.25.015)
aatgtctaacctttatccactggag

SEQ ID NO: 392 - DNA, 25 bp, synthetic construct
  source 1..25: mol_type = other DNA
  misc_feature 1..25: note = Synthetic (Hu.DMD.Exon47.25.016)
tggttaatgtctaacctttatccac

SEQ ID NO: 393 - DNA, 25 bp, synthetic construct
  source 1..25: mol_type = other DNA
  misc_feature 1..25: note = Synthetic (Hu.DMD.Exon47.25.017)
agagatggttaatgtctaaccttta

SEQ ID NO: 394 - DNA, 25 bp, synthetic construct
  source 1..25: mol_type = other DNA
  misc_feature 1..25: note = Synthetic (Hu.DMD.Exon47.25.018)
acggaagagatggttaatgtctaac

SEQ ID NO: 395 - DNA, 20 bp, synthetic construct
  source 1..20: mol_type = other DNA
  misc_feature 1..20: note = Synthetic (Hu.DMD.Exon47.20.001)
acagacaaatgcaacaacgt

SEQ ID NO: 396 - DNA, 20 bp, synthetic construct
  source 1..20: mol_type = other DNA
  misc_feature 1..20: note = Synthetic (Hu.DMD.Exon47.20.002)
ctgaaacagacaaatgcaac

SEQ ID NO: 397 - DNA, 20 bp, synthetic construct
  source 1..20: mol_type = other DNA
  misc_feature 1..20: note = Synthetic (Hu.DMD.Exon47.20.003)
agtaactgaaacagacaaat

SEQ ID NO: 398 - DNA, 20 bp, synthetic construct
  source 1..20: mol_type = other DNA
  misc_feature 1..20: note = Synthetic (Hu.DMD.Exon47.20.004)
ccaccagtaactgaaacaga

SEQ ID NO: 399 - DNA, 20 bp, synthetic construct
  source 1..20: mol_type = other DNA
  misc_feature 1..20: note = Synthetic (Hu.DMD.Exon47.20.005)
ctcttccaccagtaactgaa

SEQ ID NO: 400 - DNA, 20 bp, synthetic construct
  source 1..20: mol_type = other DNA
  misc_feature 1..20: note = Synthetic (Hu.DMD.Exon47.20.006)
ggcaactcttccaccagtaa

SEQ ID NO: 401 - DNA, 20 bp, synthetic construct
  source 1..20: mol_type = other DNA
  misc_feature 1..20: note = Synthetic (Hu.DMD.Exon47.20.007)
ctggcgcaggggcaactctt

SEQ ID NO: 402 - DNA, 20 bp, synthetic construct
  source 1..20: mol_type = other DNA
  misc_feature 1..20: note = Synthetic (Hu.DMD.Exon47.20.008)
ttgtttgagaattccctggc

SEQ ID NO: 403 - DNA, 20 bp, synthetic construct
  source 1..20: mol_type = other DNA
  misc_feature 1..20: note = Synthetic (Hu.DMD.Exon47.20.009)
tccagtttcatttaattgtt

SEQ ID NO: 404 - DNA, 20 bp, synthetic construct
  source 1..20: mol_type = other DNA
  misc_feature 1..20: note = Synthetic (Hu.DMD.Exon47.20.010)
tacaagcacgggtcctccag

SEQ ID NO: 405 - DNA, 20 bp, synthetic construct
  source 1..20: mol_type = other DNA
  misc_feature 1..20: note = Synthetic (Hu.DMD.Exon47.20.011)
gcttatgggagcacttacaa

SEQ ID NO: 406 - DNA, 20 bp, synthetic construct
  source 1..20: mol_type = other DNA
  misc_feature 1..20: note = Synthetic (Hu.DMD.Exon47.20.012)
atcttgctcttctgggctta

SEQ ID NO: 407 - DNA, 20 bp, synthetic construct
  source 1..20: mol_type = other DNA
  misc_feature 1..20: note = Synthetic (Hu.DMD.Exon47.20.013)
cttattttcaagtttatctt

SEQ ID NO: 408 - DNA, 20 bp, synthetic construct
  source 1..20: mol_type = other DNA
  misc_feature 1..20: note = Synthetic (Hu.DMD.Exon47.20.014)
atttgtctgcttgagcttat

SEQ ID NO: 409 - DNA, 20 bp, synthetic construct
  source 1..20: mol_type = other DNA
  misc_feature 1..20: note = Synthetic (Hu.DMD.Exon47.20.015)
ctttatccactggagatttg

SEQ ID NO: 410 - DNA, 20 bp, synthetic construct
  source 1..20: mol_type = other DNA
  misc_feature 1..20: note = Synthetic (Hu.DMD.Exon47.20.016)
ctaacctttatccactggag

SEQ ID NO: 411 - DNA, 20 bp, synthetic construct
  source 1..20: mol_type = other DNA
  misc_feature 1..20: note = Synthetic (Hu.DMD.Exon47.20.017)
aatgtctaacctttatccac

SEQ ID NO: 412 - DNA, 20 bp, synthetic construct
  source 1..20: mol_type = other DNA
  misc_feature 1..20: note = Synthetic (Hu.DMD.Exon47.20.018)
tggttaatgtctaaccttta

SEQ ID NO: 413 - DNA, 20 bp, synthetic construct
  source 1..20: mol_type = other DNA
  misc_feature 1..20: note = Synthetic (Hu.DMD.Exon47.20.019)
agagatggttaatgtctaac

SEQ ID NO: 414 - DNA, 20 bp, synthetic construct
  source 1..20: mol_type = other DNA
  misc_feature 1..20: note = Synthetic (Hu.DMD.Exon47.20.020)
acggaagagatggttaatgt

SEQ ID NO: 415 - DNA, 25 bp, synthetic construct
  source 1..25: mol_type = other DNA
  misc_feature 1..25: note = Synthetic (Hu.DMD.Exon48.25.001)
ctgaaaggaaaatacattttaaaaa

SEQ ID NO: 416 - DNA, 25 bp, synthetic construct
  source 1..25: mol_type = other DNA
  misc_feature 1..25: note = Synthetic (Hu.DMD.Exon48.25.002)
cctgaaaggaaaatacattttaaaa

SEQ ID NO: 417 - DNA, 25 bp, synthetic construct
  source 1..25: mol_type = other DNA
  misc_feature 1..25: note = Synthetic (Hu.DMD.Exon48.25.002.2)
gaaacctgaaaggaaaatacatttt

SEQ ID NO: 418 - DNA, 25 bp, synthetic construct
  source 1..25: mol_type = other DNA
  misc_feature 1..25: note = Synthetic (Hu.DMD.Exon48.25.003)
ggaaacctgaaaggaaaatacattt

SEQ ID NO: 419 - DNA, 25 bp, synthetic construct
  source 1..25: mol_type = other DNA
  misc_feature 1..25: note = Synthetic (Hu.DMD.Exon48.25.003.2)
ctctggaaacctgaaaggaaaatac

SEQ ID NO: 420 - DNA, 25 bp, synthetic construct
  source 1..25: mol_type = other DNA
  misc_feature 1..25: note = Synthetic (Hu.DMD.Exon48.25.004)
gctctggaaacctgaaaggaaaata

SEQ ID NO: 421 - DNA, 25 bp, synthetic construct
  source 1..25: mol_type = other DNA
  misc_feature 1..25: note = Synthetic (Hu.DMD.Exon48.25.004.2)
taaagctctggaaacctgaaaggaa

SEQ ID NO: 422 - DNA, 25 bp, synthetic construct
  source 1..25: mol_type = other DNA
  misc_feature 1..25: note = Synthetic (Hu.DMD.Exon48.25.005)
gtaaagctctggaaacctgaaagga

SEQ ID NO: 423 - DNA, 25 bp, synthetic construct
  source 1..25: mol_type = other DNA
  misc_feature 1..25: note = Synthetic (Hu.DMD.Exon48.25.005.2)
tcaggtaaagctctggaaacctgaa

SEQ ID NO: 424 - DNA, 25 bp, synthetic construct
  source 1..25: mol_type = other DNA
  misc_feature 1..25: note = Synthetic (Hu.DMD.Exon48.25.006)
ctcaggtaaagctctggaaacctga

SEQ ID NO: 425 - DNA, 25 bp, synthetic construct
  source 1..25: mol_type = other DNA
  misc_feature 1..25: note = Synthetic (Hu.DMD.Exon48.25.006.2)
gtttctcaggtaaagctctggaaac

SEQ ID NO: 426 - DNA, 25 bp, synthetic construct
  source 1..25: mol_type = other DNA
  misc_feature 1..25: note = Synthetic (Hu.DMD.Exon48.25.007)
tgtttctcaggtaaagctctggaaa

SEQ ID NO: 427 - DNA, 25 bp, synthetic construct
  source 1..25: mol_type = other DNA
  misc_feature 1..25: note = Synthetic (Hu.DMD.Exon48.25.007.2)
aatttctccttgtttctcaggtaaa

SEQ ID NO: 428 - DNA, 25 bp, synthetic construct
  source 1..25: mol_type = other DNA
  misc_feature 1..25: note = Synthetic (Hu.DMD.Exon48.25.008)
tttgagcttcaatttctccttgttt

SEQ ID NO: 429 - DNA, 25 bp, synthetic construct
  source 1..25: mol_type = other DNA
  misc_feature 1..25: note = Synthetic (Hu.DMD.Exon48.25.008)
ttttatttgagcttcaatttctcct

SEQ ID NO: 430 - DNA, 25 bp, synthetic construct
  source 1..25: mol_type = other DNA
  misc_feature 1..25: note = Synthetic (Hu.DMD.Exon48.25.009)
aagctgcccaaggtcttttatttga

SEQ ID NO: 431 - DNA, 25 bp, synthetic construct
  source 1..25: mol_type = other DNA
  misc_feature 1..25: note = Synthetic (Hu.DMD.Exon48.25.010)
aggtcttcaagctttttttcaagct

SEQ ID NO: 432 - DNA, 25 bp, synthetic construct
  source 1..25: mol_type = other DNA
  misc_feature 1..25: note = Synthetic (Hu.DMD.Exon48.25.010.2)
ttcaagctttttttcaagctgccca

SEQ ID NO: 433 - DNA, 25 bp, synthetic construct
  source 1..25: mol_type = other DNA
  misc_feature 1..25: note = Synthetic (Hu.DMD.Exon48.25.011)
gatgatttaactgctcttcaaggtc

SEQ ID NO: 434 - DNA, 25 bp, synthetic construct
  source 1..25: mol_type = other DNA
  misc_feature 1..25: note = Synthetic (Hu.DMD.Exon48.25.011.2)
ctgctcttcaaggtcttcaagcttt

SEQ ID NO: 435 - DNA, 25 bp, synthetic construct
  source 1..25: mol_type = other DNA
  misc_feature 1..25: note = Synthetic (Hu.DMD.Exon48.25.012)
aggagataaccacagcagcagatga

SEQ ID NO: 436 - DNA, 25 bp, synthetic construct
  source 1..25: mol_type = other DNA
  misc_feature 1..25: note = Synthetic (Hu.DMD.Exon48.25.012.2)
cagcagatgatttaactgctcttca

SEQ ID NO: 437 - DNA, 25 bp, synthetic construct
  source 1..25: mol_type = other DNA
  misc_feature 1..25: note = Synthetic (Hu.DMD.Exon48.25.013)
atttccaactgattcctaataggag

SEQ ID NO: 438 - DNA, 25 bp, synthetic construct
  source 1..25: mol_type = other DNA
  misc_feature 1..25: note = Synthetic (Hu.DMD.Exon48.25.014)
cttggtttggttggttataaatttc

SEQ ID NO: 439 - DNA, 25 bp, synthetic construct
  source 1..25: mol_type = other DNA
  misc_feature 1..25: note = Synthetic (Hu.DMD.Exon48.25.014.2)
caactgattcctaataggagataac

SEQ ID NO: 440 - DNA, 25 bp, synthetic construct
  source 1..25: mol_type = other DNA
  misc_feature 1..25: note = Synthetic (Hu.DMD.Exon48.25.015)
cttaacgtcaaatggtccttcttgg

SEQ ID NO: 441 - DNA, 25 bp, synthetic construct
  source 1..25: mol_type = other DNA
  misc_feature 1..25: note = Synthetic (Hu.DMD.Exon48.25.015.2)
ttggttataaatttccaactgattc

SEQ ID NO: 442 - DNA, 25 bp, synthetic construct
  source 1..25: mol_type = other DNA
  misc_feature 1..25: note = Synthetic (Hu.DMD.Exon48.25.016)
cctaccttaacgtcaaatggtcctt

SEQ ID NO: 443 - DNA, 25 bp, synthetic construct
  source 1..25: mol_type = other DNA
  misc_feature 1..25: note = Synthetic (Hu.DMD.Exon48.25.016.2)
tccttcttggtttggttggttataa

SEQ ID NO: 444 - DNA, 25 bp, synthetic construct
  source 1..25: mol_type = other DNA
  misc_feature 1..25: note = Synthetic (Hu.DMD.Exon48.25.017)
agttccctaccttaacgtcaaatgg

SEQ ID NO: 445 - DNA, 25 bp, synthetic construct
  source 1..25: mol_type = other DNA
  misc_feature 1..25: note = Synthetic (Hu.DMD.Exon48.25.018)
caaaaagttccctaccttaacgtca

SEQ ID NO: 446 - DNA, 25 bp, synthetic construct
  source 1..25: mol_type = other DNA
  misc_feature 1..25: note = Synthetic (Hu.DMD.Exon48.25.019)
taaagcaaaaagttccctaccttaa

SEQ ID NO: 447 - DNA, 25 bp, synthetic construct
  source 1..25: mol_type = other DNA
  misc_feature 1..25: note = Synthetic (Hu.DMD.Exon48.25.020)
atatttaaagcaaaaagttccctac

SEQ ID NO: 448 - DNA, 20 bp, synthetic construct
  source 1..20: mol_type = other DNA
  misc_feature 1..20: note = Synthetic (Hu.DMD.Exon48.20.001)
aggaaaatacattttaaaaa

SEQ ID NO: 449 - DNA, 20 bp, synthetic construct
  source 1..20: mol_type = other DNA
  misc_feature 1..20: note = Synthetic (Hu.DMD.Exon48.20.002)
aaggaaaatacattttaaaa

SEQ ID NO: 450 - DNA, 20 bp, synthetic construct
  source 1..20: mol_type = other DNA
  misc_feature 1..20: note = Synthetic (Hu.DMD.Exon48.20.003)
cctgaaaggaaaatacattt

SEQ ID NO: 451 - DNA, 20 bp, synthetic construct
  source 1..20: mol_type = other DNA
  misc_feature 1..20: note = Synthetic (Hu.DMD.Exon48.20.004)
ggaaacctgaaaggaaaata

SEQ ID NO: 452 - DNA, 20 bp, synthetic construct
  source 1..20: mol_type = other DNA
  misc_feature 1..20: note = Synthetic (Hu.DMD.Exon48.20.005)
gctctggaaacctgaaagga

SEQ ID NO: 453 - DNA, 20 bp, synthetic construct
  source 1..20: mol_type = other DNA
  misc_feature 1..20: note = Synthetic (Hu.DMD.Exon48.20.006)
gtaaagctctggaaacctga

SEQ ID NO: 454 - DNA, 20 bp, synthetic construct
  source 1..20: mol_type = other DNA
  misc_feature 1..20: note = Synthetic (Hu.DMD.Exon48.20.007)
ctcaggtaaagctctggaaa

SEQ ID NO: 455 - DNA, 20 bp, synthetic construct
  source 1..20: mol_type = other DNA
  misc_feature 1..20: note = Synthetic (Hu.DMD.Exon48.20.008)
aatttctccttgtttctcag

SEQ ID NO: 456 - DNA, 20 bp, synthetic construct
  source 1..20: mol_type = other DNA
  misc_feature 1..20: note = Synthetic (Hu.DMD.Exon48.20.009)
ttttatttgagcttcaattt

SEQ ID NO: 457 - DNA, 20 bp, synthetic construct
  source 1..20: mol_type = other DNA
  misc_feature 1..20: note = Synthetic (Hu.DMD.Exon48.20.010)
aagctgcccaaggtctttta

SEQ ID NO: 458 - DNA, 20 bp, synthetic construct
  source 1..20: mol_type = other DNA
  misc_feature 1..20: note = Synthetic (Hu.DMD.Exon48.20.011)
ttcaagctttttttcaagct

SEQ ID NO: 459 - DNA, 20 bp, synthetic construct
  source 1..20: mol_type = other DNA
  misc_feature 1..20: note = Synthetic (Hu.DMD.Exon48.20.012)
ctgctcttcaaggtcttcaa

SEQ ID NO: 460 - DNA, 20 bp, synthetic construct
  source 1..20: mol_type = other DNA
  misc_feature 1..20: note = Synthetic (Hu.DMD.Exon48.20.013)
cagcagatgatttaactgct

SEQ ID NO: 461 - DNA, 20 bp, synthetic construct
  source 1..20: mol_type = other DNA
  misc_feature 1..20: note = Synthetic (Hu.DMD.Exon48.20.014)
aggagataaccacagcagca

SEQ ID NO: 462 - DNA, 20 bp, synthetic construct
  source 1..20: mol_type = other DNA
  misc_feature 1..20: note = Synthetic (Hu.DMD.Exon48.20.015)
caactgattcctaataggag

SEQ ID NO: 463 - DNA, 20 bp, synthetic construct
  source 1..20: mol_type = other DNA
  misc_feature 1..20: note = Synthetic (Hu.DMD.Exon48.20.016)
ttggttataaatttccaact

SEQ ID NO: 464 - DNA, 20 bp, synthetic construct
  source 1..20: mol_type = other DNA
  misc_feature 1..20: note = Synthetic (Hu.DMD.Exon48.20.017)
tccttcttggtttggttggt

SEQ ID NO: 465 - DNA, 20 bp, synthetic construct
  source 1..20: mol_type = other DNA
  misc_feature 1..20: note = Synthetic (Hu.DMD.Exon48.20.018)
cttaacgtcaaatggtcctt

SEQ ID NO: 466 - DNA, 20 bp, synthetic construct
  source 1..20: mol_type = other DNA
  misc_feature 1..20: note = Synthetic (Hu.DMD.Exon48.20.019)
cctaccttaacgtcaaatgg

SEQ ID NO: 467 - DNA, 20 bp, synthetic construct
  source 1..20: mol_type = other DNA
  misc_feature 1..20: note = Synthetic (Hu.DMD.Exon48.20.020)
agttccctaccttaacgtca

SEQ ID NO: 468 - DNA, 20 bp, synthetic construct
  source 1..20: mol_type = other DNA
  misc_feature 1..20: note = Synthetic (Hu.DMD.Exon48.20.021)
caaaaagttccctaccttaa

SEQ ID NO: 469 - DNA, 20 bp, synthetic construct
  source 1..20: mol_type = other DNA
  misc_feature 1..20: note = Synthetic (Hu.DMD.Exon48.20.022)
taaagcaaaaagttccctac

SEQ ID NO: 470 - DNA, 20 bp, synthetic construct
  source 1..20: mol_type = other DNA
  misc_feature 1..20: note = Synthetic (Hu.DMD.Exon48.20.023)
atatttaaagcaaaaagttc

SEQ ID NO: 471 - DNA, 25 bp, synthetic construct
  source 1..25: mol_type = other DNA
  misc_feature 1..25: note = Synthetic (Hu.DMD.Exon49.25.001)
ctggggaaaagaacccatatagtgc

SEQ ID NO: 472 - DNA, 25 bp, synthetic construct
  source 1..25: mol_type = other DNA
  misc_feature 1..25: note = Synthetic (Hu.DMD.Exon49.25.002)
tcctggggaaaagaacccatatagt

SEQ ID NO: 473 - DNA, 25 bp, synthetic construct
  source 1..25: mol_type = other DNA
  misc_feature 1..25: note = Synthetic (Hu.DMD.Exon49.25.002.2)
gtttcctggggaaaagaacccatat

SEQ ID NO: 474 - DNA, 25 bp, synthetic construct
  source 1..25: mol_type = other DNA
  misc_feature 1..25: note = Synthetic (Hu.DMD.Exon49.25.003)
cagtttcctggggaaaagaacccat

SEQ ID NO: 475 - DNA, 25 bp, synthetic construct
  source 1..25: mol_type = other DNA
  misc_feature 1..25: note = Synthetic (Hu.DMD.Exon49.25.003.2)
tttcagtttcctggggaaaagaacc

SEQ ID NO: 476 - DNA, 25 bp, synthetic construct
  source 1..25: mol_type = other DNA
  misc_feature 1..25: note = Synthetic (Hu.DMD.Exon49.25.004)
tatttcagtttcctggggaaaagaa

SEQ ID NO: 477 - DNA, 25 bp, synthetic construct
  source 1..25: mol_type = other DNA
  misc_feature 1..25: note = Synthetic (Hu.DMD.Exon49.25.004.2)
tgctatttcagtttcctggggaaaa

SEQ ID NO: 478 - DNA, 25 bp, synthetic construct
  source 1..25: mol_type = other DNA
  misc_feature 1..25: note = Synthetic (Hu.DMD.Exon49.25.005)
actgctatttcagtttcctggggaa

SEQ ID NO: 479 - DNA, 25 bp, synthetic construct
  source 1..25: mol_type = other DNA
  misc_feature 1..25: note = Synthetic (Hu.DMD.Exon49.25.005.2)
tgaactgctatttcagtttcctggg

SEQ ID NO: 480 - DNA, 25 bp, synthetic construct
  source 1..25: mol_type = other DNA
  misc_feature 1..25: note = Synthetic (Hu.DMD.Exon49.25.006)
cttgaactgctatttcagtttcctg

SEQ ID NO: 481 - DNA, 25 bp, synthetic construct
  source 1..25: mol_type = other DNA
  misc_feature 1..25: note = Synthetic (Hu.DMD.Exon49.25.006.2)
tagcttgaactgctatttcagtttc

SEQ ID NO: 482 - DNA, 25 bp, synthetic construct
  source 1..25: mol_type = other DNA
  misc_feature 1..25: note = Synthetic (Hu.DMD.Exon49.25.007)
tttagcttgaactgctatttcagtt

SEQ ID NO: 483 - DNA, 25 bp, synthetic construct
  source 1..25: mol_type = other DNA
  misc_feature 1..25: note = Synthetic (Hu.DMD.Exon49.25.008)
ttccacatccggttgtttagcttga

SEQ ID NO: 484 - DNA, 25 bp, synthetic construct
  source 1..25: mol_type = other DNA
  misc_feature 1..25: note = Synthetic (Hu.DMD.Exon49.25.009)
tgccctttagacaaaatctcttcca

SEQ ID NO: 485 - DNA, 25 bp, synthetic construct
  source 1..25: mol_type = other DNA
  misc_feature 1..25: note = Synthetic (Hu.DMD.Exon49.25.009.2)
tttagacaaaatctcttccacatcc

SEQ ID NO: 486 - DNA, 25 bp, synthetic construct
  source 1..25: mol_type = other DNA
  misc_feature 1..25: note = Synthetic (Hu.DMD.Exon49.25.010)
gtttttccttgtacaaatgctgccc

SEQ ID NO: 487 - DNA, 25 bp, synthetic construct
  source 1..25: mol_type = other DNA
  misc_feature 1..25: note = Synthetic (Hu.DMD.Exon49.25.010.2)
gtacaaatgctgccctttagacaaa

SEQ ID NO: 488 - DNA, 25 bp, synthetic construct
  source 1..25: mol_type = other DNA
  misc_feature 1..25: note = Synthetic (Hu.DMD.Exon49.25.011)
cttcactggctgagtggctggtttt

SEQ ID NO: 489 - DNA, 25 bp, synthetic construct
  source 1..25: mol_type = other DNA
  misc_feature 1..25: note = Synthetic (Hu.DMD.Exon49.25.011.2)
ggctggtttttccttgtacaaatgc

SEQ ID NO: 490 - DNA, 25 bp, synthetic construct
  source 1..25: mol_type = other DNA
  misc_feature 1..25: note = Synthetic (Hu.DMD.Exon49.25.012)
attaccttcactggctgagtggctg

SEQ ID NO: 491 - DNA, 25 bp, synthetic construct
  source 1..25: mol_type = other DNA
  misc_feature 1..25: note = Synthetic (Hu.DMD.Exon49.25.013)
gcttcattaccttcactggctgagt

SEQ ID NO: 492 - DNA, 25 bp, synthetic construct
  source 1..25: mol_type = other DNA
  misc_feature 1..25: note = Synthetic (Hu.DMD.Exon49.25.014)
aggttgcttcattaccttcactggc

SEQ ID NO: 493 - DNA, 25 bp, synthetic construct
  source 1..25: mol_type = other DNA
  misc_feature 1..25: note = Synthetic (Hu.DMD.Exon49.25.015)
gctagaggttgcttcattaccttca

SEQ ID NO: 494 - DNA, 25 bp, synthetic construct
  source 1..25: mol_type = other DNA
  misc_feature 1..25: note = Synthetic (Hu.DMD.Exon49.25.016)
atattgctagaggttgcttcattac

SEQ ID NO: 495 - DNA, 20 bp, synthetic construct
  source 1..20: mol_type = other DNA
  misc_feature 1..20: note = Synthetic (Hu.DMD.Exon49.20.001)
gaaaagaacccatatagtgc

SEQ ID NO: 496 - DNA, 20 bp, synthetic construct
  source 1..20: mol_type = other DNA
  misc_feature 1..20: note = Synthetic (Hu.DMD.Exon49.20.002)
gggaaaagaacccatatagt

SEQ ID NO: 497 - DNA, 20 bp, synthetic construct
  source 1..20: mol_type = other DNA
  misc_feature 1..20: note = Synthetic (Hu.DMD.Exon49.20.003)
tcctggggaaaagaacccat

SEQ ID NO: 498 - DNA, 20 bp, synthetic construct
  source 1..20: mol_type = other DNA
  misc_feature 1..20: note = Synthetic (Hu.DMD.Exon49.20.004)
cagtttcctggggaaaagaa

SEQ ID NO: 499 - DNA, 20 bp, synthetic construct
  source 1..20: mol_type = other DNA
  misc_feature 1..20: note = Synthetic (Hu.DMD.Exon49.20.005)
tatttcagtttcctggggaa

SEQ ID NO: 500 - DNA, 20 bp, synthetic construct
  source 1..20: mol_type = other DNA
  misc_feature 1..20: note = Synthetic (Hu.DMD.Exon49.20.006)
actgctatttcagtttcctg

SEQ ID NO: 501 - DNA, 20 bp, synthetic construct
  source 1..20: mol_type = other DNA
  misc_feature 1..20: note = Synthetic (Hu.DMD.Exon49.20.007)
cttgaactgctatttcagtt

SEQ ID NO: 502 - DNA, 20 bp, synthetic construct
  source 1..20: mol_type = other DNA
  misc_feature 1..20: note = Synthetic (Hu.DMD.Exon49.20.008)
tttagcttgaactgctattt

SEQ ID NO: 503 - DNA, 20 bp, synthetic construct
  source 1..20: mol_type = other DNA
  misc_feature 1..20: note = Synthetic (Hu.DMD.Exon49.20.009)
ttccacatccggttgtttag

SEQ ID NO: 504 - DNA, 20 bp, synthetic construct
  source 1..20: mol_type = other DNA
  misc_feature 1..20: note = Synthetic (Hu.DMD.Exon49.20.010)
tttagacaaaatctcttcca

SEQ ID NO: 505 - DNA, 20 bp, synthetic construct
  source 1..20: mol_type = other DNA
  misc_feature 1..20: note = Synthetic (Hu.DMD.Exon49.20.011)
gtacaaatgctgccctttag

SEQ ID NO: 506 - DNA, 20 bp, synthetic construct
  source 1..20: mol_type = other DNA
  misc_feature 1..20: note = Synthetic (Hu.DMD.Exon49.20.012)
ggctggtttttccttgtaca

SEQ ID NO: 507 - DNA, 20 bp, synthetic construct
  source 1..20: mol_type = other DNA
  misc_feature 1..20: note = Synthetic (Hu.DMD.Exon49.20.013)
cttcactggctgagtggctg

SEQ ID NO: 508 - DNA, 20 bp, synthetic construct
  source 1..20: mol_type = other DNA
  misc_feature 1..20: note = Synthetic (Hu.DMD.Exon49.20.014)
attaccttcactggctgagt

SEQ ID NO: 509 - DNA, 20 bp, synthetic construct
  source 1..20: mol_type = other DNA
  misc_feature 1..20: note = Synthetic (Hu.DMD.Exon49.20.015)
gcttcattaccttcactggc

SEQ ID NO: 510 - DNA, 20 bp, synthetic construct
  source 1..20: mol_type = other DNA
  misc_feature 1..20: note = Synthetic (Hu.DMD.Exon49.20.016)
aggttgcttcattaccttca

SEQ ID NO: 511 - DNA, 20 bp, synthetic construct
  source 1..20: mol_type = other DNA
  misc_feature 1..20: note = Synthetic (Hu.DMD.Exon49.20.017)
gctagaggttgcttcattac

SEQ ID NO: 512 - DNA, 20 bp, synthetic construct
  source 1..20: mol_type = other DNA
  misc_feature 1..20: note = Synthetic (Hu.DMD.Exon49.20.018)
atattgctagaggttgcttc

SEQ ID NO: 513 - DNA, 25 bp, synthetic construct
  source 1..25: mol_type = other DNA
  misc_feature 1..25: note = Synthetic (Hu.DMD.Exon50.25.001)
ctttaacagaaaagcatacacatta

SEQ ID NO: 514 - DNA, 25 bp, synthetic construct
  source 1..25: mol_type = other DNA
  misc_feature 1..25: note = Synthetic (Hu.DMD.Exon50.25.002)
tcctctttaacagaaaagcatacac

SEQ ID NO: 515 - DNA, 25 bp, synthetic construct
  source 1..25: mol_type = other DNA
  misc_feature 1..25: note = Synthetic (Hu.DMD.Exon50.25.002.2)
ttcctctttaacagaaaagcataca

SEQ ID NO: 516 - DNA, 25 bp, synthetic construct
  source 1..25: mol_type = other DNA
  misc_feature 1..25: note = Synthetic (Hu.DMD.Exon50.25.003)
taacttcctctttaacagaaaagca

SEQ ID NO: 517 - DNA, 25 bp, synthetic construct
  source 1..25: mol_type = other DNA
  misc_feature 1..25: note = Synthetic (Hu.DMD.Exon50.25.003.2)
ctaacttcctctttaacagaaaagc

SEQ ID NO: 518 - DNA, 25 bp, synthetic construct
  source 1..25: mol_type = other DNA
  misc_feature 1..25: note = Synthetic (Hu.DMD.Exon50.25.004)
tcttctaacttcctctttaacagaa

SEQ ID NO: 519 - DNA, 25 bp, synthetic construct
  source 1..25: mol_type = other DNA
  misc_feature 1..25: note = Synthetic (Hu.DMD.Exon50.25.004.2)
atcttctaacttcctctttaacaga

SEQ ID NO: 520 - DNA, 25 bp, synthetic construct
  source 1..25: mol_type = other DNA
  misc_feature 1..25: note = Synthetic (Hu.DMD.Exon50.25.005)
tcagatcttctaacttcctctttaa

SEQ ID NO: 521 - DNA, 25 bp, synthetic construct
  source 1..25: mol_type = other DNA
  misc_feature 1..25: note = Synthetic (Hu.DMD.Exon50.25.005.2)
ctcagatcttctaacttcctcttta

SEQ ID NO: 522 - DNA, 25 bp, synthetic construct
  source 1..25: mol_type = other DNA
  misc_feature 1..25: note = Synthetic (Hu.DMD.Exon50.25.006)
agagctcagatcttctaacttcctc

SEQ ID NO: 523 - DNA, 25 bp, synthetic construct
  source 1..25: mol_type = other DNA
  misc_feature 1..25: note = Synthetic (Hu.DMD.Exon50.25.006.2 NG-08-0731)
cagagctcagatcttctaacttcct

SEQ ID NO: 524 - DNA, 24 bp, synthetic construct
  source 1..24: mol_type = other DNA
  misc_feature 1..24: note = Synthetic (Hu.DMD.Exon50.25.007)
cactcagagctcagatcttctact

SEQ ID NO: 525 - DNA, 25 bp, synthetic construct
  source 1..25: mol_type = other DNA
  misc_feature 1..25: note = Synthetic (Hu.DMD.Exon50.25.007.2)
ccttccactcagagctcagatcttc

SEQ ID NO: 526 - DNA, 25 bp, synthetic construct
  source 1..25: mol_type = other DNA
  misc_feature 1..25: note = Synthetic (Hu.DMD.Exon50.25.008)
gtaaacggtttaccgccttccactc

SEQ ID NO: 527 - DNA, 25 bp, synthetic construct
  source 1..25: mol_type = other DNA
  misc_feature 1..25: note = Synthetic (Hu.DMD.Exon50.25.009)
ctttgccctcagctcttgaagtaaa

SEQ ID NO: 528 - DNA, 25 bp, synthetic construct
  source 1..25: mol_type = other DNA
  misc_feature 1..25: note = Synthetic (Hu.DMD.Exon50.25.009.2)
ccctcagctcttgaagtaaacggtt

SEQ ID NO: 529 - DNA, 25 bp, synthetic construct
  source 1..25: mol_type = other DNA
  misc_feature 1..25: note = Synthetic (Hu.DMD.Exon50.25.010)
ccaggagctaggtcaggctgctttg

SEQ ID NO: 530 - DNA, 25 bp, synthetic construct
  source 1..25: mol_type = other DNA
  misc_feature 1..25: note = Synthetic (Hu.DMD.Exon50.25.010.2)
ggtcaggctgctttgccctcagctc

SEQ ID NO: 531 - DNA, 25 bp, synthetic construct
  source 1..25: mol_type = other DNA
  misc_feature 1..25: note = Synthetic (Hu.DMD.Exon50.25.011)
aggctccaatagtggtcagtccagg

SEQ ID NO: 532 - DNA, 25 bp, synthetic construct
  source 1..25: mol_type = other DNA
  misc_feature 1..25: note = Synthetic (Hu.DMD.Exon50.25.011.2)
tcagtccaggagctaggtcaggctg

SEQ ID NO: 533 - DNA, 25 bp, synthetic construct
  source 1..25: mol_type = other DNA
  misc_feature 1..25: note = Synthetic (Hu.DMD.Exon50.25.012 AVI-5038)
cttacaggctccaatagtggtcagt

SEQ ID NO: 534 - DNA, 25 bp, synthetic construct
  source 1..25: mol_type = other DNA
  misc_feature 1..25: note = Synthetic (Hu.DMD.Exon50.25.013)
gtatacttacaggctccaatagtgg

SEQ ID NO: 535 - DNA, 25 bp, synthetic construct
  source 1..25: mol_type = other DNA
  misc_feature 1..25: note = Synthetic (Hu.DMD.Exon50.25.014)
atccagtatacttacaggctccaat

SEQ ID NO: 536 - DNA, 25 bp, synthetic construct
  source 1..25: mol_type = other DNA
  misc_feature 1..25: note = Synthetic (Hu.DMD.Exon50.25.015 NG-08-0741)
atgggatccagtatacttacaggct

SEQ ID NO: 537 - DNA, 25 bp, synthetic construct
  source 1..25: mol_type = other DNA
  misc_feature 1..25: note = Synthetic (Hu.DMD.Exon50.25.016 NG-08-0742)
agagaatgggatccagtatacttac

SEQ ID NO: 538 - DNA, 20 bp, synthetic construct
  source 1..20: mol_type = other DNA
  misc_feature 1..20: note = Synthetic (Hu.DMD.Exon50.20.001)
acagaaaagcatacacatta

SEQ ID NO: 539 - DNA, 20 bp, synthetic construct
  source 1..20: mol_type = other DNA
  misc_feature 1..20: note = Synthetic (Hu.DMD.Exon50.20.002)
tttaacagaaaagcatacac

SEQ ID NO: 540 - DNA, 20 bp, synthetic construct
  source 1..20: mol_type = other DNA
  misc_feature 1..20: note = Synthetic (Hu.DMD.Exon50.20.003)
tcctctttaacagaaaagca

SEQ ID NO: 541 - DNA, 20 bp, synthetic construct
  source 1..20: mol_type = other DNA
  misc_feature 1..20: note = Synthetic (Hu.DMD.Exon50.20.004)
taacttcctctttaacagaa

SEQ ID NO: 542 - DNA, 20 bp, synthetic construct
  source 1..20: mol_type = other DNA
  misc_feature 1..20: note = Synthetic (Hu.DMD.Exon50.20.005)
tcttctaacttcctctttaa

SEQ ID NO: 543 - DNA, 20 bp, synthetic construct
  source 1..20: mol_type = other DNA
  misc_feature 1..20: note = Synthetic (Hu.DMD.Exon50.20.006)
tcagatcttctaacttcctc

SEQ ID NO: 544 - DNA, 20 bp, synthetic construct
  source 1..20: mol_type = other DNA
  misc_feature 1..20: note = Synthetic (Hu.DMD.Exon50.20.007)
ccttccactcagagctcaga

SEQ ID NO: 545 - DNA, 20 bp, synthetic construct
  source 1..20: mol_type = other DNA
  misc_feature 1..20: note = Synthetic (Hu.DMD.Exon50.20.008)
gtaaacggtttaccgccttc

SEQ ID NO: 546 - DNA, 20 bp, synthetic construct
  source 1..20: mol_type = other DNA
  misc_feature 1..20: note = Synthetic (Hu.DMD.Exon50.20.009)
ccctcagctcttgaagtaaa

SEQ ID NO: 547 - DNA, 20 bp, synthetic construct
  source 1..20: mol_type = other DNA
  misc_feature 1..20: note = Synthetic (Hu.DMD.Exon50.20.010)
ggtcaggctgctttgccctc

SEQ ID NO: 548 - DNA, 20 bp, synthetic construct
  source 1..20: mol_type = other DNA
  misc_feature 1..20: note = Synthetic (Hu.DMD.Exon50.20.011)
tcagtccaggagctaggtca

SEQ ID NO: 549 - DNA, 20 bp, synthetic construct
  source 1..20: mol_type = other DNA
  misc_feature 1..20: note = Synthetic (Hu.DMD.Exon50.20.012)
aggctccaatagtggtcagt

SEQ ID NO: 550 - DNA, 20 bp, synthetic construct
  source 1..20: mol_type = other DNA
  misc_feature 1..20: note = Synthetic (Hu.DMD.Exon50.20.013)
cttacaggctccaatagtgg

SEQ ID NO: 551 - DNA, 20 bp, synthetic construct
  source 1..20: mol_type = other DNA
  misc_feature 1..20: note = Synthetic (Hu.DMD.Exon50.20.014)
gtatacttacaggctccaat

SEQ ID NO: 552 - DNA, 20 bp, synthetic construct
  source 1..20: mol_type = other DNA
  misc_feature 1..20: note = Synthetic (Hu.DMD.Exon50.20.015)
atccagtatacttacaggct

SEQ ID NO: 553 - DNA, 20 bp, synthetic construct
  source 1..20: mol_type = other DNA
  misc_feature 1..20: note = Synthetic (Hu.DMD.Exon50.20.016)
atgggatccagtatacttac

SEQ ID NO: 554 - DNA, 20 bp, synthetic construct
  source 1..20: mol_type = other DNA
  misc_feature 1..20: note = Synthetic (Hu.DMD.Exon50.20.017)
agagaatgggatccagtata

SEQ ID NO: 555 - DNA, 26 bp, synthetic construct
  source 1..26: mol_type = other DNA
  misc_feature 1..26: note = Synthetic (Hu.DMD.Exon51.25.001-44)
ctaaaatattttgggtttttgcaaaa

SEQ ID NO: 556 - DNA, 26 bp, synthetic construct
  source 1..26: mol_type = other DNA
  misc_feature 1..26: note = Synthetic (Hu.DMD.Exon51.25.002-45)
gctaaaatattttgggtttttgcaaa

SEQ ID NO: 557 - DNA, 26 bp, synthetic construct
  source 1..26: mol_type = other DNA
  misc_feature 1..26: note = Synthetic (Hu.DMD.Exon51.25.002.2-46)
taggagctaaaatattttgggttttt

SEQ ID NO: 558 - DNA, 25 bp, synthetic construct
  source 1..25: mol_type = other DNA
  misc_feature 1..25: note = Synthetic (Hu.DMD.Exon51.25.003)
agtaggagctaaaatattttgggtt

SEQ ID NO: 559 - DNA, 25 bp, synthetic construct
  source 1..25: mol_type = other DNA
  misc_feature 1..25: note = Synthetic (Hu.DMD.Exon51.25.003.2)
tgagtaggagctaaaatattttggg

SEQ ID NO: 560 - DNA, 26 bp, synthetic construct
  source 1..26: mol_type = other DNA
  misc_feature 1..26: note = Synthetic (Hu.DMD.Exon51.25.004)
ctgagtaggagctaaaatattttggg

SEQ ID NO: 561 - DNA, 25 bp, synthetic construct
  source 1..25: mol_type = other DNA
  misc_feature 1..25: note = Synthetic (Hu.DMD.Exon51.25.004.2)
cagtctgagtaggagctaaaatatt

SEQ ID NO: 562 - DNA, 26 bp, synthetic construct
  source 1..26: mol_type = other DNA
  misc_feature 1..26: note = Synthetic (Hu.DMD.Exon51.25.005)
acagtctgagtaggagctaaaatatt

SEQ ID NO: 563 - DNA, 26 bp, synthetic construct
  source 1..26: mol_type = other DNA
  misc_feature 1..26: note = Synthetic (Hu.DMD.Exon51.25.005.2)
gagtaacagtctgagtaggagctaaa

SEQ ID NO: 564 - DNA, 25 bp, synthetic construct
  source 1..25: mol_type = other DNA
  misc_feature 1..25: note = Synthetic (Hu.DMD.Exon51.25.006)
cagagtaacagtctgagtaggagct

SEQ ID NO: 565 - DNA, 26 bp, synthetic construct
  source 1..26: mol_type = other DNA
  misc_feature 1..26: note = Synthetic (Hu.DMD.Exon51.25.006.2)
caccagagtaacagtctgagtaggag

SEQ ID NO: 566 - DNA, 25 bp, synthetic construct
  source 1..25: mol_type = other DNA
  misc_feature 1..25: note = Synthetic (Hu.DMD.Exon51.25.007)
gtcaccagagtaacagtctgagtag

SEQ ID NO: 567 - DNA, 26 bp, synthetic construct
  source 1..26: mol_type = other DNA
  misc_feature 1..26: note = Synthetic (Hu.DMD.Exon51.25.007.2)
aaccacaggttgtgtcaccagagtaa

SEQ ID NO: 568 - DNA, 25 bp, synthetic construct
  source 1..25: mol_type = other DNA
  misc_feature 1..25: note = Synthetic (Hu.DMD.Exon51.25.008)
gttgtgtcaccagagtaacagtctg

SEQ ID NO: 569 - DNA, 26 bp, synthetic construct
  source 1..26: mol_type = other DNA
  misc_feature 1..26: note = Synthetic (Hu.DMD.Exon51.25.009)
tggcagtttccttagtaaccacaggt

SEQ ID NO: 570 - DNA, 26 bp, synthetic construct
  source 1..26: mol_type = other DNA
  misc_feature 1..26: note = Synthetic (Hu.DMD.Exon51.25.010)
atttctagtttggagatggcagtttc

SEQ ID NO: 571 - DNA, 25 bp, synthetic construct
  source 1..25: mol_type = other DNA
  misc_feature 1..25: note = Synthetic (Hu.DMD.Exon51.25.010.2)
ggaagatggcatttctagtttggag

SEQ ID NO: 572 - DNA, 26 bp, synthetic construct
  source 1..26: mol_type = other DNA
  misc_feature 1..26: note = Synthetic (Hu.DMD.Exon51.25.011)
catcaaggaagatggcatttctagtt

SEQ ID NO: 573 - DNA, 26 bp, synthetic construct
  source 1..26: mol_type = other DNA
  misc_feature 1..26: note = Synthetic (Hu.DMD.Exon51.25.011.2)
gagcaggtacctccaacatcaaggaa

SEQ ID NO: 574 - DNA, 25 bp, synthetic construct
  source 1..25: mol_type = other DNA
  misc_feature 1..25: note = Synthetic (Hu.DMD.Exon51.25.012)
atctgccagagcaggtacctccaac

SEQ ID NO: 575 - DNA, 26 bp, synthetic construct
  source 1..26: mol_type = other DNA
  misc_feature 1..26: note = Synthetic (Hu.DMD.Exon51.25.013)
aagttctgtccaagcccggttgaaat

SEQ ID NO: 576 - DNA, 26 bp, synthetic construct
  source 1..26: mol_type = other DNA
  misc_feature 1..26: note = Synthetic (Hu.DMD.Exon51.25.013.2)
cggttgaaatctgccagagcaggtac

SEQ ID NO: 577 - DNA, 26 bp, synthetic construct
  source 1..26: mol_type = other DNA
  misc_feature 1..26: note = Synthetic (Hu.DMD.Exon51.25.014)
gagaaagccagtcggtaagttctgtc

SEQ ID NO: 578 - DNA, 25 bp, synthetic construct
  source 1..25: mol_type = other DNA
  misc_feature 1..25: note = Synthetic (Hu.DMD.Exon51.25.014.2)
gtcggtaagttctgtccaagcccgg

SEQ ID NO: 579 - DNA, 26 bp, synthetic construct
  source 1..26: mol_type = other DNA
  misc_feature 1..26: note = Synthetic (Hu.DMD.Exon51.25.015)
ataacttgatcaagcagagaaagcca

SEQ ID NO: 580 - DNA, 25 bp, synthetic construct
  source 1..25: mol_type = other DNA
  misc_feature 1..25: note = Synthetic (Hu.DMD.Exon51.25.015.2)
aagcagagaaagccagtcggtaagt

SEQ ID NO: 581 - DNA, 26 bp, synthetic construct
  source 1..26: mol_type = other DNA
  misc_feature 1..26: note = Synthetic (Hu.DMD.Exon51.25.016)
caccctctgtgattttataacttgat

SEQ ID NO: 582 - DNA, 26 bp, synthetic construct
  source 1..26: mol_type = other DNA
  misc_feature 1..26: note = Synthetic (Hu.DMD.Exon51.25.017)
caaggtcacccaccatcaccctctgt

SEQ ID NO: 583 - DNA, 25 bp, synthetic construct
  source 1..25: mol_type = other DNA
  misc_feature 1..25: note = Synthetic (Hu.DMD.Exon51.25.017.2)
catcaccctctgtgattttataact

SEQ ID NO: 584 - DNA, 26 bp, synthetic construct
  source 1..26: mol_type = other DNA
  misc_feature 1..26: note = Synthetic (Hu.DMD.Exon51.25.018)
cttctgcttgatgatcatctcgttga

SEQ ID NO: 585 - DNA, 26 bp, synthetic construct
  source 1..26: mol_type = other DNA
  misc_feature 1..26: note = Synthetic (Hu.DMD.Exon51.25.019)
ccttctgcttgatgatcatctcgttg

SEQ ID NO: 586 - DNA, 26 bp, synthetic construct
  source 1..26: mol_type = other DNA
  misc_feature 1..26: note = Synthetic (Hu.DMD.Exon51.25.019.2)
atctcgttgatatcctcaaggtcacc

SEQ ID NO: 587 - DNA, 26 bp, synthetic construct
  source 1..26: mol_type = other DNA
  misc_feature 1..26: note = Synthetic (Hu.DMD.Exon51.25.020)
tcataccttctgcttgatgatcatct

SEQ ID NO: 588 - DNA, 26 bp, synthetic construct
  source 1..26: mol_type = other DNA
  misc_feature 1..26: note = Synthetic (Hu.DMD.Exon51.25.020.2)
tcattttttctcataccttctgcttg

SEQ ID NO: 589 - DNA, 26 bp, synthetic construct
  source 1..26: mol_type = other DNA
  misc_feature 1..26: note = Synthetic (Hu.DMD.Exon51.25.021)
ttttctcataccttctgcttgatgat

SEQ ID NO: 590 - DNA, 26 bp, synthetic construct
  source 1..26: mol_type = other DNA
  misc_feature 1..26: note = Synthetic (Hu.DMD.Exon51.25.022)
ttttatcattttttctcataccttct

SEQ ID NO: 591 - DNA, 26 bp, synthetic construct
  source 1..26: mol_type = other DNA
  misc_feature 1..26: note = Synthetic (Hu.DMD.Exon51.25.023)
ccaacttttatcattttttctcatac

SEQ ID NO: 592 - DNA, 20 bp, synthetic construct
  source 1..20: mol_type = other DNA
  misc_feature 1..20: note = Synthetic (Hu.DMD.Exon51.20.001)
atattttgggtttttgcaaa

SEQ ID NO: 593 - DNA, 20 bp, synthetic construct
  source 1..20: mol_type = other DNA
  misc_feature 1..20: note = Synthetic (Hu.DMD.Exon51.20.002)
aaaatattttgggtttttgc

SEQ ID NO: 594 - DNA, 20 bp, synthetic construct
  source 1..20: mol_type = other DNA
  misc_feature 1..20: note = Synthetic (Hu.DMD.Exon51.20.003)
gagctaaaatattttgggtt

SEQ ID NO: 595 - DNA, 20 bp, synthetic construct
  source 1..20: mol_type = other DNA
  misc_feature 1..20: note = Synthetic (Hu.DMD.Exon51.20.004)
agtaggagctaaaatatttt

SEQ ID NO: 596 - DNA, 20 bp, synthetic construct
  source 1..20: mol_type = other DNA
  misc_feature 1..20: note = Synthetic (Hu.DMD.Exon51.20.005)
gtctgagtaggagctaaaat

SEQ ID NO: 597 - DNA, 20 bp, synthetic construct
  source 1..20: mol_type = other DNA
  misc_feature 1..20: note = Synthetic (Hu.DMD.Exon51.20.006)
taacagtctgagtaggagct

SEQ ID NO: 598 - DNA, 20 bp, synthetic construct
  source 1..20: mol_type = other DNA
  misc_feature 1..20: note = Synthetic (Hu.DMD.Exon51.20.007)
cagagtaacagtctgagtag

SEQ ID NO: 599 - DNA, 20 bp, synthetic construct
  source 1..20: mol_type = other DNA
  misc_feature 1..20: note = Synthetic (Hu.DMD.Exon51.20.008)
cacaggttgtgtcaccagag

SEQ ID NO: 600 - DNA, 20 bp, synthetic construct
  source 1..20: mol_type = other DNA
  misc_feature 1..20: note = Synthetic (Hu.DMD.Exon51.20.009)
agtttccttagtaaccacag

SEQ ID NO: 601 - DNA, 20 bp, synthetic construct
  source 1..20: mol_type = other DNA
  misc_feature 1..20: note = Synthetic (Hu.DMD.Exon51.20.010)
tagtttggagatggcagttt

SEQ ID NO: 602 - DNA, 20 bp, synthetic construct
  source 1..20: mol_type = other DNA
  misc_feature 1..20: note = Synthetic (Hu.DMD.Exon51.20.011)
ggaagatggcatttctagtt

SEQ ID NO: 603 - DNA, 20 bp, synthetic construct
  source 1..20: mol_type = other DNA
  misc_feature 1..20: note = Synthetic (Hu.DMD.Exon51.20.012)
tacctccaacatcaaggaag

SEQ ID NO: 604 - DNA, 20 bp, synthetic construct
  source 1..20: mol_type = other DNA
  misc_feature 1..20: note = Synthetic (Hu.DMD.Exon51.20.013)
atctgccagagcaggtacct

SEQ ID NO: 605 - DNA, 20 bp, synthetic construct
  source 1..20: mol_type = other DNA
  misc_feature 1..20: note = Synthetic (Hu.DMD.Exon51.20.014)
ccaagcccggttgaaatctg

SEQ ID NO: 606 - DNA, 20 bp, synthetic construct
  source 1..20: mol_type = other DNA
  misc_feature 1..20: note = Synthetic (Hu.DMD.Exon51.20.015)
gtcggtaagttctgtccaag

SEQ ID NO: 607 - DNA, 20 bp, synthetic construct
  source 1..20: mol_type = other DNA
  misc_feature 1..20: note = Synthetic (Hu.DMD.Exon51.20.016)
aagcagagaaagccagtcgg

SEQ ID NO: 608 - DNA, 20 bp, synthetic construct
  source 1..20: mol_type = other DNA
  misc_feature 1..20: note = Synthetic (Hu.DMD.Exon51.20.017)
ttttataacttgatcaagca

SEQ ID NO: 609 - DNA, 20 bp, synthetic construct
  source 1..20: mol_type = other DNA
  misc_feature 1..20: note = Synthetic (Hu.DMD.Exon51.20.018)
catcaccctctgtgatttta

SEQ ID NO: 610 - DNA, 20 bp, synthetic construct
  source 1..20: mol_type = other DNA
  misc_feature 1..20: note = Synthetic (Hu.DMD.Exon51.20.019)
ctcaaggtcacccaccatca

SEQ ID NO: 611 - DNA, 20 bp, synthetic construct
  source 1..20: mol_type = other DNA
  misc_feature 1..20: note = Synthetic (Hu.DMD.Exon51.20.020)
catctcgttgatatcctcaa

SEQ ID NO: 612 - DNA, 20 bp, synthetic construct
  source 1..20: mol_type = other DNA
  misc_feature 1..20: note = Synthetic (Hu.DMD.Exon51.20.021)
cttctgcttgatgatcatct

SEQ ID NO: 613 - DNA, 20 bp, synthetic construct
  source 1..20: mol_type = other DNA
  misc_feature 1..20: note = Synthetic (Hu.DMD.Exon51.20.022)
cataccttctgcttgatgat

SEQ ID NO: 614 - DNA, 20 bp, synthetic construct
  source 1..20: mol_type = other DNA
  misc_feature 1..20: note = Synthetic (Hu.DMD.Exon51.20.023)
tttctcataccttctgcttg

SEQ ID NO: 615 - DNA, 20 bp, synthetic construct
  source 1..20: mol_type = other DNA
  misc_feature 1..20: note = Synthetic (Hu.DMD.Exon51.20.024)
cattttttctcataccttct

SEQ ID NO: 616 - DNA, 20 bp, synthetic construct
  source 1..20: mol_type = other DNA
  misc_feature 1..20: note = Synthetic (Hu.DMD.Exon51.20.025)
tttatcattttttctcatac

SEQ ID NO: 617 - DNA, 20 bp, synthetic construct
  source 1..20: mol_type = other DNA
  misc_feature 1..20: note = Synthetic (Hu.DMD.Exon51.20.026)
caacttttatcattttttct

SEQ ID NO: 618 - DNA, 25 bp, synthetic construct
  source 1..25: mol_type = other DNA
  misc_feature 1..25: note = Synthetic (Hu.DMD.Exon52.25.001)
ctgtaagaacaaatatcccttagta

SEQ ID NO: 619 - DNA, 25 bp, synthetic construct
  source 1..25: mol_type = other DNA
  misc_feature 1..25: note = Synthetic (Hu.DMD.Exon52.25.002)
tgcctgtaagaacaaatatccctta

SEQ ID NO: 620 - DNA, 25 bp, synthetic construct
  source 1..25: mol_type = other DNA
  misc_feature 1..25: note = Synthetic (Hu.DMD.Exon52.25.002.2)
gttgcctgtaagaacaaatatccct

SEQ ID NO: 621 - DNA, 25 bp, synthetic construct
  source 1..25: mol_type = other DNA
  misc_feature 1..25: note = Synthetic (Hu.DMD.Exon52.25.003)
attgttgcctgtaagaacaaatatc

SEQ ID NO: 622 - DNA, 25 bp, synthetic construct
  source 1..25: mol_type = other DNA
  misc_feature 1..25: note = Synthetic (Hu.DMD.Exon52.25.003.2)
gcattgttgcctgtaagaacaaata

SEQ ID NO: 623 - DNA, 25 bp, synthetic construct
  source 1..25: mol_type = other DNA
  misc_feature 1..25: note = Synthetic (Hu.DMD.Exon52.25.004)
cctgcattgttgcctgtaagaacaa

SEQ ID NO: 624 - DNA, 25 bp, synthetic construct
  source 1..25: mol_type = other DNA
  misc_feature 1..25: note = Synthetic (Hu.DMD.Exon52.25.004.2)
atcctgcattgttgcctgtaagaac

SEQ ID NO: 625 - DNA, 25 bp, synthetic construct
  source 1..25: mol_type = other DNA
  misc_feature 1..25: note = Synthetic (Hu.DMD.Exon52.25.005)
caaatcctgcattgttgcctgtaag

SEQ ID NO: 626 - DNA, 25 bp, synthetic construct
  source 1..25: mol_type = other DNA
  misc_feature 1..25: note = Synthetic (Hu.DMD.Exon52.25.005.2)
tccaaatcctgcattgttgcctgta

SEQ ID NO: 627 - DNA, 25 bp, synthetic construct
  source 1..25: mol_type = other DNA
  misc_feature 1..25: note = Synthetic (Hu.DMD.Exon52.25.006)
tgttccaaatcctgcattgttgcct

SEQ ID NO: 628 - DNA, 25 bp, synthetic construct
  source 1..25: mol_type = other DNA
  misc_feature 1..25: note = Synthetic (Hu.DMD.Exon52.25.006.2)
tctgttccaaatcctgcattgttgc

SEQ ID NO: 629 - DNA, 25 bp, synthetic construct
  source 1..25: mol_type = other DNA
  misc_feature 1..25: note = Synthetic (Hu.DMD.Exon52.25.007)
aactggggacgcctctgttccaaat

SEQ ID NO: 630 - DNA, 25 bp, synthetic construct
  source 1..25: mol_type = other DNA
  misc_feature 1..25: note = Synthetic (Hu.DMD.Exon52.25.007.2)
gcctctgttccaaatcctgcattgt

SEQ ID NO: 631 - DNA, 25 bp, synthetic construct
  source 1..25: mol_type = other DNA
  misc_feature 1..25: note = Synthetic (Hu.DMD.Exon52.25.008)
cagcggtaatgagttcttccaactg

SEQ ID NO: 632 - DNA, 25 bp, synthetic construct
  source 1..25: mol_type = other DNA
  misc_feature 1..25: note = Synthetic (Hu.DMD.Exon52.25.008.2)
cttccaactggggacgcctctgttc

SEQ ID NO: 633 - DNA, 25 bp, synthetic construct
  source 1..25: mol_type = other DNA
  misc_feature 1..25: note = Synthetic (Hu.DMD.Exon52.25.009)
cttgtttttcaaattttgggcagcg

SEQ ID NO: 634 - DNA, 25 bp, synthetic construct
  source 1..25: mol_type = other DNA
  misc_feature 1..25: note = Synthetic (Hu.DMD.Exon52.25.010)
ctagcctcttgattgctggtcttgt

SEQ ID NO: 635 - DNA, 25 bp, synthetic construct
  source 1..25: mol_type = other DNA
  misc_feature 1..25: note = Synthetic (Hu.DMD.Exon52.25.010.2)
ttttcaaattttgggcagcggtaat

SEQ ID NO: 636 - DNA, 25 bp, synthetic construct
  source 1..25: mol_type = other DNA
  misc_feature 1..25: note = Synthetic (Hu.DMD.Exon52.25.011)
ttcgatccgtaatgattgttctagc

SEQ ID NO: 637 - DNA, 25 bp, synthetic construct
  source 1..25: mol_type = other DNA
  misc_feature 1..25: note = Synthetic (Hu.DMD.Exon52.25.011.2)
gattgctggtcttgtttttcaaatt

SEQ ID NO: 638 - DNA, 25 bp, synthetic construct
  source 1..25: mol_type = other DNA
  misc_feature 1..25: note = Synthetic (Hu.DMD.Exon52.25.012)
cttacttcgatccgtaatgattgtt

SEQ ID NO: 639 - DNA, 25 bp, synthetic construct
  source 1..25: mol_type = other DNA
  misc_feature 1..25: note = Synthetic (Hu.DMD.Exon52.25.012.2)
ttgttctagcctcttgattgctggt

SEQ ID NO: 640 - DNA, 25 bp, synthetic construct
  source 1..25: mol_type = other DNA
  misc_feature 1..25: note = Synthetic (Hu.DMD.Exon52.25.013)
aaaaacttacttcgatccgtaatga

SEQ ID NO: 641 - DNA, 25 bp, synthetic construct
  source 1..25: mol_type = other DNA
  misc_feature 1..25: note = Synthetic (Hu.DMD.Exon52.25.014)
tgttaaaaaacttacttcgatccgt

SEQ ID NO: 642 - DNA, 25 bp, synthetic construct
  source 1..25: mol_type = other DNA
  misc_feature 1..25: note = Synthetic (Hu.DMD.Exon52.25.015)
atgcttgttaaaaaacttacttcga

SEQ ID NO: 643 - DNA, 25 bp, synthetic construct
  source 1..25: mol_type = other DNA
  misc_feature 1..25: note = Synthetic (Hu.DMD.Exon52.25.016)
gtcccatgcttgttaaaaaacttac

SEQ ID NO: 644 - DNA, 20 bp, synthetic construct
  source 1..20: mol_type = other DNA
  misc_feature 1..20: note = Synthetic (Hu.DMD.Exon52.20.001)
agaacaaatatcccttagta

SEQ ID NO: 645 - DNA, 20 bp, synthetic construct
  source 1..20: mol_type = other DNA
  misc_feature 1..20: note = Synthetic (Hu.DMD.Exon52.20.002)
gtaagaacaaatatccctta

SEQ ID NO: 646 - DNA, 20 bp, synthetic construct
  source 1..20: mol_type = other DNA
  misc_feature 1..20: note = Synthetic (Hu.DMD.Exon52.20.003)
tgcctgtaagaacaaatatc

SEQ ID NO: 647 - DNA, 20 bp, synthetic construct
  source 1..20: mol_type = other DNA
  misc_feature 1..20: note = Synthetic (Hu.DMD.Exon52.20.004)
attgttgcctgtaagaacaa

SEQ ID NO: 648 - DNA, 20 bp, synthetic construct
  source 1..20: mol_type = other DNA
  misc_feature 1..20: note = Synthetic (Hu.DMD.Exon52.20.005)
cctgcattgttgcctgtaag

SEQ ID NO: 649 - DNA, 20 bp, synthetic construct
  source 1..20: mol_type = other DNA
  misc_feature 1..20: note = Synthetic (Hu.DMD.Exon52.20.006)
caaatcctgcattgttgcct

SEQ ID NO: 650 - DNA, 20 bp, synthetic construct
  source 1..20: mol_type = other DNA
  misc_feature 1..20: note = Synthetic (Hu.DMD.Exon52.20.007)
gcctctgttccaaatcctgc

SEQ ID NO: 651 - DNA, 20 bp, synthetic construct
  source 1..20: mol_type = other DNA
  misc_feature 1..20: note = Synthetic (Hu.DMD.Exon52.20.008)
cttccaactggggacgcctc

SEQ ID NO: 652 - DNA, 20 bp, synthetic construct
  source 1..20: mol_type = other DNA
  misc_feature 1..20: note = Synthetic (Hu.DMD.Exon52.20.009)
cagcggtaatgagttcttcc

SEQ ID NO: 653 - DNA, 20 bp, synthetic construct
  source 1..20: mol_type = other DNA
  misc_feature 1..20: note = Synthetic (Hu.DMD.Exon52.20.010)
ttttcaaattttgggcagcg

SEQ ID NO: 654 - DNA, 20 bp, synthetic construct
  source 1..20: mol_type = other DNA
  misc_feature 1..20: note = Synthetic (Hu.DMD.Exon52.20.011)
gattgctggtcttgtttttc

SEQ ID NO: 655 - DNA, 20 bp, synthetic construct
  source 1..20: mol_type = other DNA
  misc_feature 1..20: note = Synthetic (Hu.DMD.Exon52.20.012)
ttgttctagcctcttgattg

SEQ ID NO: 656 - DNA, 20 bp, synthetic construct
  source 1..20: mol_type = other DNA
  misc_feature 1..20: note = Synthetic (Hu.DMD.Exon52.20.013)
ttcgatccgtaatgattgtt

SEQ ID NO: 657 - DNA, 20 bp, synthetic construct
  source 1..20: mol_type = other DNA
  misc_feature 1..20: note = Synthetic (Hu.DMD.Exon52.20.014)
cttacttcgatccgtaatga

SEQ ID NO: 658 - DNA, 20 bp, synthetic construct
  source 1..20: mol_type = other DNA
  misc_feature 1..20: note = Synthetic (Hu.DMD.Exon52.20.015)
aaaaacttacttcgatccgt

SEQ ID NO: 659 - DNA, 20 bp, synthetic construct
  source 1..20: mol_type = other DNA
  misc_feature 1..20: note = Synthetic (Hu.DMD.Exon52.20.016)
tgttaaaaaacttacttcga

SEQ ID NO: 660 - DNA, 20 bp, synthetic construct
  source 1..20: mol_type = other DNA
  misc_feature 1..20: note = Synthetic (Hu.DMD.Exon52.20.017)
atgcttgttaaaaaacttac

SEQ ID NO: 661 - DNA, 20 bp, synthetic construct
  source 1..20: mol_type = other DNA
  misc_feature 1..20: note = Synthetic (Hu.DMD.Exon52.20.018)
gtcccatgcttgttaaaaaa

SEQ ID NO: 662 - DNA, 25 bp, synthetic construct
  source 1..25: mol_type = other DNA
  misc_feature 1..25: note = Synthetic (Hu.DMD.Exon53.25.001)
ctagaataaaaggaaaaataaatat

SEQ ID NO: 663 - DNA, 25 bp, synthetic construct
  source 1..25: mol_type = other DNA
  misc_feature 1..25: note = Synthetic (Hu.DMD.Exon53.25.002)
aactagaataaaaggaaaaataaat

SEQ ID NO: 664 - DNA, 25 bp, synthetic construct
  source 1..25: mol_type = other DNA
  misc_feature 1..25: note = Synthetic (Hu.DMD.Exon53.25.002.2)
ttcaactagaataaaaggaaaaata

SEQ ID NO: 665 - DNA, 25 bp, synthetic construct
  source 1..25: mol_type = other DNA
  misc_feature 1..25: note = Synthetic (Hu.DMD.Exon53.25.003)
ctttcaactagaataaaaggaaaaa

SEQ ID NO: 666 - DNA, 25 bp, synthetic construct
  source 1..25: mol_type = other DNA
  misc_feature 1..25: note = Synthetic (Hu.DMD.Exon53.25.003.2)
attctttcaactagaataaaaggaa

SEQ ID NO: 667 - DNA, 25 bp, synthetic construct
  source 1..25: mol_type = other DNA
  misc_feature 1..25: note = Synthetic (Hu.DMD.Exon53.25.004)
gaattctttcaactagaataaaagg

SEQ ID NO: 668 - DNA, 25 bp, synthetic construct
  source 1..25: mol_type = other DNA
  misc_feature 1..25: note = Synthetic (Hu.DMD.Exon53.25.004.2)
tctgaattctttcaactagaataaa

SEQ ID NO: 669 - DNA, 25 bp, synthetic construct
  source 1..25: mol_type = other DNA
  misc_feature 1..25: note = Synthetic (Hu.DMD.Exon53.25.005)
attctgaattctttcaactagaata

SEQ ID NO: 670 - DNA, 25 bp, synthetic construct
  source 1..25: mol_type = other DNA
  misc_feature 1..25: note = Synthetic (Hu.DMD.Exon53.25.005.2)
ctgattctgaattctttcaactaga

SEQ ID NO: 671 - DNA, 25 bp, synthetic construct
  source 1..25: mol_type = other DNA
  misc_feature 1..25: note = Synthetic (Hu.DMD.Exon53.25.006)
cactgattctgaattctttcaacta

SEQ ID NO: 672 - DNA, 25 bp, synthetic construct
  source 1..25: mol_type = other DNA
  misc_feature 1..25: note = Synthetic (Hu.DMD.Exon53.25.006.2)
tcccactgattctgaattctttcaa

SEQ ID NO: 673 - DNA, 25 bp, synthetic construct
  source 1..25: mol_type = other DNA
  misc_feature 1..25: note = Synthetic (Hu.DMD.Exon53.25.007)
catcccactgattctgaattctttc

SEQ ID NO: 674 - DNA, 25 bp, synthetic construct
  source 1..25: mol_type = other DNA
  misc_feature 1..25: note = Synthetic (Hu.DMD.Exon53.25.008)
tacttcatcccactgattctgaatt

SEQ ID NO: 675 - DNA, 24 bp, synthetic construct
  source 1..24: mol_type = other DNA
  misc_feature 1..24: note = Synthetic (Hu.DMD.Exon53.25.009)
cggttctgaaggtgttcttgtact

SEQ ID NO: 676 - DNA, 25 bp, synthetic construct
  source 1..25: mol_type = other DNA
  misc_feature 1..25: note = Synthetic (Hu.DMD.Exon53.25.009.2)
ctgttgcctccggttctgaaggtgt

SEQ ID NO: 677 - DNA, 25 bp, synthetic construct
  source 1..25: mol_type = other DNA
  misc_feature 1..25: note = Synthetic (Hu.DMD.Exon53.25.010)
tttcattcaactgttgcctccggtt

SEQ ID NO: 678 - DNA, 25 bp, synthetic construct
  source 1..25: mol_type = other DNA
  misc_feature 1..25: note = Synthetic (Hu.DMD.Exon53.25.010.2)
taacatttcattcaactgttgcctc

SEQ ID NO: 679 - DNA, 25 bp, synthetic construct
  source 1..25: mol_type = other DNA
  misc_feature 1..25: note = Synthetic (Hu.DMD.Exon53.25.011)
ttgtgttgaatcctttaacatttca

SEQ ID NO: 680 - DNA, 25 bp, synthetic construct
  source 1..25: mol_type = other DNA
  misc_feature 1..25: note = Synthetic (Hu.DMD.Exon53.25.012)
tcttccttagcttccagccattgtg

SEQ ID NO: 681 - DNA, 25 bp, synthetic construct
  source 1..25: mol_type = other DNA
  misc_feature 1..25: note = Synthetic (Hu.DMD.Exon53.25.012.2)
cttagcttccagccattgtgttgaa

SEQ ID NO: 682 - DNA, 25 bp, synthetic construct
  source 1..25: mol_type = other DNA
  misc_feature 1..25: note = Synthetic (Hu.DMD.Exon53.25.013)
gtcctaagacctgctcagcttcttc

SEQ ID NO: 683 - DNA, 25 bp, synthetic construct
  source 1..25: mol_type = other DNA
  misc_feature 1..25: note = Synthetic (Hu.DMD.Exon53.25.013.2)
ctgctcagcttcttccttagcttcc

SEQ ID NO: 684 - DNA, 25 bp, synthetic construct
  source 1..25: mol_type = other DNA
  misc_feature 1..25: note = Synthetic (Hu.DMD.Exon53.25.014)
ctcaagcttggctctggcctgtcct

SEQ ID NO: 685 - DNA, 25 bp, synthetic construct
  source 1..25: mol_type = other DNA
  misc_feature 1..25: note = Synthetic (Hu.DMD.Exon53.25.014.2)
ggcctgtcctaagacctgctcagct

SEQ ID NO: 686 - DNA, 25 bp, synthetic construct
  source 1..25: mol_type = other DNA
  misc_feature 1..25: note = Synthetic (Hu.DMD.Exon53.25.015)
tagggaccctccttccatgactcaa

SEQ ID NO: 687 - DNA, 25 bp, synthetic construct
  source 1..25: mol_type = other DNA
  misc_feature 1..25: note = Synthetic (Hu.DMD.Exon53.25.016)
tttggattgcatctactgtataggg

SEQ ID NO: 688 - DNA, 25 bp, synthetic construct
  source 1..25: mol_type = other DNA
  misc_feature 1..25: note = Synthetic (Hu.DMD.Exon53.25.016.2)
accctccttccatgactcaagcttg

SEQ ID NO: 689 - DNA, 25 bp, synthetic construct
  source 1..25: mol_type = other DNA
  misc_feature 1..25: note = Synthetic (Hu.DMD.Exon53.25.017)
cttggtttctgtgattttcttttgg

SEQ ID NO: 690 - DNA, 25 bp, synthetic construct
  source 1..25: mol_type = other DNA
  misc_feature 1..25: note = Synthetic (Hu.DMD.Exon53.25.017.2)
atctactgtatagggaccctccttc

SEQ ID NO: 691 - DNA, 25 bp, synthetic construct
  source 1..25: mol_type = other DNA
  misc_feature 1..25: note = Synthetic (Hu.DMD.Exon53.25.018)
ctaaccttggtttctgtgattttct

SEQ ID NO: 692 - DNA, 25 bp, synthetic construct
  source 1..25: mol_type = other DNA
  misc_feature 1..25: note = Synthetic (Hu.DMD.Exon53.25.018.2)
tttcttttggattgcatctactgta

SEQ ID NO: 693 - DNA, 25 bp, synthetic construct
  source 1..25: mol_type = other DNA
  misc_feature 1..25: note = Synthetic (Hu.DMD.Exon53.25.019)
tgatactaaccttggtttctgtgat

SEQ ID NO: 694 - DNA, 25 bp, synthetic construct
  source 1..25: mol_type = other DNA
  misc_feature 1..25: note = Synthetic (Hu.DMD.Exon53.25.020)
atctttgatactaaccttggtttct

SEQ ID NO: 695 - DNA, 25 bp, synthetic construct
  source 1..25: mol_type = other DNA
  misc_feature 1..25: note = Synthetic (Hu.DMD.Exon53.25.021)
aaggtatctttgatactaaccttgg

SEQ ID NO: 696 - DNA, 25 bp, synthetic construct
  source 1..25: mol_type = other DNA
  misc_feature 1..25: note = Synthetic (Hu.DMD.Exon53.25.022)
ttaaaaaggtatctttgatactaac

SEQ ID NO: 697 - DNA, 20 bp, synthetic construct
  source 1..20: mol_type = other DNA
  misc_feature 1..20: note = Synthetic (Hu.DMD.Exon53.20.001)
ataaaaggaaaaataaatat

SEQ ID NO: 698 - DNA, 20 bp, synthetic construct
  source 1..20: mol_type = other DNA
  misc_feature 1..20: note = Synthetic (Hu.DMD.Exon53.20.002)
gaataaaaggaaaaataaat

SEQ ID NO: 699 - DNA, 20 bp, synthetic construct
  source 1..20: mol_type = other DNA
  misc_feature 1..20: note = Synthetic (Hu.DMD.Exon53.20.003)
aactagaataaaaggaaaaa

SEQ ID NO: 700 - DNA, 20 bp, synthetic construct
  source 1..20: mol_type = other DNA
  misc_feature 1..20: note = Synthetic (Hu.DMD.Exon53.20.004)
ctttcaactagaataaaagg

SEQ ID NO: 701 - DNA, 20 bp, synthetic construct
  source 1..20: mol_type = other DNA
  misc_feature 1..20: note = Synthetic (Hu.DMD.Exon53.20.005)
gaattctttcaactagaata

SEQ ID NO: 702 - DNA, 20 bp, synthetic construct
  source 1..20: mol_type = other DNA
  misc_feature 1..20: note = Synthetic (Hu.DMD.Exon53.20.006)
attctgaattctttcaacta

SEQ ID NO: 703 - DNA, 20 bp, synthetic construct
  source 1..20: mol_type = other DNA
  misc_feature 1..20: note = Synthetic (Hu.DMD.Exon53.20.007)
tacttcatcccactgattct

SEQ ID NO: 704 - DNA, 19 bp, synthetic construct
  source 1..19: mol_type = other DNA
  misc_feature 1..19: note = Synthetic (Hu.DMD.Exon53.20.008)
ctgaaggtgttcttgtact

SEQ ID NO: 705 - DNA, 20 bp, synthetic construct
  source 1..20: mol_type = other DNA
  misc_feature 1..20: note = Synthetic (Hu.DMD.Exon53.20.009)
ctgttgcctccggttctgaa

SEQ ID NO: 706 - DNA, 20 bp, synthetic construct
  source 1..20: mol_type = other DNA
  misc_feature 1..20: note = Synthetic (Hu.DMD.Exon53.20.010)
taacatttcattcaactgtt

SEQ ID NO: 707 - DNA, 20 bp, synthetic construct
  source 1..20: mol_type = other DNA
  misc_feature 1..20: note = Synthetic (Hu.DMD.Exon53.20.011)
ttgtgttgaatcctttaaca

SEQ ID NO: 708 - DNA, 20 bp, synthetic construct
  source 1..20: mol_type = other DNA
  misc_feature 1..20: note = Synthetic (Hu.DMD.Exon53.20.012)
cttagcttccagccattgtg

SEQ ID NO: 709 - DNA, 20 bp, synthetic construct
  source 1..20: mol_type = other DNA
  misc_feature 1..20: note = Synthetic (Hu.DMD.Exon53.20.013)
ctgctcagcttcttccttag

SEQ ID NO: 710 - DNA, 20 bp, synthetic construct
  source 1..20: mol_type = other DNA
  misc_feature 1..20: note = Synthetic (Hu.DMD.Exon53.20.014)
ggcctgtcctaagacctgct

SEQ ID NO: 711 - DNA, 20 bp, synthetic construct
  source 1..20: mol_type = other DNA
  misc_feature 1..20: note = Synthetic (Hu.DMD.Exon53.20.015)
ctcaagcttggctctggcct

SEQ ID NO: 712 - DNA, 20 bp, synthetic construct
  source 1..20: mol_type = other DNA
  misc_feature 1..20: note = Synthetic (Hu.DMD.Exon53.20.016)
accctccttccatgactcaa

SEQ ID NO: 713 - DNA, 20 bp, synthetic construct
  source 1..20: mol_type = other DNA
  misc_feature 1..20: note = Synthetic (Hu.DMD.Exon53.20.017)
atctactgtatagggaccct

SEQ ID NO: 714 - DNA, 20 bp, synthetic construct
  source 1..20: mol_type = other DNA
  misc_feature 1..20: note = Synthetic (Hu.DMD.Exon53.20.018)
tttcttttggattgcatcta

SEQ ID NO: 715 - DNA, 20 bp, synthetic construct
  source 1..20: mol_type = other DNA
  misc_feature 1..20: note = Synthetic (Hu.DMD.Exon53.20.019)
cttggtttctgtgattttct

SEQ ID NO: 716 - DNA, 20 bp, synthetic construct
  source 1..20: mol_type = other DNA
  misc_feature 1..20: note = Synthetic (Hu.DMD.Exon53.20.020)
ctaaccttggtttctgtgat

SEQ ID NO: 717 - DNA, 20 bp, synthetic construct
  source 1..20: mol_type = other DNA
  misc_feature 1..20: note = Synthetic (Hu.DMD.Exon53.20.021)
tgatactaaccttggtttct

SEQ ID NO: 718 - DNA, 20 bp, synthetic construct
  source 1..20: mol_type = other DNA
  misc_feature 1..20: note = Synthetic (Hu.DMD.Exon53.20.022)
atctttgatactaaccttgg

SEQ ID NO: 719 - DNA, 20 bp, synthetic construct
  source 1..20: mol_type = other DNA
  misc_feature 1..20: note = Synthetic (Hu.DMD.Exon53.20.023)
aaggtatctttgatactaac

SEQ ID NO: 720 - DNA, 20 bp, synthetic construct
  source 1..20: mol_type = other DNA
  misc_feature 1..20: note = Synthetic (Hu.DMD.Exon53.20.024)
ttaaaaaggtatctttgata

SEQ ID NO: 721 - DNA, 24 bp, synthetic construct
  source 1..24: mol_type = other DNA
  misc_feature 1..24: note = Synthetic (Hu.DMD.Exon54.25.001)
ctatagatttttatgagaaagaga

SEQ ID NO: 722 - DNA, 25 bp, synthetic construct
  source 1..25: mol_type = other DNA
  misc_feature 1..25: note = Synthetic (Hu.DMD.Exon54.25.002)
aactgctatagatttttatgagaaa

SEQ ID NO: 723 - DNA, 25 bp, synthetic construct
  source 1..25: mol_type = other DNA
  misc_feature 1..25: note = Synthetic (Hu.DMD.Exon54.25.003)
tggccaactgctatagatttttatg

SEQ ID NO: 724 - DNA, 25 bp, synthetic construct
  source 1..25: mol_type = other DNA
  misc_feature 1..25: note = Synthetic (Hu.DMD.Exon54.25.004)
gtctttggccaactgctatagattt

SEQ ID NO: 725 - DNA, 25 bp, synthetic construct
  source 1..25: mol_type = other DNA
  misc_feature 1..25: note = Synthetic (Hu.DMD.Exon54.25.005)
cggaggtctttggccaactgctata

SEQ ID NO: 726 - DNA, 25 bp, synthetic construct
  source 1..25: mol_type = other DNA
  misc_feature 1..25: note = Synthetic (Hu.DMD.Exon54.25.006)
actggcggaggtctttggccaactg

SEQ ID NO: 727 - DNA, 25 bp, synthetic construct
  source 1..25: mol_type = other DNA
  misc_feature 1..25: note = Synthetic (Hu.DMD.Exon54.25.007)
tttgtctgccactggcggaggtctt

SEQ ID NO: 728 - DNA, 25 bp, synthetic construct
  source 1..25: mol_type = other DNA
  misc_feature 1..25: note = Synthetic (Hu.DMD.Exon54.25.008)
agtcatttgccacatctacatttgt

SEQ ID NO: 729 - DNA, 25 bp, synthetic construct
  source 1..25: mol_type = other DNA
  misc_feature 1..25: note = Synthetic (Hu.DMD.Exon54.25.008.2)
tttgccacatctacatttgtctgcc

SEQ ID NO: 730 - DNA, 25 bp, synthetic construct
  source 1..25: mol_type = other DNA
  misc_feature 1..25: note = Synthetic (Hu.DMD.Exon54.25.009)
ccggagaagtttcagggccaagtca

SEQ ID NO: 731 - DNA, 25 bp, synthetic construct
  source 1..25: mol_type = other DNA
  misc_feature 1..25: note = Synthetic (Hu.DMD.Exon54.25.010)
gtatcatctgcagaataatcccgga

SEQ ID NO: 732 - DNA, 25 bp, synthetic construct
  source 1..25: mol_type = other DNA
  misc_feature 1..25: note = Synthetic (Hu.DMD.Exon54.25.010.2)
taatcccggagaagtttcagggcca

SEQ ID NO: 733 - DNA, 25 bp, synthetic construct
  source 1..25: mol_type = other DNA
  misc_feature 1..25: note = Synthetic (Hu.DMD.Exon54.25.011)
ttatcatgtggacttttctggtatc

SEQ ID NO: 734 - DNA, 25 bp, synthetic construct
  source 1..25: mol_type = other DNA
  misc_feature 1..25: note = Synthetic (Hu.DMD.Exon54.25.012)
agaggcattgatattctctgttatc

SEQ ID NO: 735 - DNA, 25 bp, synthetic construct
  source 1..25: mol_type = other DNA
  misc_feature 1..25: note = Synthetic (Hu.DMD.Exon54.25.012.2)
atgtggacttttctggtatcatctg

SEQ ID NO: 736 - DNA, 25 bp, synthetic construct
  source 1..25: mol_type = other DNA
  misc_feature 1..25: note = Synthetic (Hu.DMD.Exon54.25.013)
cttttatgaatgcttctccaagagg

SEQ ID NO: 737 - DNA, 25 bp, synthetic construct
  source 1..25: mol_type = other DNA
  misc_feature 1..25: note = Synthetic (Hu.DMD.Exon54.25.013.2)
atattctctgttatcatgtggactt

SEQ ID NO: 738 - DNA, 25 bp, synthetic construct
  source 1..25: mol_type = other DNA
  misc_feature 1..25: note = Synthetic (Hu.DMD.Exon54.25.014)
cataccttttatgaatgcttctcca

SEQ ID NO: 739 - DNA, 25 bp, synthetic construct
  source 1..25: mol_type = other DNA
  misc_feature 1..25: note = Synthetic (Hu.DMD.Exon54.25.014.2)
ctccaagaggcattgatattctctg

SEQ ID NO: 740 - DNA, 25 bp, synthetic construct
  source 1..25: mol_type = other DNA
  misc_feature 1..25: note = Synthetic (Hu.DMD.Exon54.25.015)
taattcataccttttatgaatgctt

SEQ ID NO: 741 - DNA, 25 bp, synthetic construct
  source 1..25: mol_type = other DNA
  misc_feature 1..25: note = Synthetic (Hu.DMD.Exon54.25.016)
taatgtaattcataccttttatgaa

SEQ ID NO: 742 - DNA, 25 bp, synthetic construct
  source 1..25: mol_type = other DNA
  misc_feature 1..25: note = Synthetic (Hu.DMD.Exon54.25.017)
agaaataatgtaattcatacctttt

SEQ ID NO: 743 - DNA, 25 bp, synthetic construct
  source 1..25: mol_type = other DNA
  misc_feature 1..25: note = Synthetic (Hu.DMD.Exon54.25.018)
gttttagaaataatgtaattcatac

SEQ ID NO: 744 - DNA, 19 bp, synthetic construct
  source 1..19: mol_type = other DNA
  misc_feature 1..19: note = Synthetic (Hu.DMD.Exon54.20.001)
gatttttatgagaaagaga

SEQ ID NO: 745 - DNA, 20 bp, synthetic construct
  source 1..20: mol_type = other DNA
  misc_feature 1..20: note = Synthetic (Hu.DMD.Exon54.20.002)
ctatagatttttatgagaaa

SEQ ID NO: 746 - DNA, 20 bp, synthetic construct
  source 1..20: mol_type = other DNA
  misc_feature 1..20: note = Synthetic (Hu.DMD.Exon54.20.003)
aactgctatagatttttatg

SEQ ID NO: 747 - DNA, 20 bp, synthetic construct
  source 1..20: mol_type = other DNA
  misc_feature 1..20: note = Synthetic (Hu.DMD.Exon54.20.004)
tggccaactgctatagattt

SEQ ID NO: 748 - DNA, 20 bp, synthetic construct
  source 1..20: mol_type = other DNA
  misc_feature 1..20: note = Synthetic (Hu.DMD.Exon54.20.005)
gtctttggccaactgctata

SEQ ID NO: 749 - DNA, 20 bp, synthetic construct
  source 1..20: mol_type = other DNA
  misc_feature 1..20: note = Synthetic (Hu.DMD.Exon54.20.006)
cggaggtctttggccaactg

SEQ ID NO: 750 - DNA, 20 bp, synthetic construct
  source 1..20: mol_type = other DNA
  misc_feature 1..20: note = Synthetic (Hu.DMD.Exon54.20.007)
tttgtctgccactggcggag

SEQ ID NO: 751 - DNA, 20 bp, synthetic construct
  source 1..20: mol_type = other DNA
  misc_feature 1..20: note = Synthetic (Hu.DMD.Exon54.20.008)
tttgccacatctacatttgt

SEQ ID NO: 752 - DNA, 20 bp, synthetic construct
  source 1..20: mol_type = other DNA
  misc_feature 1..20: note = Synthetic (Hu.DMD.Exon54.20.009)
ttcagggccaagtcatttgc

SEQ ID NO: 753 - DNA, 20 bp, synthetic construct
  source 1..20: mol_type = other DNA
  misc_feature 1..20: note = Synthetic (Hu.DMD.Exon54.20.010)
taatcccggagaagtttcag

SEQ ID NO: 754 - DNA, 20 bp, synthetic construct
  source 1..20: mol_type = other DNA
  misc_feature 1..20: note = Synthetic (Hu.DMD.Exon54.20.011)
gtatcatctgcagaataatc

SEQ ID NO: 755 - DNA, 20 bp, synthetic construct
  source 1..20: mol_type = other DNA
  misc_feature 1..20: note = Synthetic (Hu.DMD.Exon54.20.012)
atgtggacttttctggtatc

SEQ ID NO: 756 - DNA, 20 bp, synthetic construct
  source 1..20: mol_type = other DNA
  misc_feature 1..20: note = Synthetic (Hu.DMD.Exon54.20.013)
atattctctgttatcatgtg

SEQ ID NO: 757 - DNA, 20 bp, synthetic construct
  source 1..20: mol_type = other DNA
  misc_feature 1..20: note = Synthetic (Hu.DMD.Exon54.20.014)
ctccaagaggcattgatatt

SEQ ID NO: 758 - DNA, 20 bp, synthetic construct
  source 1..20: mol_type = other DNA
  misc_feature 1..20: note = Synthetic (Hu.DMD.Exon54.20.015)
cttttatgaatgcttctcca

SEQ ID NO: 759 - DNA, 20 bp, synthetic construct
  source 1..20: mol_type = other DNA
  misc_feature 1..20: note = Synthetic (Hu.DMD.Exon54.20.016)
cataccttttatgaatgctt

SEQ ID NO: 760 - DNA, 20 bp, synthetic construct
  source 1..20: mol_type = other DNA
  misc_feature 1..20: note = Synthetic (Hu.DMD.Exon54.20.017)
taattcataccttttatgaa

SEQ ID NO: 761 - DNA, 20 bp, synthetic construct
  source 1..20: mol_type = other DNA
  misc_feature 1..20: note = Synthetic (Hu.DMD.Exon54.20.018)
taatgtaattcatacctttt

SEQ ID NO: 762 - DNA, 20 bp, synthetic construct
  source 1..20: mol_type = other DNA
  misc_feature 1..20: note = Synthetic (Hu.DMD.Exon54.20.019)
agaaataatgtaattcatac

SEQ ID NO: 763 - DNA, 20 bp, synthetic construct
  source 1..20: mol_type = other DNA
  misc_feature 1..20: note = Synthetic (Hu.DMD.Exon54.20.020)
gttttagaaataatgtaatt

SEQ ID NO: 764 - DNA, 25 bp, synthetic construct
  source 1..25: mol_type = other DNA
  misc_feature 1..25: note = Synthetic (Hu.DMD.Exon55.25.001)
ctgcaaaggaccaaatgttcagatg

SEQ ID NO: 765 - DNA, 25 bp, synthetic construct
  source 1..25: mol_type = other DNA
  misc_feature 1..25: note = Synthetic (Hu.DMD.Exon55.25.002)
tcaccctgcaaaggaccaaatgttc

SEQ ID NO: 766 - DNA, 25 bp, synthetic construct
  source 1..25: mol_type = other DNA
  misc_feature 1..25: note = Synthetic (Hu.DMD.Exon55.25.003)
ctcactcaccctgcaaaggaccaaa

SEQ ID NO: 767 - DNA, 25 bp, synthetic construct
  source 1..25: mol_type = other DNA
  misc_feature 1..25: note = Synthetic (Hu.DMD.Exon55.25.004)
tctcgctcactcaccctgcaaagga

SEQ ID NO: 768 - DNA, 25 bp, synthetic construct
  source 1..25: mol_type = other DNA
  misc_feature 1..25: note = Synthetic (Hu.DMD.Exon55.25.005)
cagcctctcgctcactcaccctgca

SEQ ID NO: 769 - DNA, 25 bp, synthetic construct
  source 1..25: mol_type = other DNA
  misc_feature 1..25: note = Synthetic (Hu.DMD.Exon55.25.006)
caaagcagcctctcgctcactcacc

SEQ ID NO: 770 - DNA, 25 bp, synthetic construct
  source 1..25: mol_type = other DNA
  misc_feature 1..25: note = Synthetic (Hu.DMD.Exon55.25.007)
tcttccaaagcagcctctcgctcac

SEQ ID NO: 771 - DNA, 25 bp, synthetic construct
  source 1..25: mol_type = other DNA
  misc_feature 1..25: note = Synthetic (Hu.DMD.Exon55.25.007.2)
tctatgagtttcttccaaagcagcc

SEQ ID NO: 772 - DNA, 25 bp, synthetic construct
  source 1..25: mol_type = other DNA
  misc_feature 1..25: note = Synthetic (Hu.DMD.Exon55.25.008)
gttgcagtaatctatgagtttcttc

SEQ ID NO: 773 - DNA, 25 bp, synthetic construct
  source 1..25: mol_type = other DNA
  misc_feature 1..25: note = Synthetic (Hu.DMD.Exon55.25.008.2)
gaactgttgcagtaatctatgagtt

SEQ ID NO: 774 - DNA, 25 bp, synthetic construct
  source 1..25: mol_type = other DNA
  misc_feature 1..25: note = Synthetic (Hu.DMD.Exon55.25.009)
ttccaggtccagggggaactgttgc

SEQ ID NO: 775 - DNA, 25 bp, synthetic construct
  source 1..25: mol_type = other DNA
  misc_feature 1..25: note = Synthetic (Hu.DMD.Exon55.25.010)
gtaagccaggcaagaaacttttcca

SEQ ID NO: 776 - DNA, 25 bp, synthetic construct
  source 1..25: mol_type = other DNA
  misc_feature 1..25: note = Synthetic (Hu.DMD.Exon55.25.010.2)
ccaggcaagaaacttttccaggtcc

SEQ ID NO: 777 - DNA, 25 bp, synthetic construct
  source 1..25: mol_type = other DNA
  misc_feature 1..25: note = Synthetic (Hu.DMD.Exon55.25.011)
tggcagttgtttcagcttctgtaag

SEQ ID NO: 778 - DNA, 25 bp, synthetic construct
  source 1..25: mol_type = other DNA
  misc_feature 1..25: note = Synthetic (Hu.DMD.Exon55.25.011.2)
ttcagcttctgtaagccaggcaaga

SEQ ID NO: 779 - DNA, 25 bp, synthetic construct
  source 1..25: mol_type = other DNA
  misc_feature 1..25: note = Synthetic (Hu.DMD.Exon55.25.012)
ggtagcatcctgtaggacattggca

SEQ ID NO: 780 - DNA, 25 bp, synthetic construct
  source 1..25: mol_type = other DNA
  misc_feature 1..25: note = Synthetic (Hu.DMD.Exon55.25.012.2)
gacattggcagttgtttcagcttct

SEQ ID NO: 781 - DNA, 25 bp, synthetic construct
  source 1..25: mol_type = other DNA
  misc_feature 1..25: note = Synthetic (Hu.DMD.Exon55.25.013)
tctaggagcctttccttacgggtag

SEQ ID NO: 782 - DNA, 25 bp, synthetic construct
  source 1..25: mol_type = other DNA
  misc_feature 1..25: note = Synthetic (Hu.DMD.Exon55.25.014)
cttttactcccttggagtcttctag

SEQ ID NO: 783 - DNA, 25 bp, synthetic construct
  source 1..25: mol_type = other DNA
  misc_feature 1..25: note = Synthetic (Hu.DMD.Exon55.25.014.2)
gagcctttccttacgggtagcatcc

SEQ ID NO: 784 - DNA, 25 bp, synthetic construct
  source 1..25: mol_type = other DNA
  misc_feature 1..25: note = Synthetic (Hu.DMD.Exon55.25.015)
ttgccattgtttcatcagctctttt

SEQ ID NO: 785 - DNA, 25 bp, synthetic construct
  source 1..25: mol_type = other DNA
  misc_feature 1..25: note = Synthetic (Hu.DMD.Exon55.25.015.2)
cttggagtcttctaggagcctttcc

SEQ ID NO: 786 - DNA, 25 bp, synthetic construct
  source 1..25: mol_type = other DNA
  misc_feature 1..25: note = Synthetic (Hu.DMD.Exon55.25.016)
cttacttgccattgtttcatcagct

SEQ ID NO: 787 - DNA, 25 bp, synthetic construct
  source 1..25: mol_type = other DNA
  misc_feature 1..25: note = Synthetic (Hu.DMD.Exon55.25.016.2)
cagctcttttactcccttggagtct

SEQ ID NO: 788 - DNA, 25 bp, synthetic construct
  source 1..25: mol_type = other DNA
  misc_feature 1..25: note = Synthetic (Hu.DMD.Exon55.25.017)
cctgacttacttgccattgtttcat

SEQ ID NO: 789 - DNA, 25 bp, synthetic construct
  source 1..25: mol_type = other DNA
  misc_feature 1..25: note = Synthetic (Hu.DMD.Exon55.25.018)
aaatgcctgacttacttgccattgt

SEQ ID NO: 790 - DNA, 25 bp, synthetic construct
  source 1..25: mol_type = other DNA
  misc_feature 1..25: note = Synthetic (Hu.DMD.Exon55.25.019)
agcggaaatgcctgacttacttgcc

SEQ ID NO: 791 - DNA, 25 bp, synthetic construct
  source 1..25: mol_type = other DNA
  misc_feature 1..25: note = Synthetic (Hu.DMD.Exon55.25.020)
gctaaagcggaaatgcctgacttac

SEQ ID NO: 792 - DNA, 20 bp, synthetic construct
  source 1..20: mol_type = other DNA
  misc_feature 1..20: note = Synthetic (Hu.DMD.Exon55.20.001)
aaggaccaaatgttcagatg

SEQ ID NO: 793 - DNA, 20 bp, synthetic construct
  source 1..20: mol_type = other DNA
  misc_feature 1..20: note = Synthetic (Hu.DMD.Exon55.20.002)
ctgcaaaggaccaaatgttc

SEQ ID NO: 794 - DNA, 20 bp, synthetic construct
  source 1..20: mol_type = other DNA
  misc_feature 1..20: note = Synthetic (Hu.DMD.Exon55.20.003)
tcaccctgcaaaggaccaaa

SEQ ID NO: 795 - DNA, 20 bp, synthetic construct
  source 1..20: mol_type = other DNA
  misc_feature 1..20: note = Synthetic (Hu.DMD.Exon55.20.004)
ctcactcaccctgcaaagga

SEQ ID NO: 796 - DNA, 20 bp, synthetic construct
  source 1..20: mol_type = other DNA
  misc_feature 1..20: note = Synthetic (Hu.DMD.Exon55.20.005)
tctcgctcactcaccctgca

SEQ ID NO: 797 - DNA, 20 bp, synthetic construct
  source 1..20: mol_type = other DNA
  misc_feature 1..20: note = Synthetic (Hu.DMD.Exon55.20.006)
cagcctctcgctcactcacc

SEQ ID NO: 798 - DNA, 20 bp, synthetic construct
  source 1..20: mol_type = other DNA
  misc_feature 1..20: note = Synthetic (Hu.DMD.Exon55.20.007)
caaagcagcctctcgctcac

SEQ ID NO: 799 - DNA, 20 bp, synthetic construct
  source 1..20: mol_type = other DNA
  misc_feature 1..20: note = Synthetic (Hu.DMD.Exon55.20.008)
tctatgagtttcttccaaag

SEQ ID NO: 800 - DNA, 20 bp, synthetic construct
  source 1..20: mol_type = other DNA
  misc_feature 1..20: note = Synthetic (Hu.DMD.Exon55.20.009)
gaactgttgcagtaatctat

SEQ ID NO: 801 - DNA, 20 bp, synthetic construct
  source 1..20: mol_type = other DNA
  misc_feature 1..20: note = Synthetic (Hu.DMD.Exon55.20.010)
ttccaggtccagggggaact

SEQ ID NO: 802 - DNA, 20 bp, synthetic construct
  source 1..20: mol_type = other DNA
  misc_feature 1..20: note = Synthetic (Hu.DMD.Exon55.20.011)
ccaggcaagaaacttttcca

SEQ ID NO: 803 - DNA, 20 bp, synthetic construct
  source 1..20: mol_type = other DNA
  misc_feature 1..20: note = Synthetic (Hu.DMD.Exon55.20.012)
ttcagcttctgtaagccagg

SEQ ID NO: 804 - DNA, 20 bp, synthetic construct
  source 1..20: mol_type = other DNA
  misc_feature 1..20: note = Synthetic (Hu.DMD.Exon55.20.013)
gacattggcagttgtttcag

SEQ ID NO: 805 - DNA, 20 bp, synthetic construct
  source 1..20: mol_type = other DNA
  misc_feature 1..20: note = Synthetic (Hu.DMD.Exon55.20.014)
ggtagcatcctgtaggacat

SEQ ID NO: 806 - DNA, 20 bp, synthetic construct
  source 1..20: mol_type = other DNA
  misc_feature 1..20: note = Synthetic (Hu.DMD.Exon55.20.015)
gagcctttccttacgggtag

SEQ ID NO: 807 - DNA, 20 bp, synthetic construct
  source 1..20: mol_type = other DNA
  misc_feature 1..20: note = Synthetic (Hu.DMD.Exon55.20.016)
cttggagtcttctaggagcc

SEQ ID NO: 808 - DNA, 20 bp, synthetic construct
  source 1..20: mol_type = other DNA
  misc_feature 1..20: note = Synthetic (Hu.DMD.Exon55.20.017)
cagctcttttactcccttgg

SEQ ID NO: 809 - DNA, 20 bp, synthetic construct
  source 1..20: mol_type = other DNA
  misc_feature 1..20: note = Synthetic (Hu.DMD.Exon55.20.018)
ttgccattgtttcatcagct

SEQ ID NO: 810 - DNA, 20 bp, synthetic construct
  source 1..20: mol_type = other DNA
  misc_feature 1..20: note = Synthetic (Hu.DMD.Exon55.20.019)
cttacttgccattgtttcat

SEQ ID NO: 811 - DNA, 20 bp, synthetic construct
  source 1..20: mol_type = other DNA
  misc_feature 1..20: note = Synthetic (Hu.DMD.Exon55.20.020)
cctgacttacttgccattgt

SEQ ID NO: 812 - DNA, 20 bp, synthetic construct
  source 1..20: mol_type = other DNA
  misc_feature 1..20: note = Synthetic (Hu.DMD.Exon55.20.021)
aaatgcctgacttacttgcc

SEQ ID NO: 813 - DNA, 20 bp, synthetic construct
  source 1..20: mol_type = other DNA
  misc_feature 1..20: note = Synthetic (Hu.DMD.Exon55.20.022)
agcggaaatgcctgacttac

SEQ ID NO: 814 - DNA, 20 bp, synthetic construct
  source 1..20: mol_type = other DNA
  misc_feature 1..20: note = Synthetic (Hu.DMD.Exon55.20.023)
gctaaagcggaaatgcctga

SEQ ID NO: 815 - DNA, 29 bp, synthetic construct
  source 1..29: mol_type = other DNA
  misc_feature 1..29: note = Synthetic (H50A(+02+30)-AVI-5656)
ccactcagagctcagatcttctaacttcc

SEQ ID NO: 816 - DNA, 25 bp, synthetic construct
  source 1..25: mol_type = other DNA
  misc_feature 1..25: note = Synthetic (H50D(+07-18)-AVI-5915)
gggatccagtatacttacaggctcc

SEQ ID NO: 817 - DNA, 27 bp, synthetic construct
  source 1..27: mol_type = other DNA
  misc_feature 1..27: note = Synthetic (H50A(+07+33))
cttccactcagagctcagatcttctaa

SEQ ID NO: 818 - DNA, 30 bp, synthetic construct
  source 1..30: mol_type = other DNA
  misc_feature 1..30: note = Synthetic (H51A(+61+90)-AVI-4657)
acatcaaggaagatggcatttctagtttgg

SEQ ID NO: 819 - DNA, 30 bp, synthetic construct; identical to SEQ ID NO: 1
  source 1..30: mol_type = other DNA
  misc_feature 1..30: note = Synthetic (H51A(+66+95)-AVI-4658)

SEQ ID NO: 820 - DNA, 24 bp, synthetic construct
  source 1..24: mol_type = other DNA
  misc_feature 1..24: note = Synthetic (H51A(+111+134))
ttctgtccaagcccggttgaaatc

SEQ ID NO: 821 - DNA, 21 bp, synthetic construct
  source 1..21: mol_type = other DNA
  misc_feature 1..21: note = Synthetic (H51A(+175+195))
cacccaccatcaccctcygtg

SEQ ID NO: 822 - DNA, 22 bp, synthetic construct
  source 1..22: mol_type = other DNA
  misc_feature 1..22: note = Synthetic (H51A(+199+220))
atcatctcgttgatatcctcaa

SEQ ID NO: 823 - DNA, 25 bp, synthetic construct
  source 1..25: mol_type = other DNA
  misc_feature 1..25: note = Synthetic (H51A(+66+90))
acatcaaggaagatggcatttctag

SEQ ID NO: 824 - DNA, 26 bp, synthetic construct
  source 1..26: mol_type = other DNA
  misc_feature 1..26: note = Synthetic (H51A(-01+25))
accagagtaacagtctgagtaggagc

SEQ ID NO: 825 - DNA, 20 bp, synthetic construct
  source 1..20: mol_type = other DNA
  misc_feature 1..20: note = Synthetic (h51AON1)
tcaaggaagatggcatttct

SEQ ID NO: 826 - DNA, 23 bp, synthetic construct
  source 1..23: mol_type = other DNA
  misc_feature 1..23: note = Synthetic (h51AON2)
cctctgtgattttataacttgat

SEQ ID NO: 827 - DNA, 25 bp, synthetic construct
  source 1..25: mol_type = other DNA
  misc_feature 1..25: note = Synthetic (H51D(+08-17))
atcattttttctcataccttctgct

SEQ ID NO: 828 - DNA, 23 bp, synthetic construct
  source 1..23: mol_type = other DNA
  misc_feature 1..23: note = Synthetic (H51D(+16-07))
ctcataccttctgcttgatgatc

SEQ ID NO: 829 - DNA, 17 bp, synthetic construct
  source 1..17: mol_type = other DNA
  misc_feature 1..17: note = Synthetic (hAON#23)
tggcatttctagtttgg

SEQ ID NO: 830 - DNA, 23 bp, synthetic construct
  source 1..23: mol_type = other DNA
  misc_feature 1..23: note = Synthetic (hAON#24)
ccagagcaggtacctccaacatc

SEQ ID NO: 831 - DNA, 19 bp, synthetic construct
  source 1..19: mol_type = other DNA
  misc_feature 1..19: note = Synthetic (h44AON1)
cgccgccatttctcaacag

SEQ ID NO: 832 - DNA, 20 bp, synthetic construct
  source 1..20: mol_type = other DNA
  misc_feature 1..20: note = Synthetic (H45A(+71+90))
tgtttttgaggattgctgaa

SEQ ID NO: 833 - DNA, 19 bp, synthetic construct
  source 1..19: mol_type = other DNA
  misc_feature 1..19: note = Synthetic (h45AON1)
gctgaattatttcttcccc

SEQ ID NO: 834 - DNA, 17 bp, synthetic construct
  source 1..17: mol_type = other DNA
  misc_feature 1..17: note = Synthetic (h45AON5)
gcccaatgccatcctgg

SEQ ID NO: 835 - DNA, 26 bp, synthetic construct
  source 1..26: mol_type = other DNA
  misc_feature 1..26: note = Synthetic (H45A(-06+20))
ccaatgccatcctggagttcctgtaa

SEQ ID NO: 836 - DNA, 31 bp, synthetic construct
  source 1..31: mol_type = other DNA
  misc_feature 1..31: note = Synthetic (H53A(+39+69))
cattcaactgttgcctccggttctgaaggtg

SEQ ID NO: 837 - DNA, 18 bp, synthetic construct
  source 1..18: mol_type = other DNA
  misc_feature 1..18: note = Synthetic (h53AON1)
ctgttgcctccggttctg

SEQ ID NO: 838 - DNA, 22 bp, synthetic construct
  source 1..22: mol_type = other DNA
  misc_feature 1..22: note = Synthetic (H53A(-12+10))
attctttcaactagaataaaag

SEQ ID NO: 839 - DNA, 30 bp, synthetic construct
  source 1..30: mol_type = other DNA
  misc_feature 1..30: note = Synthetic (huEx45.30.66)
gccatcctggagttcctgtaagataccaaa

SEQ ID NO: 840 - DNA, 30 bp, synthetic construct
  source 1..30: mol_type = other DNA
  misc_feature 1..30: note = Synthetic (huEx45.30.71)
ccaatgccatcctggagttcctgtaagata

SEQ ID NO: 841 - DNA, 30 bp, synthetic construct
  source 1..30: mol_type = other DNA
  misc_feature 1..30: note = Synthetic (huEx45.30.79)
gccgctgcccaatgccatcctggagttcct

SEQ ID NO: 842 - DNA, 30 bp, synthetic construct
  source 1..30: mol_type = other DNA
  misc_feature 1..30: note = Synthetic (huEx45.30.83)
gtttgccgctgcccaatgccatcctggagt

SEQ ID NO: 843 - DNA, 30 bp, synthetic construct
  source 1..30: mol_type = other DNA
  misc_feature 1..30: note = Synthetic (huEx45.30.88)
caacagtttgccgctgcccaatgccatcct

SEQ ID NO: 844 - DNA, 30 bp, synthetic construct
  source 1..30: mol_type = other DNA
  misc_feature 1..30: note = Synthetic (huEx45.30.92)
ctgacaacagtttgccgctgcccaatgcca

SEQ ID NO: 845 - DNA, 30 bp, synthetic construct
  source 1..30: mol_type = other DNA
  misc_feature 1..30: note = Synthetic (huEx45.30.96)
tgttctgacaacagtttgccgctgcccaat

SEQ ID NO: 846 - DNA, 30 bp, synthetic construct
  source 1..30: mol_type = other DNA
  misc_feature 1..30: note = Synthetic (huEx45.30.99)
caatgttctgacaacagtttgccgctgccc

SEQ ID NO: 847 - DNA, 30 bp, synthetic construct
  source 1..30: mol_type = other DNA
  misc_feature 1..30: note = Synthetic (huEx45.30.103)
cattcaatgttctgacaacagtttgccgct

SEQ ID NO: 848 - DNA, 30 bp, synthetic construct
  source 1..30: mol_type = other DNA
  misc_feature 1..30: note = Synthetic (huEx45.30.120)
tatttcttccccagttgcattcaatgttct

SEQ ID NO: 849 - DNA, 30 bp, synthetic construct
  source 1..30: mol_type = other DNA
  misc_feature 1..30: note = Synthetic (huEx45.30.127)
gctgaattatttcttccccagttgcattca

SEQ ID NO: 850 - DNA, 30 bp, synthetic construct
  source 1..30: mol_type = other DNA
  misc_feature 1..30: note = Synthetic (huEx45.30.132)
ggattgctgaattatttcttccccagttgc

SEQ ID NO: 851 - DNA, 30 bp, synthetic construct
  source 1..30: mol_type = other DNA
  misc_feature 1..30: note = Synthetic (huEx45.30.137)
tttgaggattgctgaattatttcttcccca

SEQ ID NO: 852 - DNA, 30 bp, synthetic construct
  source 1..30: mol_type = other DNA
  misc_feature 1..30: note = Synthetic (huEx53.30.84)
gtacttcatcccactgattctgaattcttt

SEQ ID NO: 853 - DNA, 30 bp, synthetic construct
  source 1..30: mol_type = other DNA
  misc_feature 1..30: note = Synthetic (huEx53.30.88)
tcttgtacttcatcccactgattctgaatt

SEQ ID NO: 854 - DNA, 30 bp, synthetic construct
  source 1..30: mol_type = other DNA
  misc_feature 1..30: note = Synthetic (huEx53.30.91)
tgttcttgtacttcatcccactgattctga

SEQ ID NO: 855 - DNA, 30 bp, synthetic construct
  source 1..30: mol_type = other DNA
  misc_feature 1..30: note = Synthetic (huEx53.30.103)
cggttctgaaggtgttcttgtacttcatcc

SEQ ID NO: 856 - DNA, 30 bp, synthetic construct
  source 1..30: mol_type = other DNA
  misc_feature 1..30: note = Synthetic (huEx53.30.106)
ctccggttctgaaggtgttcttgtacttca

SEQ ID NO: 857 - DNA, 30 bp, synthetic construct
  source 1..30: mol_type = other DNA
  misc_feature 1..30: note = Synthetic (huEx53.30.109)
tgcctccggttctgaaggtgttcttgtact

SEQ ID NO: 858 - DNA, 30 bp, synthetic construct
  source 1..30: mol_type = other DNA
  misc_feature 1..30: note = Synthetic (huEx53.30.112)
tgttgcctccggttctgaaggtgttcttgt

SEQ ID NO: 859 - DNA, 30 bp, synthetic construct
  source 1..30: mol_type = other DNA
  misc_feature 1..30: note = Synthetic (huEx53.30.115)
aactgttgcctccggttctgaaggtgttct

SEQ ID NO: 860 - DNA, 30 bp, synthetic construct
  source 1..30: mol_type = other DNA
  misc_feature 1..30: note = Synthetic (huEx53.30.118)
ttcaactgttgcctccggttctgaaggtgt

SEQ ID NO: 861 - protein, 10 aa, synthetic construct
  REGION 1..10: note = Synthetic (rTAT)
RRRQRRKKRC

SEQ ID NO: 862 - protein, 12 aa, synthetic construct
  REGION 1..12: note = Synthetic (R9F2)
RRRRRRRRRFFC

SEQ ID NO: 863 - protein, 13 aa, synthetic construct
  REGION 1..13: note = Synthetic ((RRAhx)4B)
  SITE 3: note = misc_feature - Xaa is 6-aminohexanoic acid (Ahx)
  SITE 6: note = misc_feature - Xaa is 6-aminohexanoic acid (Ahx)
  SITE 9: note = misc_feature - Xaa is 6-aminohexanoic acid (Ahx)
  SITE 12: note = misc_feature - Xaa is 6-aminohexanoic acid (Ahx)
  SITE 13: note = misc_feature - Xaa is beta-alanine
RRXRRXRRXRRXX

SEQ ID NO: 864 - protein, 14 aa, synthetic construct
  REGION 1..14: note = Synthetic ((RAhxR)4AhxB; (P007) )
  SITE 2: note = misc_feature - Xaa is 6-aminohexanoic acid (Ahx)
  SITE 5: note = misc_feature - Xaa is 6-aminohexanoic acid (Ahx)
  SITE 8: note = misc_feature - Xaa is 6-aminohexanoic acid (Ahx)
  SITE 11: note = misc_feature - Xaa is 6-aminohexanoic acid (Ahx)
  SITE 13: note = misc_feature - Xaa is 6-aminohexanoic acid (Ahx)
  SITE 14: note = misc_feature - Xaa is beta-alanine
RXRRXRRXRRXRXX

SEQ ID NO: 865 - protein, 14 aa, synthetic construct
  REGION 1..14: note = Synthetic ((AhxRR)4AhxB)
  SITE 1: note = misc_feature - Xaa is 6-aminohexanoic acid (Ahx)
  SITE 4: note = misc_feature - Xaa is 6-aminohexanoic acid (Ahx)
  SITE 7: note = misc_feature - Xaa is 6-aminohexanoic acid (Ahx)
  SITE 10: note = misc_feature - Xaa is 6-aminohexanoic acid (Ahx)
  SITE 13: note = misc_feature - Xaa is 6-aminohexanoic acid (Ahx)
  SITE 14: note = misc_feature - Xaa is beta-alanine
XRRXRRXRRXRRXX

SEQ ID NO: 866 - protein, 13 aa, synthetic construct
  REGION 1..13: note = Synthetic ((RAhx)6B)
  SITE 2: note = misc_feature - Xaa is 6-aminohexanoic acid (Ahx)
  SITE 4: note = misc_feature - Xaa is 6-aminohexanoic acid (Ahx)
  SITE 6: note = misc_feature - Xaa is 6-aminohexanoic acid (Ahx)
  SITE 8: note = misc_feature - Xaa is 6-aminohexanoic acid (Ahx)
  SITE 10: note = misc_feature - Xaa is 6-aminohexanoic acid (Ahx)
  SITE 12: note = misc_feature - Xaa is 6-aminohexanoic acid (Ahx)
  SITE 13: note = misc_feature - Xaa is beta-alanine
RXRXRXRXRXRXX

SEQ ID NO: 867 - protein, 15 aa, synthetic construct
  REGION 1..15: note = Synthetic ((RAhx)8B)
  SITE 2: note = misc_feature - Xaa is 6-aminohexanoic acid (Ahx)
  SITE 4: note = misc_feature - Xaa is 6-aminohexanoic acid (Ahx)
  SITE 6: note = misc_feature - Xaa is 6-aminohexanoic acid (Ahx)
  SITE 8: note = misc_feature - Xaa is 6-aminohexanoic acid (Ahx)
  SITE 10: note = misc_feature - Xaa is 6-aminohexanoic acid (Ahx)
  SITE 12: note = misc_feature - Xaa is 6-aminohexanoic acid (Ahx)
  SITE 14: note = misc_feature - Xaa is 6-aminohexanoic acid (Ahx)
  SITE 15: note = misc_feature - Xaa is beta-alanine
RXRXRXRXRXRXRXX

SEQ ID NO: 868 - protein, 17 aa, synthetic construct
  REGION 1..17: note = Synthetic ((RAhxR)5AhxB)
  SITE 2: note = misc_feature - Xaa is 6-aminohexanoic acid (Ahx)
  SITE 5: note = misc_feature - Xaa is 6-aminohexanoic acid (Ahx)
  SITE 8: note = misc_feature - Xaa is 6-aminohexanoic acid (Ahx)
  SITE 11: note = misc_feature - Xaa is 6-aminohexanoic acid (Ahx)
  SITE 14: note = misc_feature - Xaa is 6-aminohexanoic acid (Ahx)
  SITE 16: note = misc_feature - Xaa is 6-aminohexanoic acid (Ahx)
  SITE 17: note = misc_feature - Xaa is beta-alanine
RXRRXRRXRRXRRXRXX

SEQ ID NO: 869 - protein, 14 aa, synthetic construct
  REGION 1..14: note = Synthetic ((RAhxRRBR)2AhxB; (CPO6062))
  SITE 2: note = misc_feature - Xaa is 6-aminohexanoic acid (Ahx)
  SITE 5: note = misc_feature - Xaa is beta-alanine
  SITE 8: note = misc_feature - Xaa is 6-aminohexanoic acid (Ahx)
  SITE 11: note = misc_feature - Xaa is beta-alanine
  SITE 13: note = misc_feature - Xaa is 6-aminohexanoic acid (Ahx)
  SITE 14: note = misc_feature - Xaa is beta-alanine
RXRRXRRXRRXRXX

SEQ ID NO: 870 - protein, 7 aa, synthetic construct
  REGION 1..7: note = Synthetic (MSP)
ASSLNIA

SEQ ID NO: 871 - protein, 9 aa, synthetic construct
  REGION 1..9: note = Synthetic (MSP-PMO)
  REGION 8..9: note = misc_feature - Xaa can be any naturally occurring amino acid
ASSLNIAXX

SEQ ID NO: 872 - protein, 22 aa, synthetic construct
  REGION 1..22: note = Synthetic (CP06062-MSP-PMO)
  SITE 2: note = misc_feature - Xaa can be any naturally occurring amino acid
  SITE 5: note = misc_feature - Xaa is beta-alanine
  SITE 8: note = misc_feature - Xaa can be any naturally occurring amino acid
  SITE 11: note = misc_feature - Xaa is beta-alanine
  SITE 13: note = misc_feature - Xaa can be any naturally occurring amino acid
  SITE 14: note = misc_feature - Xaa is beta-alanine
  SITE 22: note = misc_feature - Xaa can be any naturally occurring amino acid
RXRRXRRXRRXRXXASSLNIAX

SEQ ID NO: 873 - protein, 21 aa, synthetic construct
  REGION 1..21: note = Synthetic (MSP-CP06062-PMO)
  SITE 8: note = misc_feature - Xaa can be any naturally occurring amino acid
  SITE 10: note = misc_feature - Xaa can be any naturally occurring amino acid
  SITE 13: note = misc_feature - Xaa is beta-alanine
  SITE 16: note = misc_feature - Xaa can be any naturally occurring amino acid
  SITE 19: note = misc_feature - Xaa is beta-alanine
  SITE 21: note = misc_feature - Xaa is beta-alanine
ASSLNIAXRXRRXRRXRRXRX

SEQ ID NO: 874 - protein, 14 aa, synthetic construct
  REGION 1..14: note = Synthetic (CP06062-PMO)
  SITE 2: note = misc_feature - Xaa can be any naturally occurring amino acid
  SITE 5: note = misc_feature - Xaa is beta-alanine
  SITE 8: note = misc_feature - Xaa can be any naturally occurring amino acid
  SITE 11: note = misc_feature - Xaa is beta-alanine
  SITE 13: note = misc_feature - Xaa can be any naturally occurring amino acid
  SITE 14: note = misc_feature - Xaa is beta-alanine
RXRRXRRXRRXRXX

SEQ ID NO: 875 - DNA, 25 bp, synthetic construct
  source 1..25: mol_type = other DNA
  misc_feature 1..25: note = Synthetic (PMO)
ggccaaacctcggcttacctgaaat

SEQ ID NO: 876 - protein, 9 aa, synthetic construct
  REGION 1..9: note = Synthetic (rTAT)
RRRQRRKKR

SEQ ID NO: 877 - protein, 9 aa, synthetic construct
  REGION 1..9: note = Synthetic (Tat)
RKKRRQRRR

SEQ ID NO: 878 - protein, 11 aa, synthetic construct
  REGION 1..11: note = Synthetic (R9F2)
RRRRRRRRRFF

SEQ ID NO: 879 - protein, 11 aa, synthetic construct
  REGION 1..11: note = Synthetic (R5F2R4)
RRRRRFFRRRR

SEQ ID NO: 880 - protein, 4 aa, synthetic construct
  REGION 1..4: note = Synthetic (R4)
RRRR

SEQ ID NO: 881 - protein, 5 aa, synthetic construct
  REGION 1..5: note = Synthetic (R5)
RRRRR

SEQ ID NO: 882 - protein, 6 aa, synthetic construct
  REGION 1..6: note = Synthetic (R6)
RRRRRR

SEQ ID NO: 883 - protein, 7 aa, synthetic construct
  REGION 1..7: note = Synthetic (R7)
RRRRRRR

SEQ ID NO: 884 - protein, 8 aa, synthetic construct
  REGION 1..8: note = Synthetic (R8)
RRRRRRRR

SEQ ID NO: 885 - protein, 9 aa, synthetic construct
  REGION 1..9: note = Synthetic (R9)
RRRRRRRRR

SEQ ID NO: 886 - protein, 16 aa, synthetic construct
  REGION 1..16: note = Synthetic ((RX)8)
  SITE 2: note = misc_feature - Xaa can be any naturally occurring amino acid
  SITE 4: note = misc_feature - Xaa can be any naturally occurring amino acid
  SITE 6: note = misc_feature - Xaa can be any naturally occurring amino acid
  SITE 8: note = misc_feature - Xaa can be any naturally occurring amino acid
  SITE 10: note = misc_feature - Xaa can be any naturally occurring amino acid
  SITE 12: note = misc_feature - Xaa can be any naturally occurring amino acid
  SITE 14: note = misc_feature - Xaa can be any naturally occurring amino acid
  SITE 16: note = misc_feature - Xaa can be any naturally occurring amino acid
RXRXRXRXRXRXRXRX

SEQ ID NO: 887 - protein, 12 aa, synthetic construct
  REGION 1..12: note = Synthetic ((RAhxR)4; (P007) )
  SITE 2: note = misc_feature - Xaa is 6-aminohexanoic acid
  SITE 5: note = misc_feature - Xaa is 6-aminohexanoic acid
  SITE 8: note = misc_feature - Xaa is 6-aminohexanoic acid
  SITE 11: note = misc_feature - Xaa is 6-aminohexanoic acid
RXRRXRRXRRXR

SEQ ID NO: 888 - protein, 15 aa, synthetic construct
  REGION 1..15: note = Synthetic ((RAhxR)5; (CP04057))
  SITE 2: note = misc_feature - Xaa is 6-aminohexanoic acid
  SITE 5: note = misc_feature - Xaa is 6-aminohexanoic acid
  SITE 8: note = misc_feature - Xaa is 6-aminohexanoic acid
  SITE 11: note = misc_feature - Xaa is 6-aminohexanoic acid
  SITE 14: note = misc_feature - Xaa is 6-aminohexanoic acid
RXRRXRRXRRXRRXR

SEQ ID NO: 889 - protein, 12 aa, synthetic construct
  REGION 1..12: note = Synthetic ((RAhxRRBR)2; (CP06062))
  SITE 2: note = misc_feature - Xaa is 6-aminohexanoic acid
  SITE 5: note = misc_feature - Xaa is beta-alanine
  SITE 8: note = misc_feature - Xaa is 6-aminohexanoic acid
  SITE 11: note = misc_feature - Xaa is beta-alanine
RXRRXRRXRRXR

SEQ ID NO: 890 - protein, 14 aa, synthetic construct
  REGION 1..14: note = Synthetic ((RAR)4F2)
RARRARRARRARFF

SEQ ID NO: 891 - protein, 14 aa, synthetic construct
  REGION 1..14: note = Synthetic ((RGR)4F2))
RGRRGRRGRRGRFF